(19) United States
(12) Patent Application Publication
(10) Pub. No.: US 2010/0035803 A1
(43) Pub. Date: Feb. 11, 2010

(54) Polypeptides and Polynucleotides Encoding the same

(75) Inventors: Elie Khalil, Victoria (AU); Matthew R. Digby, Victoria (AU); Kevin Roy Nicholas, Victoria (AU); Christophe M. Lefevre, Victoria (AU); Yvan Strahm, Vancouver (CA)

Correspondence Address: SCHWEGMAN, LUNDBERG & WOESSNER, P.A., P.O. BOX 2938, MINNEAPOLIS, MN, 55402, US

(73) Assignee: Dairy Australia Limited, Southbank ,Victoria (AU)

(21) Appl. No.: 12/227,413

(22) PCT Filed: May 17, 2007
(86) PCT No.: PCT/AU2007/000671

§ 371 Date: Sep. 25, 2009

(30) Foreign Application Priority Data
May 17, 2006 (AU) 2006902639

(51) Int. Cl. A61K 38/16; C07K 14/435; C12N 5/10

(52) U.S. Cl. 514/12; 530/350; 435/369

(57) ABSTRACT

Provided herein are lactation-associated polypeptides and polynucleotides, expression vectors a host cells for expressing lactation-associated polypeptides and polynucleotides, and methods of producing said polypeptides and polynucleotides.

### TECHNICAL FIELD

The present invention relates generally to polypeptides the expression of which is altered during lactation in mammals. The invention also relates to polynucleotides encoding the same and to uses of these polypeptides and polynucleotides.

### CROSS REFERENCE TO RELATED APPLICATION(S)

This application claims the benefit of Australian Provisional Patent Application No. 2006902639 which is herein incorporated by reference in its entirety.

### BACKGROUND OF THE INVENTION

Mammalian milk is composed primarily of proteins, sugars, lipids and a variety of trace minerals and vitamins. Milk proteins not only provide nutrition for the developing offspring, but a complex range of biological activities tailored to age-specific needs of the offspring.

It is well recognized that milk composition changes during lactation, the most striking change being that from colostrum to milk shortly after parturition in most mammals. However a variety of other changes in milk composition occur throughout lactation. The extent and full biological significance of the changes is presently unknown although it is accepted that milk composition alterations at least in part reflect the changing needs of the offspring through stages of development and/or regulate such developmental changes.

The major protein constituents of milk are the casein proteins, α-casein and βcasein, α-lactalbumin and β-lactoglobulin. Milk also contains significant antimicrobial and immune-response mediators. Well known constituents include antibodies, lysozyme, lactoferrin complement proteins C3/C4, defensins, and interleukins including IL-1, IL-10 and IL-12. In addition to these a vast array of other proteins are also present in milk, many of which remain to be identified and characterized. A significant number of these uncharacterized proteins are likely to play a regulatory role and/or contribute to the development or protection of the offspring, for example by providing antimicrobial activities, anti-inflammatory activities or by boosting the immune system of the offspring. There is a clear need to elucidate the identities and activities of such proteins.

Marsupials have a number of unique features in their modes of reproduction and lactation which make them excellent model organisms for the study of changes in milk composition, and specifically milk proteins. Lactation in marsupials has been studied extensively; one of most widely studied marsupials being the tammar wallaby (Macropus eugenii). The lactation cycle in the tammar wallaby can be divided into 4 phases, phase 1, phase 2A, phase 2B and phase 4 (see Nicholas et al., 1997, J Mammary Gland Biol Neoplasia 2: 299-310). The transition from one phase to the next correlates with significant alterations in milk composition, in particular in milk protein concentrations. Milk composition is specifically matched for the developmental stage of the offspring. Macropodids such as the tammar wallaby are capable of concurrent asynchronous lactation whereby individual teats produce milk with different compositions for pouch young of different ages. As such lactation can be independently regulated locally rather than systemically, determining the rate of growth and development of the young irrespective of the age of the young (Nicholas et al., 1997; Trott et al., 2003, Biol Reprod 68:929-936). Additionally, marsupial young are altricial and thus totally dependent on maternal milk in the early stages of life. For example, tammar wallaby pouch young have no immune system of their own for approximately the first 70 days and depend entirely on the protection offered by maternal milk. The above features, inter alia, make marsupials excellent experimental model organisms for the investigation of regulatory and bioactive proteins in milk.

Further, with the rapid progress of comparative gene mapping techniques and genome sequencing technology, genetic studies in marsupials have already proven instrumental in the identification of novel genes in other species. For example, studies in the tammar wallaby led to the discovery of a candidate gene for mental retardation, RBMX, in humans (Delbridge et al., 1999, Nat Genet 22: 223-224).

The present invention is predicated on the inventors' use of the tammar wallaby as a model system for the identification of lactation-associated polypeptides secreted in mammalian milk.

### SUMMARY OF THE INVENTION

In a first aspect, the present invention provides a lactation-associated polypeptide comprising an amino acid sequence selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450 and 452, or variant thereof.

The polypeptide may be a secreted polypeptide.

In a second aspect of the invention there is provided a polynucleotide encoding a polypeptide of the first aspect.

A third aspect of the invention provides a lactation-associated polynucleotide comprising a nucleotide sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451 and 453 to 502, or variant thereof.

A fourth aspect of the invention provides polypeptides encoded by the polynucleotides of the third aspect.

A fifth aspect of the present invention provides an expression vector comprising a polynucleotide of the second or third aspect. The polynucleotide may be operably linked to a promoter.

A sixth aspect of the invention provides a host cell transformed with an expression vector of the fifth aspect.

A seventh aspect of the invention provides a method for isolating a bioactive molecule comprising the steps of:

(a) introducing into a suitable host cell a polynucleotide of the second or third aspect or expression vector of the fifth aspect;

(b) culturing the cell under conditions suitable for expression of a polypeptide encoded by the polynucleotide;

(c) recovering the polypeptide; and

(d) assaying the recovered polypeptide for biological activity.

An eighth aspect of the invention provides a method for isolating a bioactive molecule comprising the steps of:

(a) introducing into a suitable host cell a polynucleotide of the second or third aspect or expression vector of the fifth aspect;

(b) culturing the cell under conditions suitable for expression of a polypeptide encoded by the polynucleotide and for secretion of the polypeptide into the extra cellular medium;

(c) recovering the polypeptide; and

(d) assaying the recovered polypeptide for biological activity.

In embodiments of the seventh and eighth aspects, the assaying in step (d) may comprise assaying for anti-inflammatory, pro-inflammatory, anti-microbial, anti-apoptotic or cell proliferative activity. Polypeptides may also be assayed to determine their ability to influence the differentiation of embryonic stem cells or mammary epithelium, to stimulate transcription from the trefoil gene promoter, to stimulate transcription from the OCT4 gene promoter, to stimulate the expression of secreted proteins or influence mammary gland development, such as the mammary epithelium.

In a ninth aspect of the invention there is provided a bioactive molecule isolated according to the method of the seventh or eighth aspect.

According to a tenth aspect of the present invention there is provided a method of screening for compounds that modulate the expression or activity of polypeptides and/or polynucleotides of the invention, comprising:

(a) contacting a polypeptide of the first or fourth aspect or polynucleotide of the second or third aspect with a candidate compound under conditions suitable to enable interaction of the candidate compound to the polypeptide or the polynucleotide; and

(b) assaying for activity of the polypeptide or polynucleotide.

The modulation may be in the form of an inhibition of expression or activity or an activation or stimulation of expression or activity. Accordingly, the modulator compound may be an antagonist or agonist of the polypeptide or polynucleotide.

According to an eleventh aspect of the present invention there is provided a method for isolating lactation-associated polynucleotides in a eutherian mammalian species comprising:
(a) obtaining a biological sample from the eutherian mammalian species, the sample containing nucleic acid molecules;
(b) contacting the biological sample with one or more polynucleotides of the second or third aspect;
(c) detecting hybridization between nucleic acid molecules in the biological sample and the one or more polynucleotides; and
(d) isolating the hybridizing nucleic acid molecules.

The hybridization may occur and be detected through techniques that are standard and routine amongst those skilled in the art, including southern and northern hybridization, polymerase chain reaction and ligase chain reaction.

The hybridization may be conducted under conditions of low stringency. The hybridization may be conducted under conditions of medium or high stringency.

According to a twelfth aspect of the invention there is provided a lactation-associated polynucleotide isolated according to the method of the twelfth aspect.

According to a thirteenth aspect of the invention there is provided a polypeptide encoded by a polynucleotide of the twelfth aspect.

The present invention also provides compositions comprising polypeptides of the first, fourth or thirteenth aspects, polynucleotides of the second, third or twelfth aspects, or bioactive molecules of the ninth aspect, together with one or more pharmaceutically acceptable carriers, diluents or adjuvants. Compositions comprising antagonists or agonists of bioactive molecules of the invention are also contemplated.

The present invention also provides methods of treatment, comprising administering to a mammal in need thereof and effective amount of a composition of the invention.

### DEFINITIONS

The term “comprising” means “including principally, but not necessarily solely”. Furthermore, variations of the word “comprising”, such as “comprise” and “comprises”, have correspondingly varied meanings.

The term “polypeptide” means a polymer made up of amino acids linked together by peptide bonds. The term “polynucleotide” as used herein refers to a single- or double-stranded polymer of deoxyribonucleotide, ribonucleotide bases or known analogues or natural nucleotides, or mixtures thereof.

The term “lactation-associated” as used herein in relation to a polypeptide or polynucleotide means that expression of the polypeptide or polynucleotide is altered during lactation as compared to basal levels of expression before or after lactation. Expression of the polypeptide or polynucleotide may be increased or decreased during lactation, either at one point during the lactation cycle or over the course of lactation. For example, an increase or decrease in expression of the polypeptide or polynucleotide during lactation may be observed by comparing the level of expression prior to lactation initiation with the level of expression at involution, by comparing the level of expression across a lactation phase change, or by comparing the level of expression between any two timepoints in lactation.

The term “isolating” as used herein as it pertains to methods of isolating bioactive molecules means recovering the molecule from the cell culture medium substantially free of cellular material, although the molecule need not be free of all components of the media. For example a secreted polypeptide may be recovered in the extracellular media, such as the supernatant, and still be “isolated”.

The term “bioactive molecule” as used herein refers a polypeptide or polynucleotide disclosed herein having a defined biological activity. Biological activities include, for example, regulatory activities including regulation of mammary gland development, lactation, milk production and/or milk composition, or any other defined biological activity, including growth-promoting activity, anti- or pro-inflammatory activity, ant- or pro-apoptotic activity or anti-microbial activity.

The term “secreted” as used herein means that the polypeptide is secreted from the cytoplasm of a cell, either as a cell membrane-associated polypeptide with an extracellular portion or is secreted entirely into the extracellular space.

### BRIEF DESCRIPTION OF THE DRAWINGS

A preferred form of the present invention will now be described by way of example with reference to the accompanying drawings:

FIG. 1. Sequences of lactation associated polynucleotides and polypeptides identified herein (SEQ ID NOs: 1 to 502).

FIG. 2. Microarray expression profiles. Each graph shows normalized expression intensities for ESTs across lactation. Three lines of varying darkness are depicted on each graph. The light grey lines represent single channel normalization of the average intensity from Cy3 fluorescence. The dark grey lines represent single channel normalization of the average intensity from Cy5 fluorescence. The black lines represent the average of these Cy3 and Cy5 channel intensities. The scale for each EST intensity is relative, the highest individual spot intensity being 100 percent. All lines pass through the origin of the graph. Lactation phases are indicated as P (pregnancy), 2A, 2B and 3.

FIG. 3. Activation of ERK by secreted polypeptides. Each graph shows the relative fluorescence units (RFU) detected for each sample (coded by plate well number).

FIG. 4 Graph showing the normalized spot intensities for SGT20R3_C12, SGT20R1_B04 and SGT20K1_B08 from 21 days before parturition (day five pregnant) to day 260 of lactation.

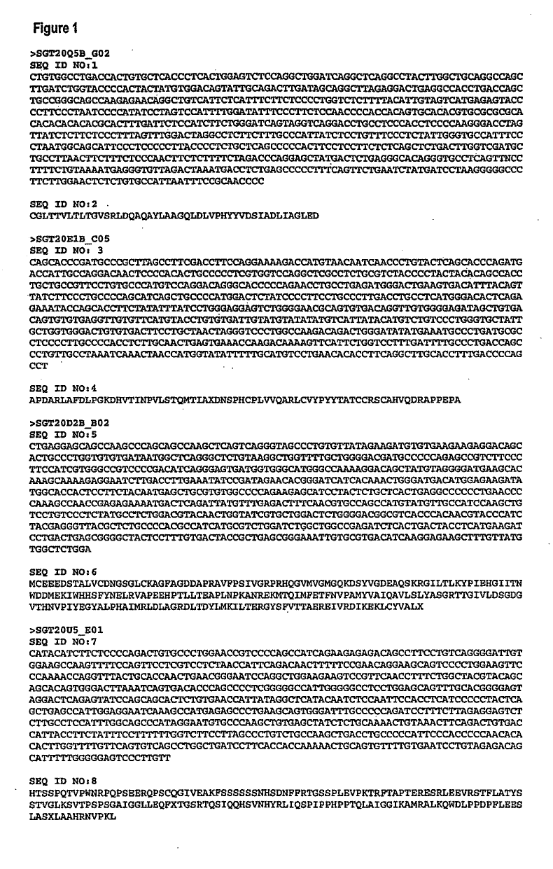

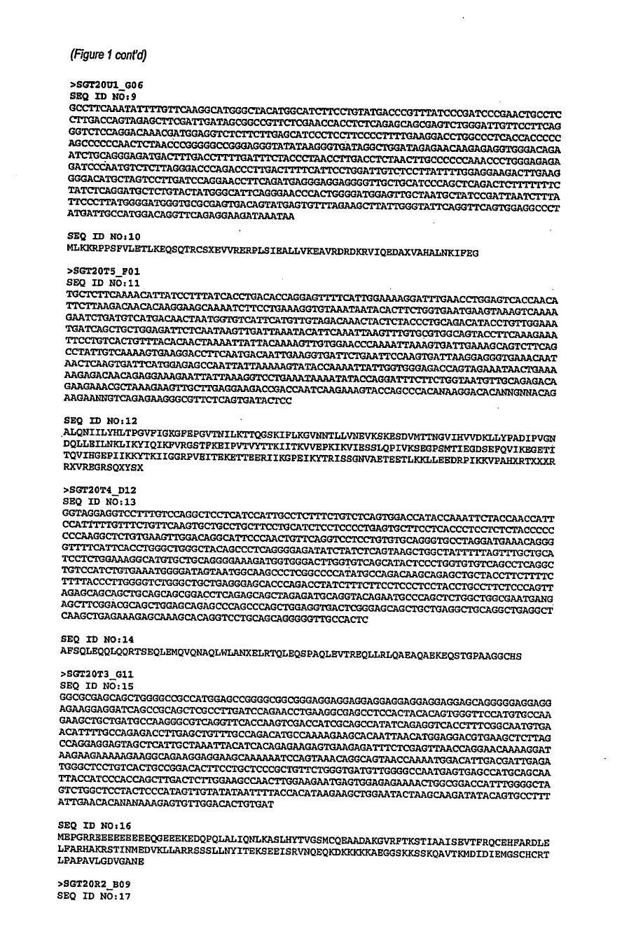

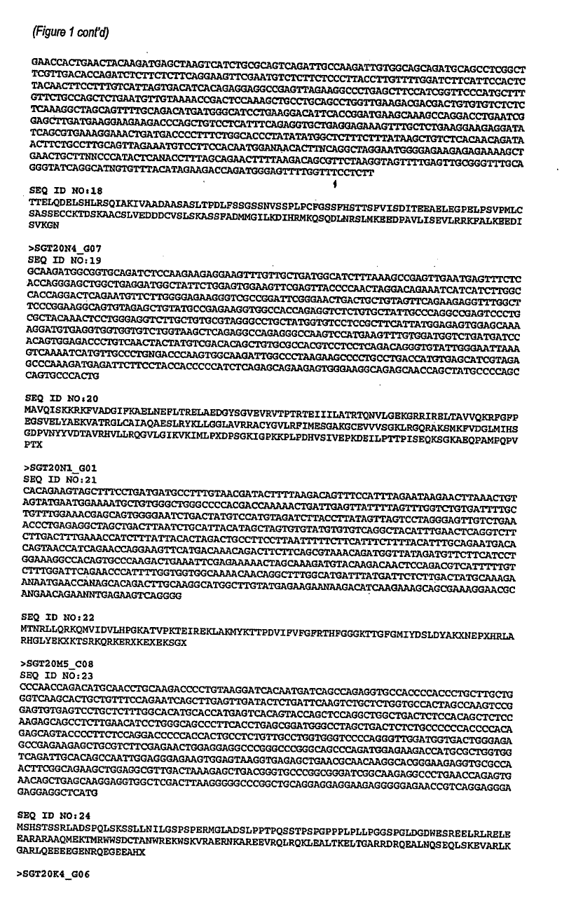

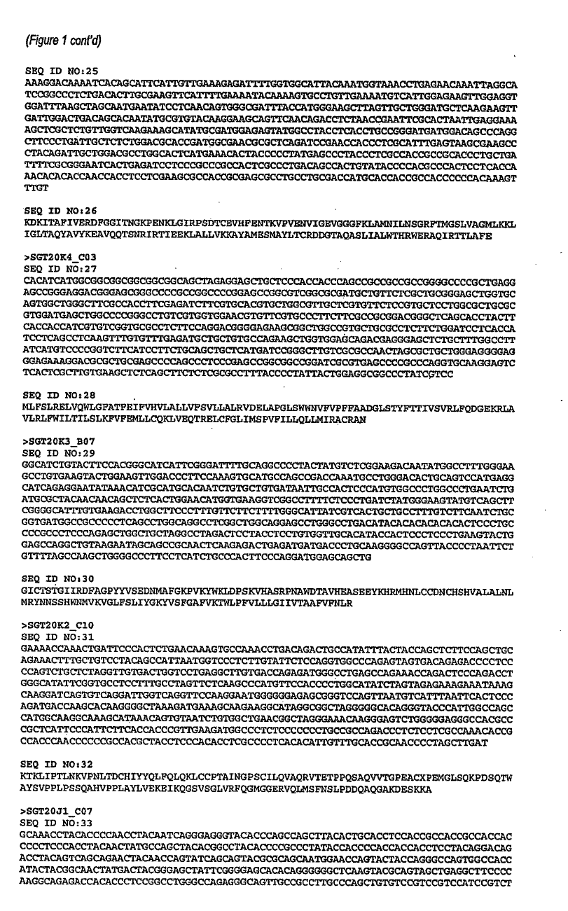

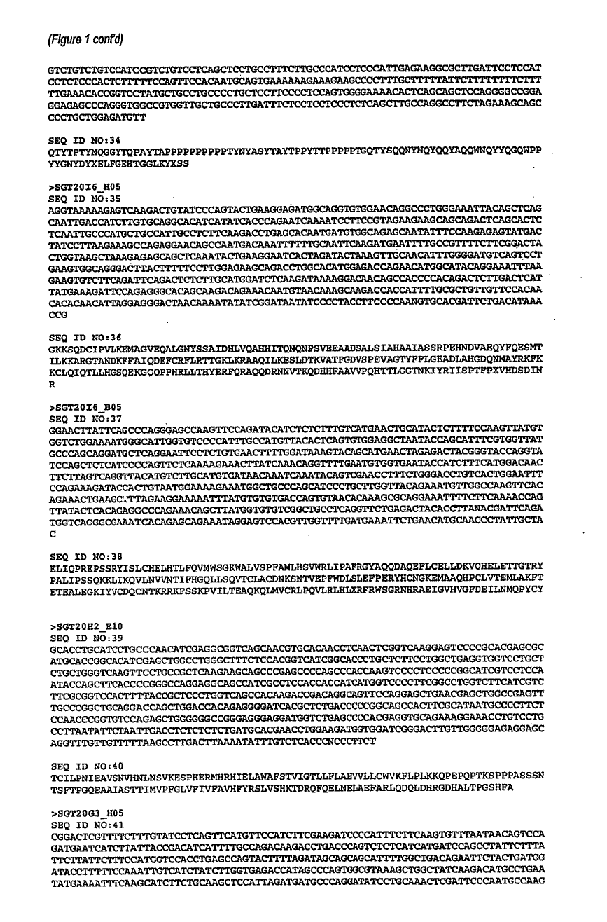

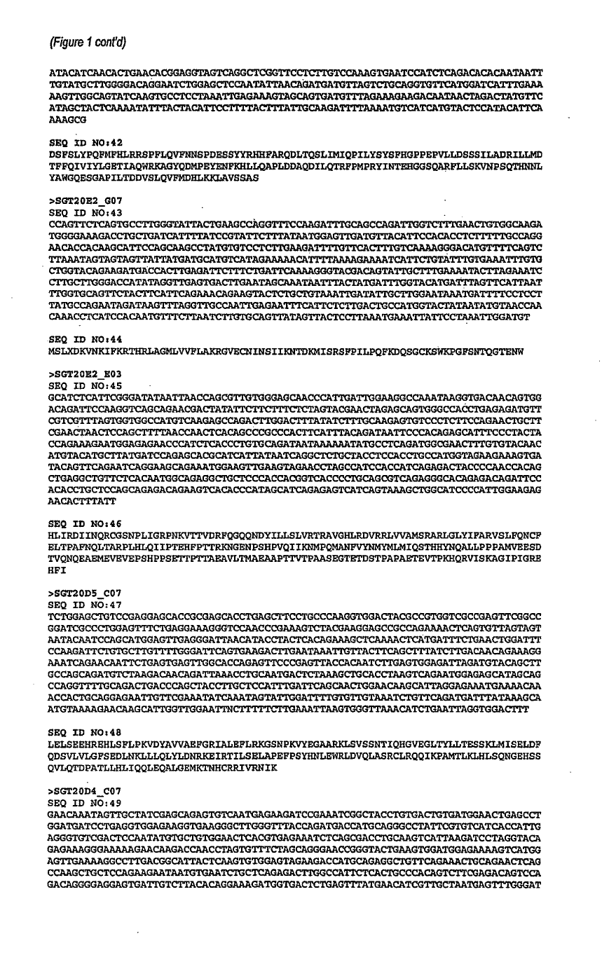

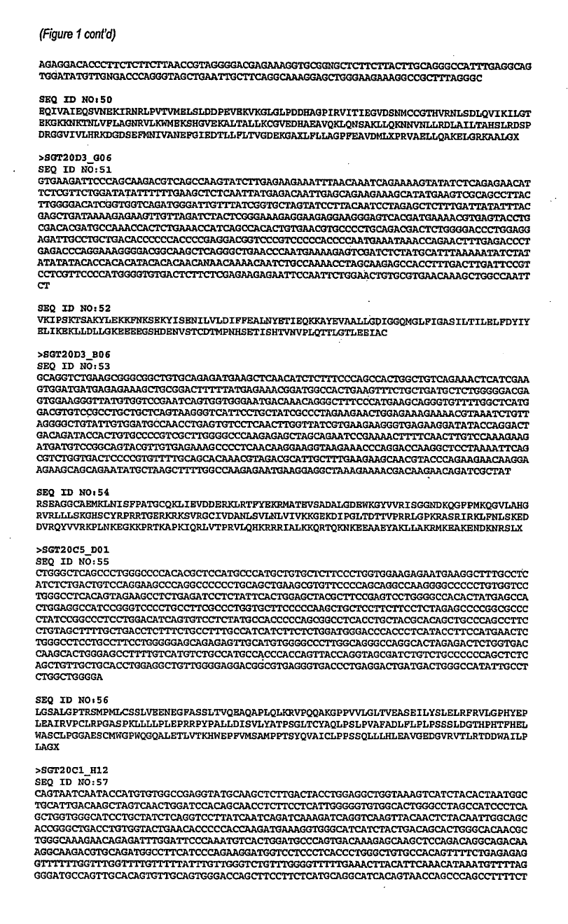

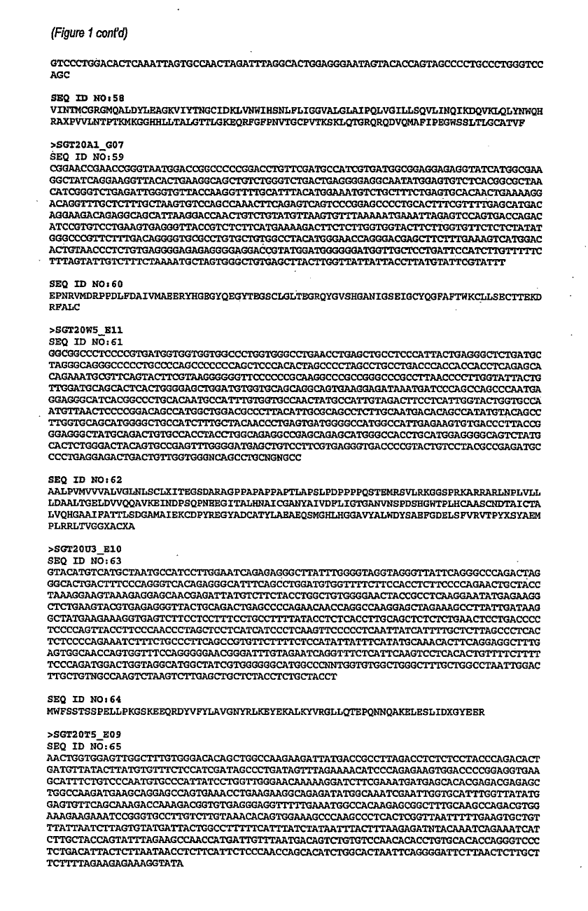

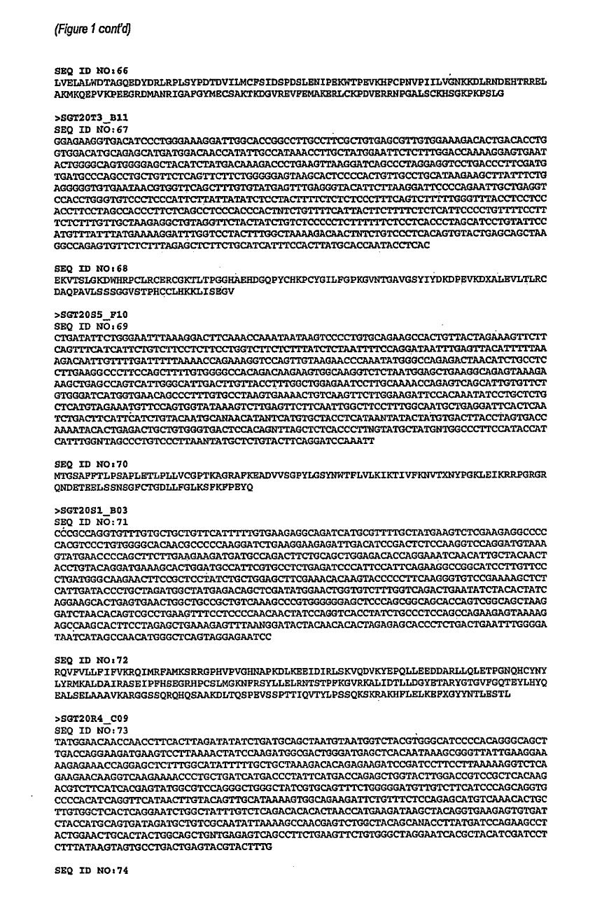

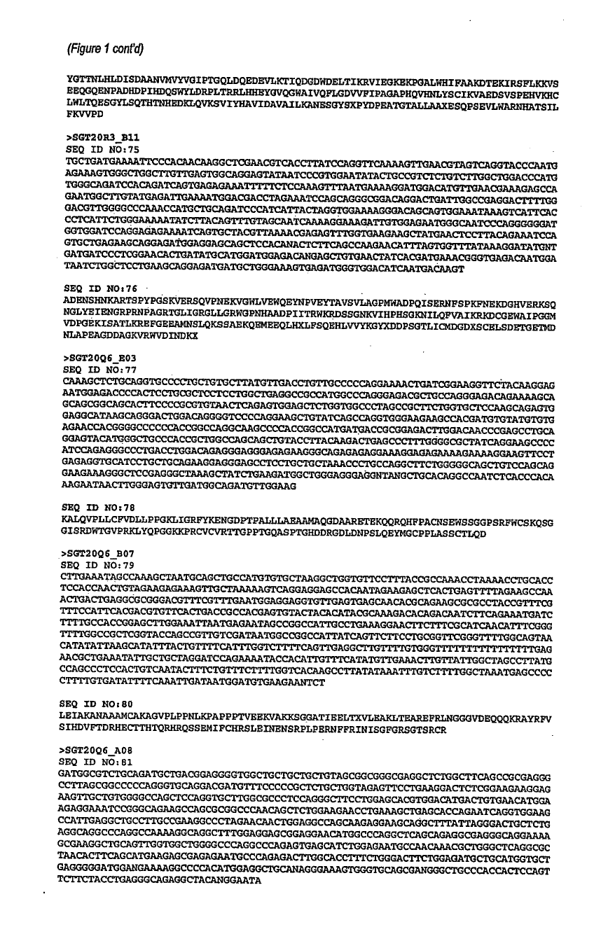

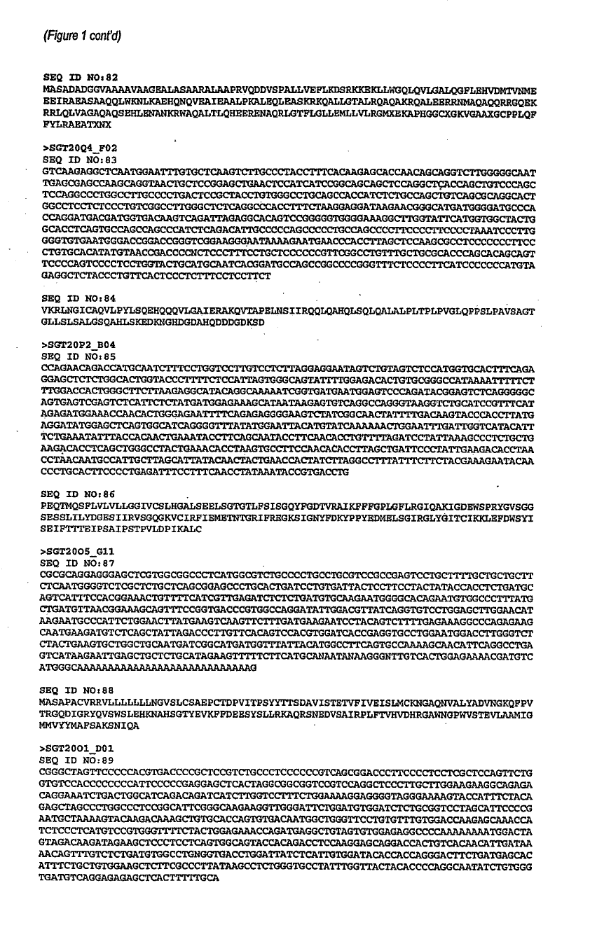

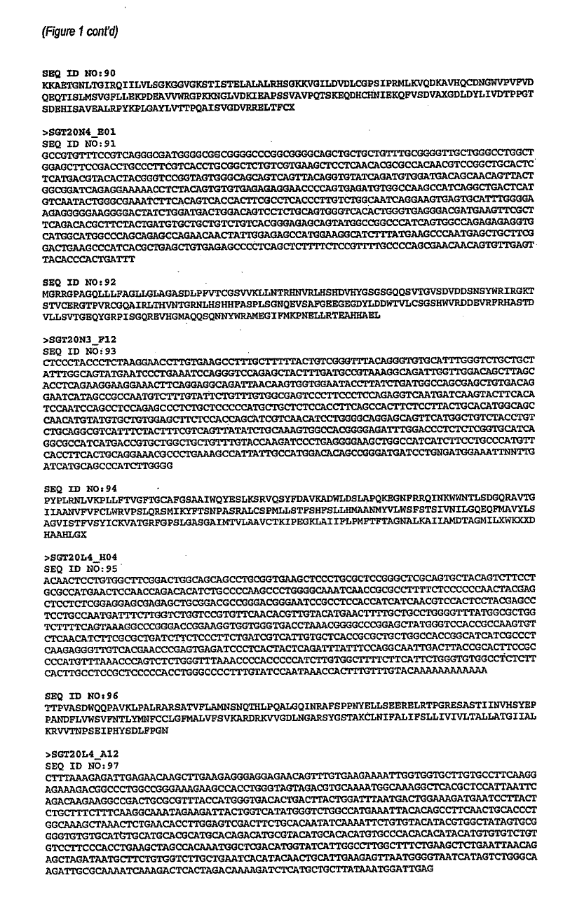

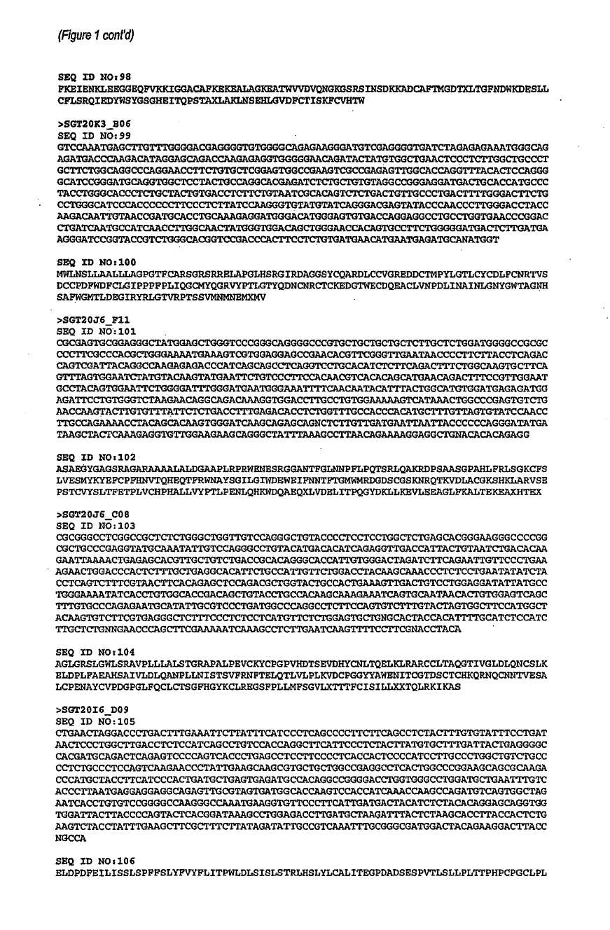

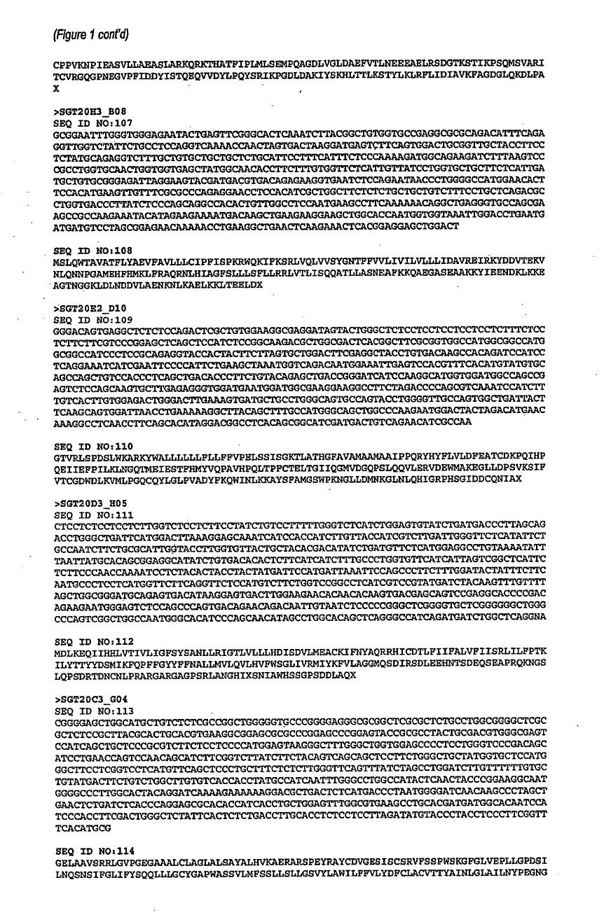

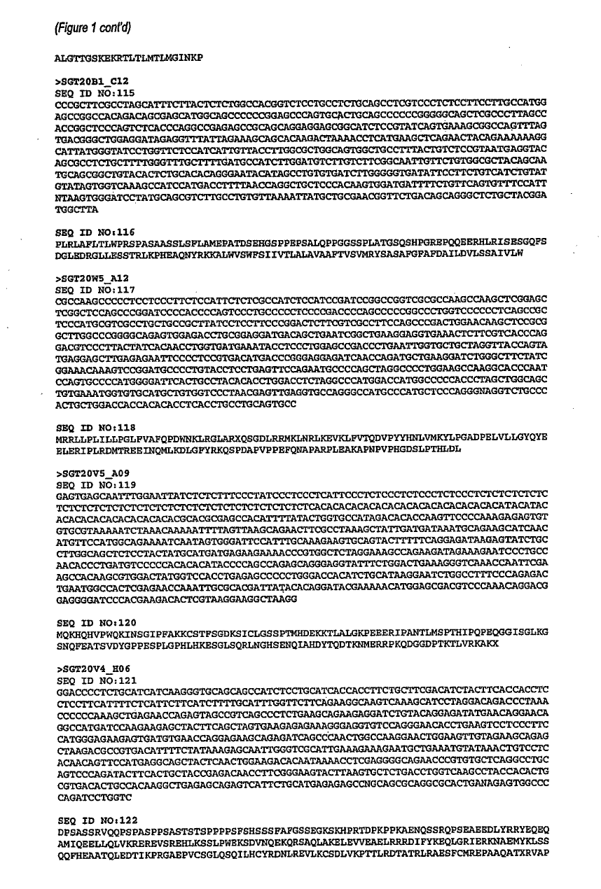

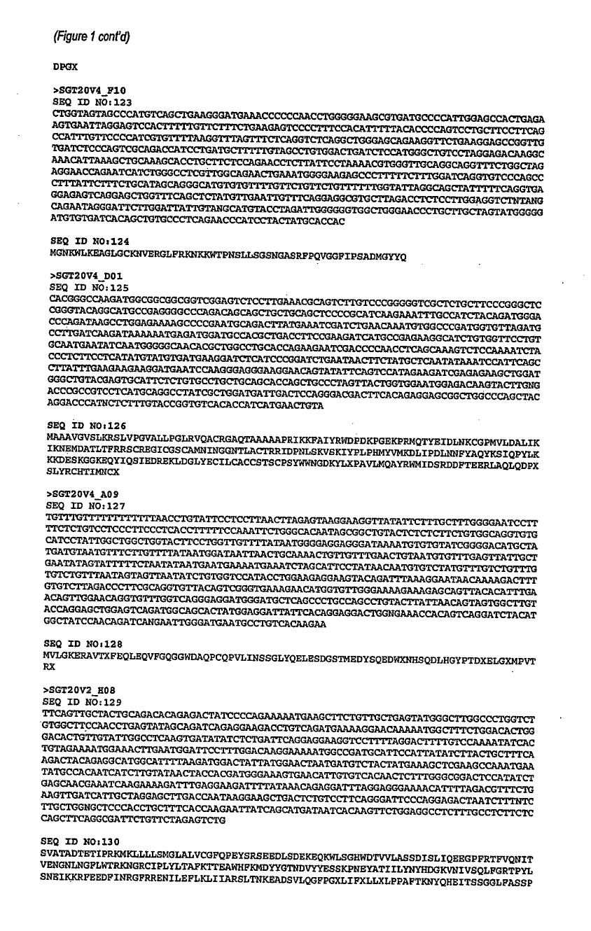

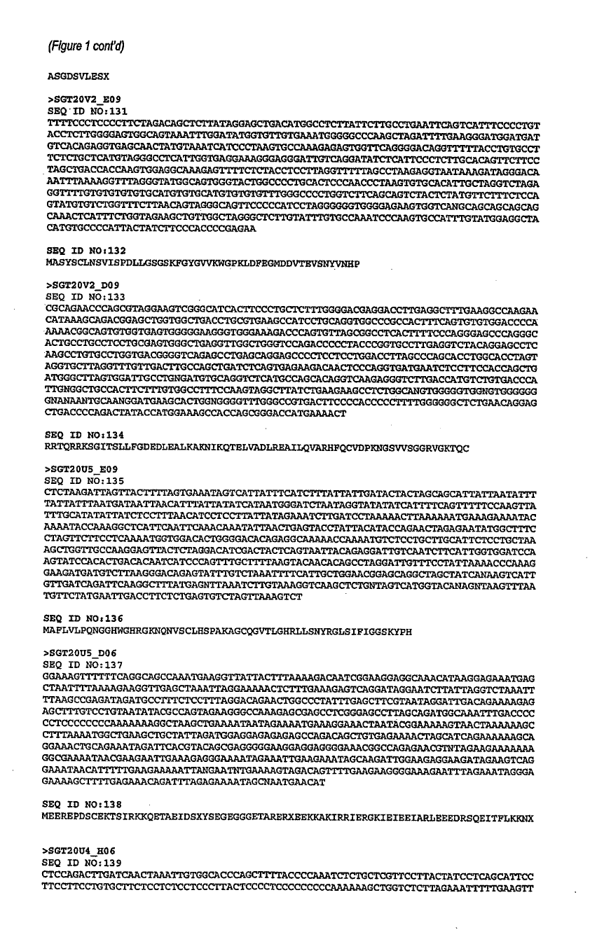

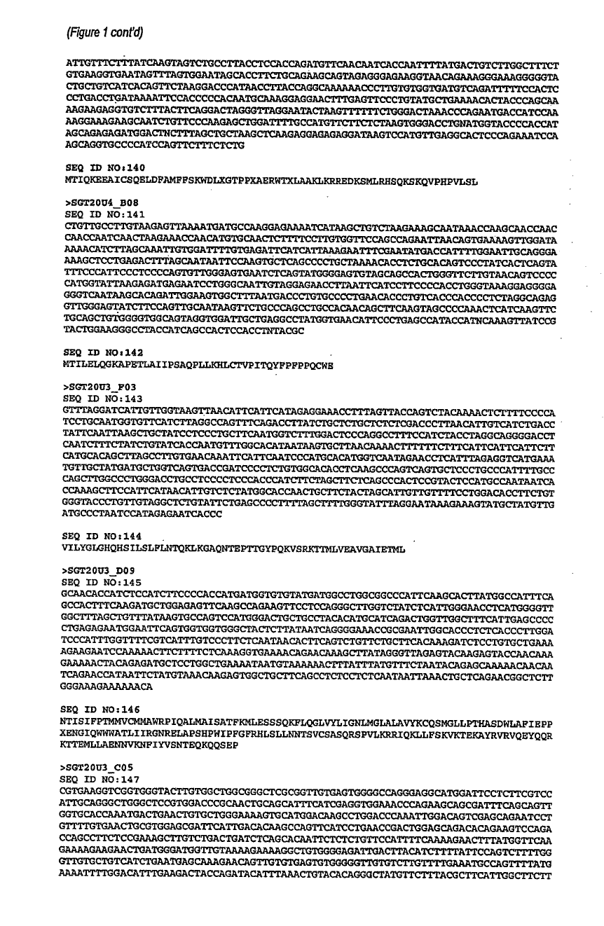

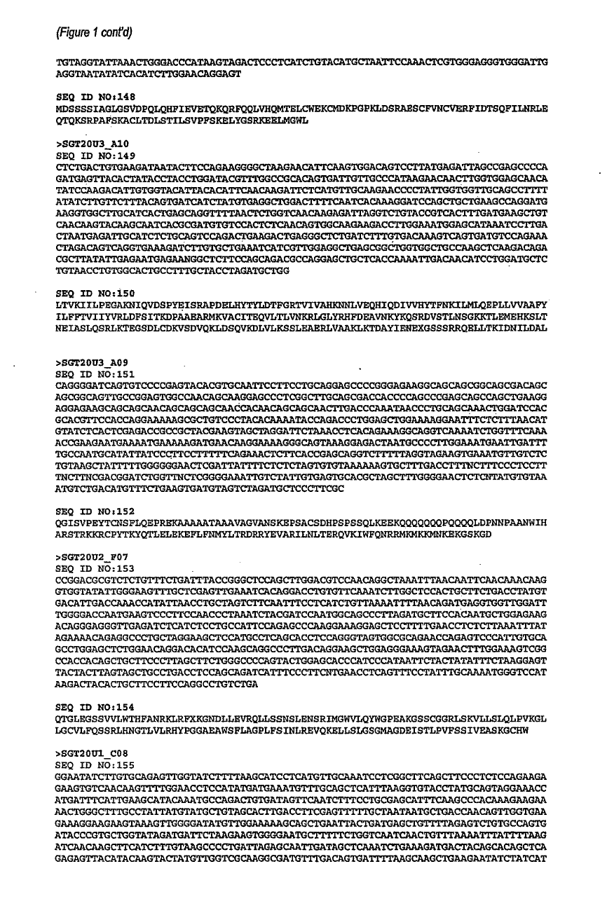

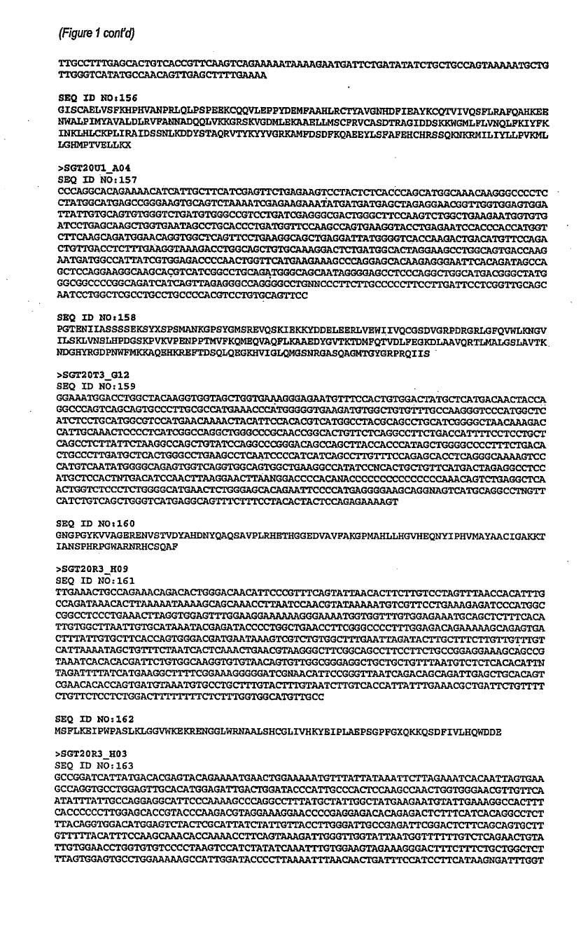

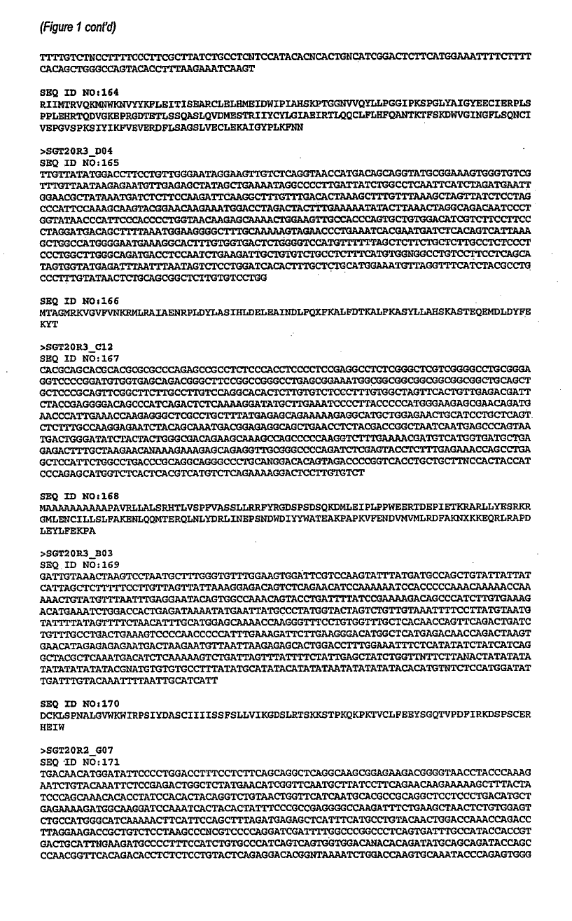

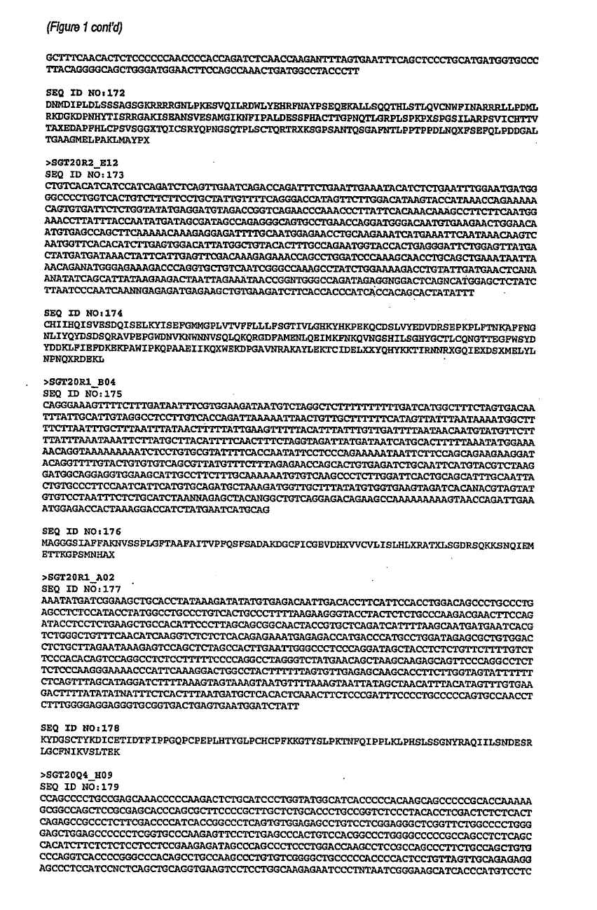

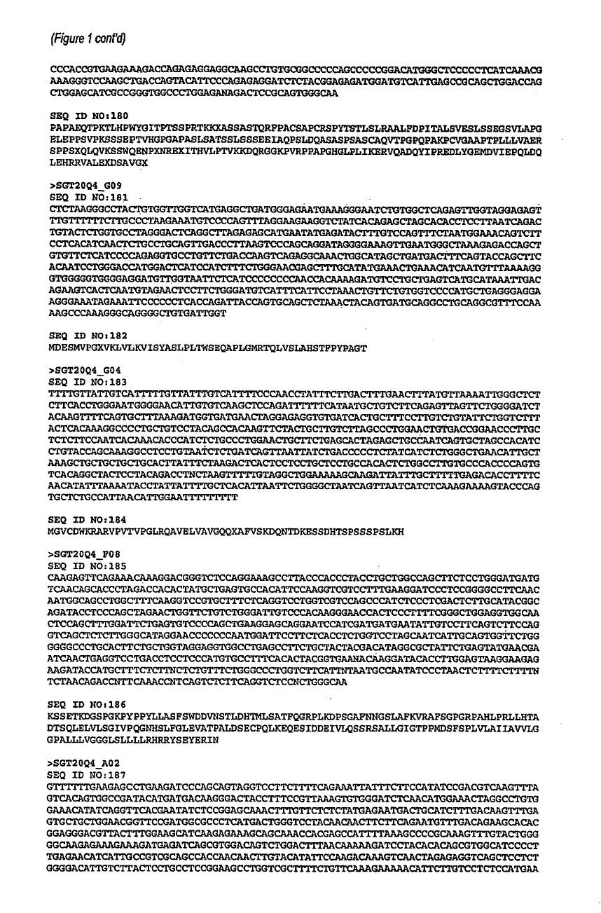

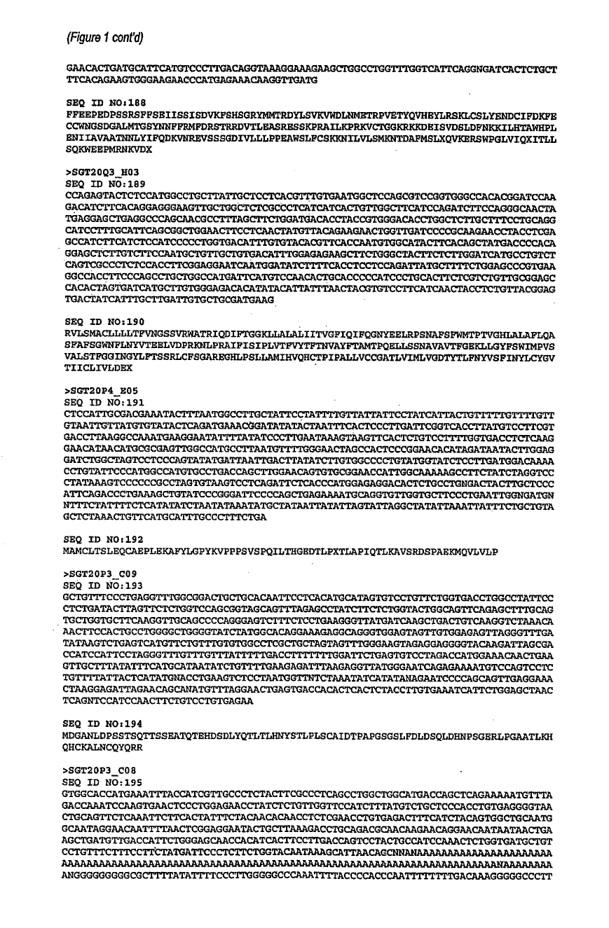

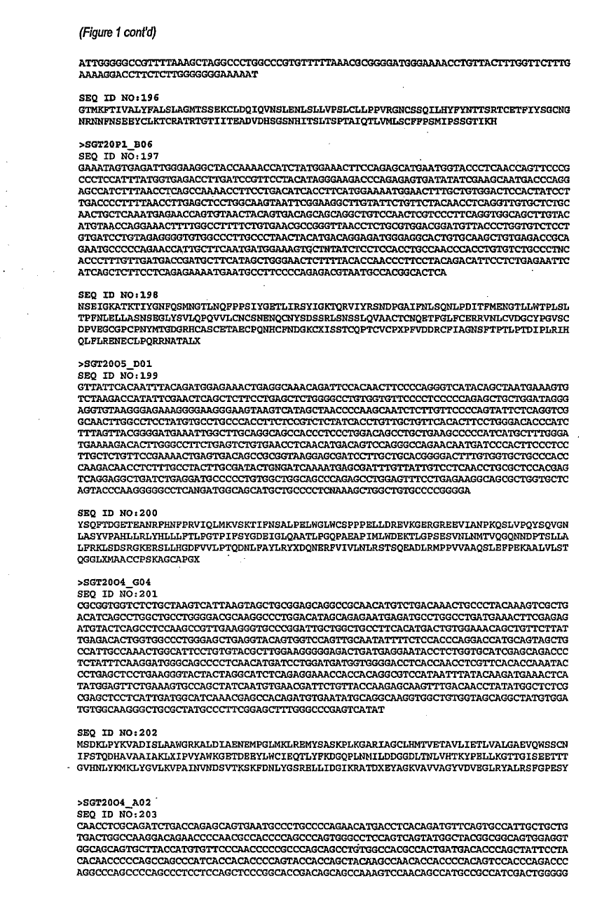

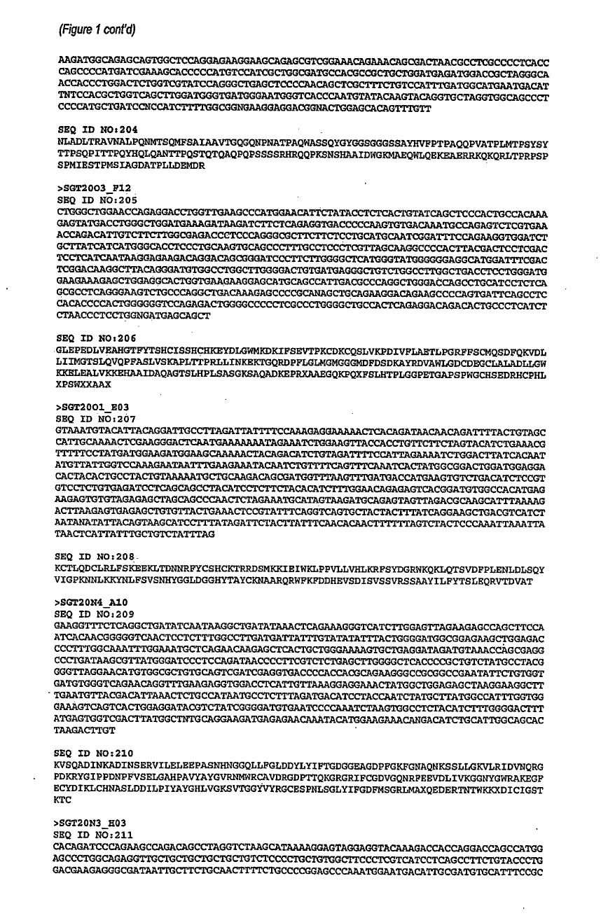

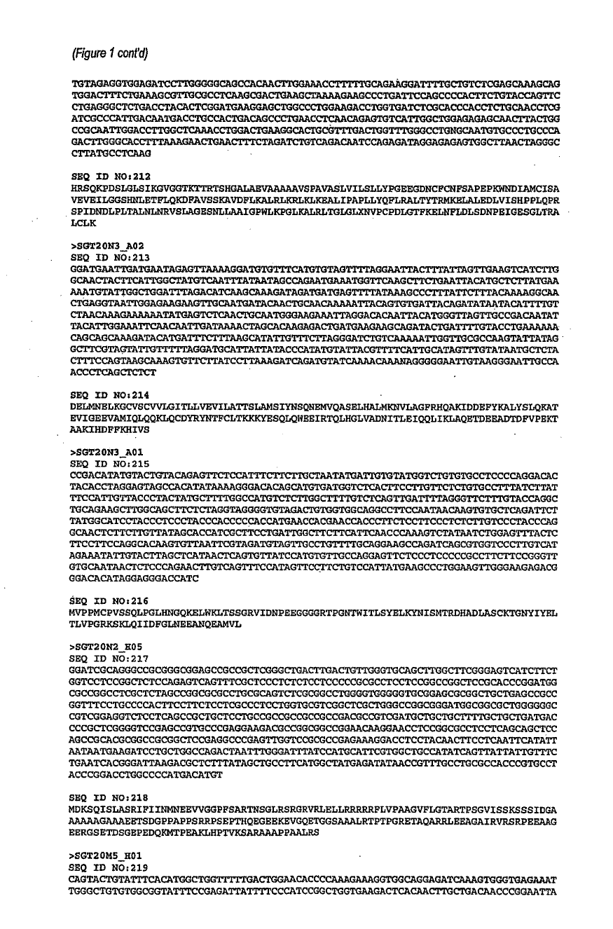

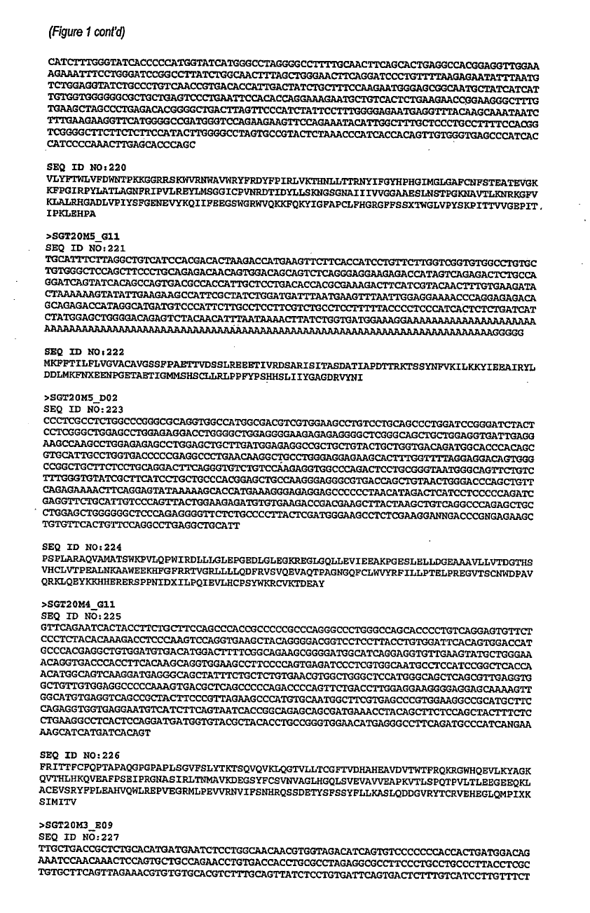

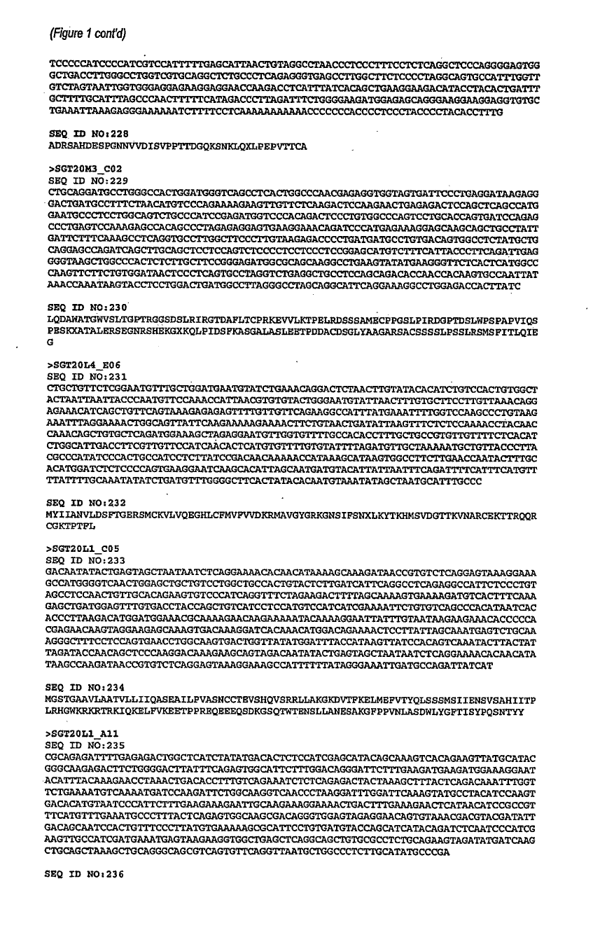

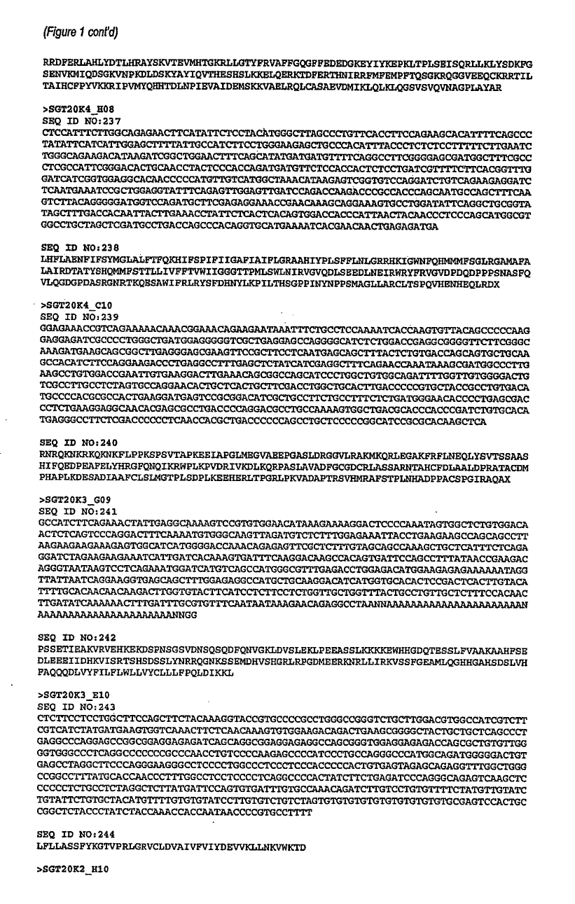

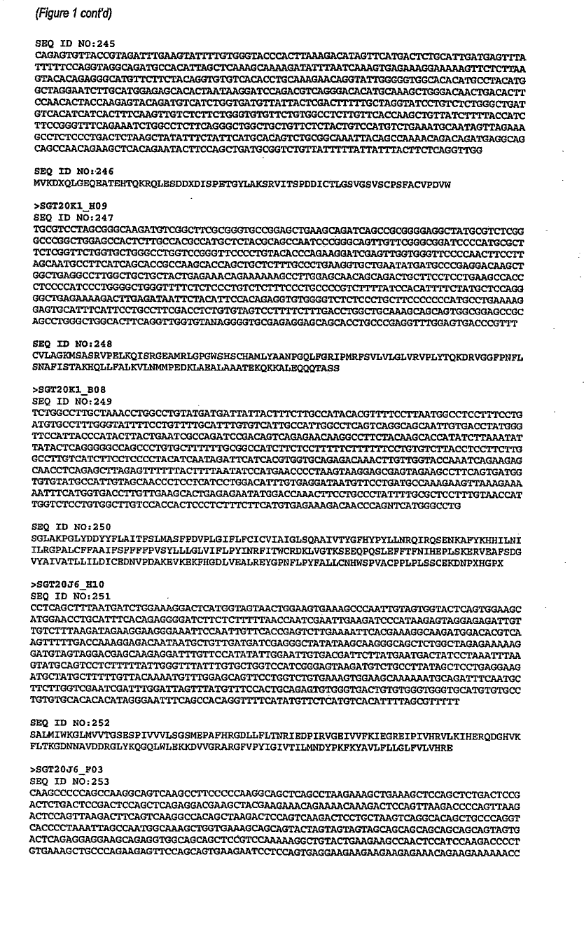

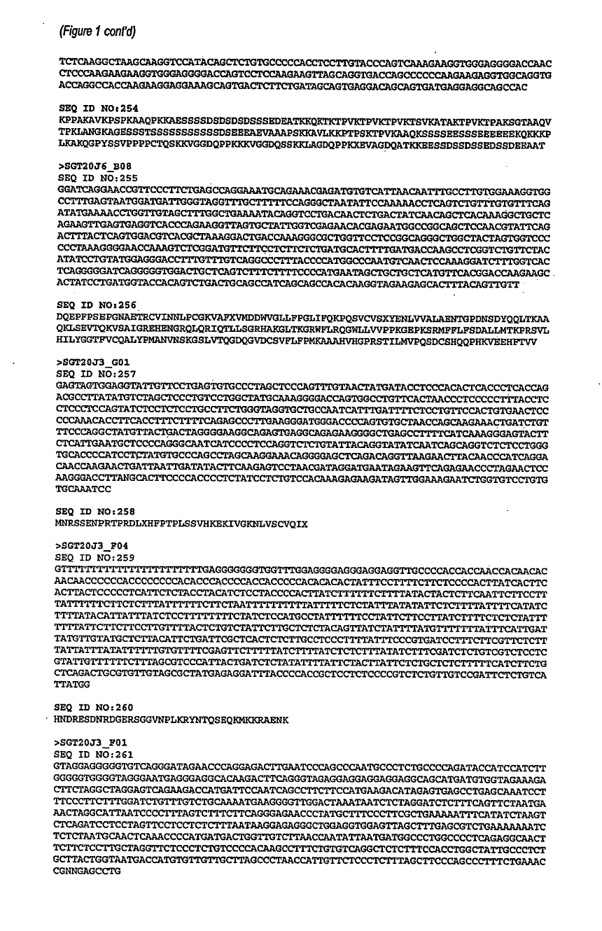

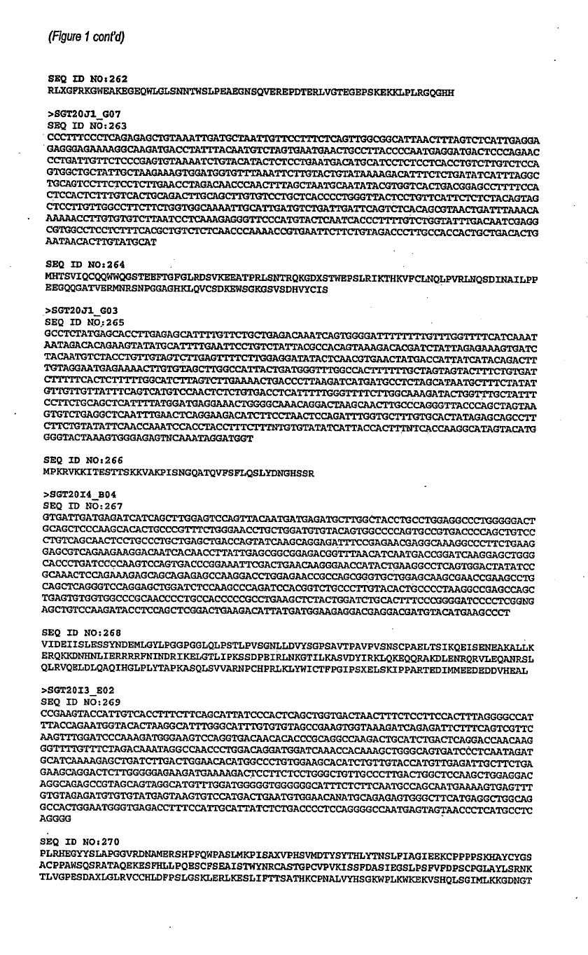

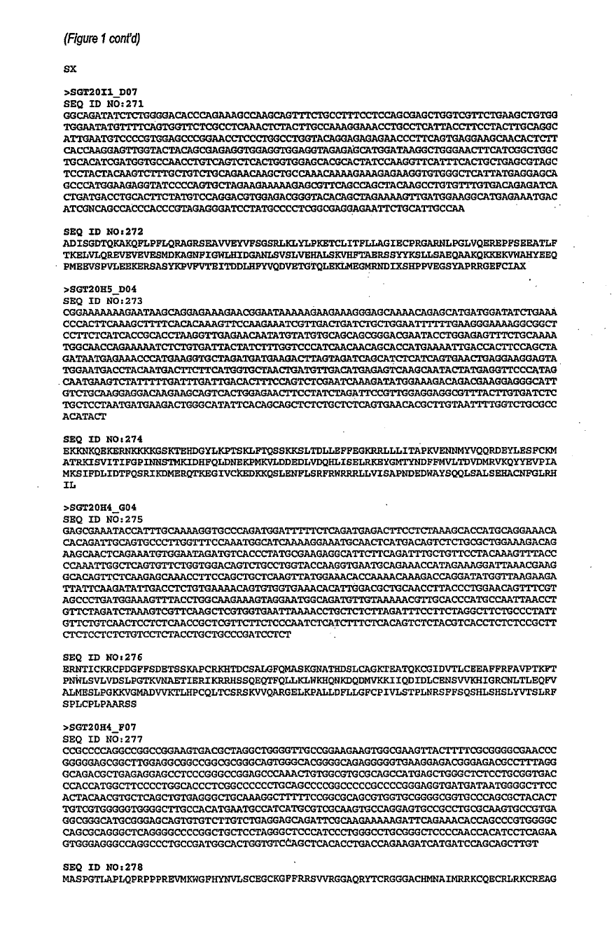

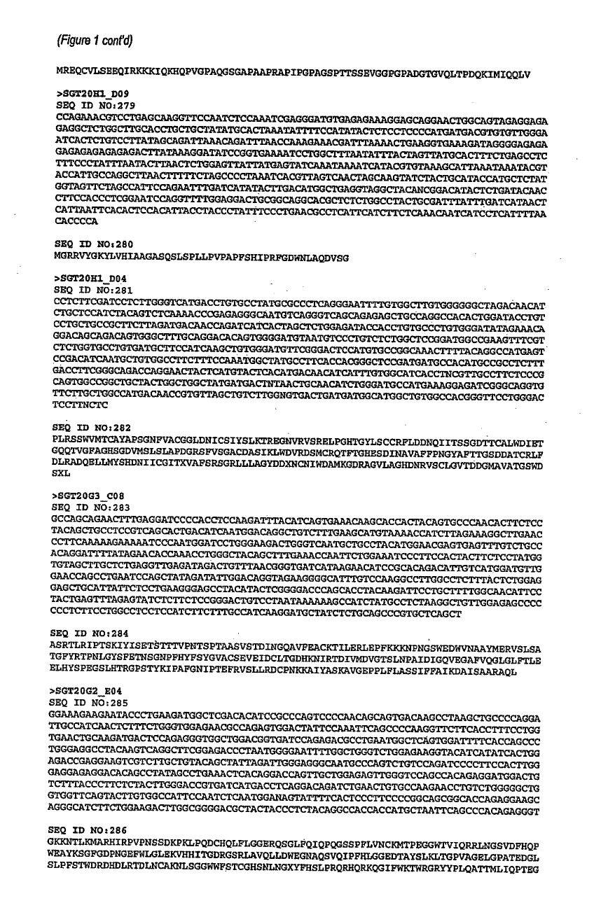

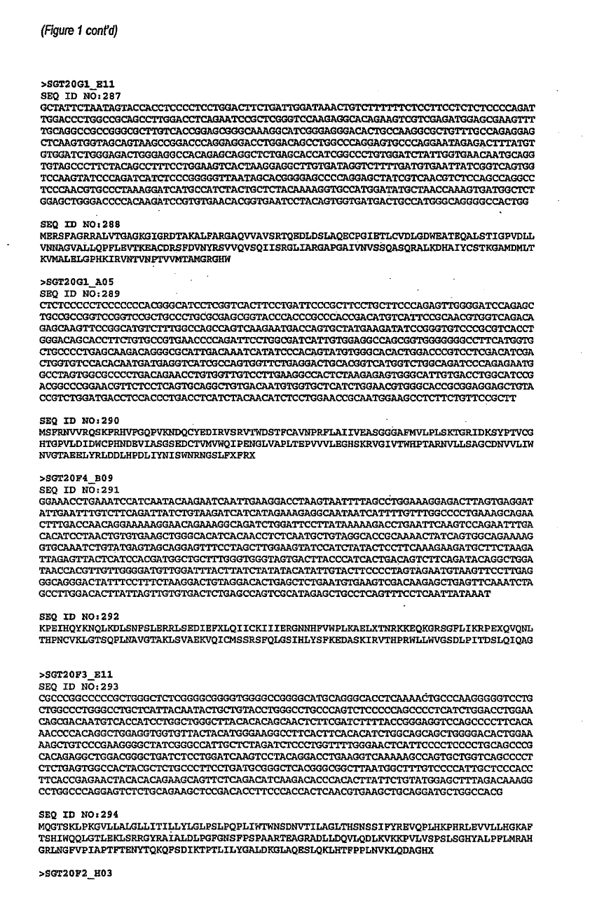

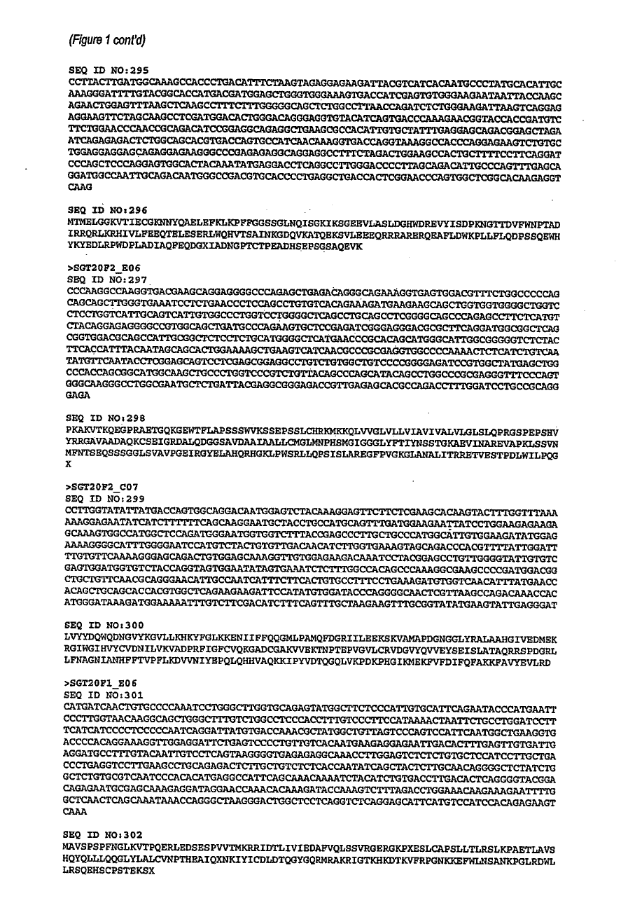

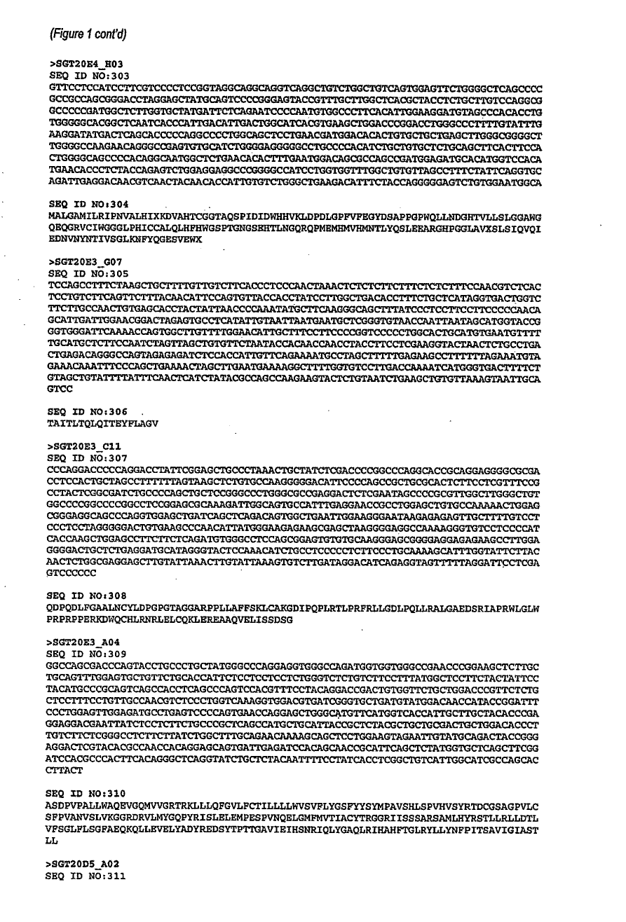

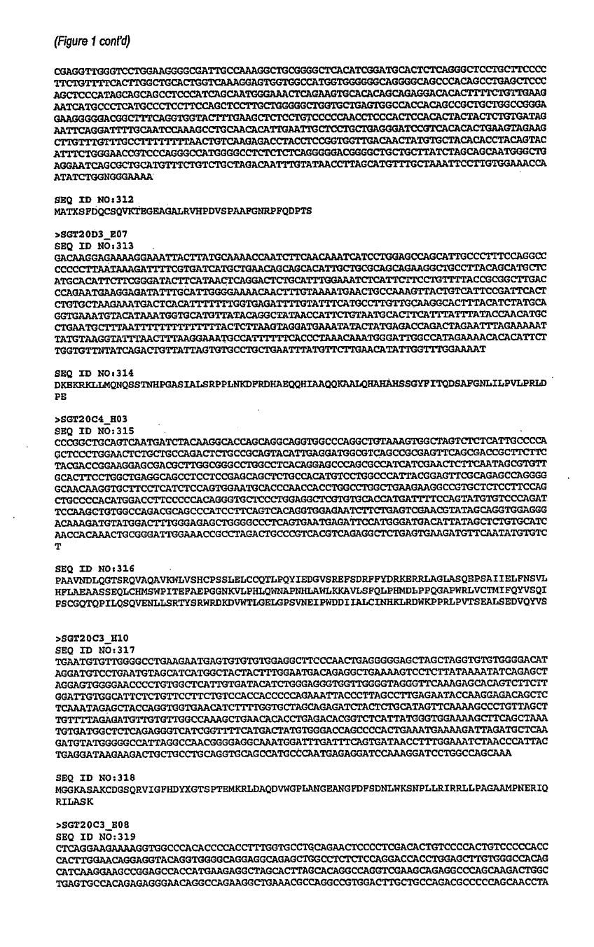

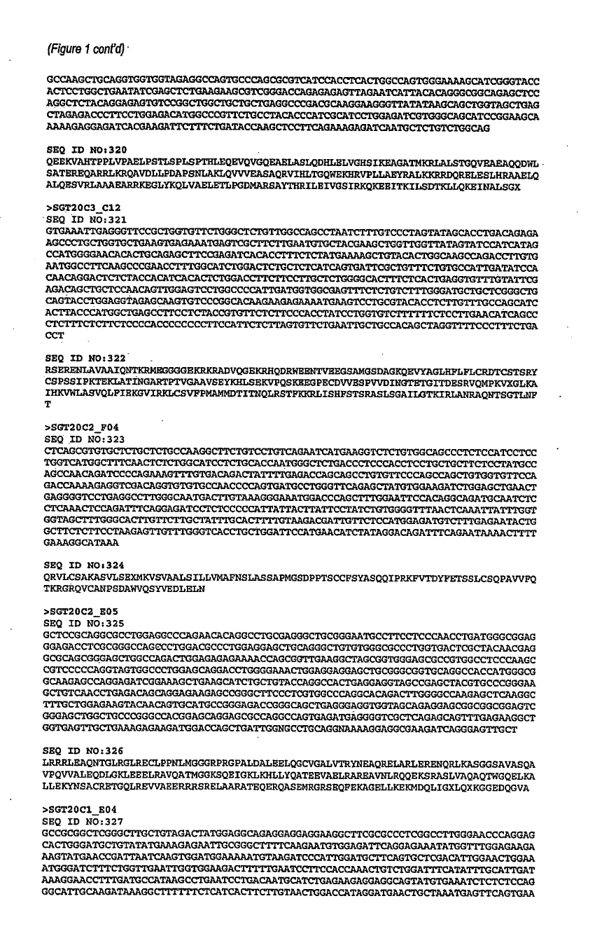

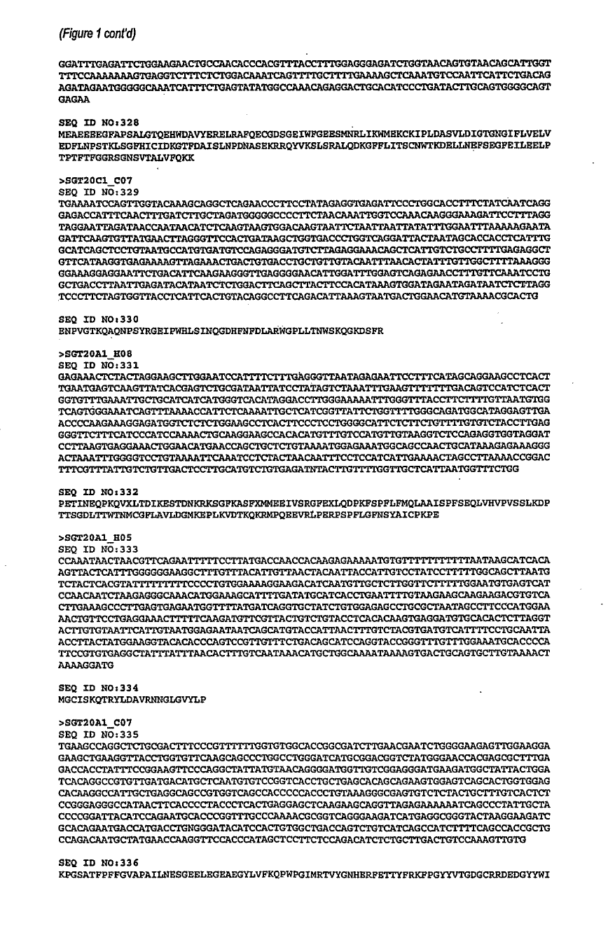

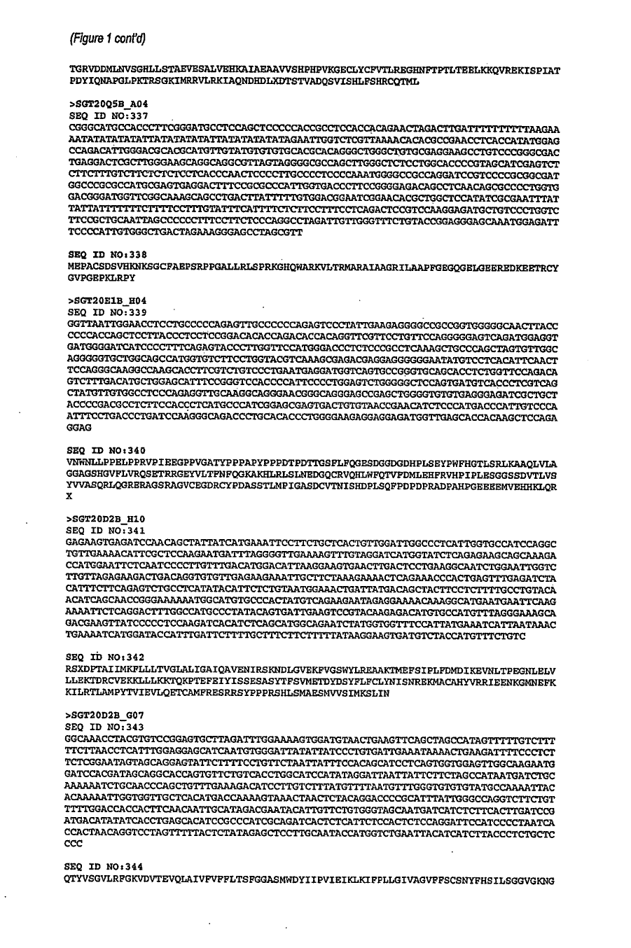

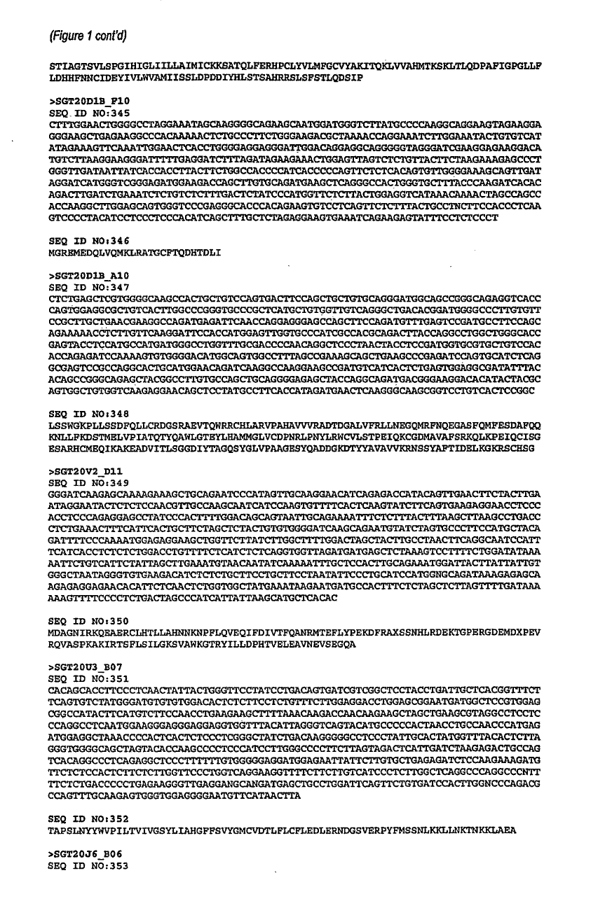

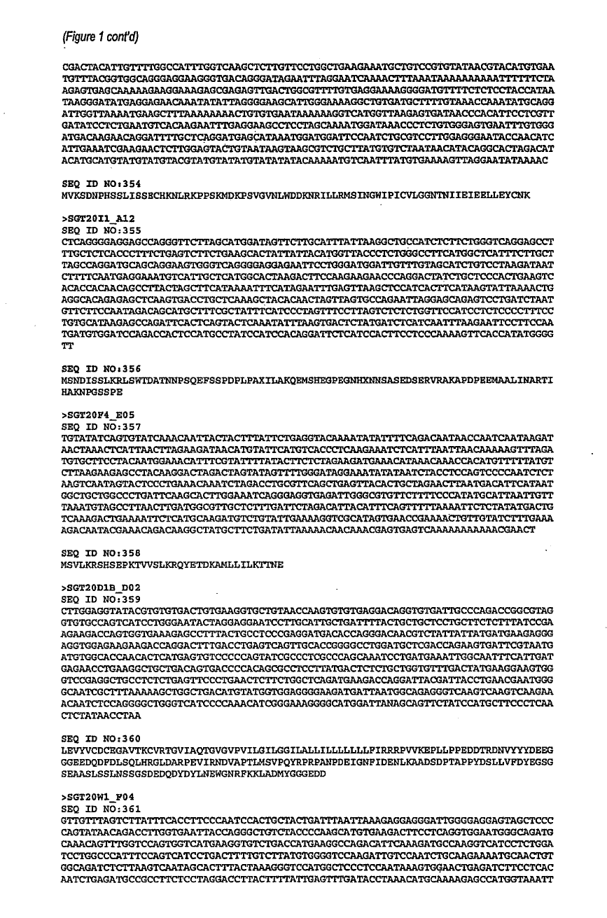

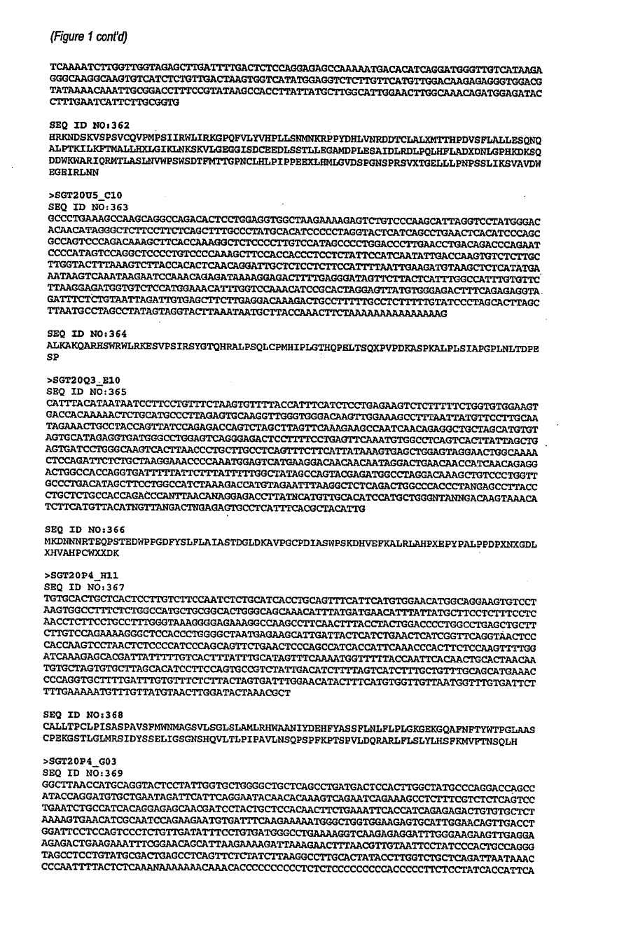

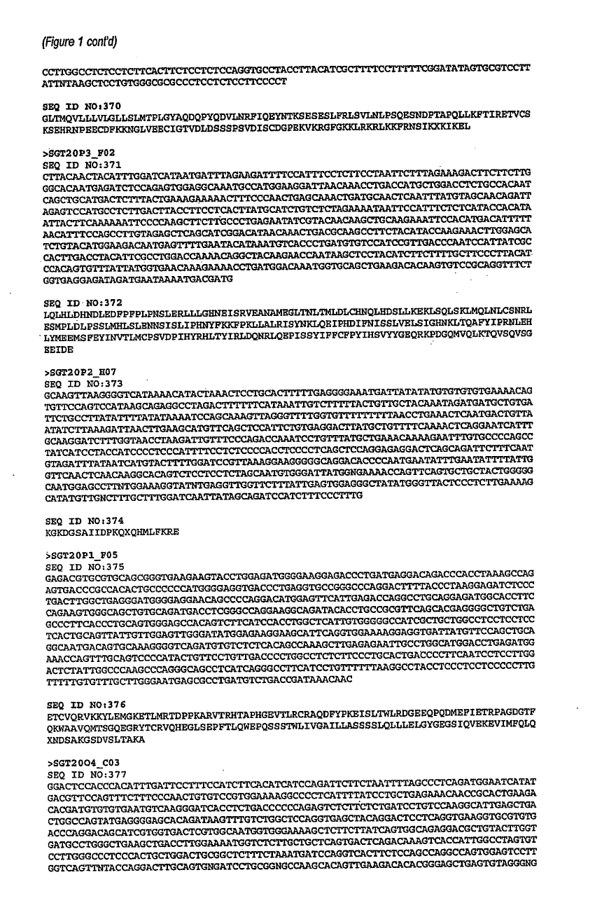

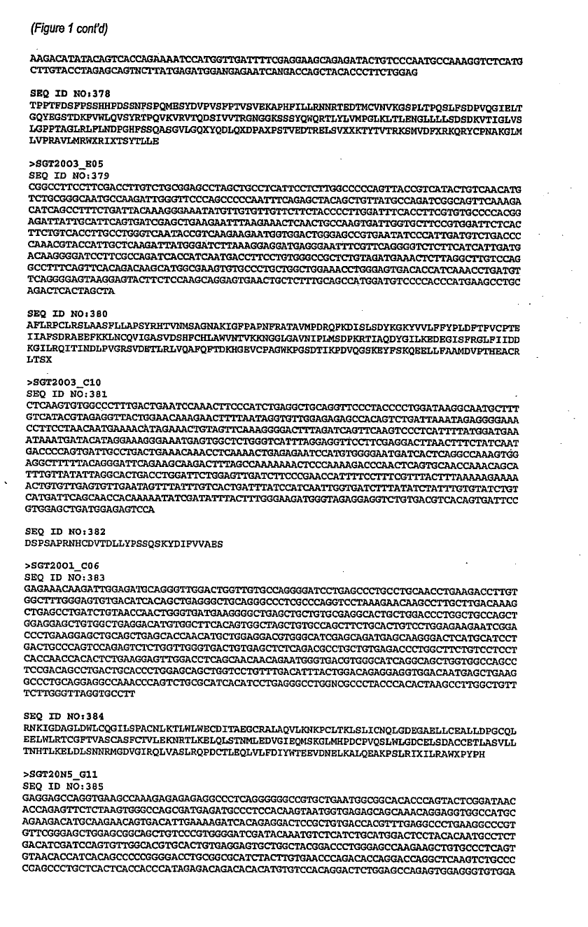

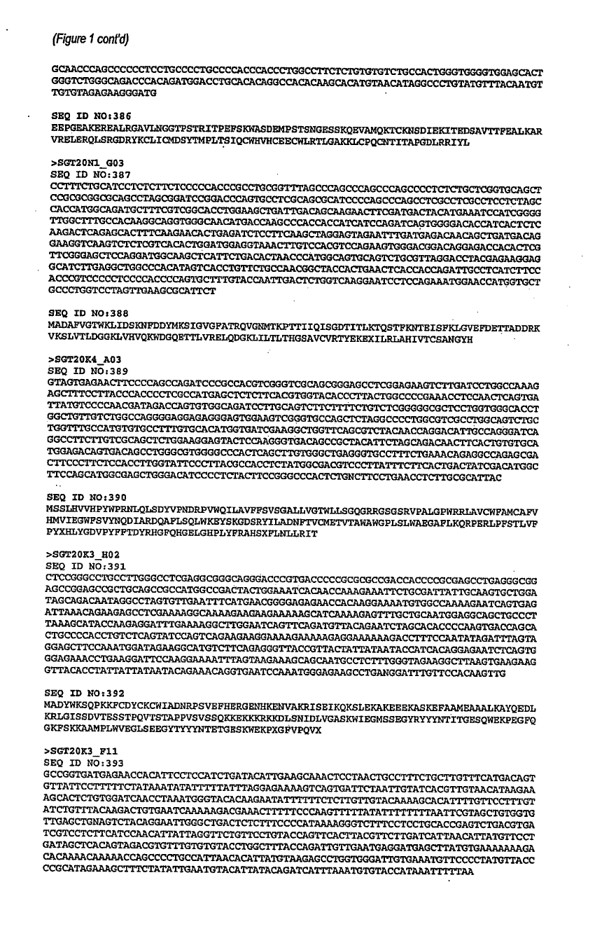

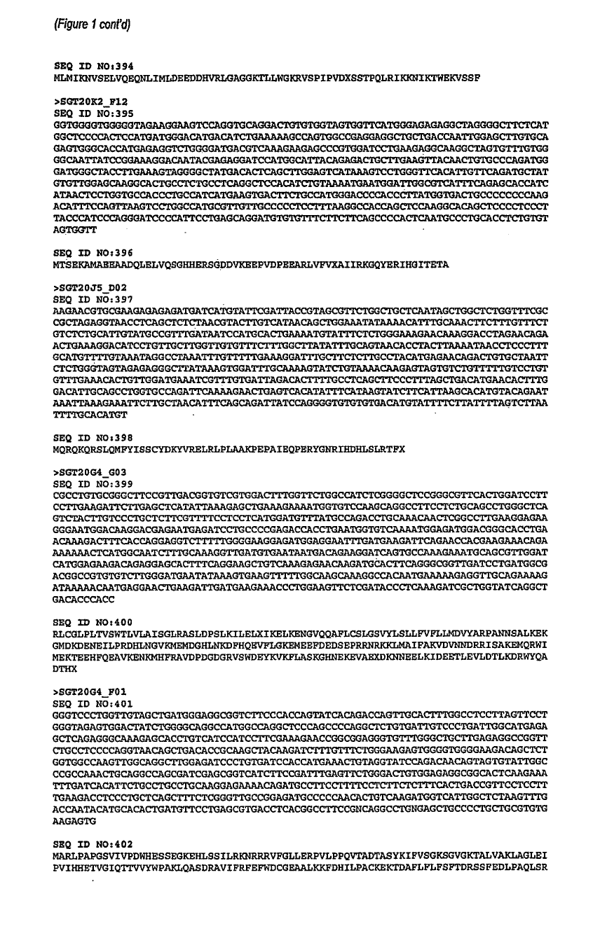

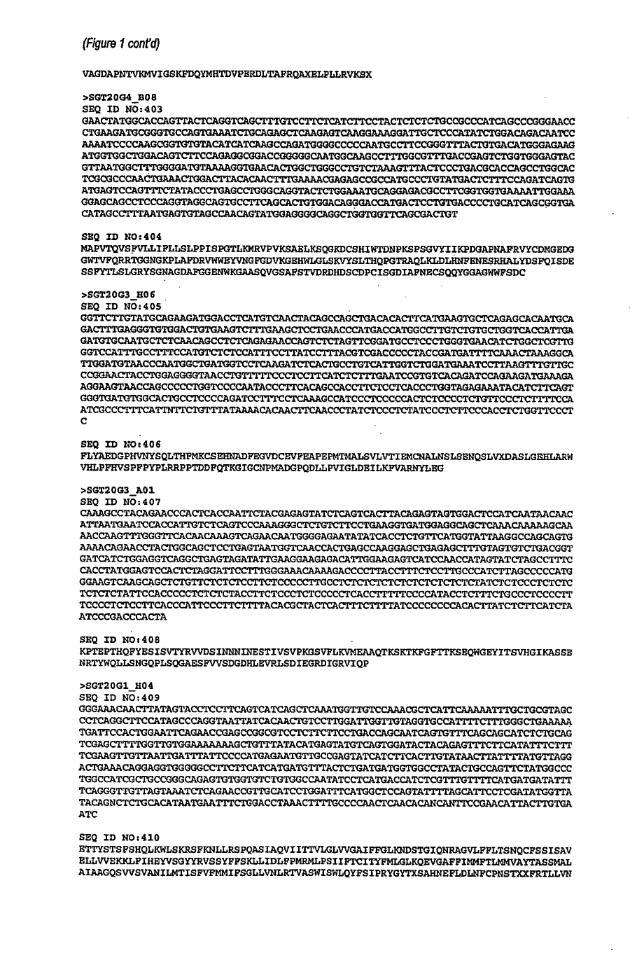

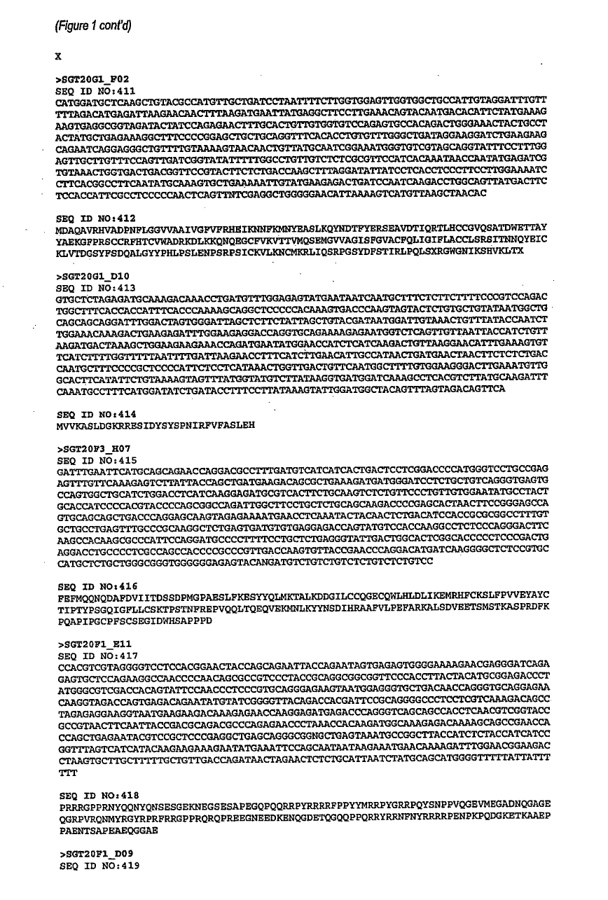

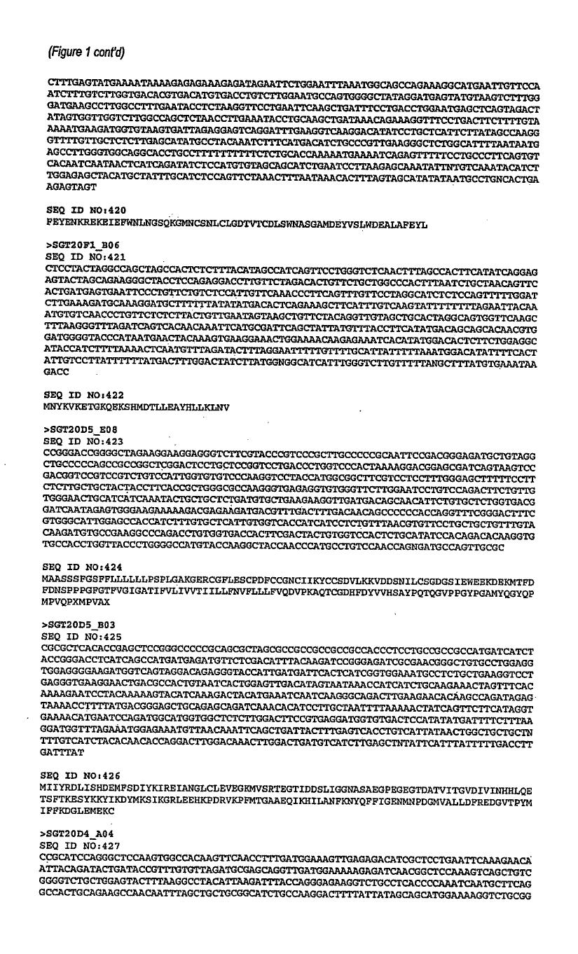

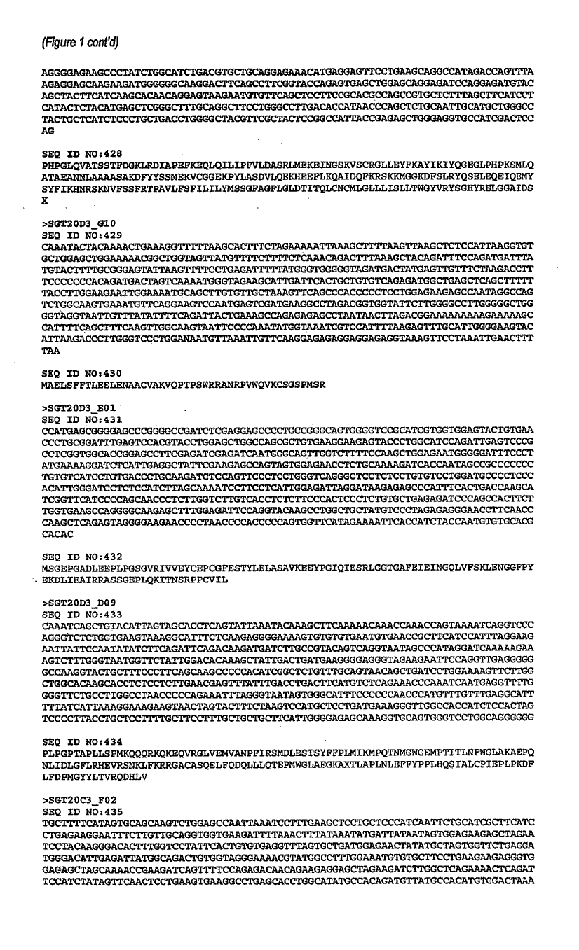

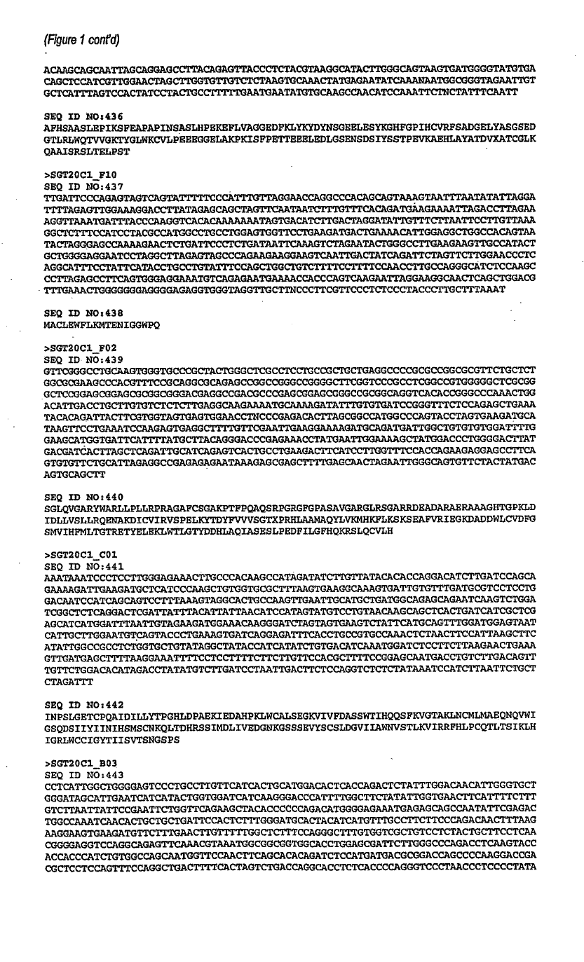

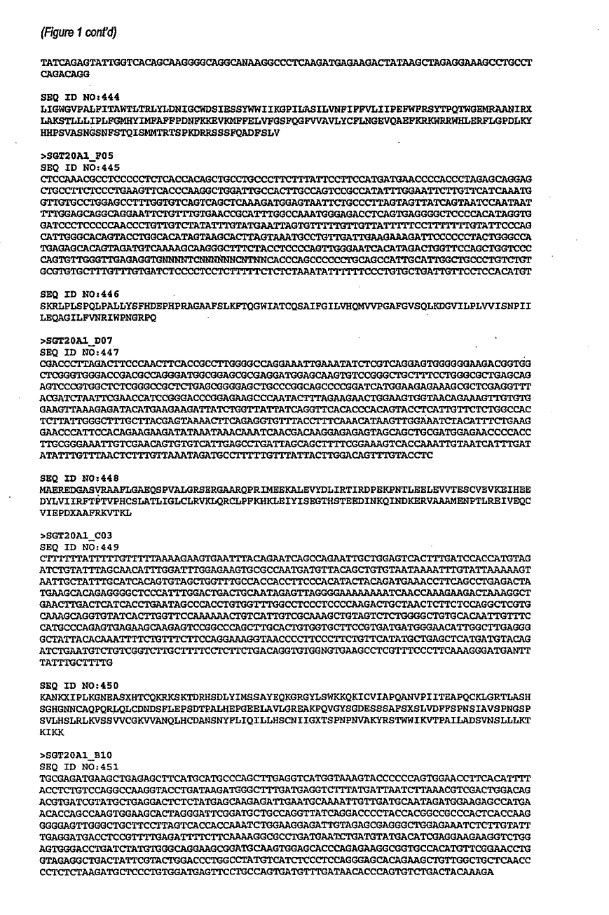

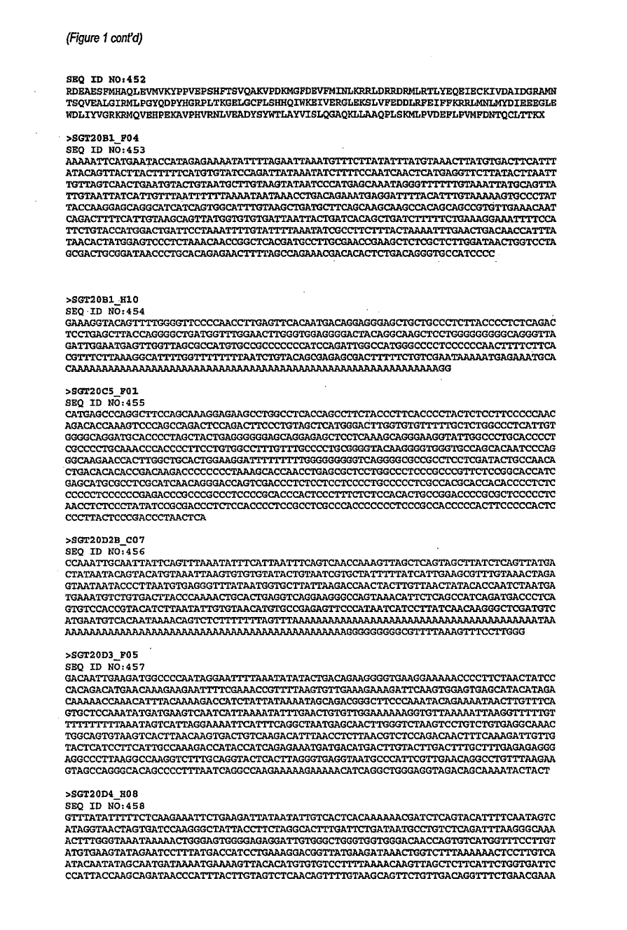

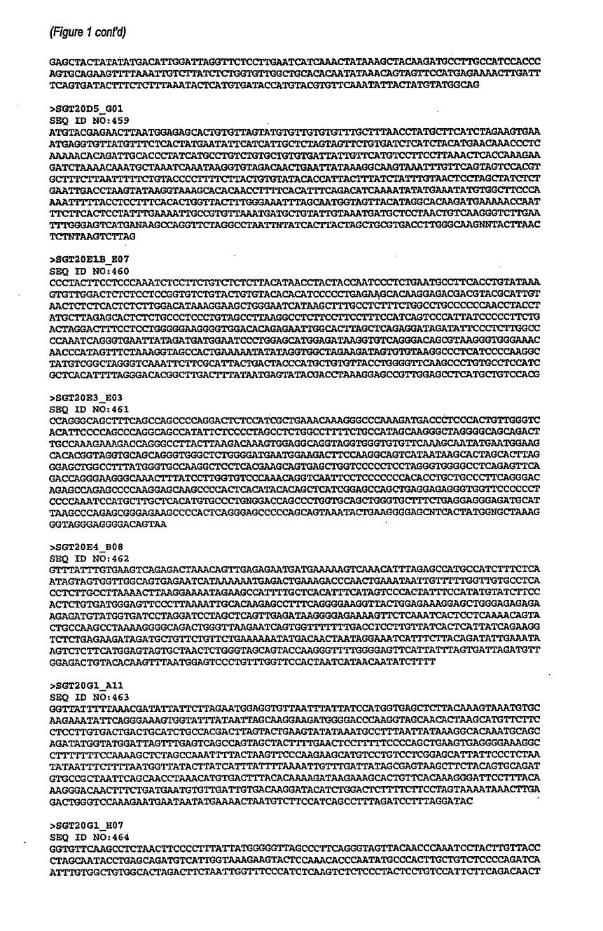

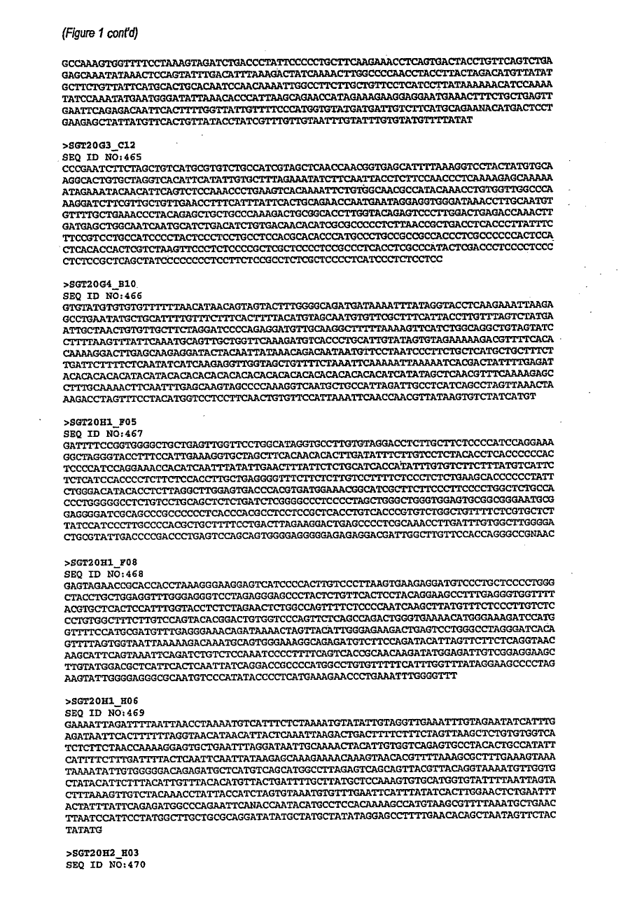

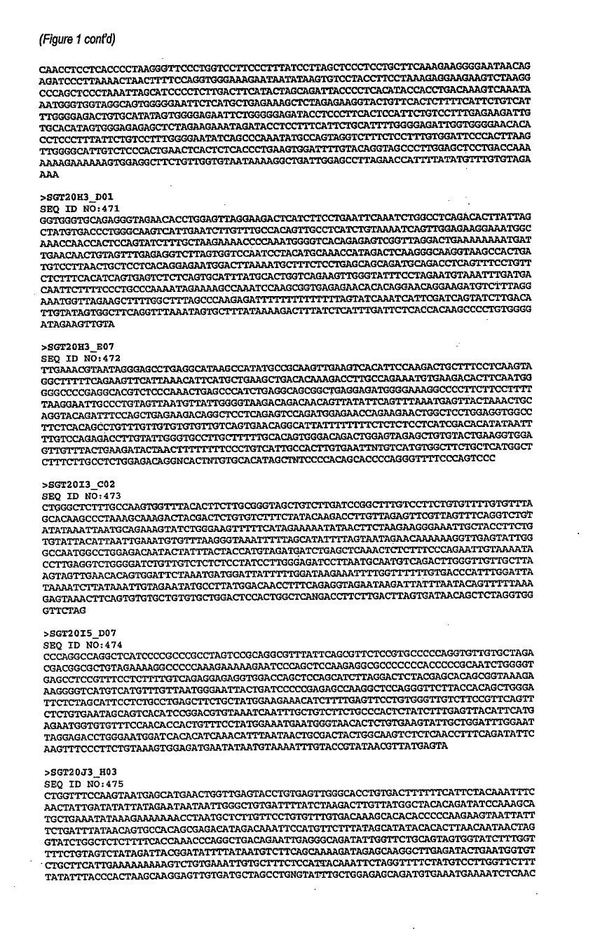

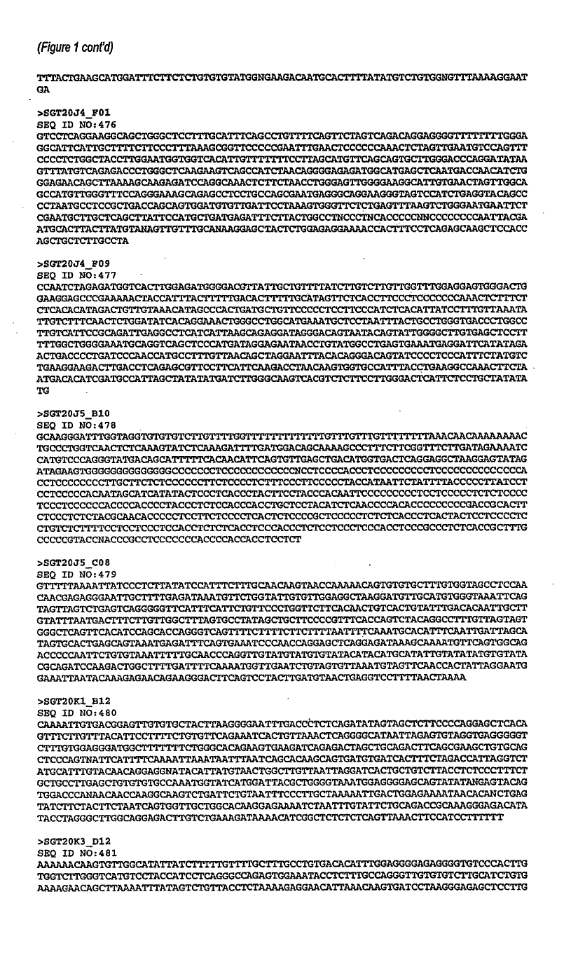

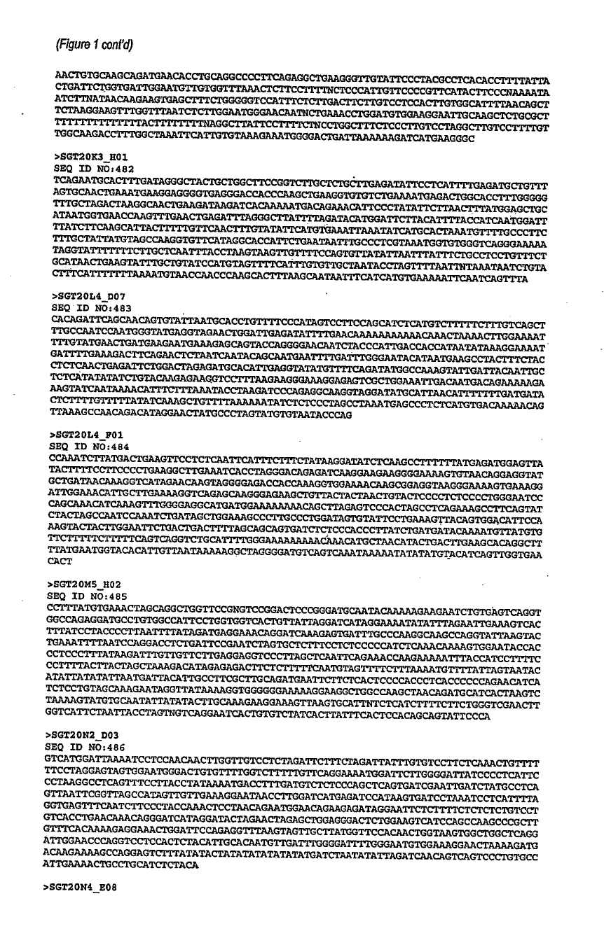

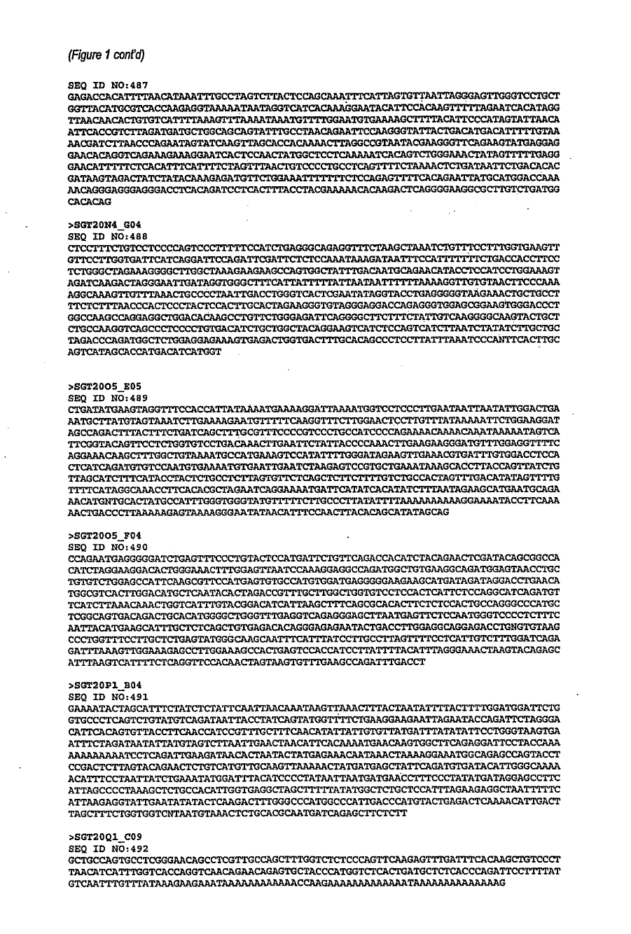

### BEST MODE OF PERFORMING THE INVENTION

A variety of approaches have been adopted in an attempt to elucidate the identity of bioactive proteins in milk. However these approaches have met with limited success and it is accepted that the extent of bioactive proteins in milk has not been fully realized. Our understanding of not only human nutrition and development, but also our ability to manipulate milk production in domestic animals, will depend largely on increasing our understanding of milk composition.

With the tammar wallaby as an experimental model organism, the inventors have used a combination of microarray expression profiling and bioinformatics to identify lactation-associated polypeptides. The present invention is based on this identification of novel polypeptides and polynucleotides encoding the same, the expression of which is altered during lactation.

A polypeptide identified according to the present invention as being lactation-associated may comprise an amino acid sequence as set forth in any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450 or 452. Where an amino acid sequence disclosed herein is the partial sequence of a lactation-associated polypeptide, the corresponding complete sequence may be readily obtained using molecular biology techniques well known to those skilled in the art. Accordingly, the scope of the present invention extends to the complete lactation-associated polypeptides comprising the partial sequences identified herein. The present invention also provides polynucleotides, identified herein as being lactation-associated. A polynucleotide of the invention may comprise a nucleotide sequence as set forth in any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451 or 453 to, 502. Where a nucleotide sequence disclosed herein is the partial sequence of a lactation-associated polynucleotide, the corresponding complete sequence may be readily obtained using molecular biology techniques well known to those skilled in the art. Accordingly, the scope of the present invention extends to the complete lactation-associated polynucleotides comprising the partial sequences identified herein.

The invention also provides methods for the identification and isolation of bioactivities of the polypeptides disclosed herein.

Also contemplated are methods and compositions for treating mammals in need of treatment with effective amounts of polypeptides or polynucleotides of the invention. Such treatment may be for the therapy or prevention of a medical condition in which case an “effective amount” refers to a non-toxic but sufficient amount to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

Polypeptides

Lactation-associated polypeptides of the invention may be regulatory proteins, involved in, for example, regulation of lactogenesis, regulation of lactation phase changes including those relating to changes in milk composition, or regulation of the timing of initiation of milk secretion or involution. Polypeptides of the invention may be bioactive molecules with biological activities of significance to the offspring, including providing nutrition, developmental cues or protection. For example, the bioactive molecules may have anti-microbial activity, anti-inflammatory activity, pro-inflammatory activity or immune response mediator activity. Accordingly, the invention provides methods of identifying such activities in polypeptides of the invention and compositions comprising polypeptides of the invention.

Polypeptides of the invention may have signal or leader sequences to direct their transport across a membrane of a cell, for example to secrete the polypeptide into the extracellular space. The leader sequence may be naturally present on the polypeptide amino acid sequence or may be added to the polypeptide amino acid sequence by recombinant techniques known to those skilled in the art.

In addition to the lactation-associated polypeptides comprising amino acid sequences set forth herein, also included within the scope of the present invention are variants and fragments thereof.

The term “variant” as used herein refers to substantially similar sequences. Generally, polypeptide sequence variants possess qualitative biological activity in common. Further, these polypeptide sequence variants may share at least 50%, 55%, 60%, 65%, 70%, 75%, 80%, 85%, 90%, 95%, 96%, 97%, 98% or 99% sequence identity. Also included within the meaning of the term “variant” are homologues of polypeptides of the invention. A homologue is typically a polypeptide from a different mammalian species but sharing substantially the same biological function or activity as the corresponding polypeptide disclosed herein. For example, homologues of polypeptides disclosed herein may be from bovine species or humans. Such homologues can be located and isolated using standard techniques in molecular biology well known to those skilled in the art, without undue trial or experimentation. Typically homologues are identified and isolated by virtue of the sequence of the polynucleotide encoding the polypeptide, as discussed below.

Further, the term “variant” also includes analogues of the polypeptides of the invention, wherein the term “analogue” means a polypeptide which is a derivative of a polypeptide of the invention, which derivative comprises addition, deletion, substitution of one or more amino acids, such that the polypeptide retains substantially the same function. The term “conservative amino acid substitution” refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

The present invention also contemplates fragments of the polypeptides disclosed herein. The term “fragment” refers to a polypeptide molecule that encodes a constituent or is a constituent of a polypeptide of the invention or variant thereof. Typically the fragment possesses qualitative biological activity in common with the polypeptide of which it is a constituent. The peptide fragment may be between about 5 to about 150 amino acids in length, between about 5 to about 100 amino acids in length, between about 5 to about 50 amino acids in length, or between about 5 to about 25 amino acids in length. Alternatively, the peptide fragment may be between about 5 to about 15 amino acids in length.

Polynucleotides

Embodiments of the present invention provide isolated polynucleotides the expression of which is altered during lactation.

In addition to the lactation-associated polynucleotides comprising nucleotide sequences set forth herein, also included within the scope of the present invention are variants and fragments thereof.

As for polypeptides discussed above, the term “variant” as used herein refers to substantially similar sequences. Generally, polynucleotide sequence variants encode polypeptides which possess qualitative biological activity in common. Further, these polynucleotide sequence variants may share at least 50%, 55%, 60%, 65%, 70%, 75%, 80%, 85%, 90%, 95%, 96%, 97%, 98% or 99% sequence identity. Also included within the meaning of the term variant are homologues of polynucleotides of the invention. A homologue is typically a polynucleotide from a different mammalian species but sharing substantially the same biological function or activity as the corresponding polynucleotide disclosed herein. For example, homologues of polynucleotides disclosed herein may be from bovine species or humans. Such homologues can be located and isolated using standard techniques in molecular biology well known to those skilled in the art, without undue trial or experimentation. Typically homologues are identified and isolated by virtue of the sequence of a polynucleotide disclosed herein.

Fragments of polynucleotides of the invention are also contemplated. The term “fragment” refers to a nucleic acid molecule that encodes a constituent or is a constituent of a polynucleotide of the invention. Fragments of a polynucleotide, do not necessarily need to encode polypeptides which retain biological activity. Rather the fragment may, for example, be useful as a hybridization probe or PCR primer. The fragment may be derived from a polynucleotide of the invention or alternatively may be synthesized by some other means, for example chemical synthesis.

The present invention contemplates the use of polynucleotides disclosed herein and fragments thereof to identify and obtain corresponding partial and complete sequences from other species, such as bovine species and humans using methods of recombinant DNA well known to those of skill in the art, including, but not limited to southern hybridization, northern hybridization, polymerase chain reaction (PCR), ligase chain reaction (LCR) and gene mapping techniques. Polynucleotides of the invention and fragments thereof may also be used in the production of antisense molecules using techniques known to those skilled in the art.

Accordingly, the present invention contemplates oligonucleotides and fragments based on the sequences of the polynucleotides disclosed herein for use as primers and probes for the identification of homologous sequences. Oligonucleotides are short stretches of nucleotide residues suitable for use in nucleic acid amplification reactions such as PCR, typically being at least about 10 nucleotides to about 50 nucleotides in length, more typically about 15 to about 30 nucleotides in length. Probes are nucleotide sequences of variable length, for example between about 10 nucleotides and several thousand nucleotides, for use in detection of homologous sequences, typically by hybridization. The level of homology (sequence identity) between sequences will largely be determined by the stringency of hybridization conditions. In particular the nucleotide sequence used as a probe may hybridize to a homologue or other variant of a polynucleotide disclosed herein under conditions of low stringency, medium stringency or high stringency. Low stringency hybridization conditions may correspond to hybridization performed at 50° C. in 2×SSC. There are numerous conditions and factors, well known to those skilled in the art, which may be employed to alter the stringency of hybridization. For instance, the length and nature (DNA, RNA, base composition) of the nucleic add to be hybridized to a specified nucleic acid; concentration of salts and other components, such as the presence or absence of formamide, dextran sulfate, polyethylene glycol etc; and altering the temperature of the hybridization and/or washing steps. For example, a hybridization filter may be washed twice for 30 minutes in 2×SSC, 0.5% SDS and at least 55° C. (low stringency), at least 60° C. (medium stringency), at least 65° C. (medium/high stringency), at least 70° C. (high stringency) or at least 75° C. (very high stringency).

In particular embodiments, the polynucleotides of the invention may be cloned into a vector. The vector may be a plasmid vector, a viral vector, or any other suitable vehicle adapted for the insertion of foreign sequences, their introduction into eukaryotic cells and the expression of the introduced sequences. Typically the vector is a eukaryotic expression vector and may include expression control and processing sequences such as a promoter, an enhancer, ribosome binding sites, polyadenylation signals and transcription termination sequences.

Modulators

The polypeptides and polynucleotides of the present invention, and fragments and analogues thereof are useful for the screening and identification of compounds and agents that interact with these molecules. In particular, desirable compounds are those that modulate the activity of these polypeptides and polynucleotides. Such compounds may exert a modulatory effect by activating, stimulating, increasing, inhibiting or preventing expression or activity of the polypeptides and/or polynucleotides. Suitable compounds may exert their effect by virtue of either a direct (for example binding) or indirect interaction.

Compounds which bind, or otherwise interact with the polypeptides and polynucleotides of the invention, and specifically compounds which modulate their activity, may be identified by a variety of suitable methods. Interaction and/or binding may be determined using standard competitive binding assays or two-hybrid assay systems.

For example, the two-hybrid assay is a yeast-based genetic assay system typically used for detecting protein-protein interactions. Briefly, this assay takes advantage of the multi-domain nature of transcriptional activators. For example, the DNA-binding domain of a known transcriptional activator may be fused to a polypeptide, or fragment or analogue thereof, and the activation domain of the transcriptional activator fused to a candidate protein. Interaction between the candidate protein and the polypeptide, or fragment or analogue thereof, will bring the DNA-binding and activation domains of the transcriptional activator into close proximity. Interaction can thus be detected by virtue of transcription of a specific reporter gene activated by the transcriptional activator.

Alternatively, affinity chromatography may be used to identify polypeptide binding partners. For example, a polypeptide, or fragment or analogue thereof, may be immobilised on a support (such as sepharose) and cell lysates passed over the column. Proteins binding to the immobilised polypeptide, fragment or analogue can then be eluted from the column and identified. Initially such proteins may be identified by N-terminal amino acid sequencing for example.

Alternatively, in a modification of the above technique, a fusion protein may be generated by fusing a polypeptide, fragment or analogue to a detectable tag, such as alkaline phosphatase, and using a modified form of immunoprecipitation as described by Flanagan and Leder (1990).

Methods for detecting compounds that modulate activity of a polypeptide of the invention may involve combining the polypeptide with a candidate compound and a suitable labelled substrate and monitoring the effect of the compound on the polypeptide by changes in the substrate (may be determined as a function of time). Suitable labelled substrates include those labelled for colourimetric, radiometric, fluorimetric or fluorescent resonance energy transfer (FRET) based methods, for example. Alternatively, compounds that modulate the activity of the polypeptide may be identified by comparing the catalytic activity of the polypeptide in the presence of a candidate compound with the catalytic activity of the polypeptide in the absence of the candidate compound.

The present invention also contemplates compounds which may exert their modulatory effect on polypeptides of the invention by altering expression of the polypeptide. In this case, such compounds may be identified by comparing the level of expression of the polypeptide in the presence of a candidate compound with the level of expression in the absence of the candidate compound.

Polypeptides of the invention and appropriate fragments and analogues can be used in high-throughput screens to assay candidate compounds for the ability to bind to, or otherwise interact therewith. These candidate compounds can be further screened against functional polypeptides to determine the effect of the compound on polypeptide activity.

It will be appreciated that the above described methods are merely examples of the types of methods which may be employed to identify compounds that are capable of interacting with, or modulating the activity of, polypeptides of the invention, and fragments and analogues thereof, of the present invention. Other suitable methods will be known to persons skilled in the art and are within the scope of the present invention.

Potential modulators, for screening by the above methods, may be generated by a number of techniques known to those skilled in the art. For example, various forms of combinatorial chemistry may be used to generate putative non-peptide modulators. Additionally, techniques such as nuclear magnetic resonance (NMR) and X ray crystallography, may be used to model the structure of polypeptides of the invention and computer predictions used to generate possible modulators (in particular inhibitors) that will fit the shape of the substrate binding cleft of the polypeptide.

By the above methods, compounds can be identified which either activate (agonists) or inhibit (antagonists) the expression or activity of polypeptides of the invention. Such compounds may be, for example, antibodies, low molecular weight peptides, nucleic acids or non-proteinaceous organic molecules.

Antagonists or agonists of polypeptides of the invention may include antibodies. Suitable antibodies include, but are not limited to polyclonal antibodies, monoclonal antibodies, chimeric antibodies, humanised antibodies, single chain antibodies and Fab fragments.

Antibodies may be prepared from discrete regions or fragments of the polypeptide of interest. An antigenic polypeptide contains at least about 5, and preferably at least about 10, amino acids. Methods for the generation of suitable antibodies will be readily appreciated by those skilled in the art. For example, a suitable monoclonal antibody, typically containing Fab portions, may be prepared using the hybridoma technology described in Antibodies—A Laboratory Manual, (Harlow and Lane, eds.) Cold Spring Harbor Laboratory, N.Y. (1988), the disclosure of which is incorporated herein by reference.

Similarly, there are various procedures known in the art which may be used for the production of polyclonal antibodies to polypeptides of interest as disclosed herein. For the production of polyclonal antibodies, various host animals, including but not limited to rabbits, mice, rats, sheep, goats, etc, can be immunized by injection with a polypeptide, or fragment or analogue thereof. Further, the polypeptide or fragment or analogue thereof can be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminium hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Screening for the desired antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., eds, 1994, Current Protocols in Molecular Biology, Vol. 1, John Wiley & Sons, Inc., New York). Antibody binding may be detected by virtue of a detectable label on the primary antibody. Alternatively, the primary antibody may be detected by virtue of its binding with a secondary antibody or reagent which is appropriately labelled. A variety of methods are known in the art for detecting binding in an immunoassay and are within the scope of the present invention.

Embodiments of the invention may utilise antisense technology to inhibit the expression of a polynucleotide by blocking translation of the encoded polypeptide. Antisense technology takes advantage of the fact that nucleic acids pair with complementary sequences. Suitable antisense molecules can be manufactured by chemical synthesis or, in the case of antisense RNA, by transcription in vitro or in vivo when linked to a promoter, by methods known to those skilled in the art.

For example, antisense oligonucleotides, typically of 18-30 nucleotides in length, may be generated which are at least substantially complementary across their length to a region of the nucleotide sequence of the polynucleotide of interest. Binding of the antisense oligonucleotide to their complementary cellular nucleotide sequences may interfere with transcription, RNA processing, transport, translation and/or mRNA stability. Suitable antisense oligonucleotides may be prepared by methods well known to those of skill in the art and may be designed to target and bind to regulatory regions of the nucleotide sequence or to coding (exon) or non-coding (intron) sequences. Typically antisense oligonucleotides will be synthesized on automated synthesizers. Suitable antisense oligonucleotides may include modifications designed to improve their delivery into cells, their stability once inside a cell, and/or their binding to the appropriate target. For example, the antisense oligonucleotide may be modified by the addition of one or more phosphorothioate linkages, or the inclusion of one or morpholine rings into the backbone (so-called ‘morpholino’ oligonucleotides).

An alternative antisense technology, known as RNA interference (RNAi), may be used, according to known methods in the art (for example WO 99/49029 and WO 01/70949, the disclosures of which are incorporated herein by reference), to inhibit the expression of a polynucleotide. RNAi refers to a means of selective post-transcriptional gene silencing by destruction of specific mRNA by small interfering RNA molecules (siRNA). The siRNA is generated by cleavage of double stranded RNA, where one strand is identical to the message to be inactivated. Double-stranded RNA molecules may be synthesised in which one strand is identical to a specific region of the p53 mRNA transcript and introduced directly. Alternatively corresponding dsDNA can be employed, which, once presented intracellularly is converted into dsRNA. Methods for the synthesis of suitable molecule for use in RNAi and for achieving post-transcriptional gene silencing are known to those of skill in the art.

A further means of inhibiting expression may be achieved by introducing catalytic antisense nucleic acid constructs, such as ribozymes, which are capable of cleaving mRNA transcripts and thereby preventing the production of wildtype protein. Ribozymes are targeted to and anneal with a particular sequence by virtue of two regions of sequence complementarity to the target flanking the ribozyme catalytic site. After binding the ribozyme cleaves the target in a site-specific manner. The design and testing of ribozymes which specifically recognise and cleave sequences of interest can be achieved by techniques well known to those in the art (for example Lieber and Strauss, 1995, Molecular and Cellular Biology, 15:540-551, the disclosure of which is incorporated herein by reference).

Compositions

Compositions according to embodiments of the invention may be prepared according to methods which are known to those of ordinary skill in the art containing the suitable agents. Such compositions may include a pharmaceutically acceptable carrier, diluent and/or adjuvant. The carriers, diluents and adjuvants must be “acceptable” in terms of being compatible with the other ingredients of the composition, and not deleterious to the recipient thereof. These compositions can be administered by standard routes. In general, the compositions may be administered by the parenteral, topical or oral route.

It will be understood that the specific dose level for any particular individual will depend upon a variety of factors including, for example, the activity of the specific agents employed, the age, body weight, general health, diet the time of administration, rate of excretion, and combination with any other treatment or therapy. Single or multiple administrations of the agents or compositions can be carried out with dose levels and pattern being selected by the treating physician.

Generally, an effective dosage may be to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range may be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 11.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage may be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar, carrageenan; gum tragacanth or gum acacia, and petroleum jelly. Typically, the carrier or carriers will form from 10% to 99.9% by weight of the compositions.

The compositions of the invention may be in a form suitable for parenteral administration, or in the form of a formulation suitable for oral ingestion (such as capsules, tablets, caplets, elixirs, for example).

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

Formulations suitable for topical administration comprise active ingredients together with one or more acceptable carriers, and optionally any other therapeutic ingredients. Formulations suitable for topical administration include liquid or semi-liquid preparations suitable for penetration through the skin to the site of where treatment is required, such as lotions, creams, ointments, pastes or gels.

Creams, ointments or pastes according to the present invention are semi-solid formulations of the active ingredient for external application or for intra-vaginal application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols. The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which are incorporated herein by reference.

The present invention will now be further described in greater detail by reference to the following specific examples, which should not be construed as in anyway limiting the scope of the invention.

### EXAMPLES

### Example 1

### Tammar Wallaby cDNA Libraries

Library Construction

cDNA libraries were prepared from tammar wallaby mammary gland tissue as described below in Table 1. These libraries were derived from tissue isolated at different stages during pregnancy or the lactation cycles of wallabies. In some instances (see Table 1) the cDNA was treated, for example for size selection purposes or to remove known milk proteins, prior to ligation into the vector.

Library T20 represents a normalized library prepared (by LifeTechnologies) from equal parts of RNA isolated from pregnant tammar mammary gland at day 23 of gestation, lactating tammar mammary gland at days 55, 87, 130, 180, 220, 260 and from mammary gland after 5 days of involution (preceded by 45 days of lactation). The library was constructed from the pooled RNA using SuperScript II Rnase H-RT, directionally ligated into pCMV Sport 6.0 vector and transformed into ElectroMax DH10B cells.

TABLE 1
Tammar cDNA libraries generated in the present study

| | | | | Ligation |
|---|---|---|---|---|
| | Mammary Gland Tissue | | | insert:vector |
| Library | source | RNA purity | Treatment | ratio |
| T01 | Day 130 lactation | total RNA | none ¹ | 1:1 |
| T02 | Day 130 lactation | total RNA | none ¹ | 3:1 |
| T03 | Day 130 lactation | polyA + RNA | none ¹ | 1:1 |
| T04 | Day 130 lactation | polyA + RNA | none ¹ | 3:1 |
| T05 | Day 130 lactation | polyA + RNA | cDNA size selected | 1:1 |
| | | | 0.5-1.0 kbp ¹ | |
| T06 | Day 130 lactation | polyA + RNA | cDNA size selected | 3:1 |
| | | | 0.5-1.0 kbp ¹ | |
| T07 | Day 130 lactation | polyA + RNA | cDNA size selected | 1:1 |
| | | | 1.0-2.0 kbp ¹ | |
| T08 | Day 130 lactation | polyA + RNA | cDNA size selected | 3:1 |
| | | | 1.0-2.0 kbp ¹ | |
| T09 | Day 130 lactation | polyA + RNA | cDNA size selected | 1:1 |
| | | | 2.0-4.0 kbp ¹ | |
| T10 | Day 130 lactation | polyA + RNA | cDNA size selected | 3:1 |
| | | | 2.0-4.0 kbp ¹ | |
| T11 | Day 130 lactation | polyA + RNA | Subtracted for α-casein, β-casein, | 1:1 |
| | | | κ-casein, α-lactalbumin, β- | |
| | | | lactoglobulin ² | |
| T12 | Day 130 lactation | polyA + RNA | Subtracted for α-casein, β-casein, | 3:1 |
| | | | κ-casein, α-lactalbumin, β- | |
| | | | lactoglobulin ² | |
| T13 | Day 23 pregnancy | polyA + RNA | none ¹ | 1:1 and 3:1 |
| | | | | combined |
| T14 | Day 260 lactation | polyA + RNA | none ¹ | 1:1 and 3:1 |
| | | | | combined |
| T15 | Day 23 pregnancy | polyA + RNA | cDNA synthesized using | 1:1 and 3:1 |
| | | | Thermoscript RT ¹ | combined |
| T16 | Day 23 pregnancy | polyA + RNA | cDNA fragments purified though | 1:1 |
| | | | column as per manufacturers | |
| | | | instructions ³ | |
| T17 | Day 23 pregnancy | polyA + RNA | cDNA fragments purified though | 3:1 |
| | | | column as per manufacturers | |
| | | | instructions ³ | |
| T18 | Day 4 lactation, non- | polyA + RNA | cDNA fragments purified though | 1:1 |
| | sucked gland | | column as per manufacturers | |
| | | | instructions ³ | |
| T19 | Day 4 lactation, non- | polyA + RNA | cDNA fragments purified though | 3:1 |
| | sucked gland | | column as per manufacturers | |
| | | | instructions ³ | |

| | |
|---|---|
| T20 | normalized library (printed on microarray) |

¹ Prepared using Clontech Smart cDNA Synthesis kit, cDNA cloned in pGEM-T
² Prepared using Clontech DNA-Select Subtraction kit, cDNA cloned in pGEM-T
³ Prepared using Clontech Smart cDNA Library Construction kit

DNA Sequencing

The cDNA libraries were transformed into either DN 10B or JM109 E. coli cells and plated on LB agar containing ampicillin. Individual colonies were picked and grown in LB media containing ampicillin for plasmid preparation and sequencing. The cDNA insert was sequenced using primers specific to either the T7 or SP6 RNA polymerase promoters in the vector. Alternatively, and where appropriate, the smart oligonucleotide (used in the preparation of the cDNA) was used to sequence specifically from the 5′ end of the cDNA. Sequencing was performed on an Applied Biosystems ABI 3700 automated sequencer, used Big-Dye Terminator reactions. The DNA base calling algorithm PHRED and sequence assembly algorithm PHRAP were used to generate the final sequence files.

### Example 2

### Microarray Expression Profiling

Spotted cDNA microarrays were prepared using clones from the normalized library T20. The cDNA inserts were amplified using T7 and SP6 primers and Perkin-Elmer Taq polymerase. The resulting 9984 amplified DNA samples and Amersham's Lucidia scorecard DNA were spotted onto glass slides by the Peter MacCallum Microarray Facility (under contract). Total RNA from pregnant and lactating tammar wallaby mammary gland was extracted from tissues using Tripure Isolation Reagent (Roche), and further purified using Qiagen RNeasy columns. RNA was labeled using amino allyl reverse transcription followed by Cy3 and Cy5 coupling. Samples of 50 ug total RNA and Amersham's Lucidia Scorcard Mix were reverse transcribed in 87 ng/ul oligo dT Promega MMLV reverse transcriptase, RNAseH and 1× buffer at 42° C. for 2.5 hours. The resultant products were hydrolyzed by incubation at 65° C. for 15 minutes in the presence of 33 mM NaOH, 33 mM EDTA and 40 mM acetic acid. The cDNA was then adsorbed to a Qiagen QIAquick PCR Purification column.

Coupling of either Cy3 or Cy5 dye was performed by incubation with adsorbed cDNA in 0.1M sodium bicarbonate for 1 hour at room temperature in darkness, followed by elution in 80 ul water. Labeled cDNA was further purified using a second Qiagen QIAquick PCR Purification column. Cy3 and Cy5 labeled probes in a final concentration of 400 ug/ml yeast tRNA, 1 mg/ml human Cot-1 DNA, 200 ug/ml polydT₅₀, 1.2×Denhart's, 1 mg/ml herring sperm DNA, 3.2×SSC, 50% formamide and 0.1% SDS were heated to 100° C. for 3 minutes and then hybridized with microarray spotted cDNAs at 42° C. for 16 hours.

Microarrays were washed in 0.5×SSC, 0.01% SDS for 1 minute, 0.5×SSC for 3 minutes then 0.006×SSC for 3 minutes at room temperature in the dark.

Slides were scanned and the resulting images processed using Biorad Versarray software.

Data from spot intensities was either cross channel Loess normalized or single channel normalized. Cross channel normalization was performed using the Versarray software using the following parameters:

Background method “Local ring, Offset: 1, Width: 2, Filter: 0 Erosion: 0”
Net intensity measurement method Raw intensity—Median background (Ignore negatives)
Net intensity normalization “Cross-channel,Local regression (Loess),Median”
Cell shape Ellipse
Cell size 30×30 pixels
Single channel normalization used the Bioconductor software (Smyth and Speed, 2003, Normalization of cDNA microarray data, Methods 2003 31:265-73, see LIMMA http://bioinf.wehi.edu.au/limma) on data generated from the Versarray image analysis.

Microarray analysis of gene expression was performed using the following cross phase comparisons.

### Mammary Tissue Samples

### Phase 1 Tissue

### day 5 Pregnancy

### day 22 Pregnancy

### day 25 Pregnancy

### Phase 2A Tissue

### day 1 Lactation

### day 5 Lactation

### day 80 Lactation

### Phase 2B Tissue

### day 130 Lactation

### day 168 Lactation

### day 180 Lactation

### Phase 3 Tissue

### day 213 Lactation

### day 220 Lactation

### day 260 Lactation

### Phase 1-2A Comparisons

| Cy3 | | Cy5 |
|---|---|---|
| 5P | versus | 80L |
| 5P | versus | 1L |
| 22P | versus | 5L |
| 22P | versus | 80L |
| 25P | versus | 1L |
| 25P | versus | 5L |
| 5L | versus | 22P |
| 80L | versus | 22P |
| 1L | versus | 25P |
| 5L | versus | 25P |

### Phase 2A-2B Comparisons

| Cy3 | | Cy5 |
|---|---|---|
| 80L | versus | 168L |
| 130L | versus | 1L |
| 168L | versus | 80L |

### Phase 2B-3 Comparisons

| Cy3 | | Cy5 |
|---|---|---|
| 130L | versus | 260L |
| 130L | versus | 213L |
| 168L | versus | 220L |
| 168L | versus | 260L |
| 180L | versus | 213L |
| 168L | versus | 213L |
| 260L | versus | 130L |
| 213L | versus | 130L |
| 220L | versus | 168L |
| 260L | versus | 168L |
| 213L | versus | 168L |

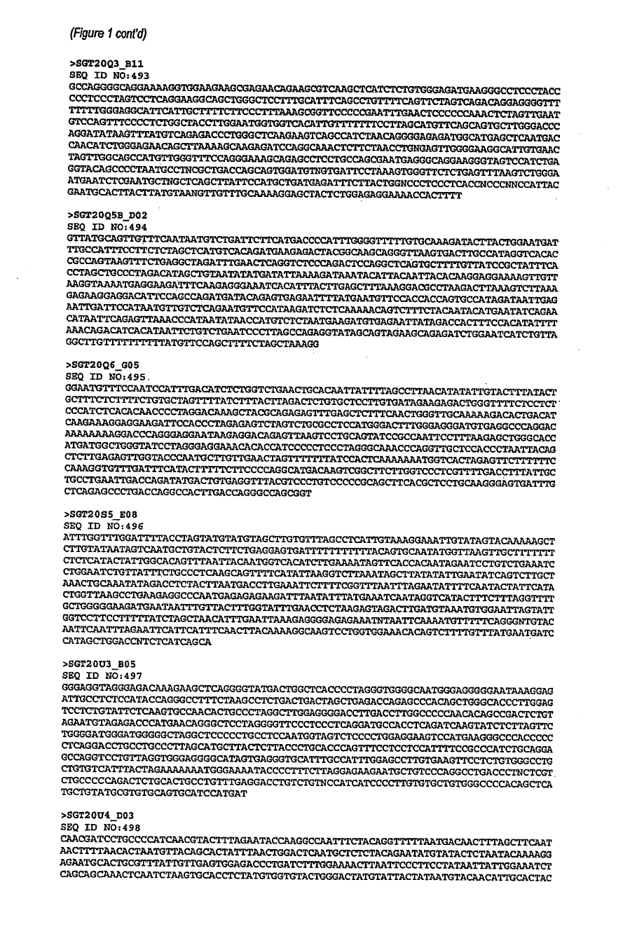

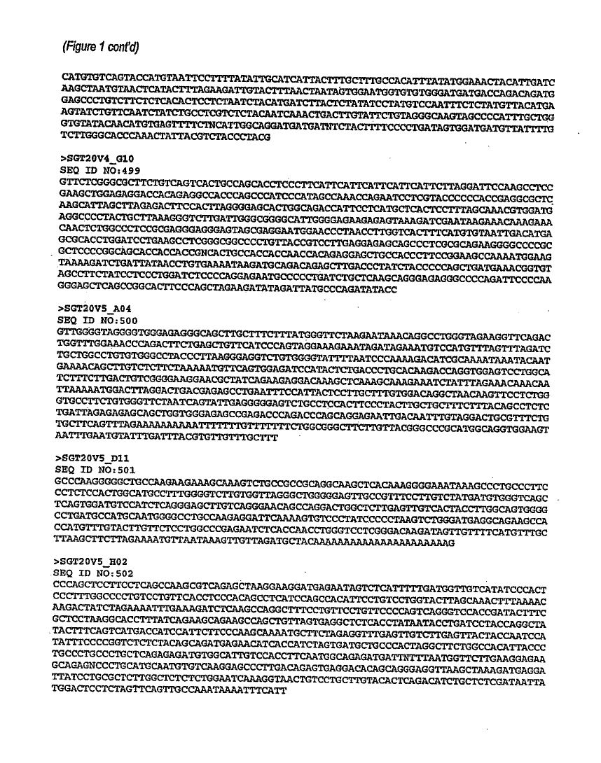

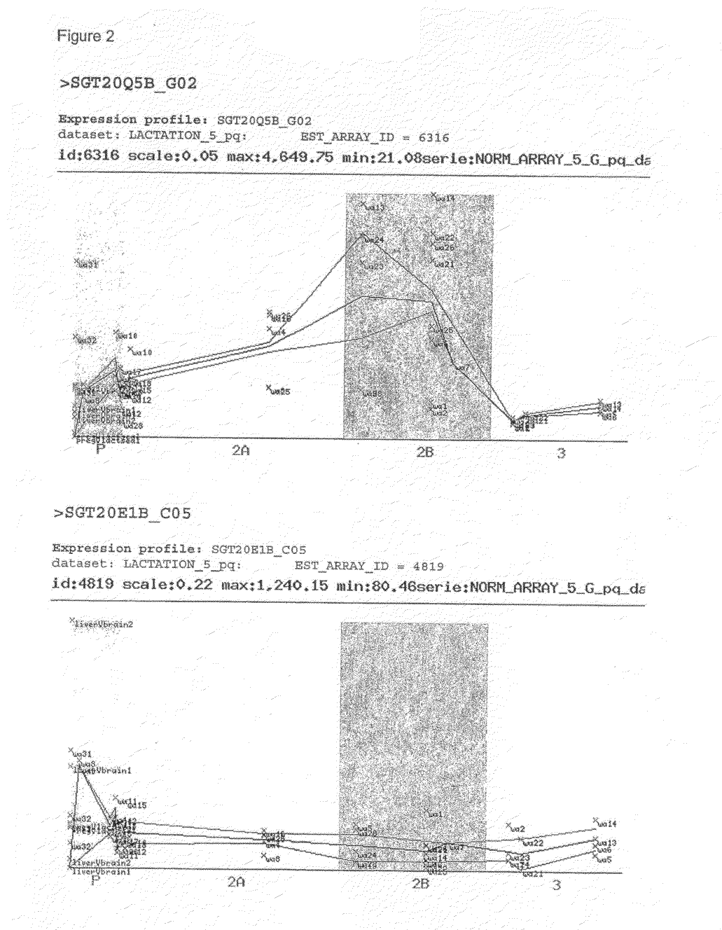

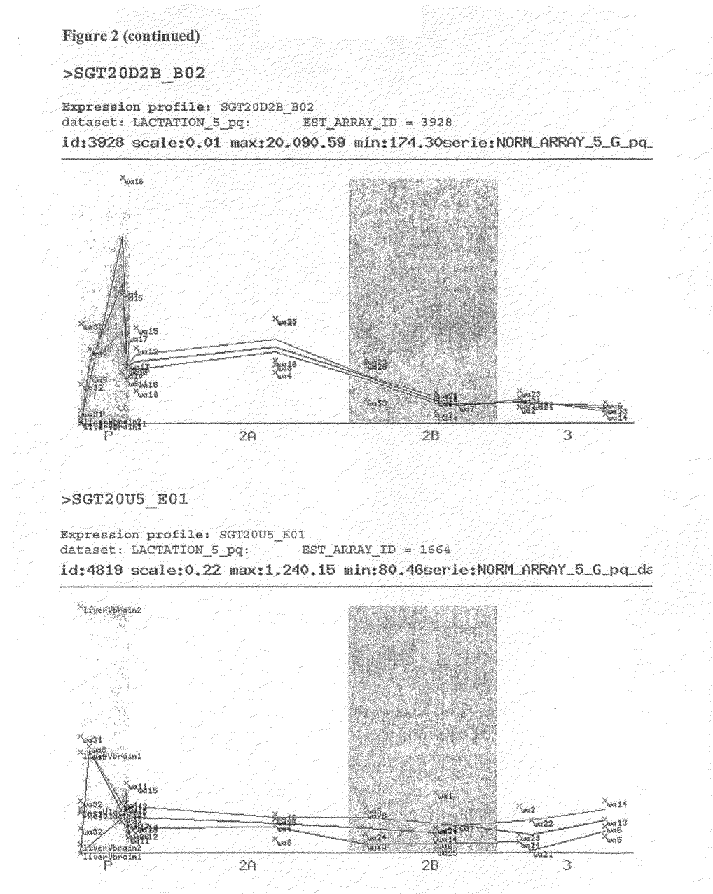

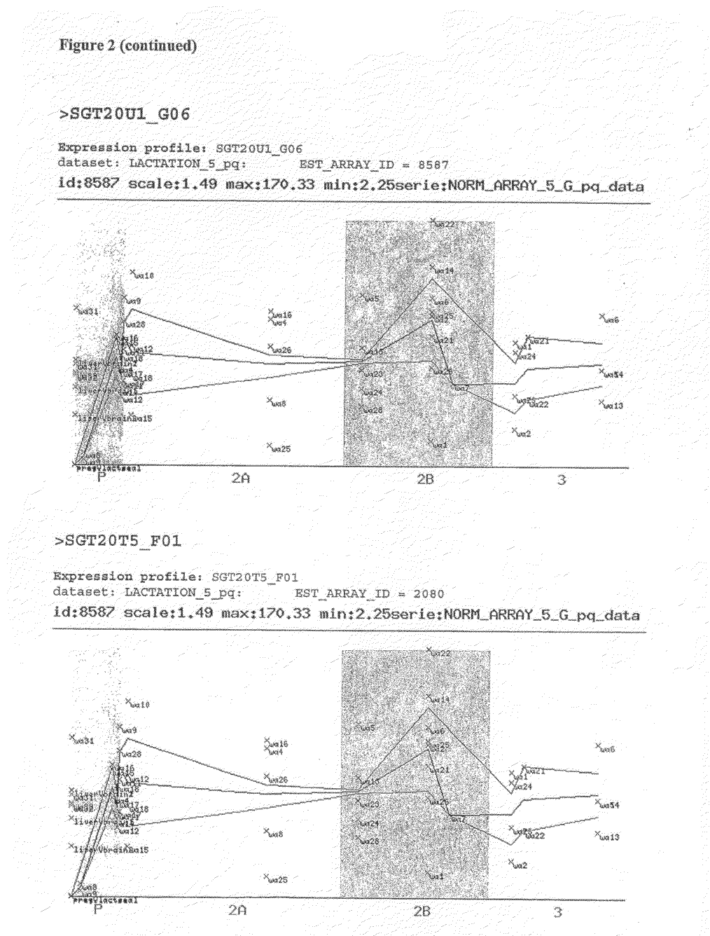

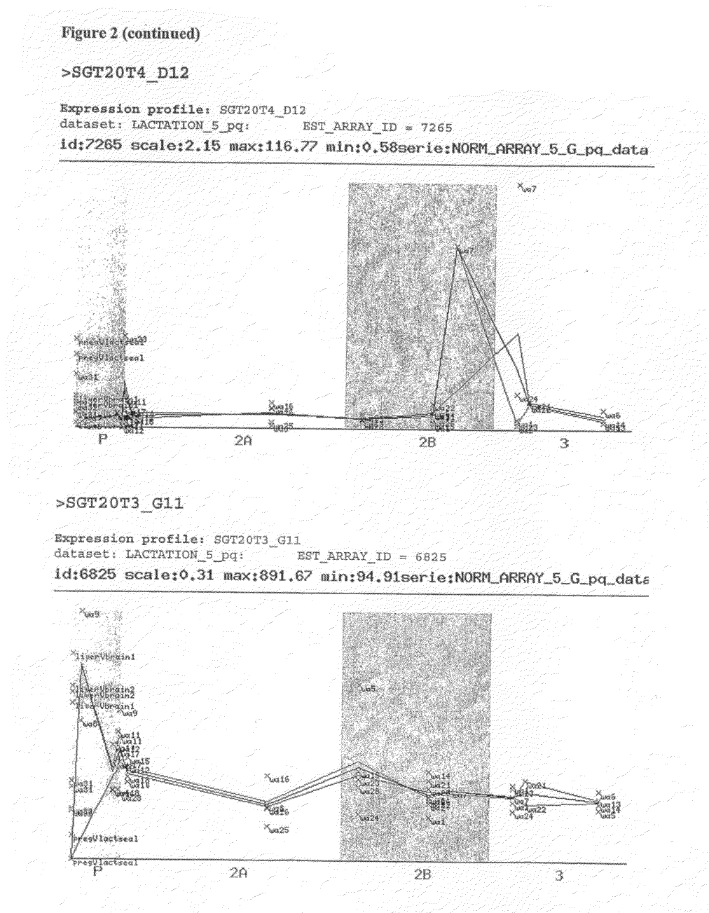

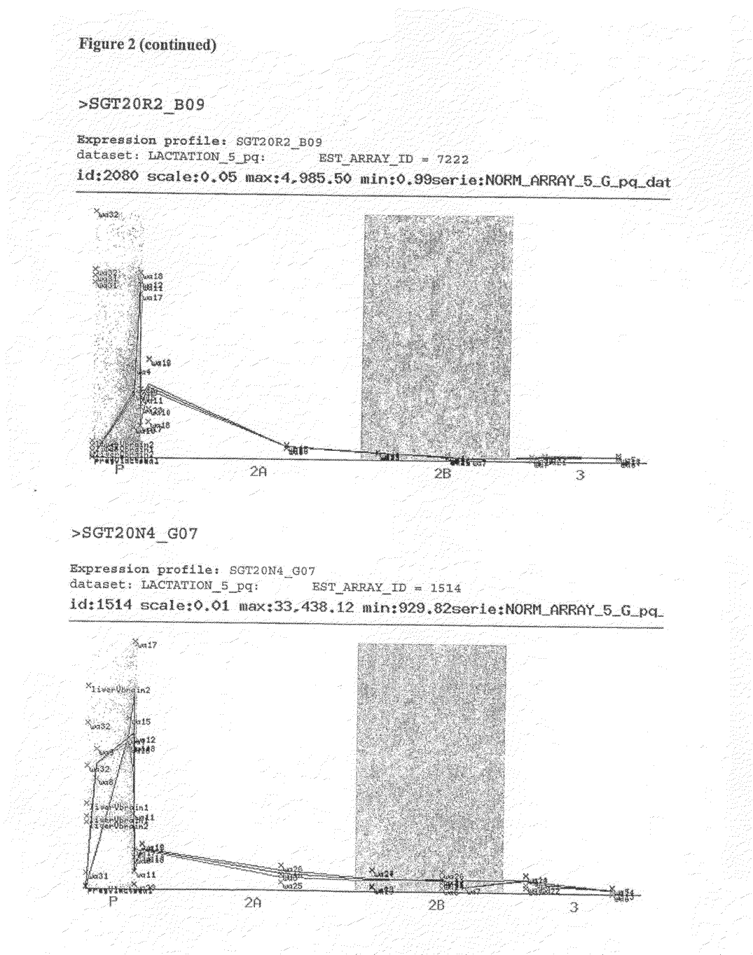

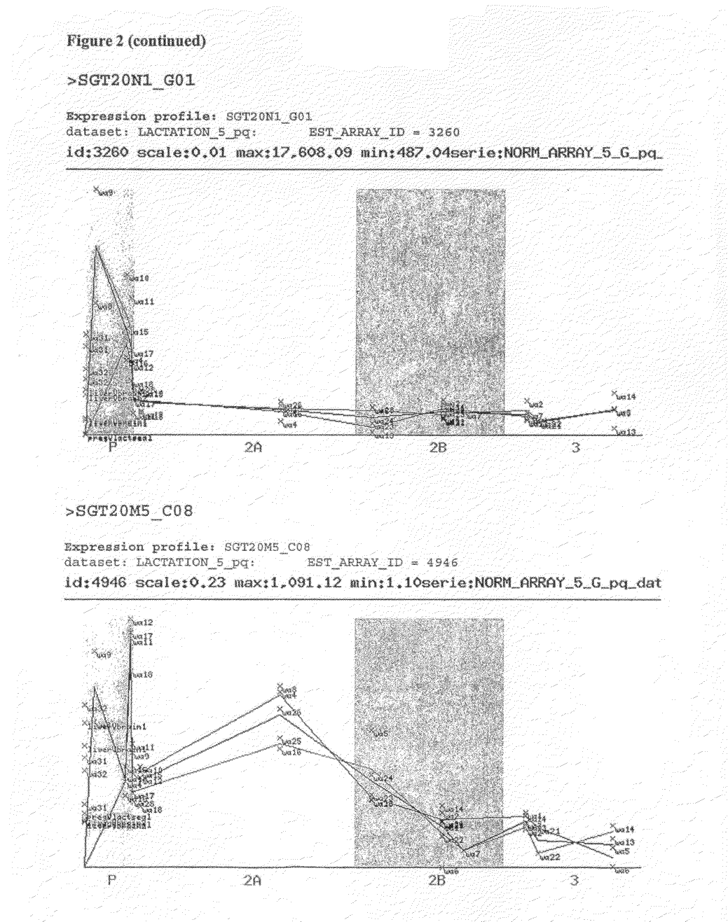

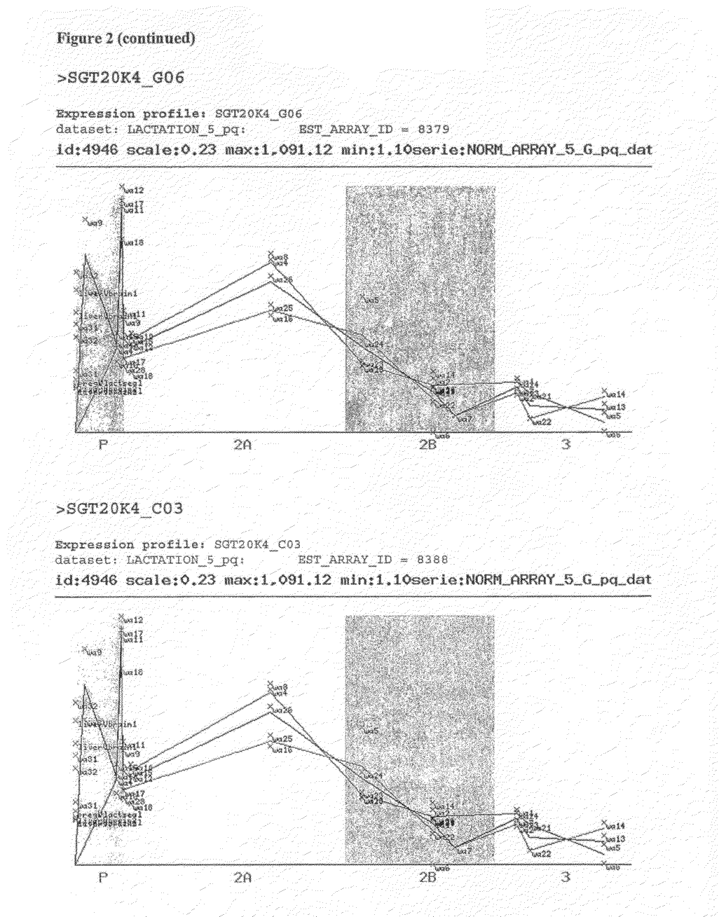

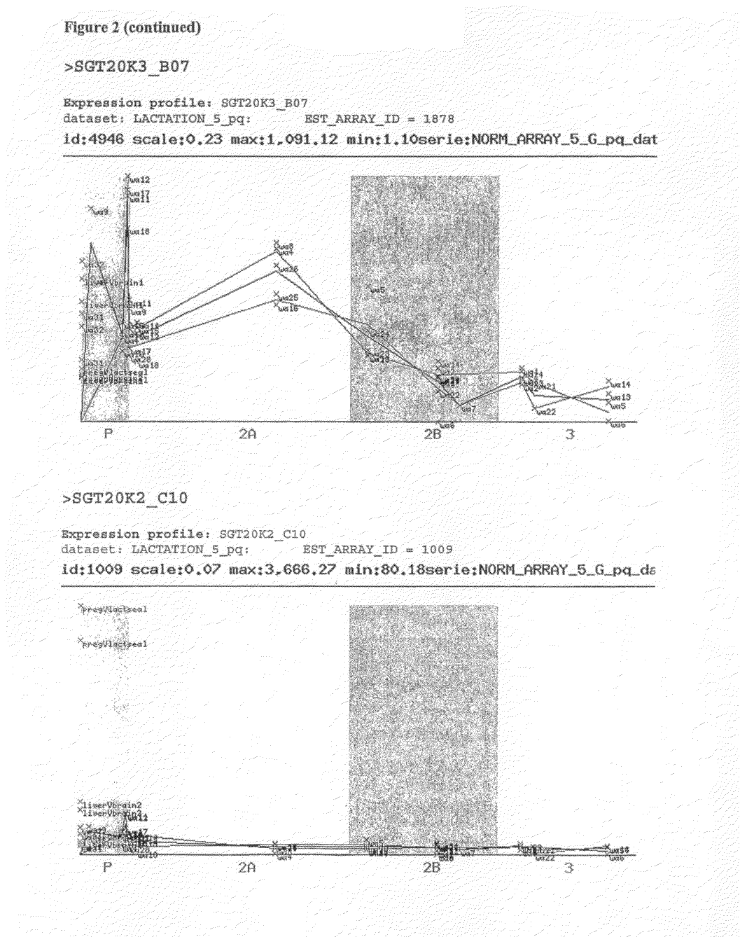

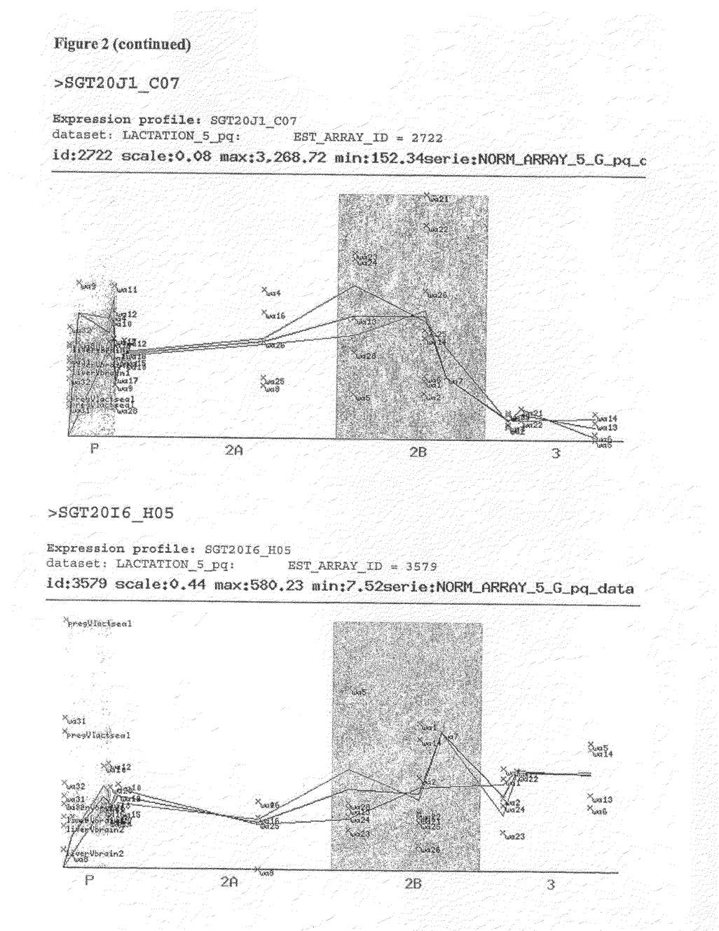

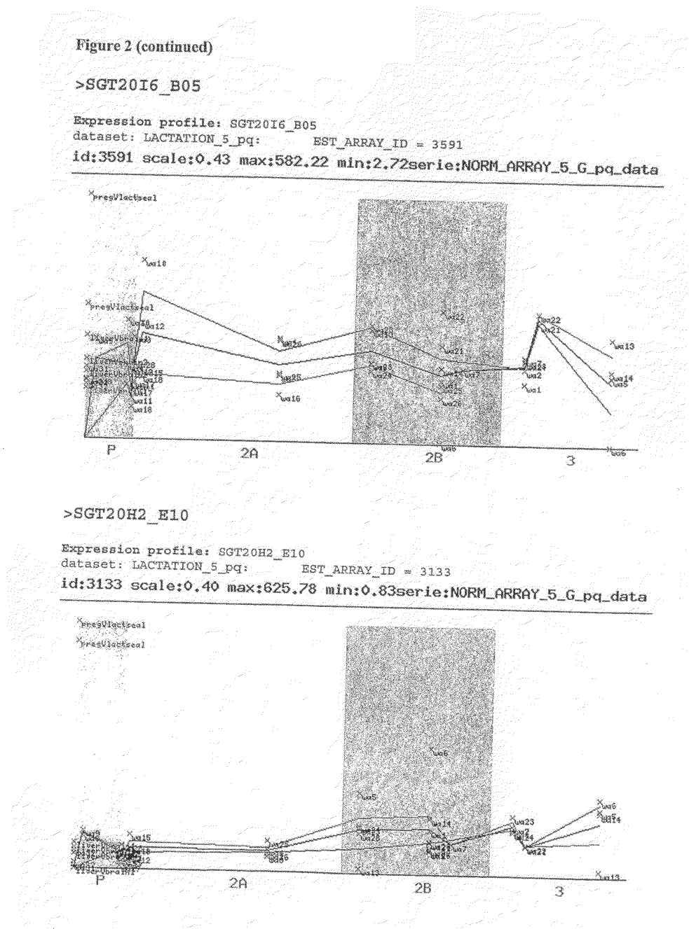

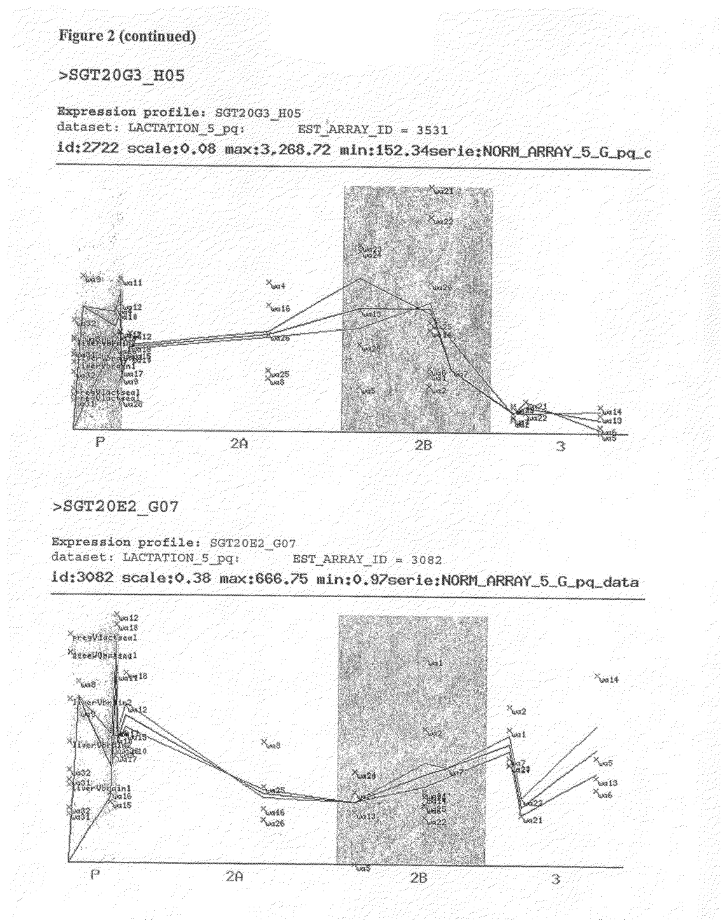

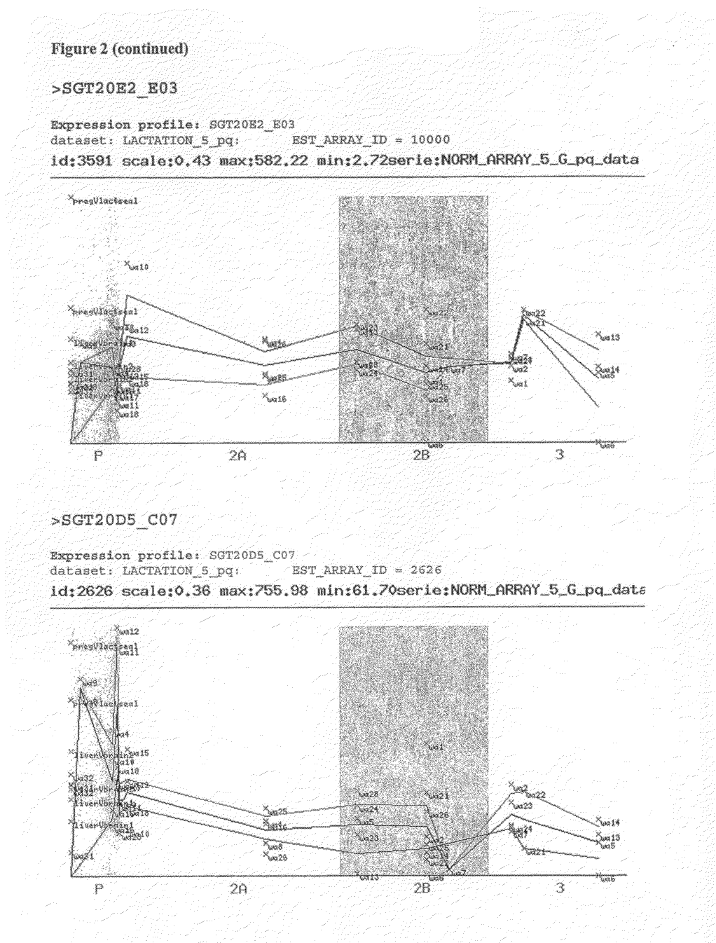

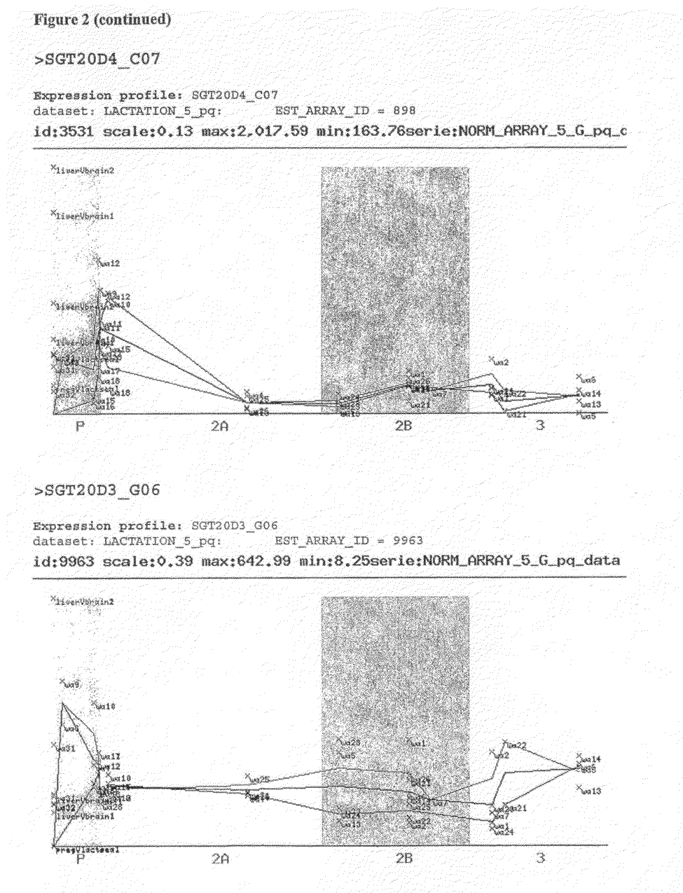

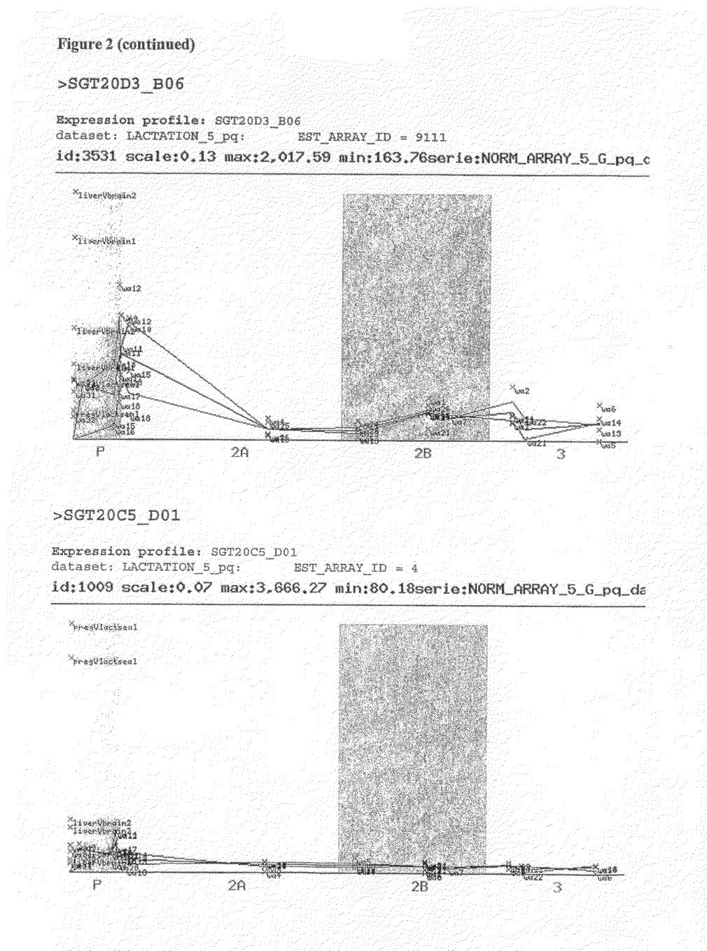

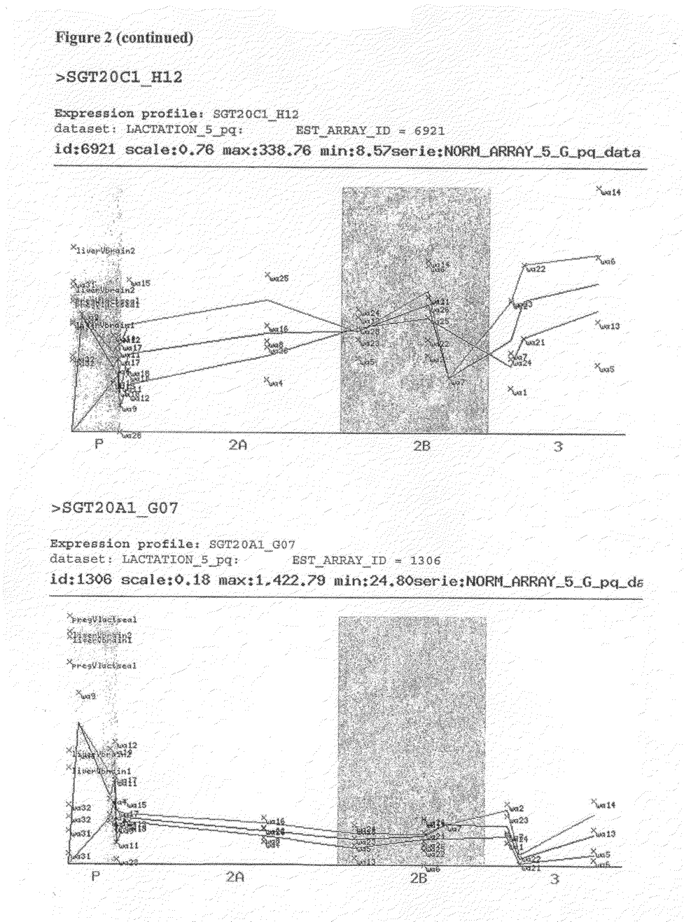

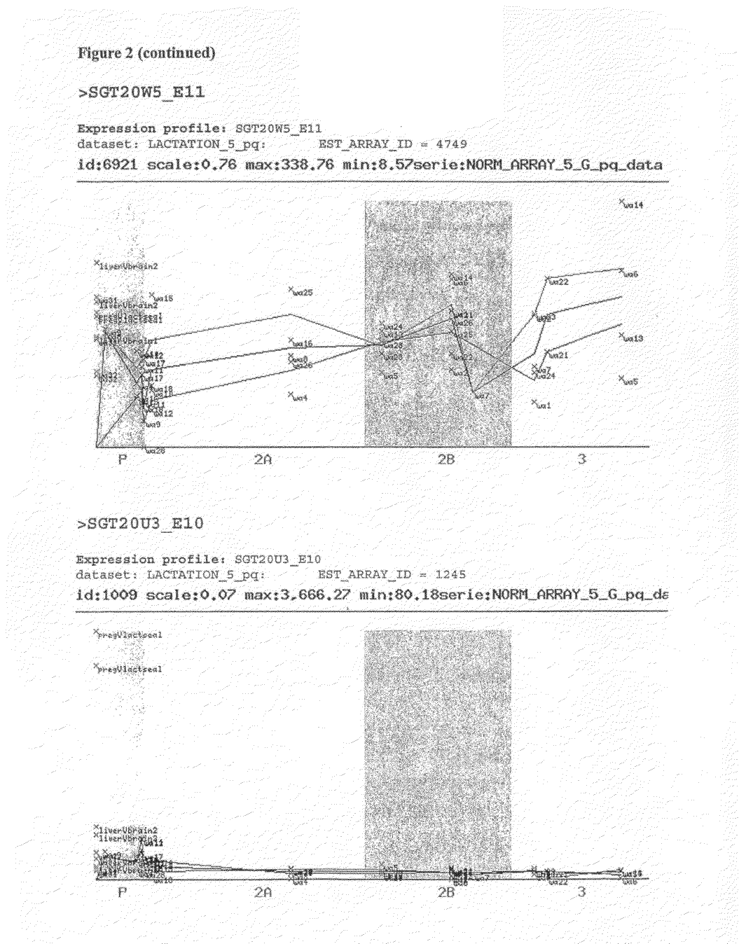

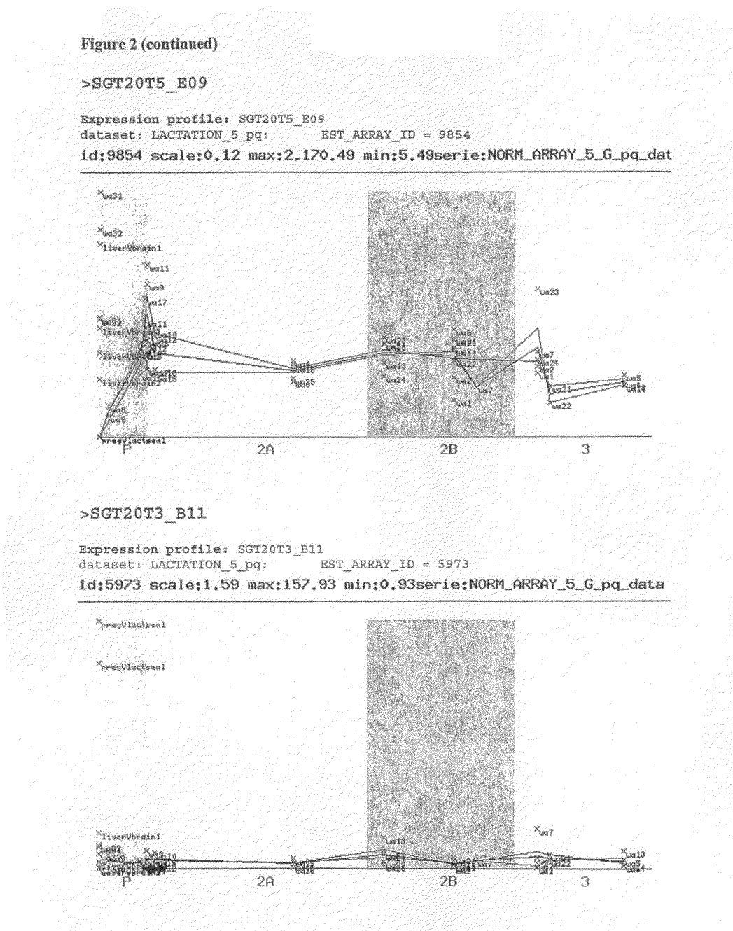

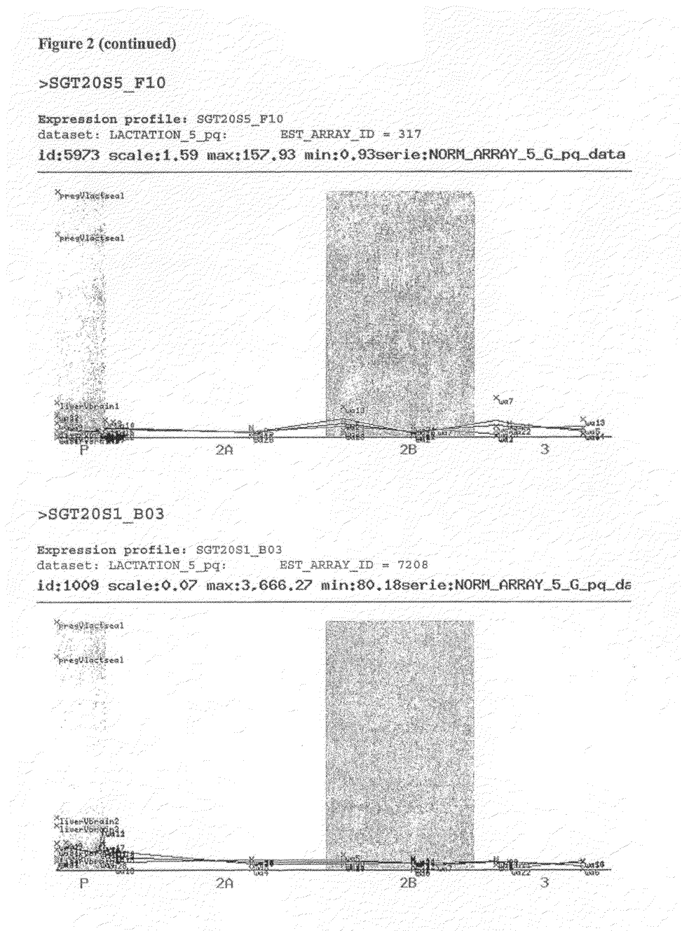

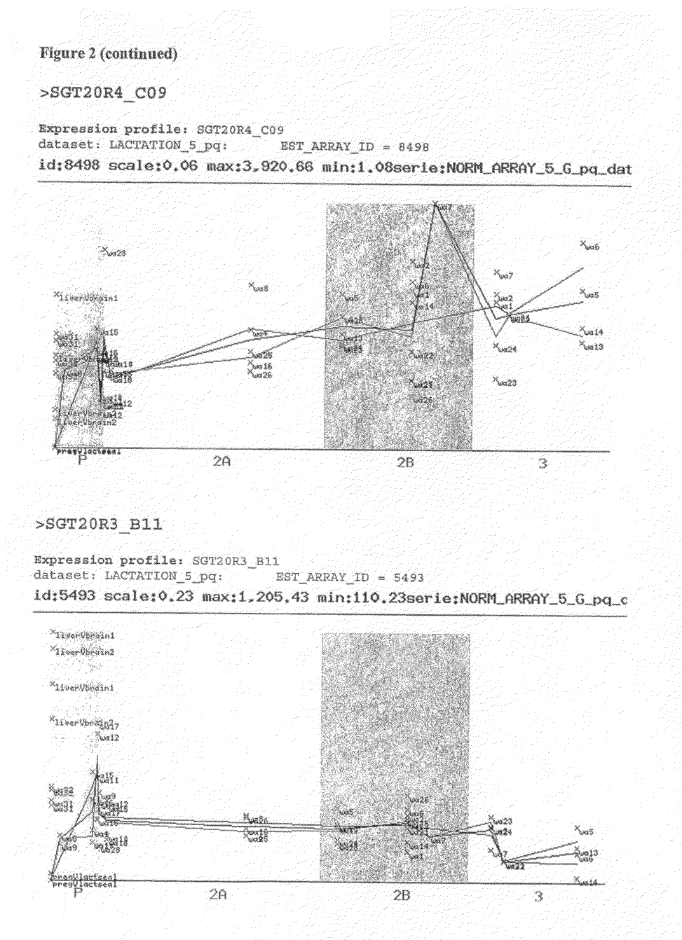

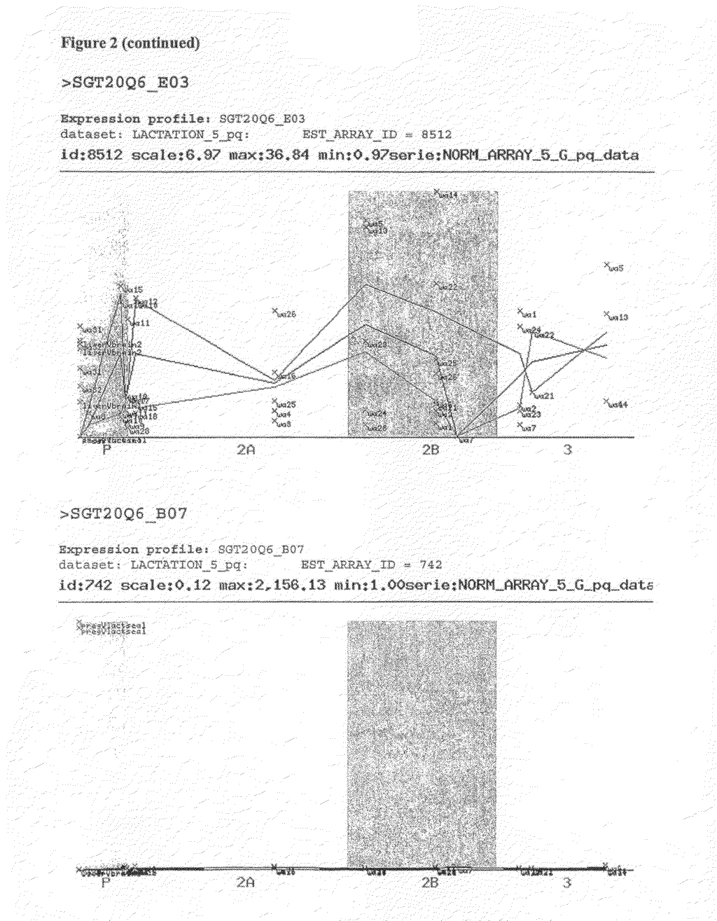

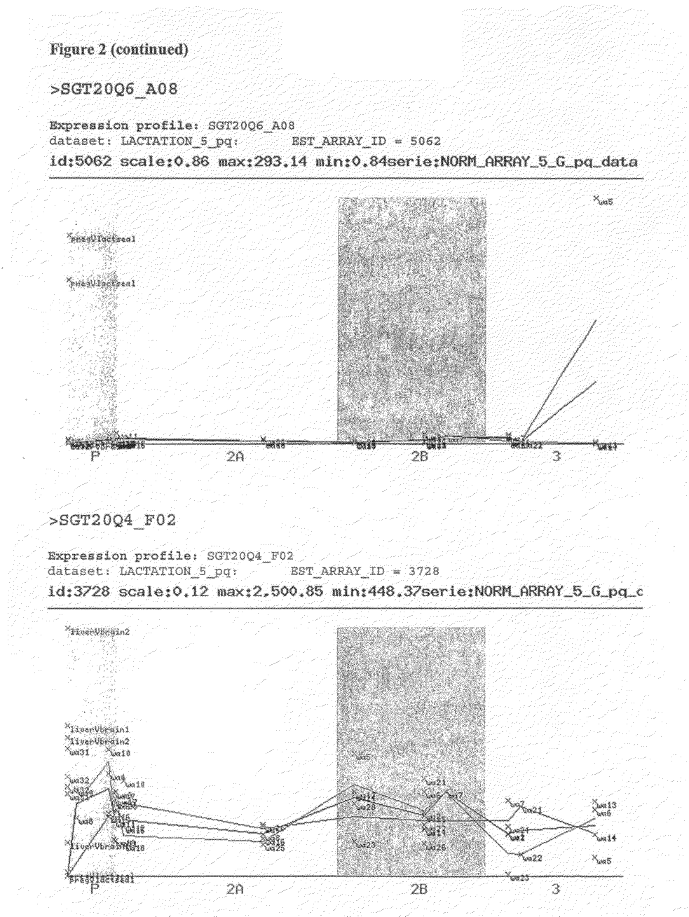

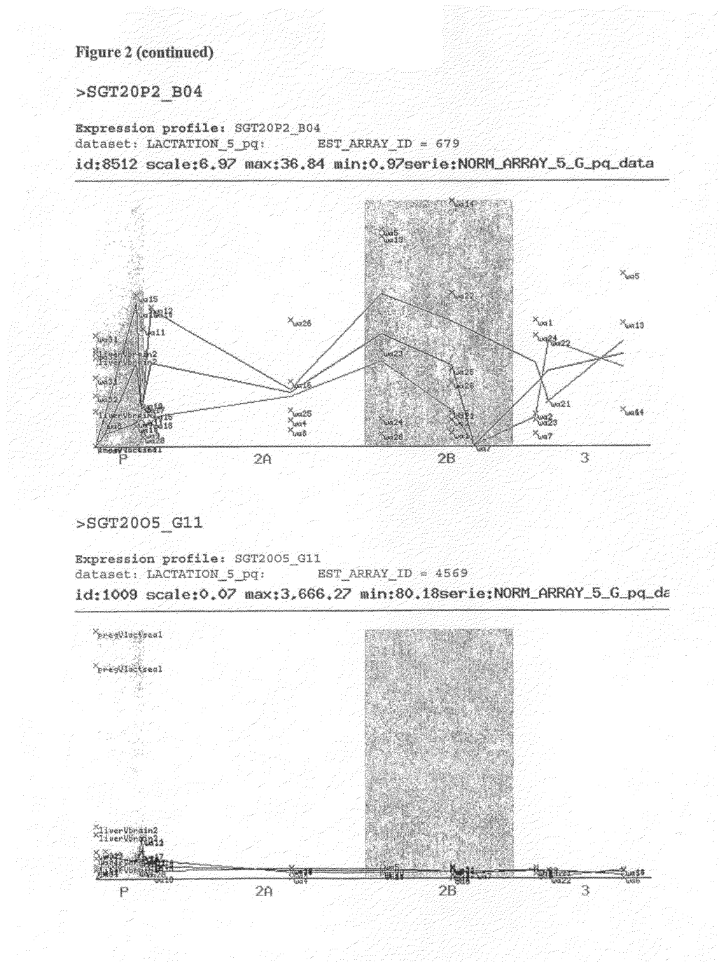

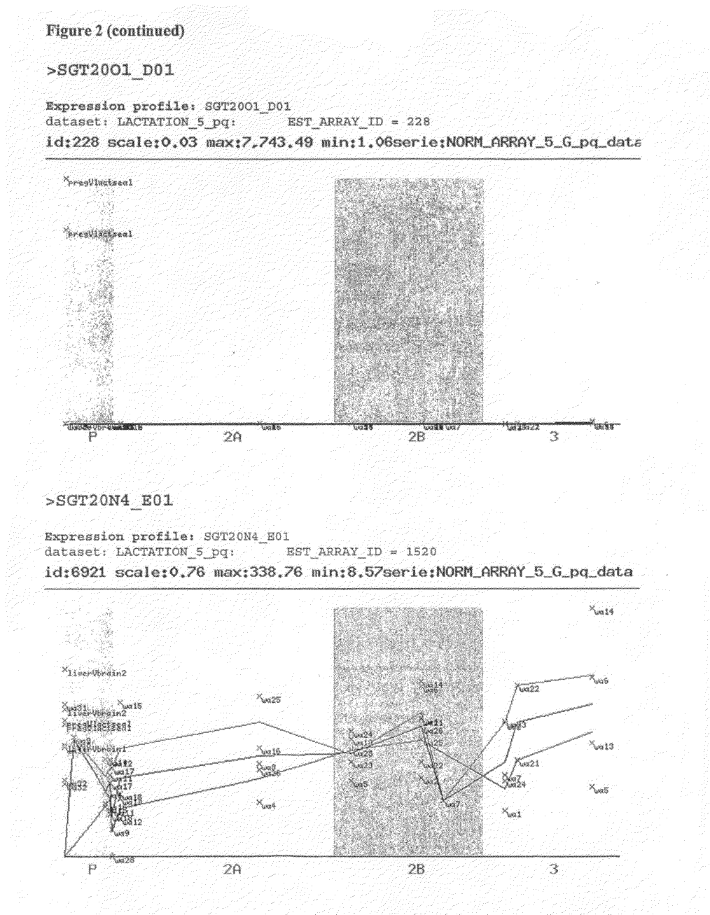

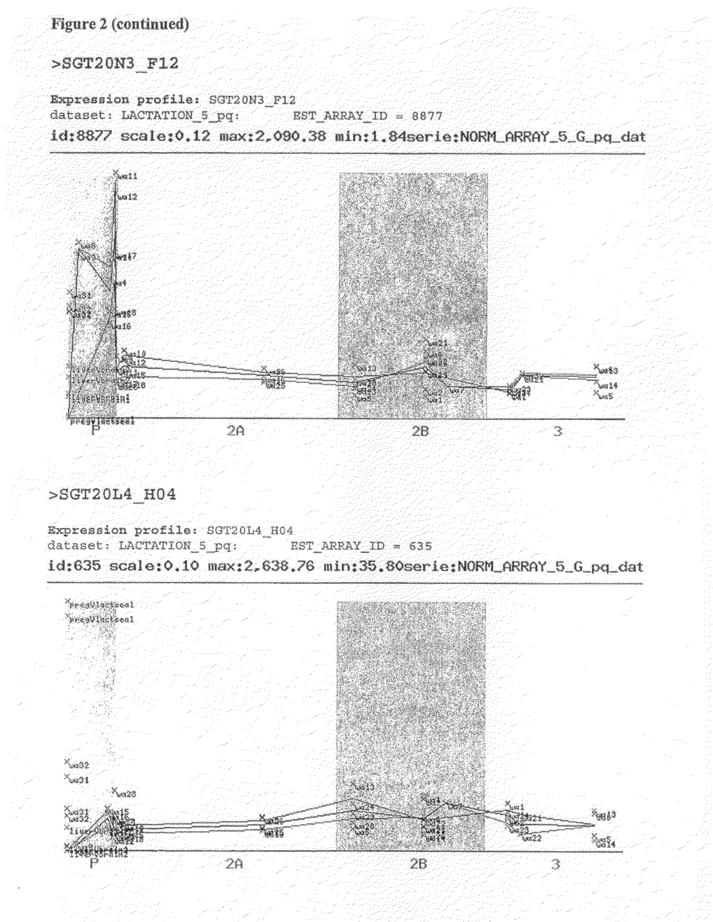

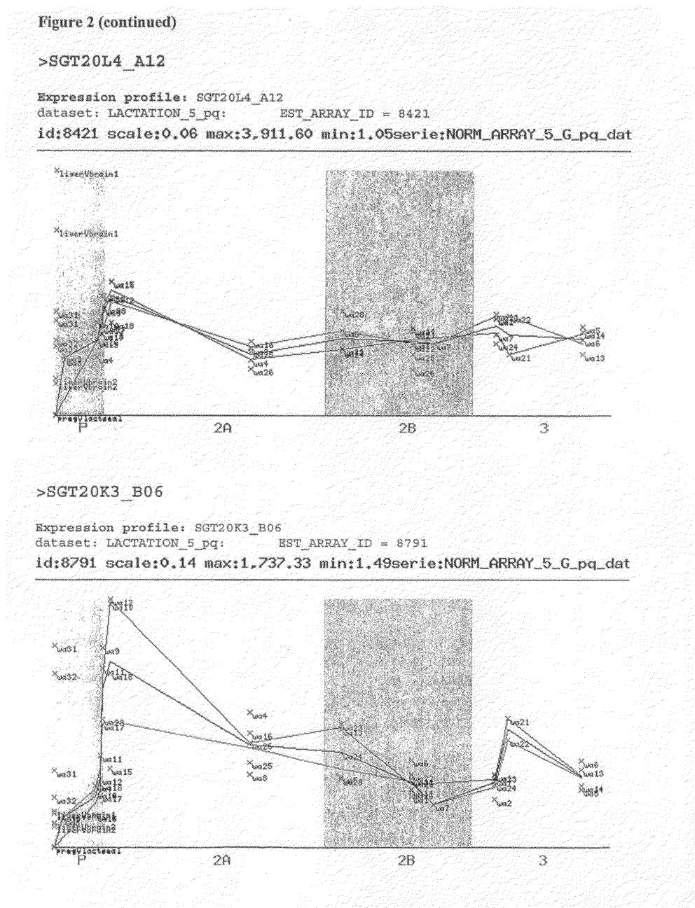

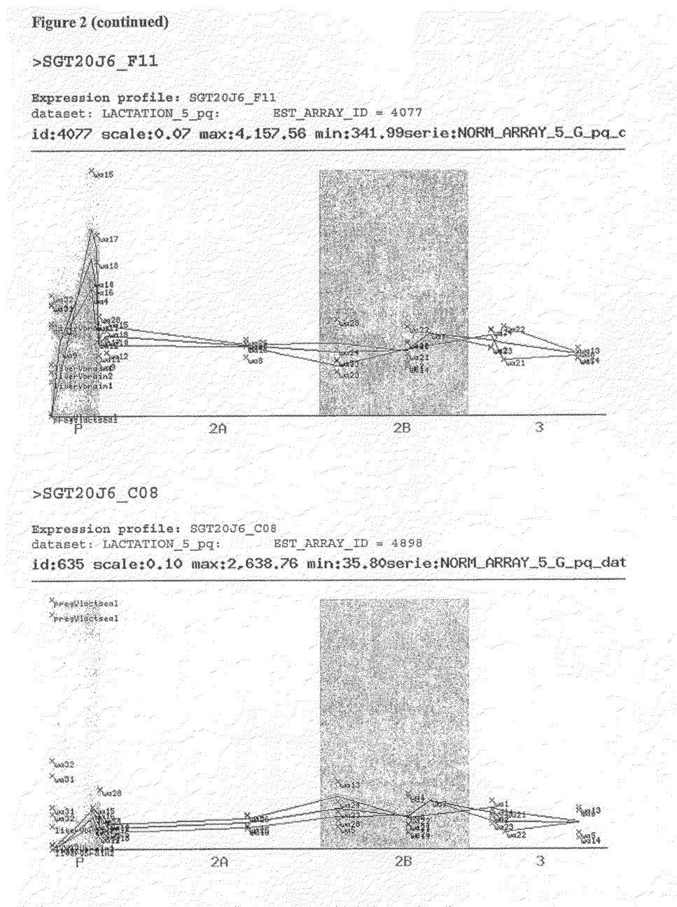

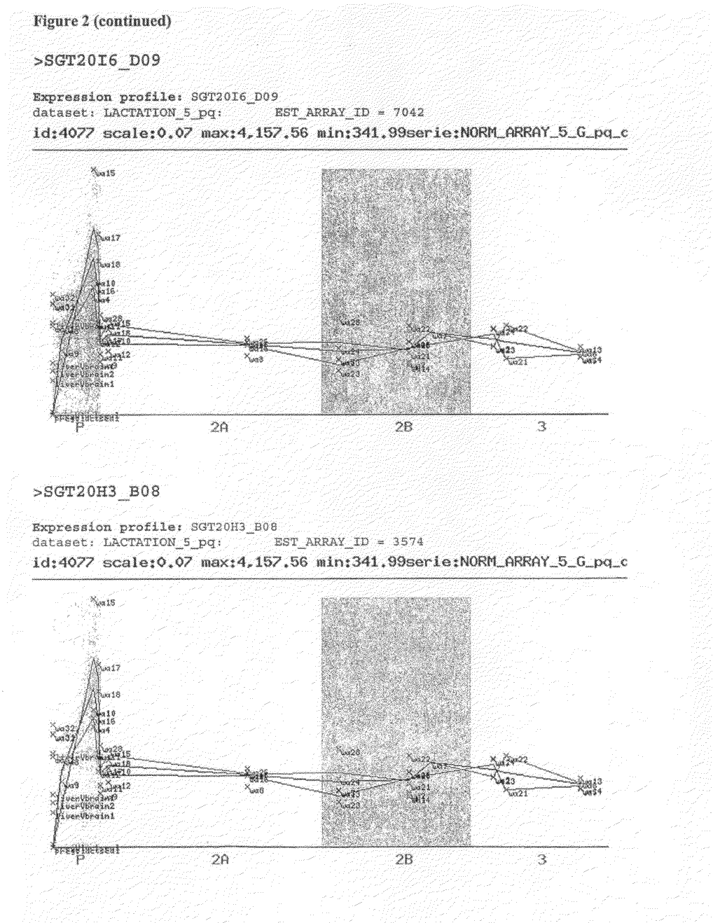

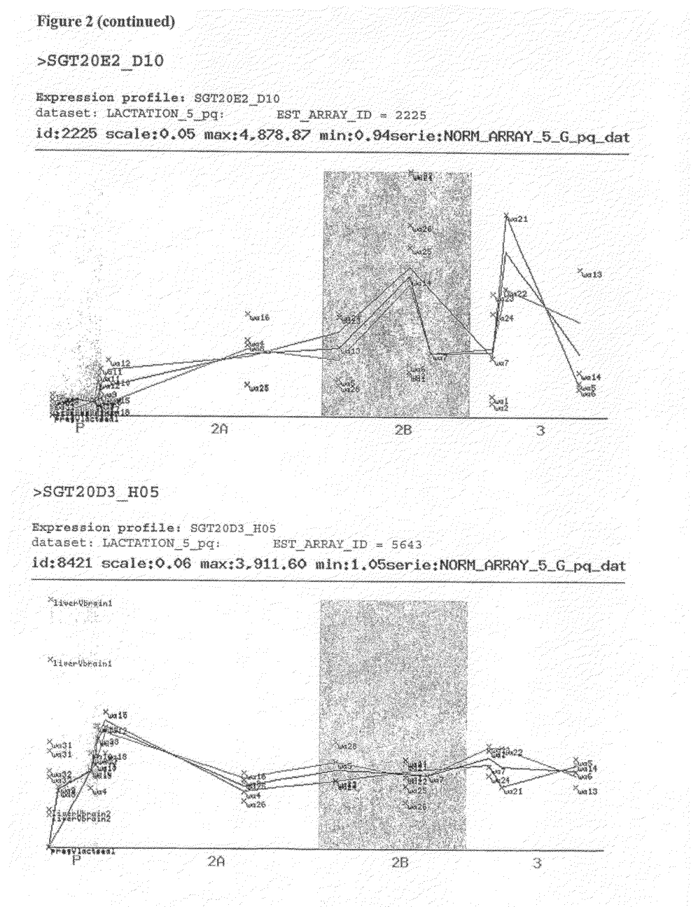

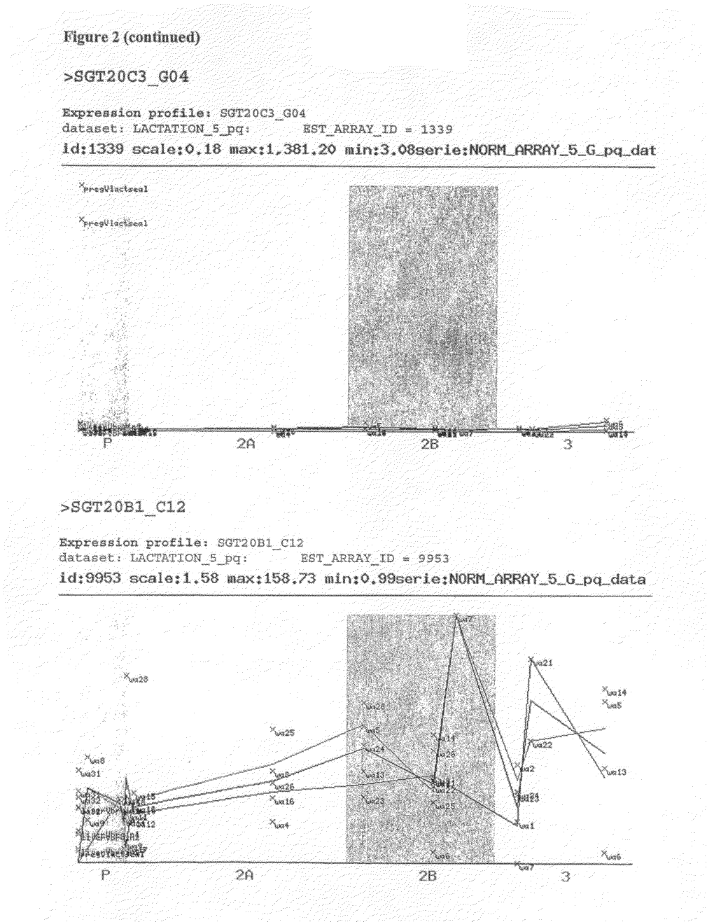

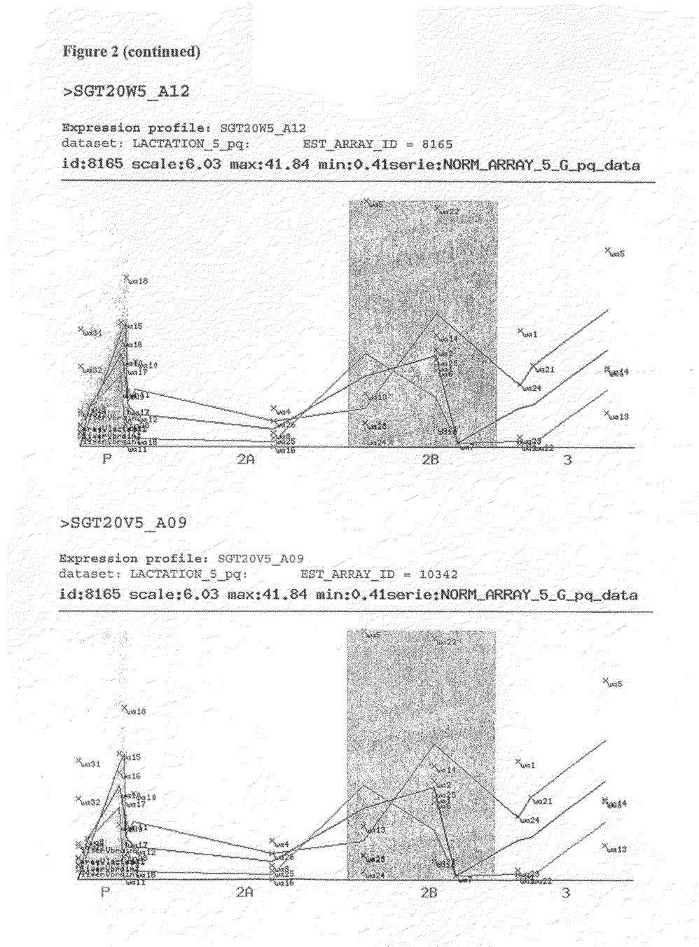

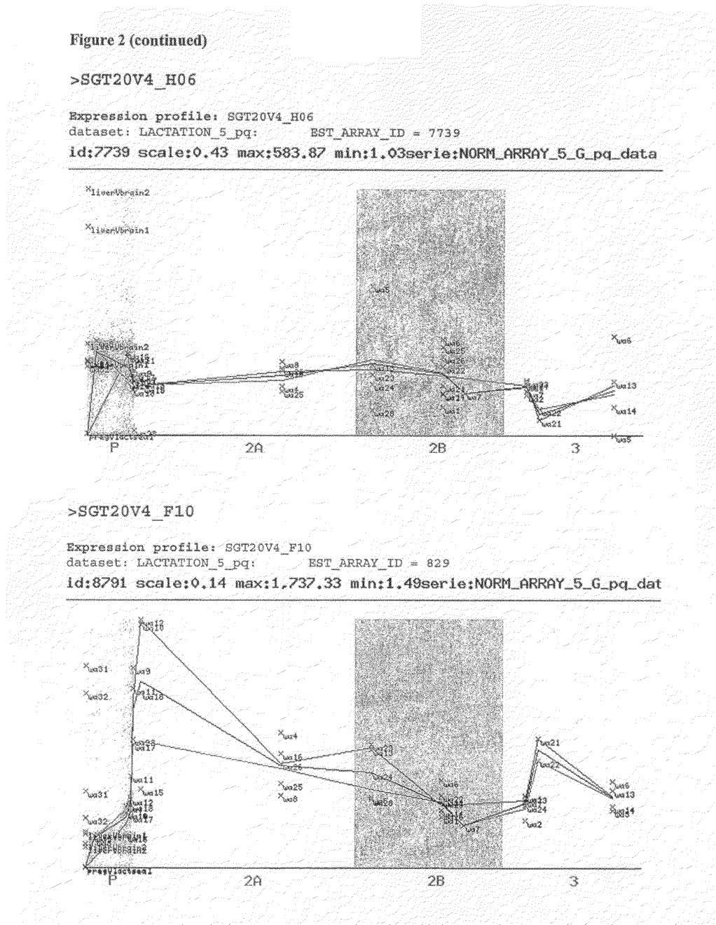

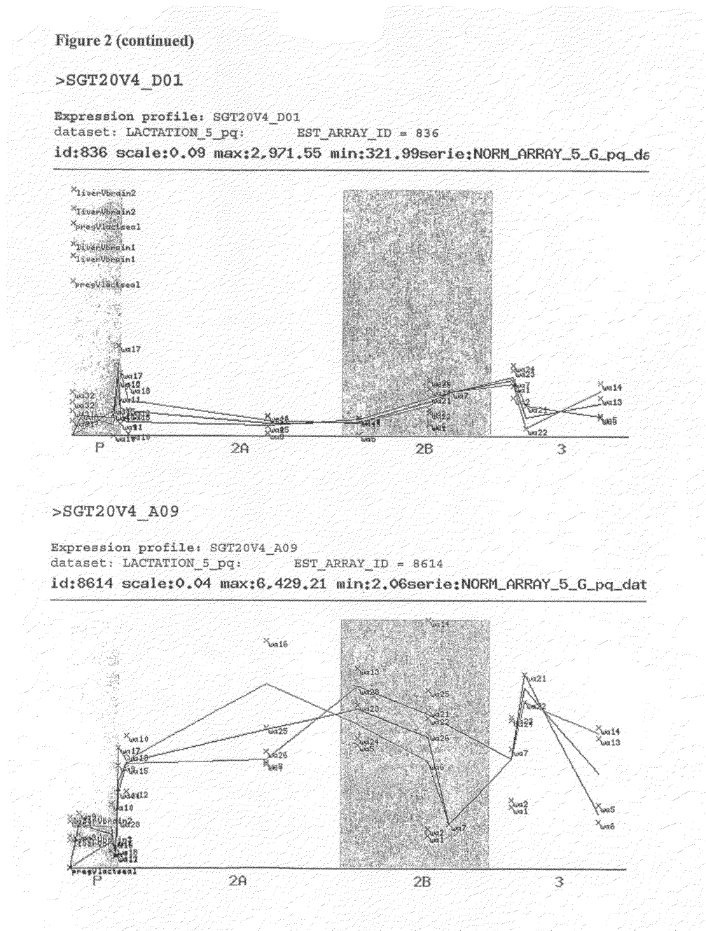

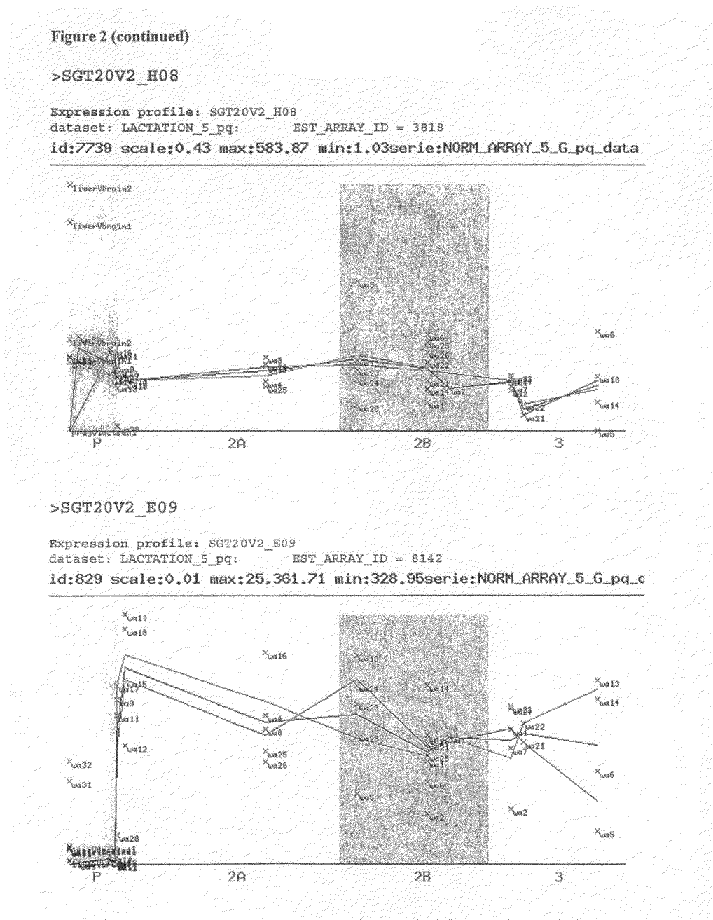

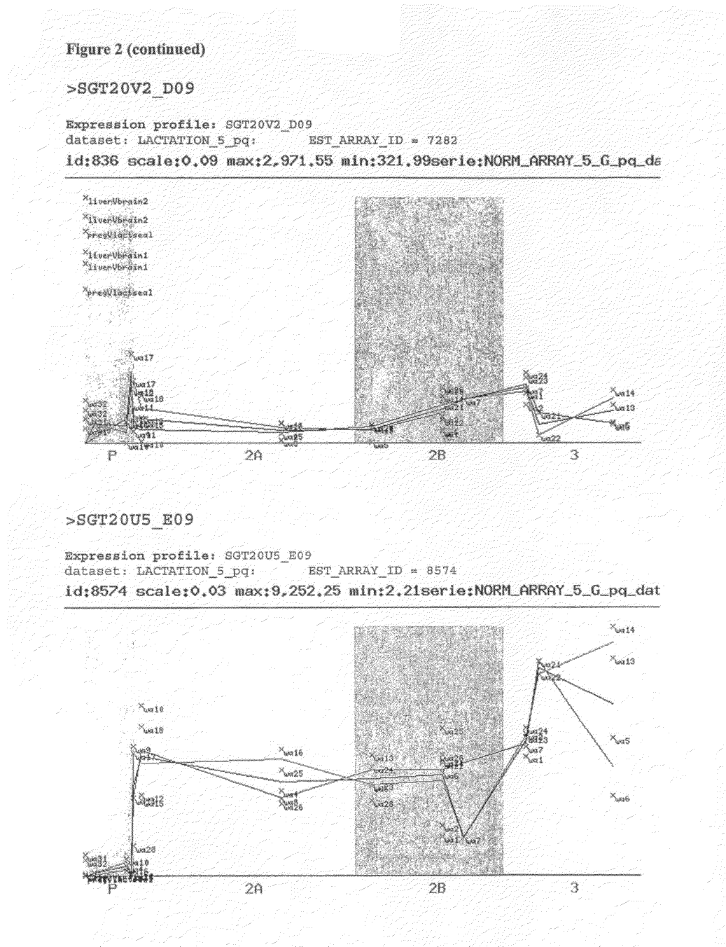

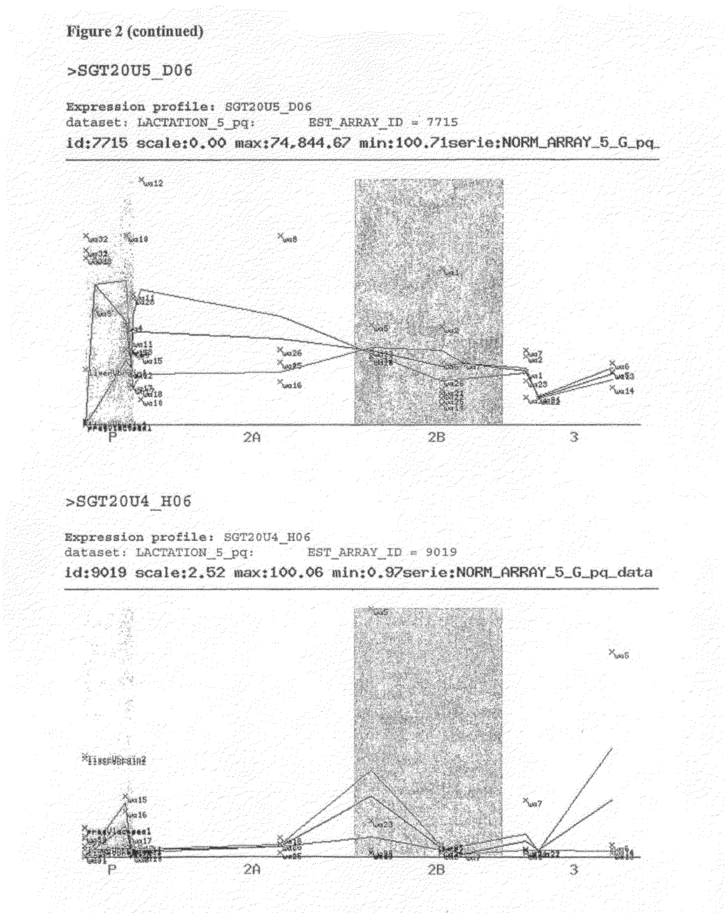

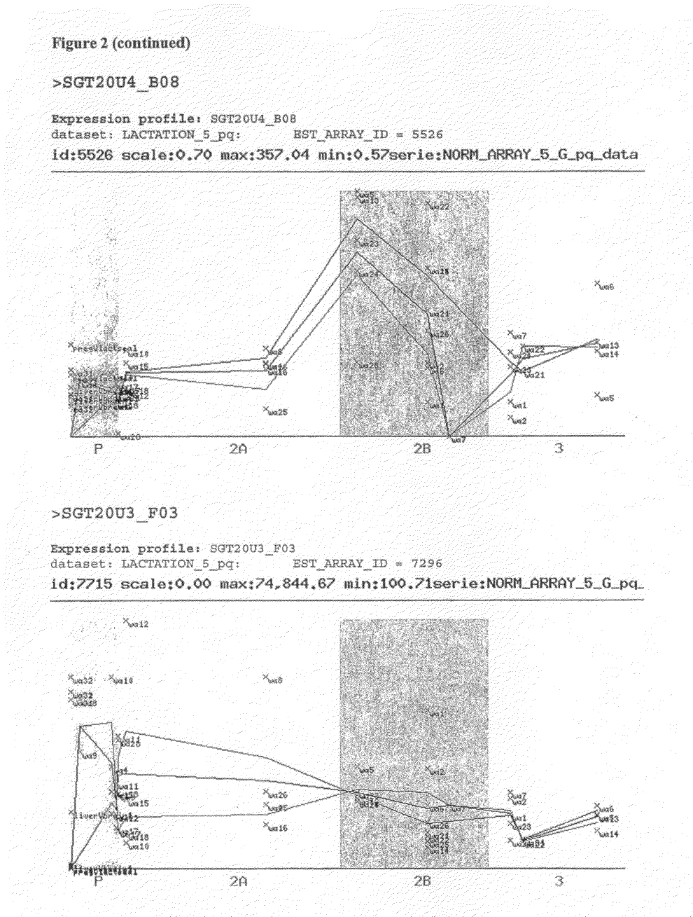

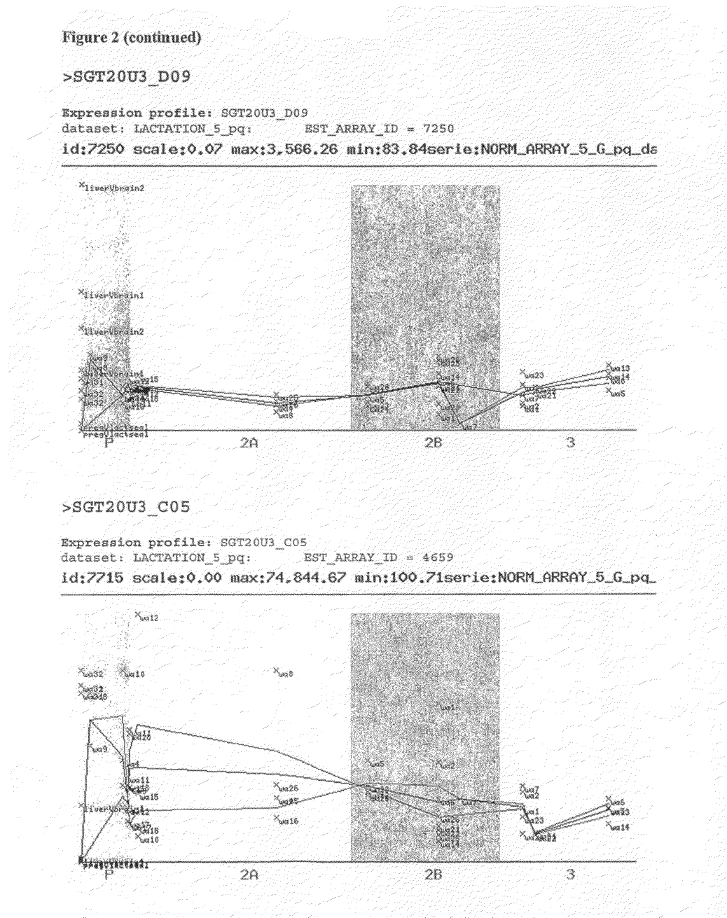

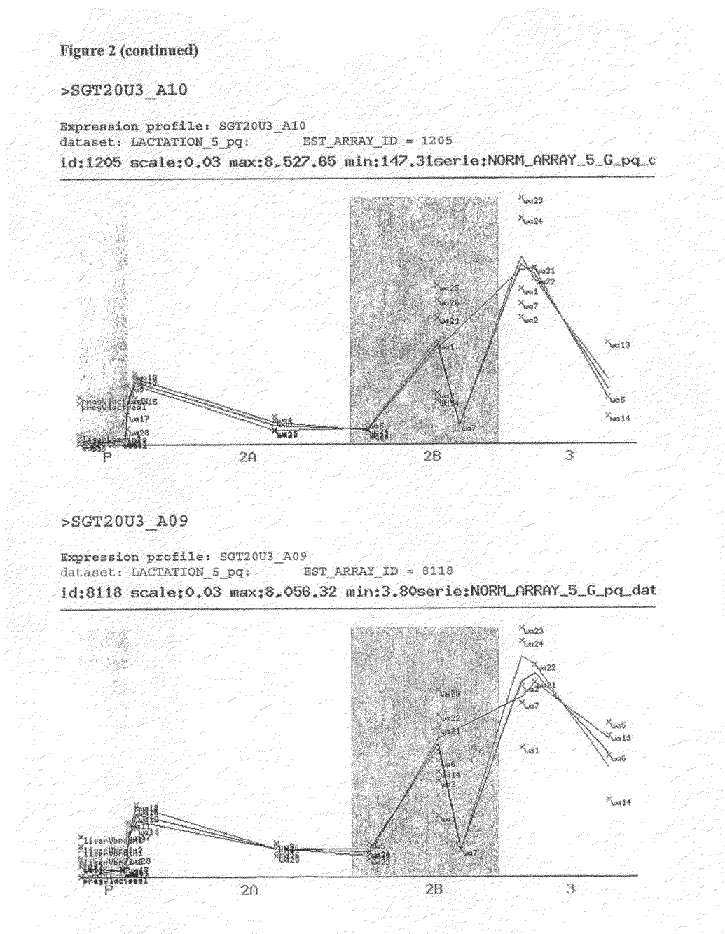

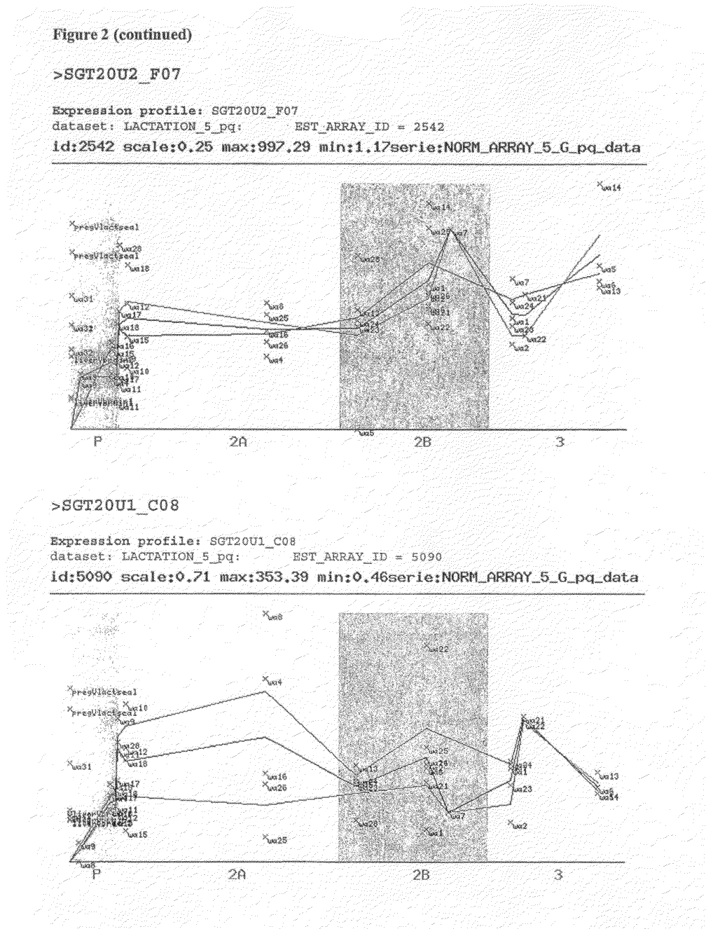

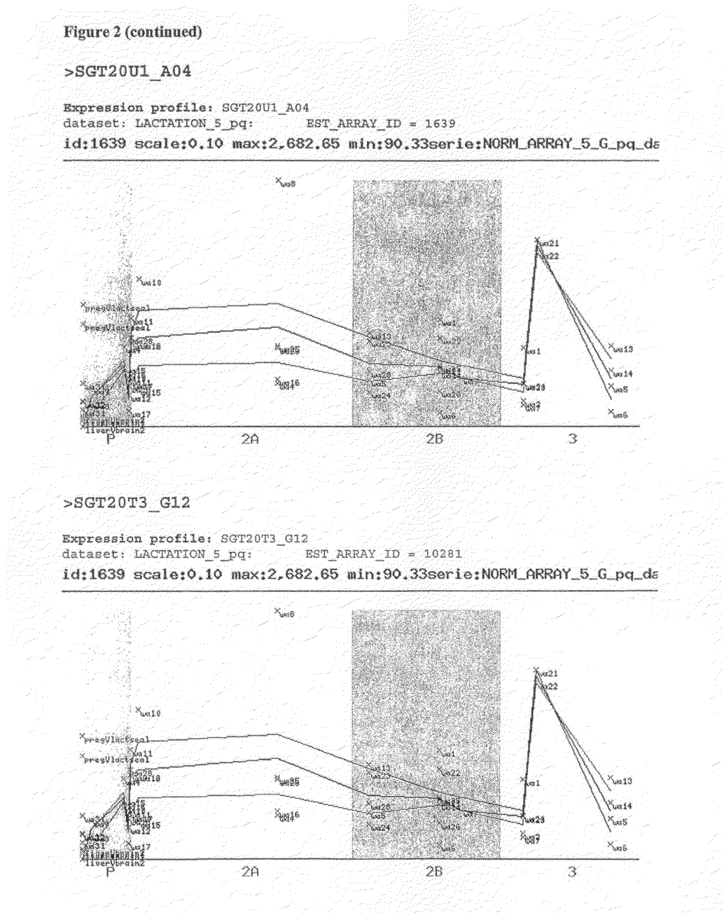

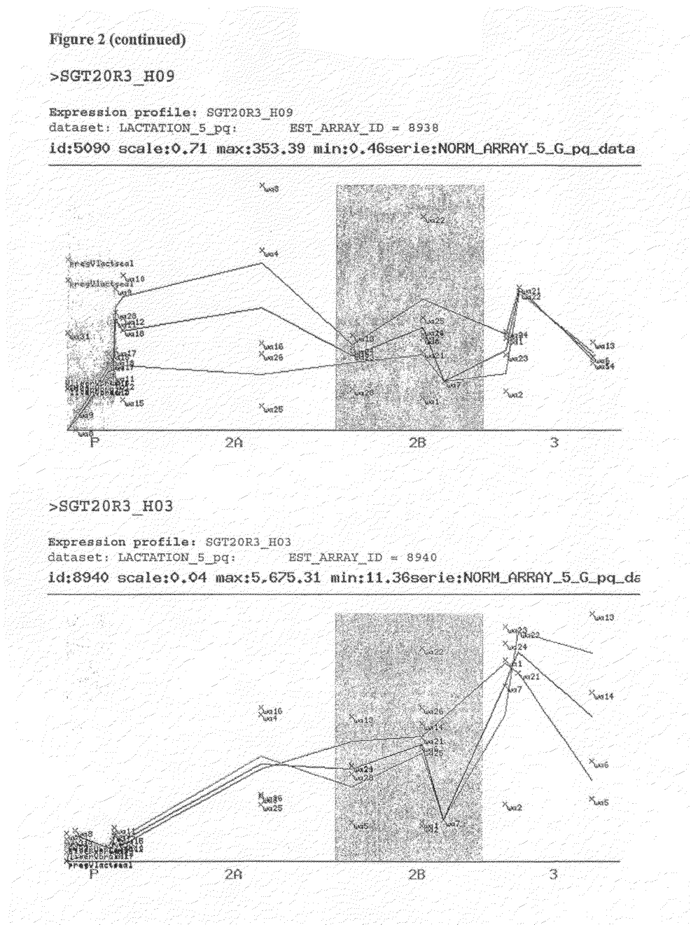

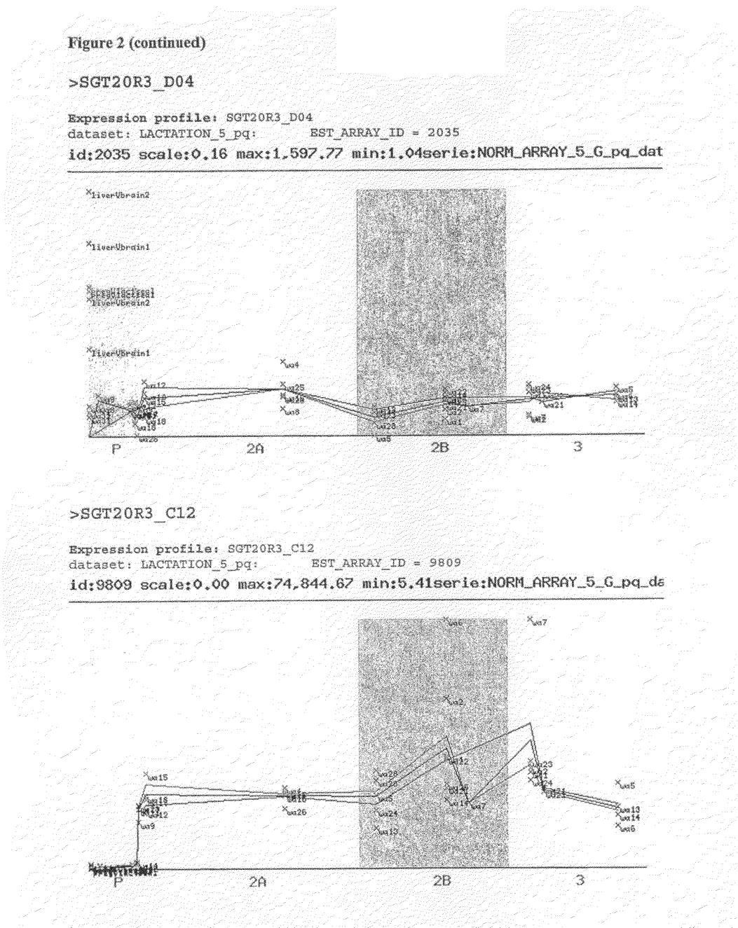

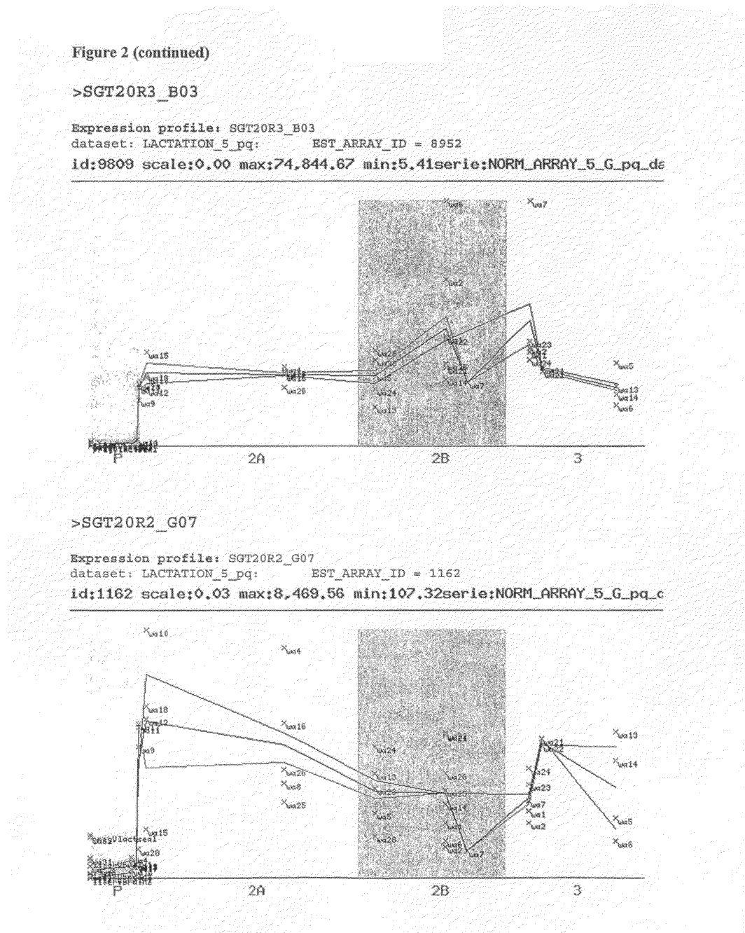

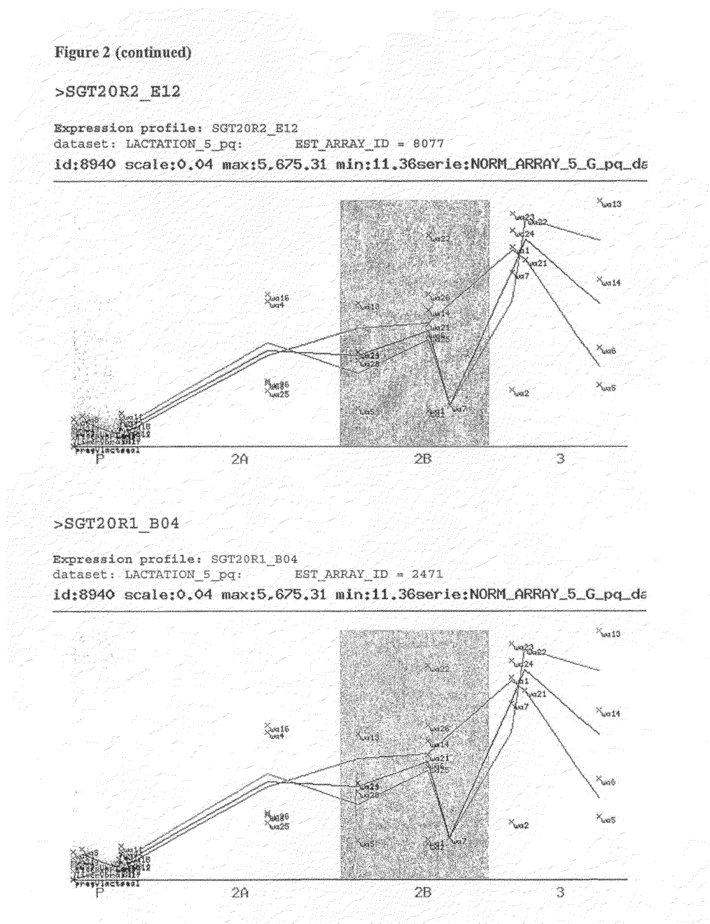

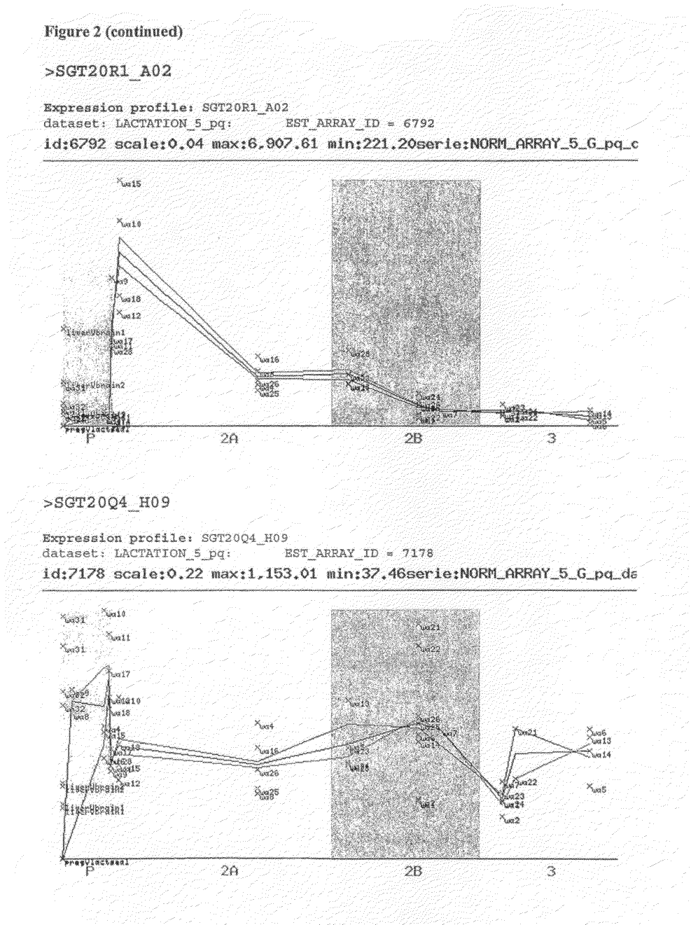

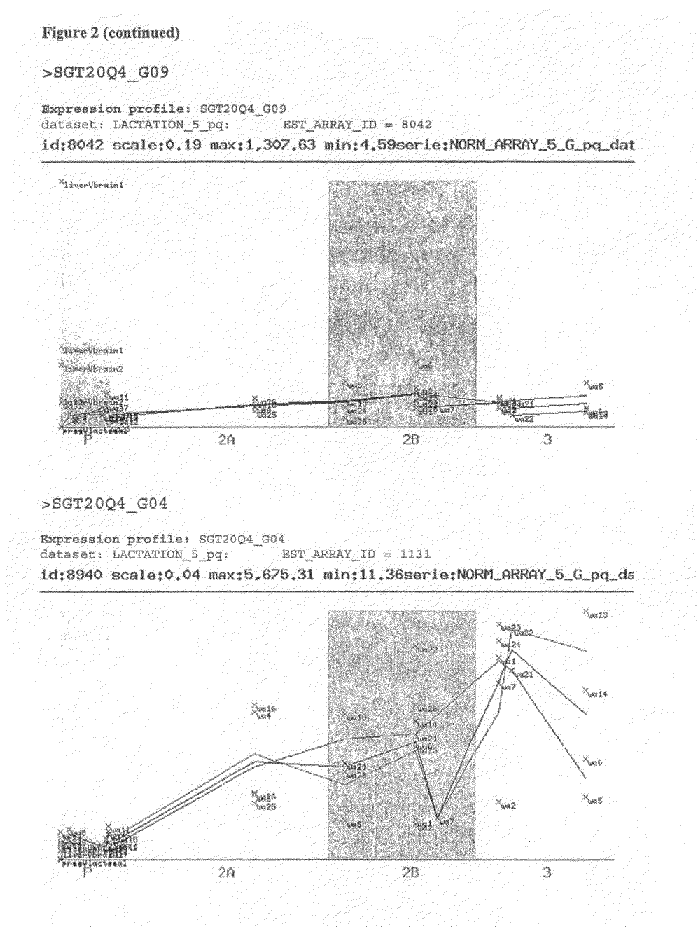

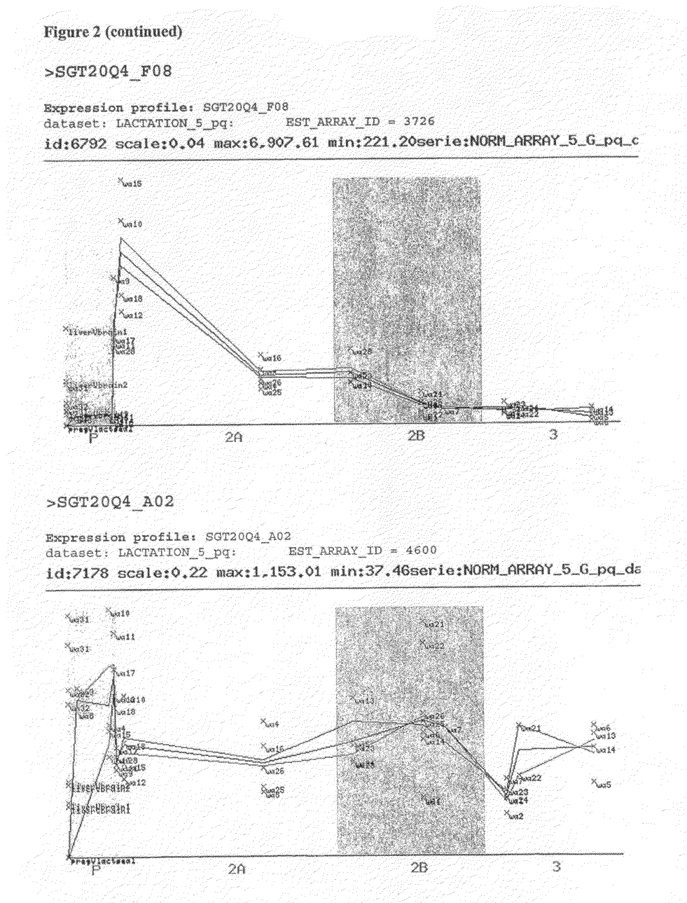

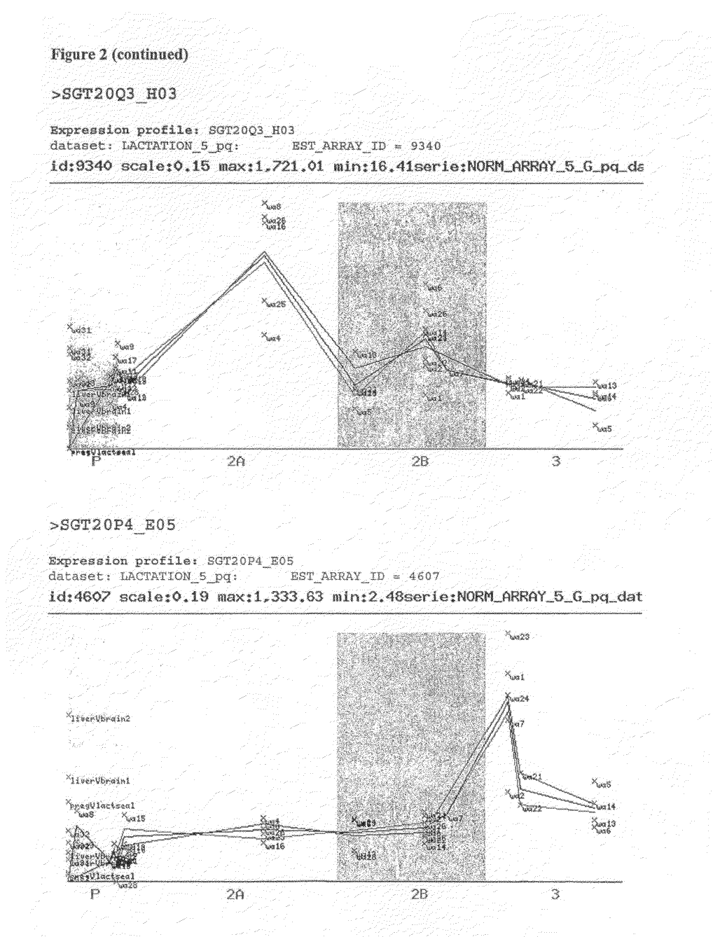

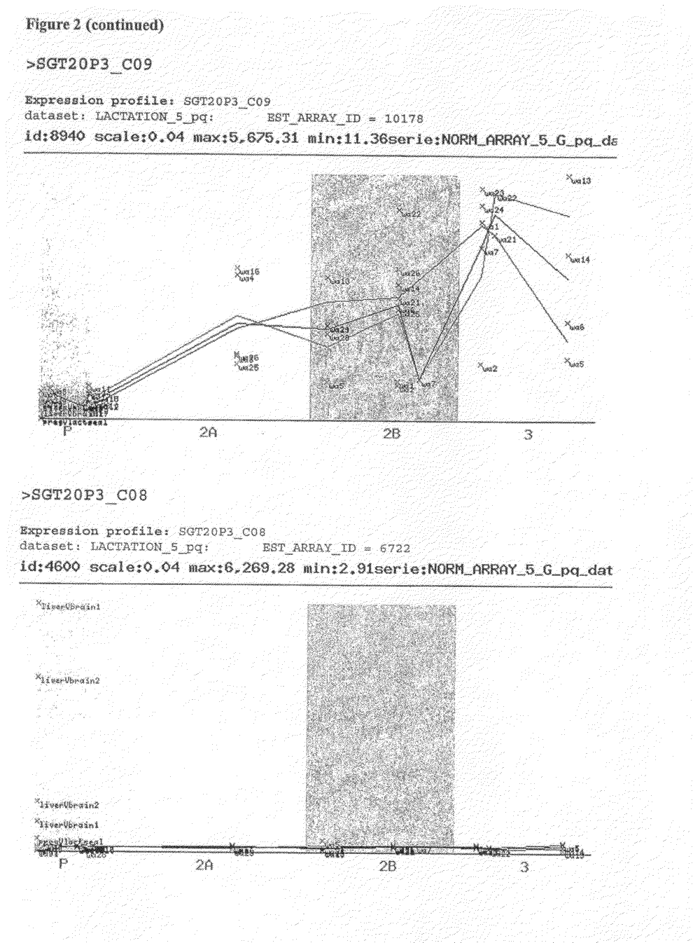

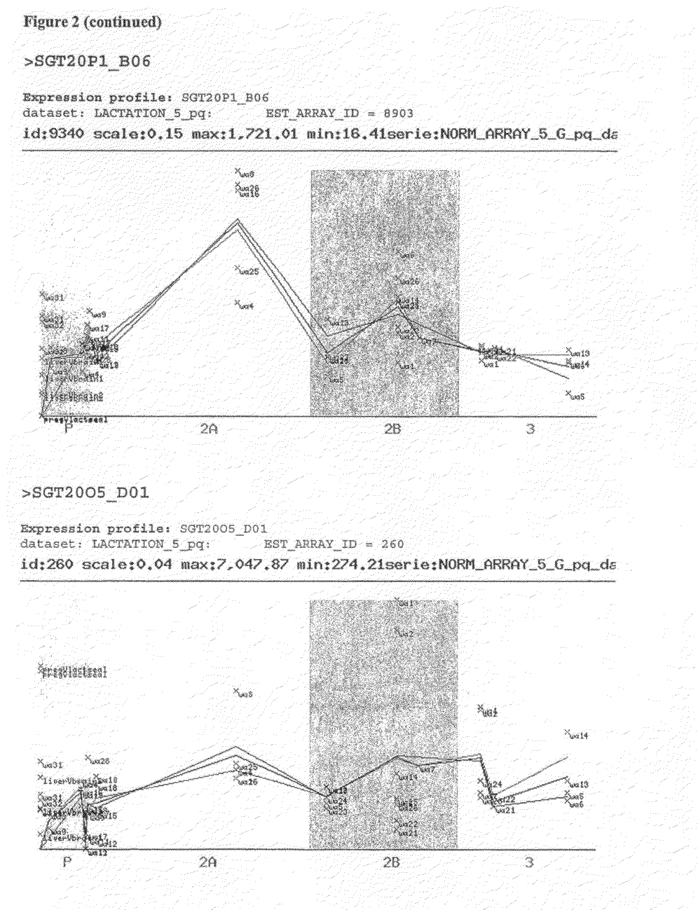

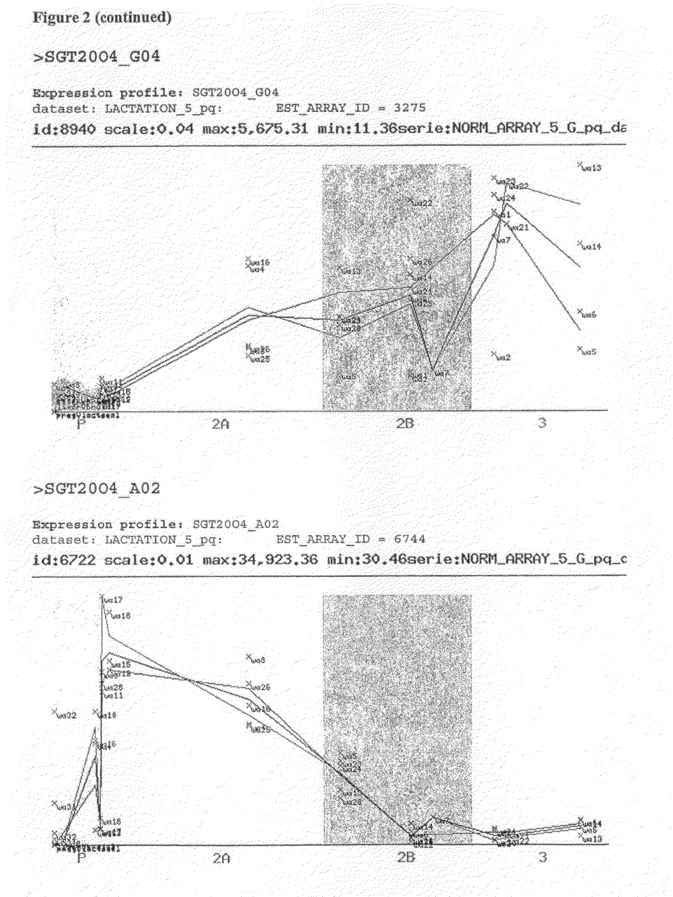

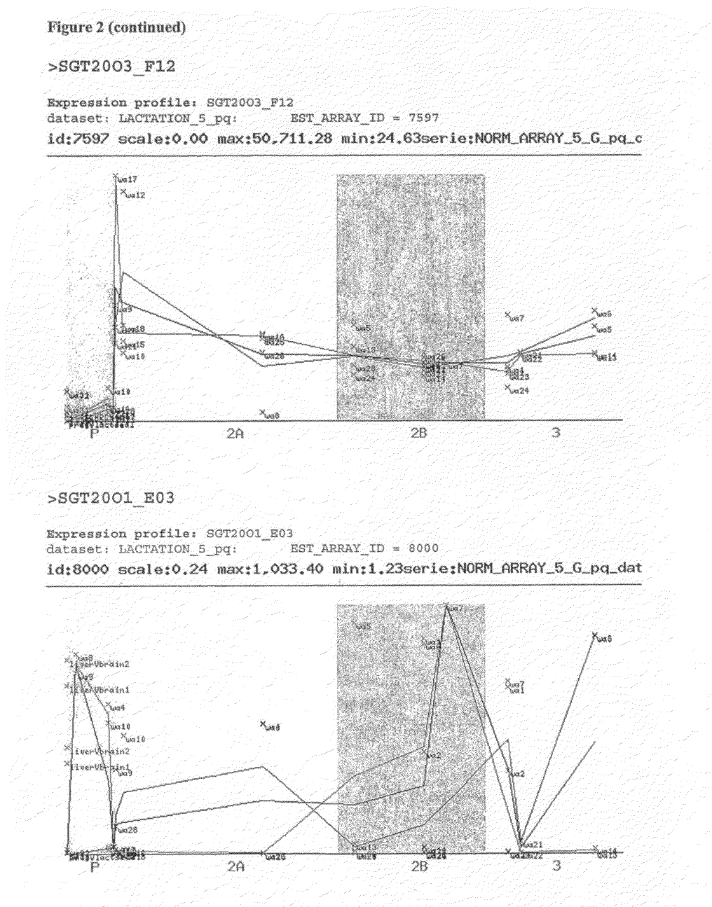

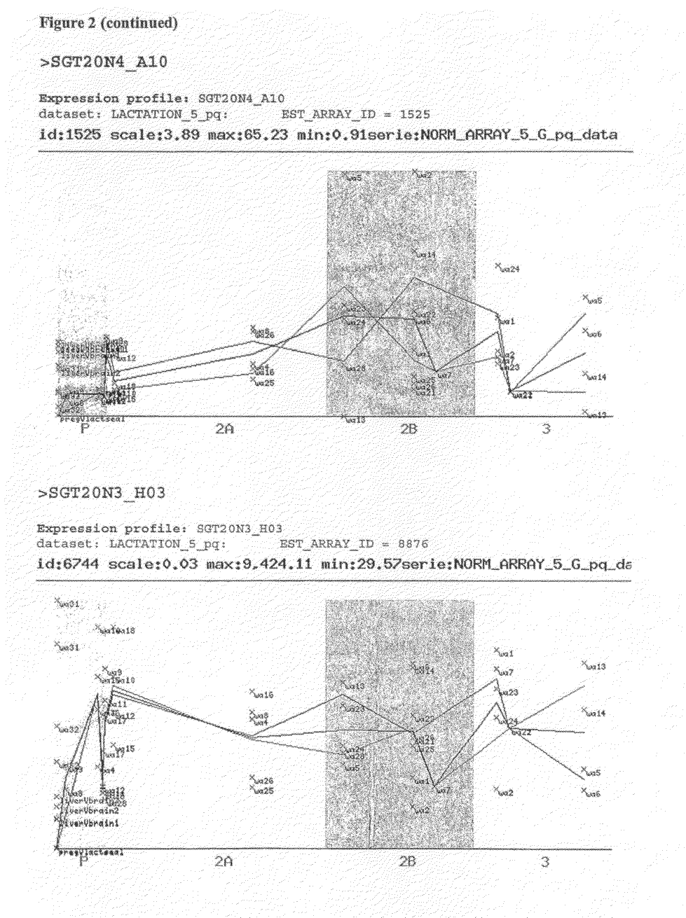

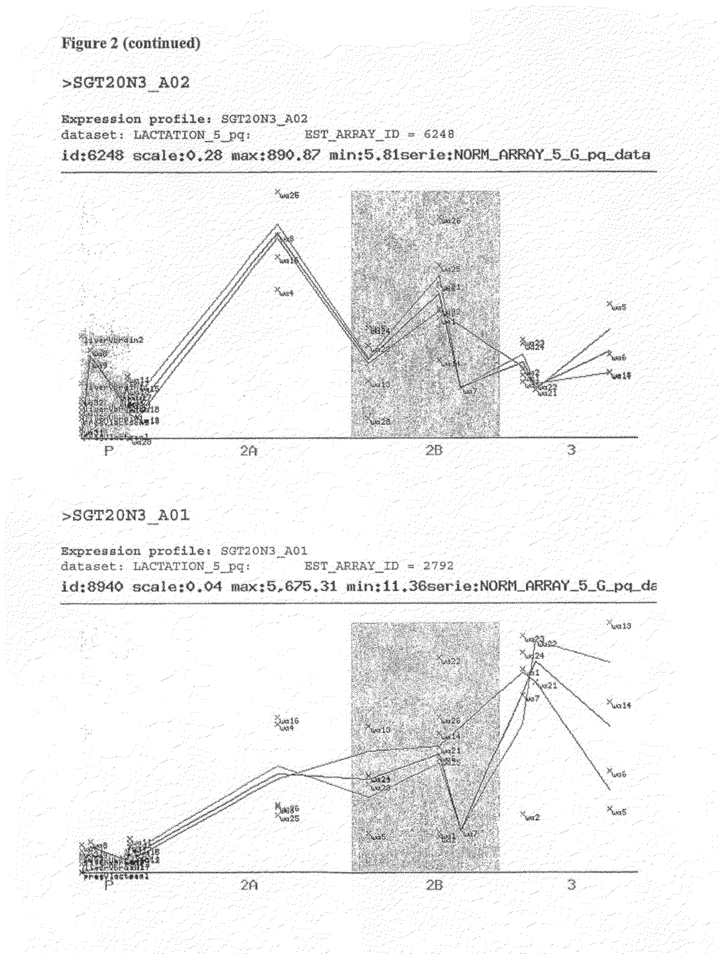

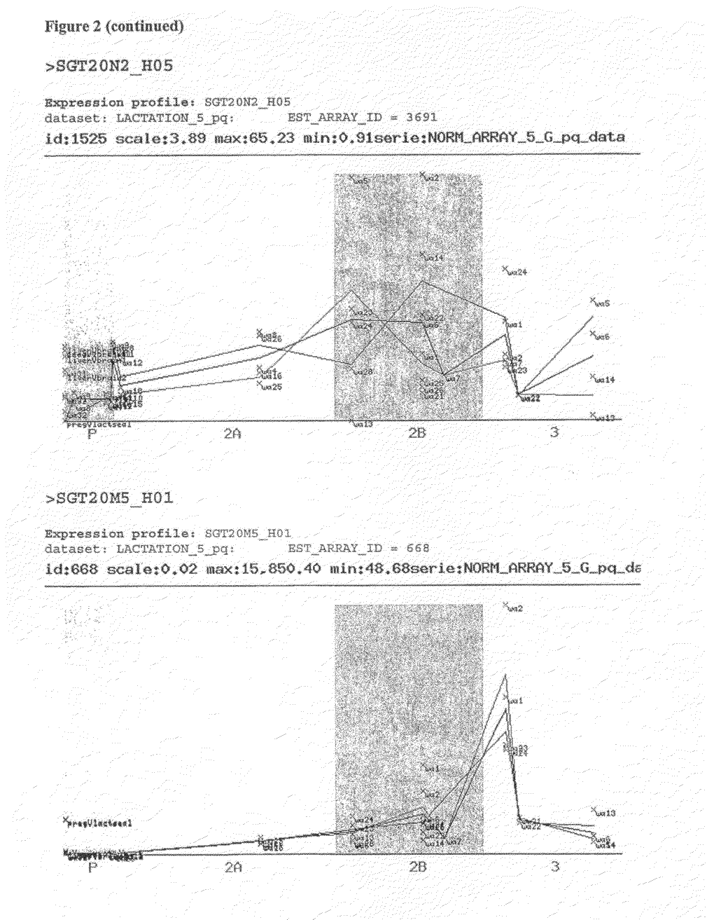

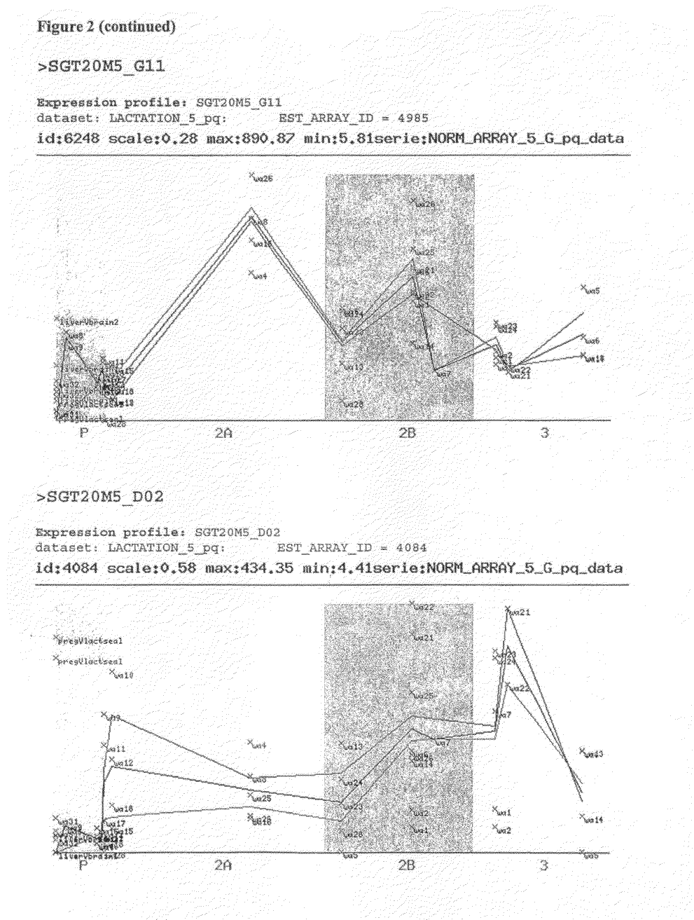

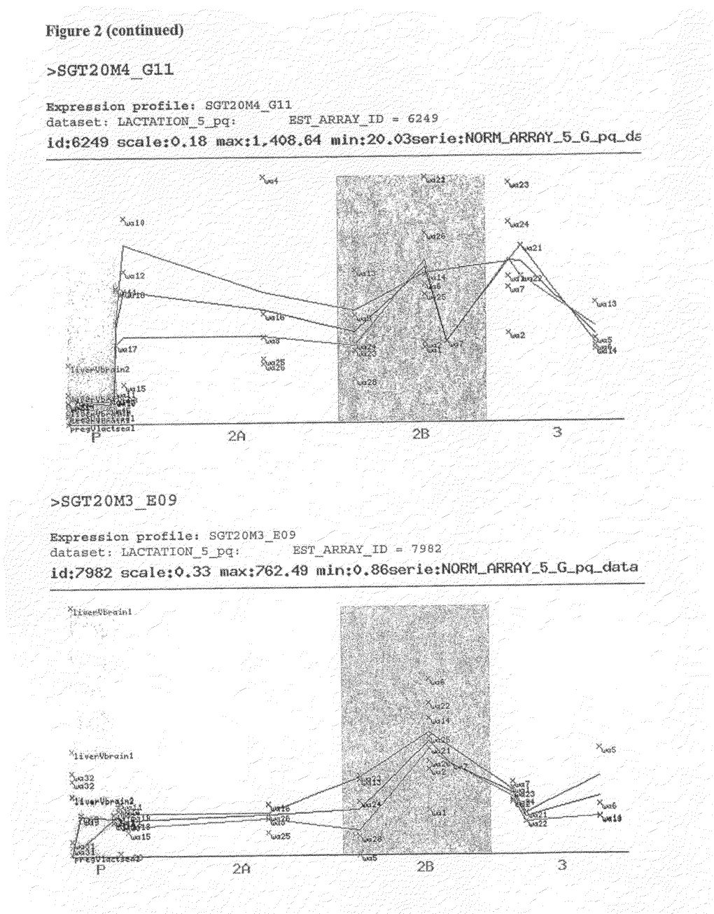

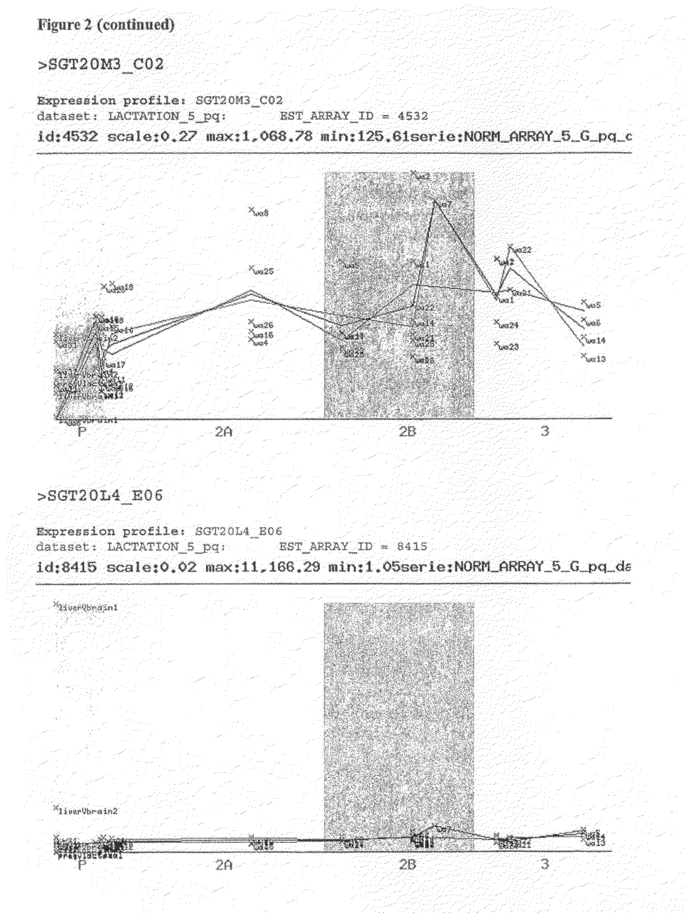

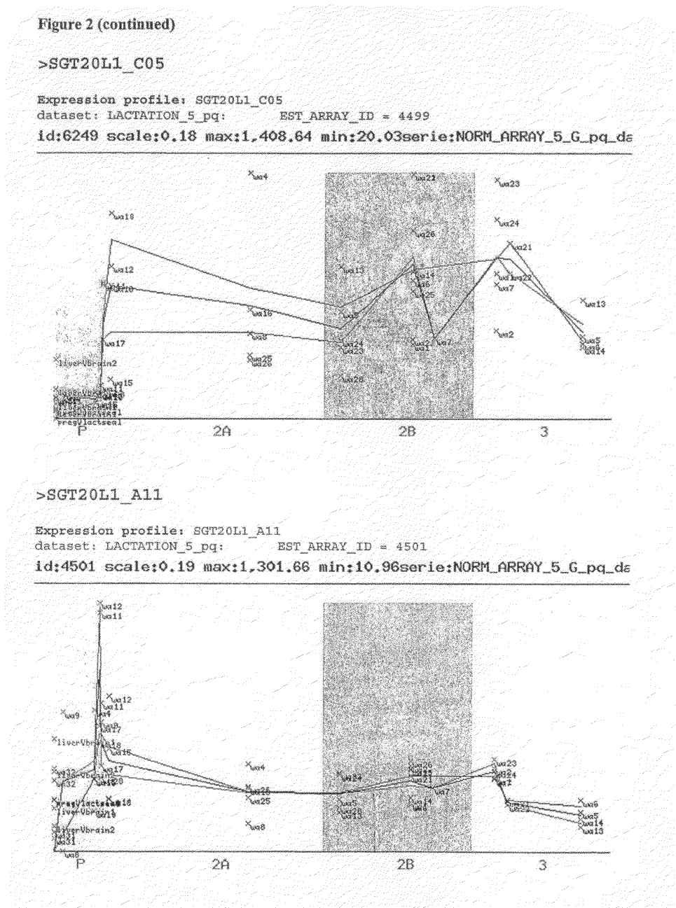

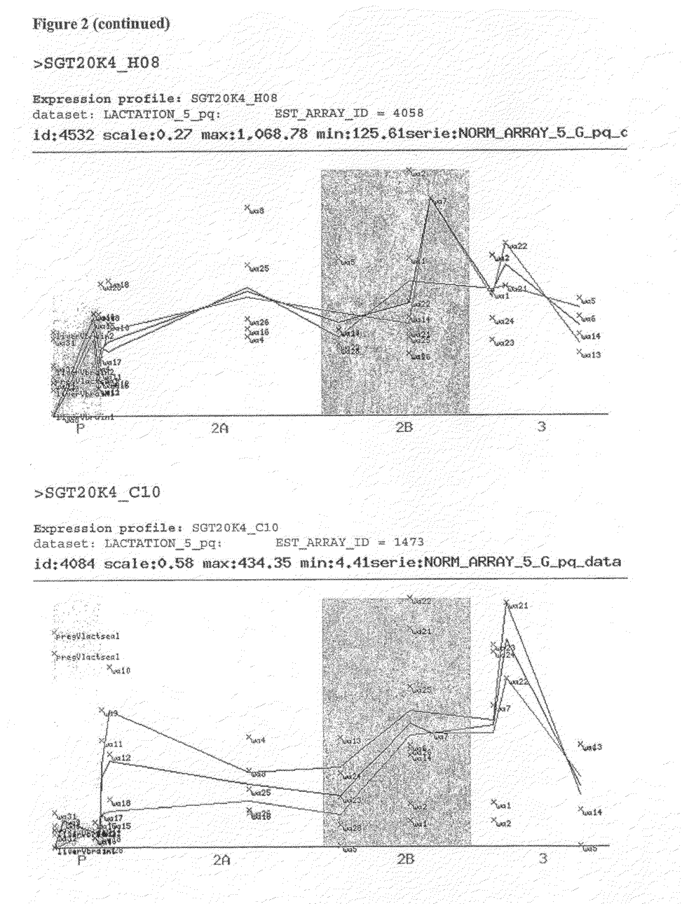

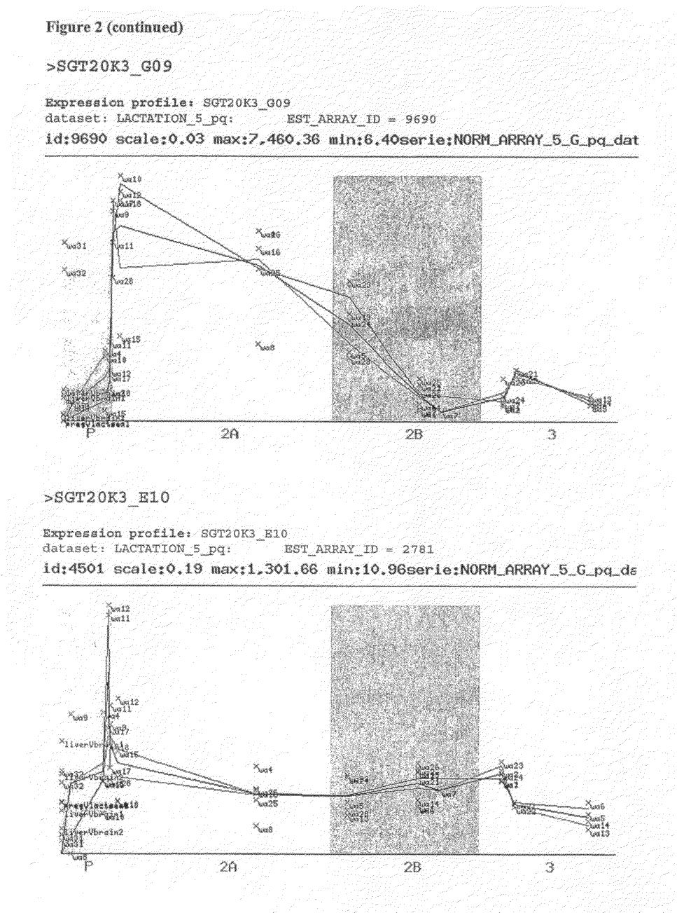

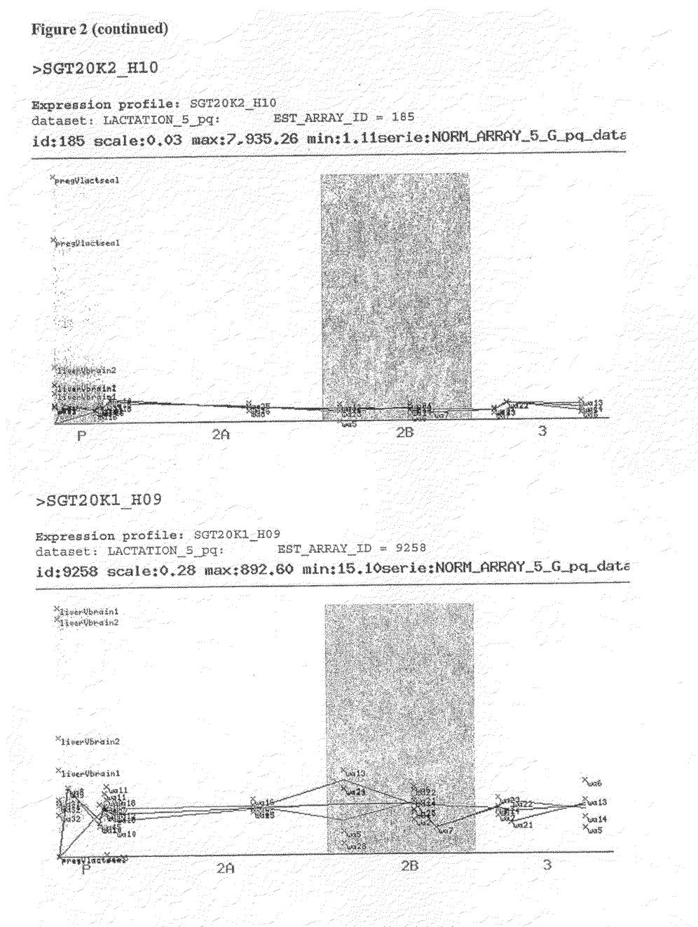

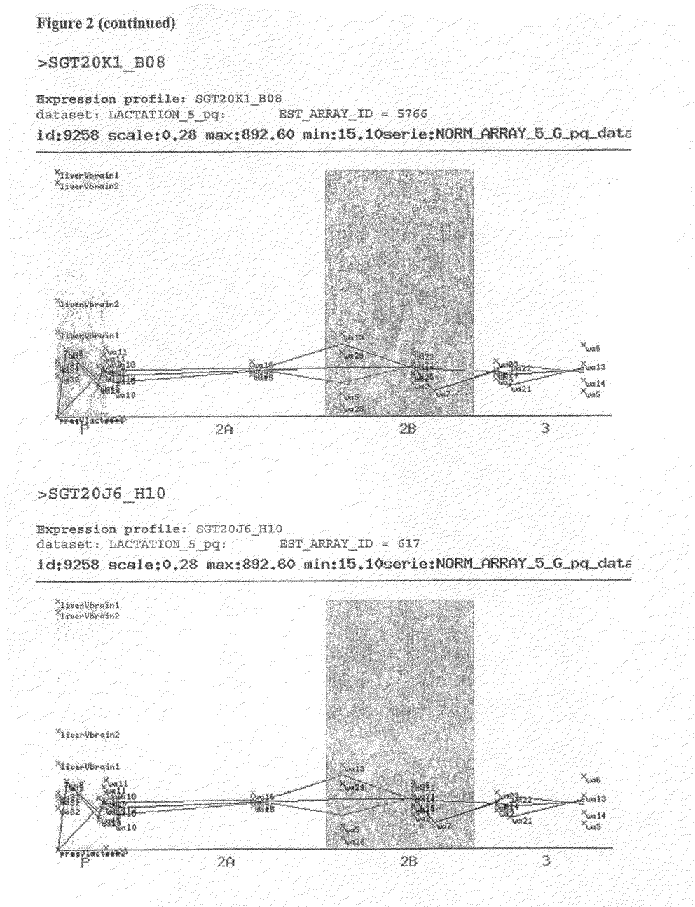

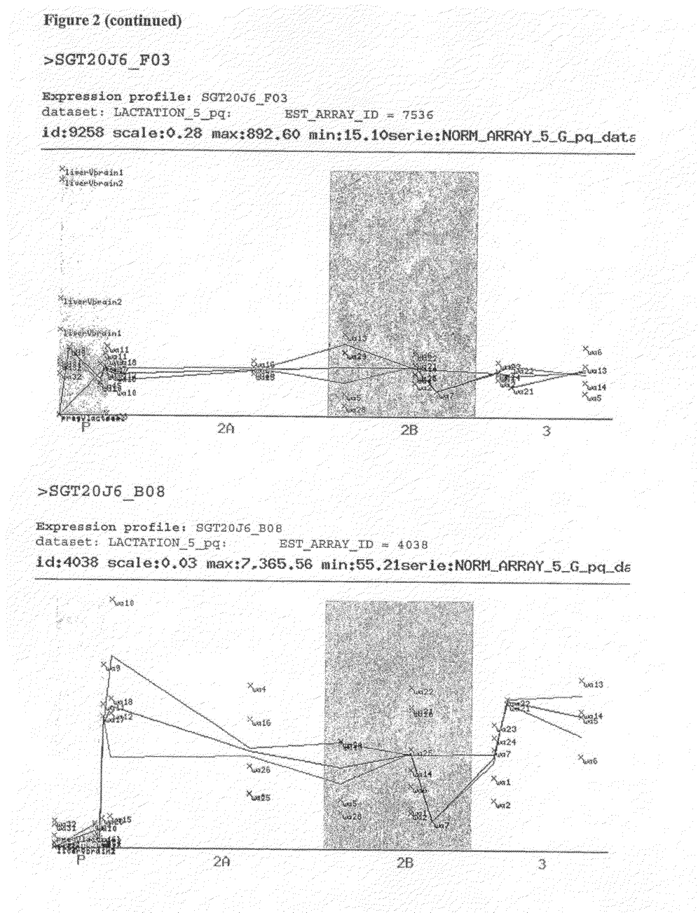

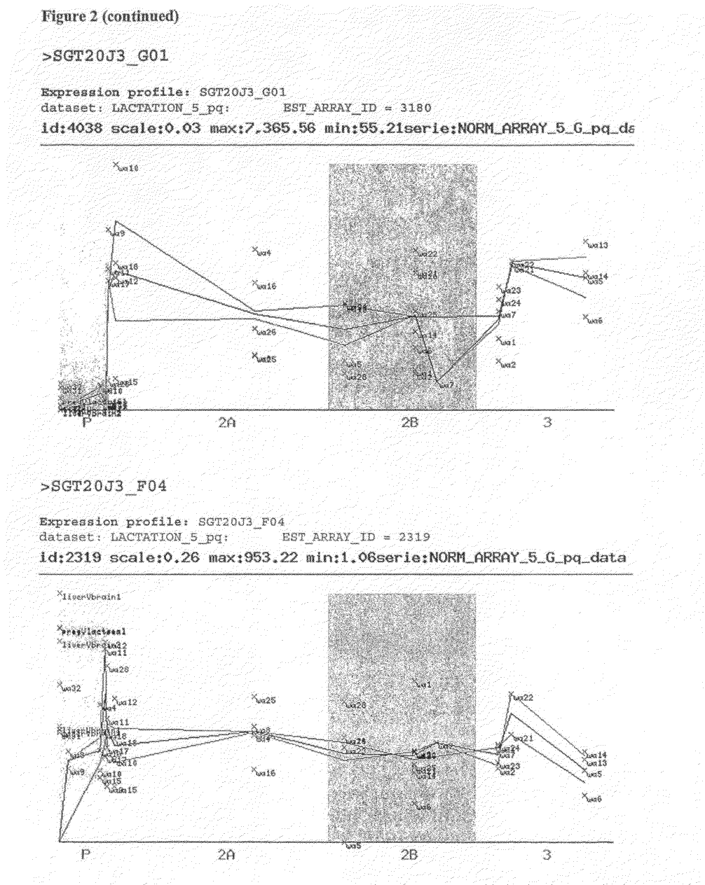

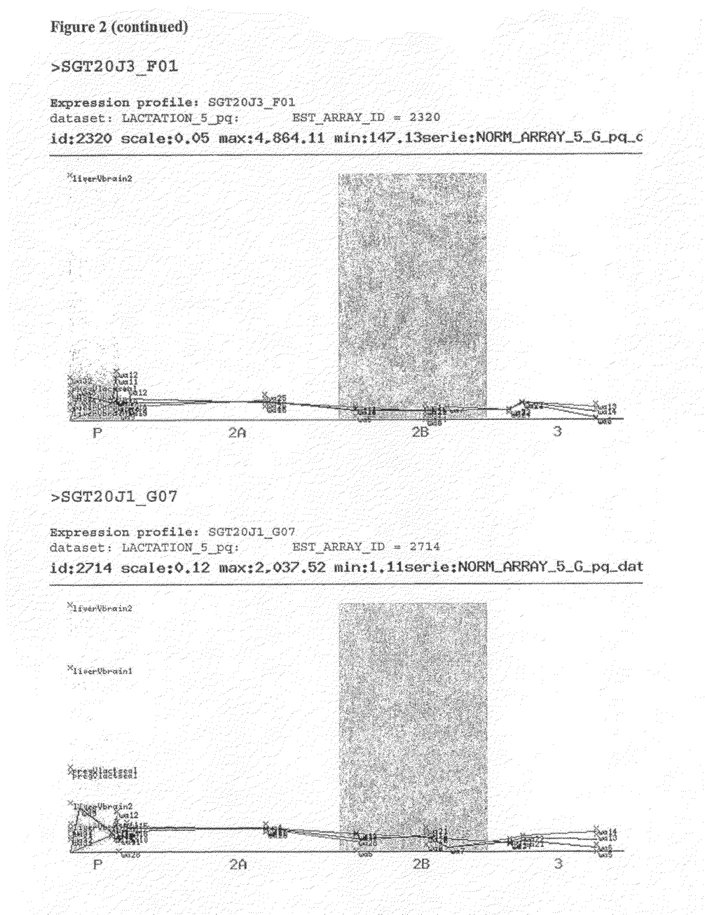

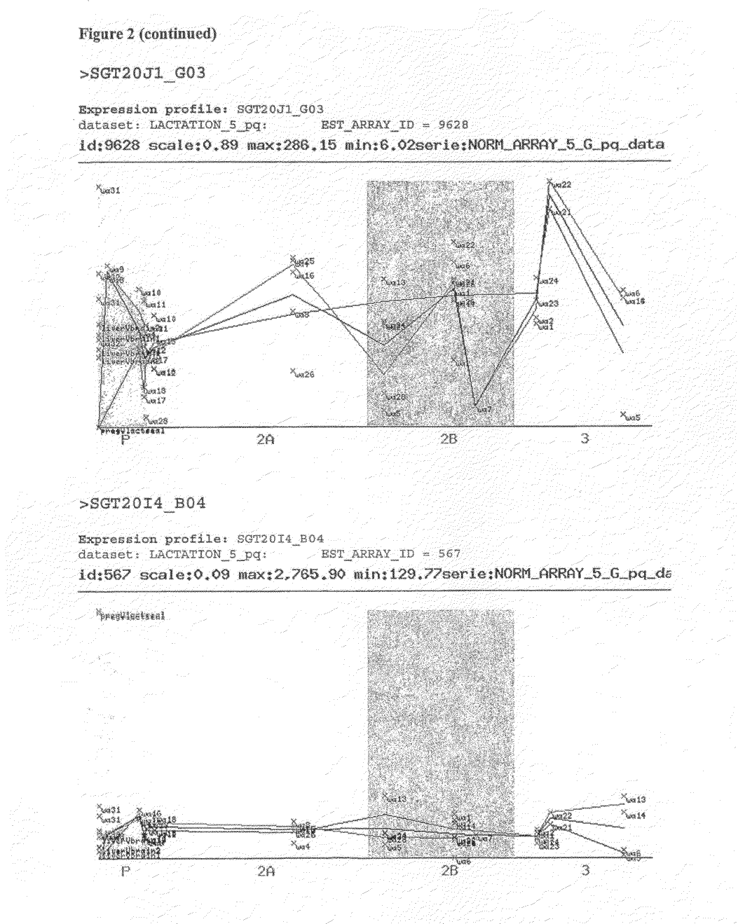

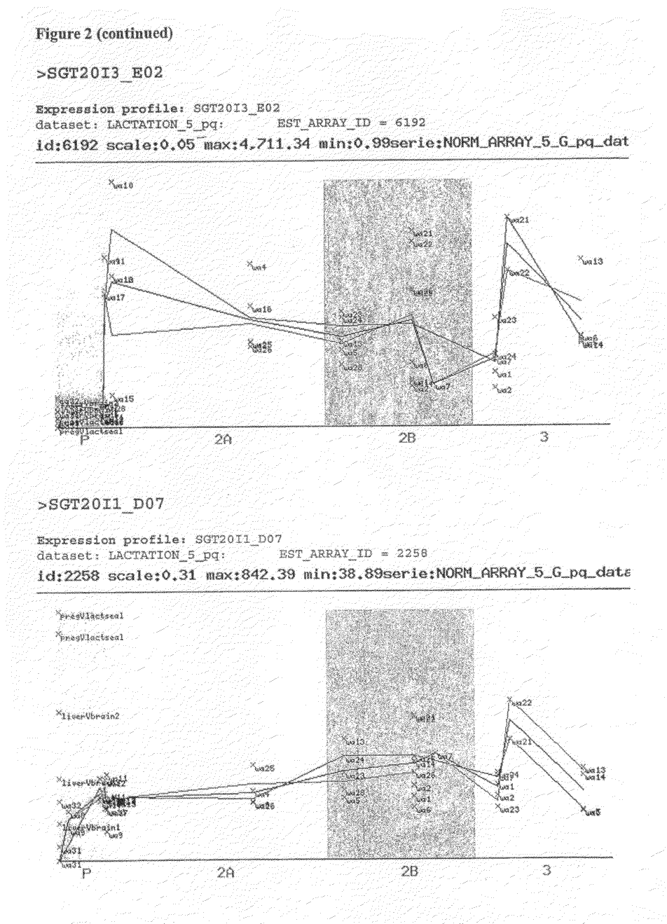

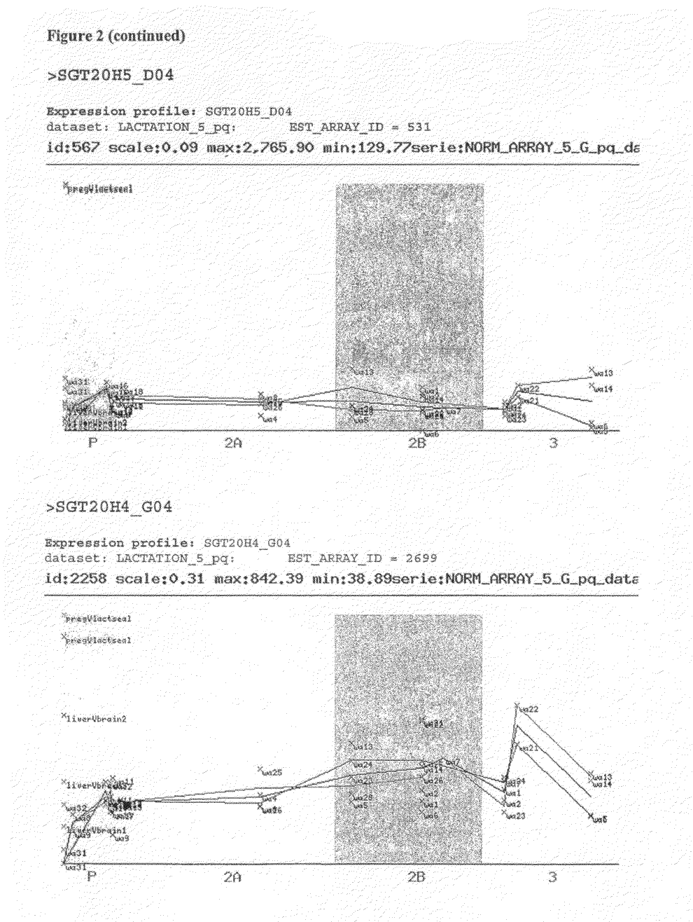

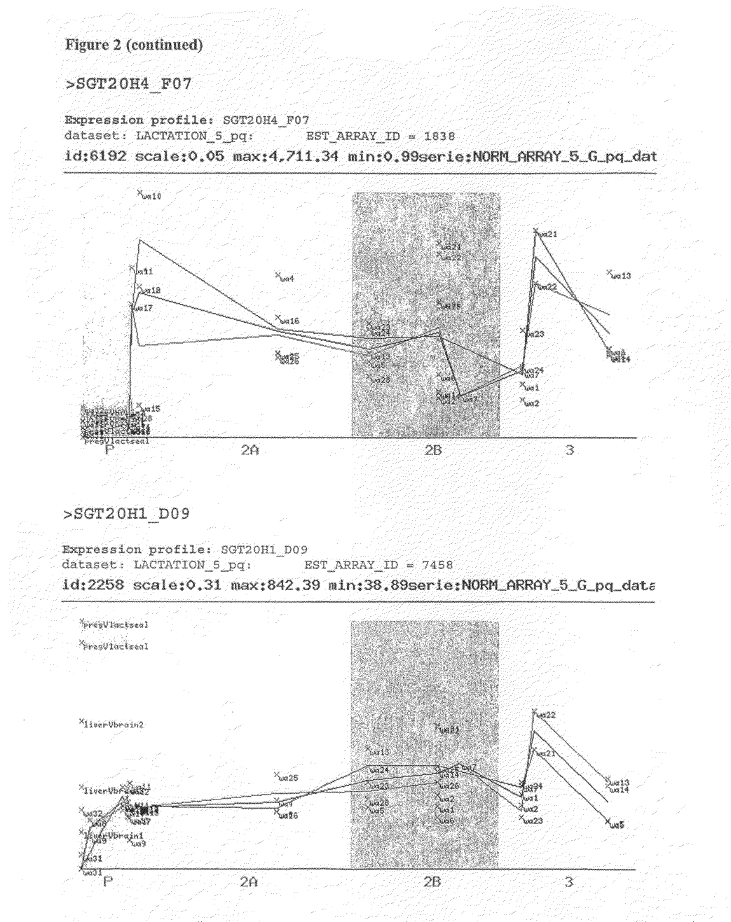

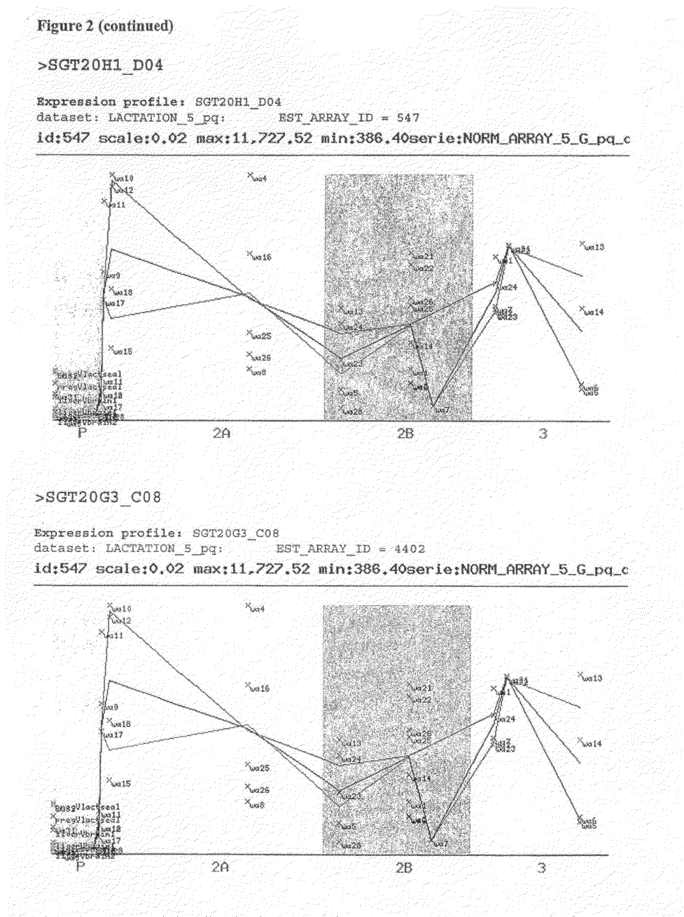

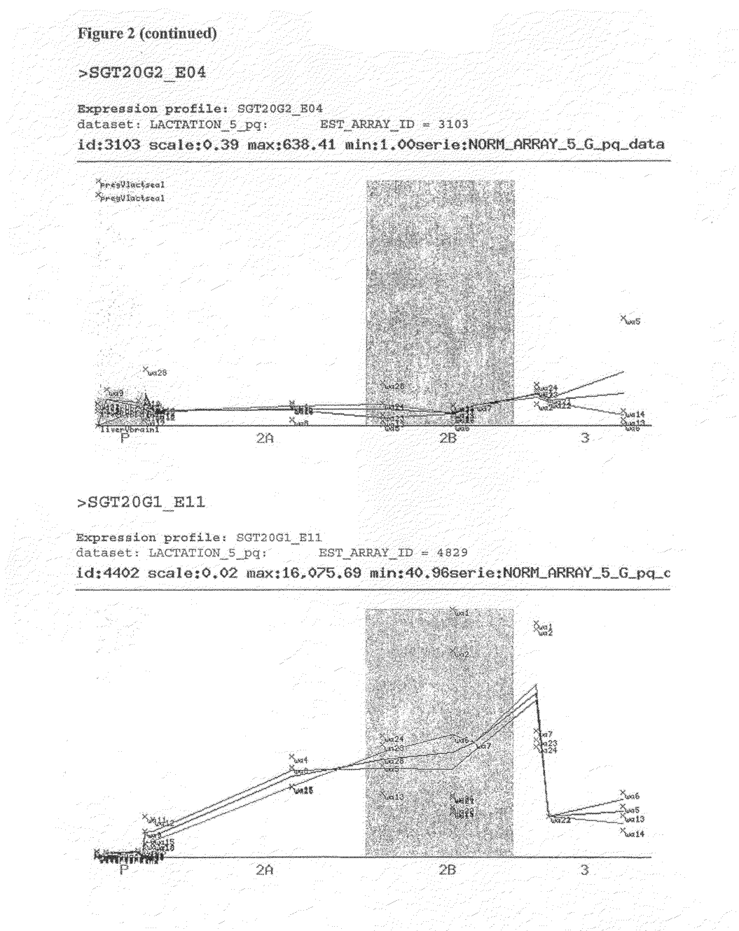

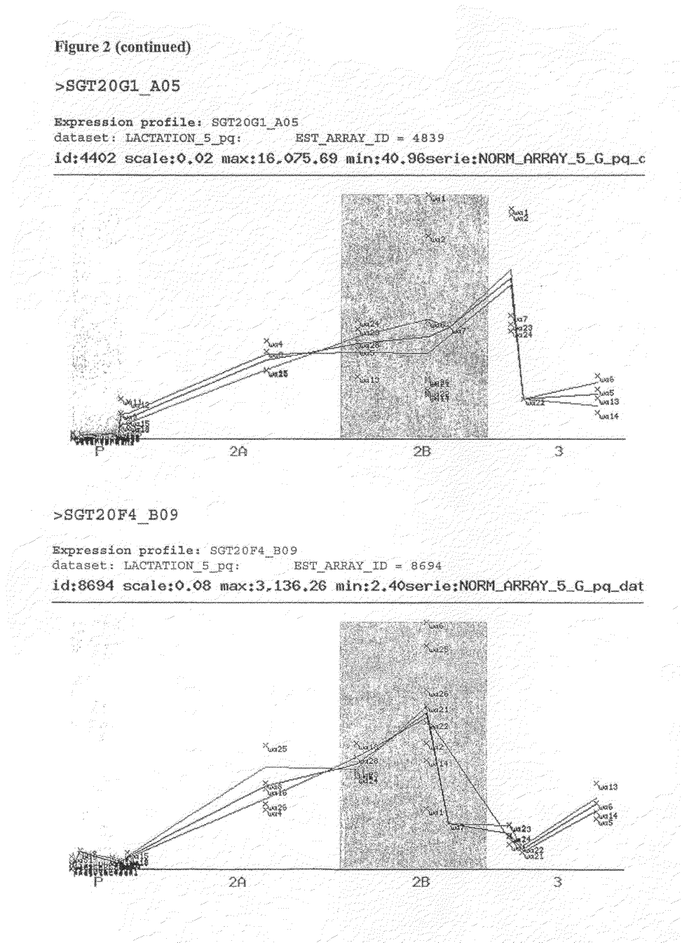

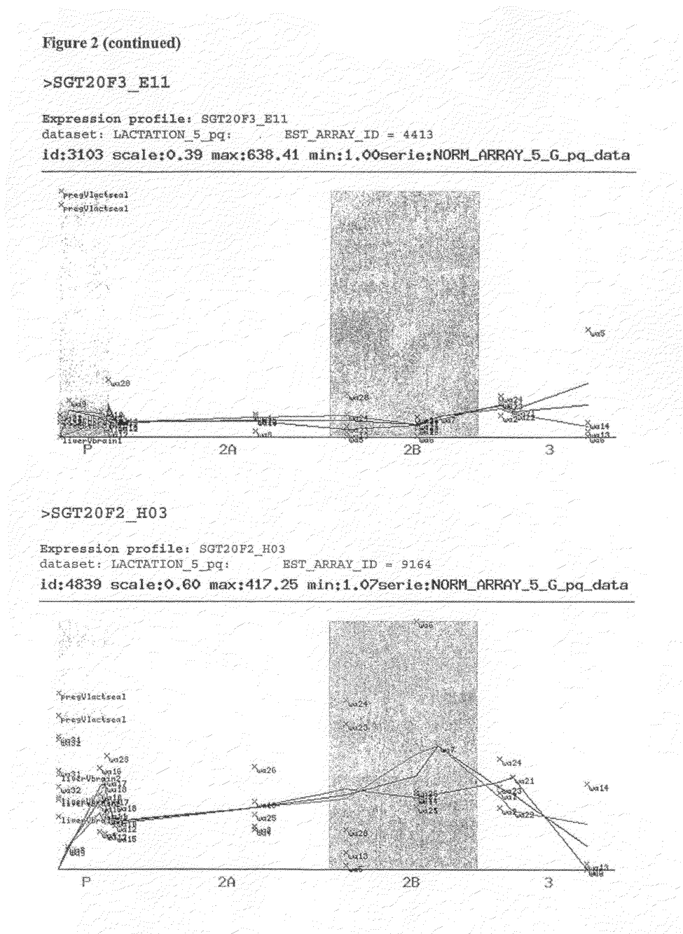

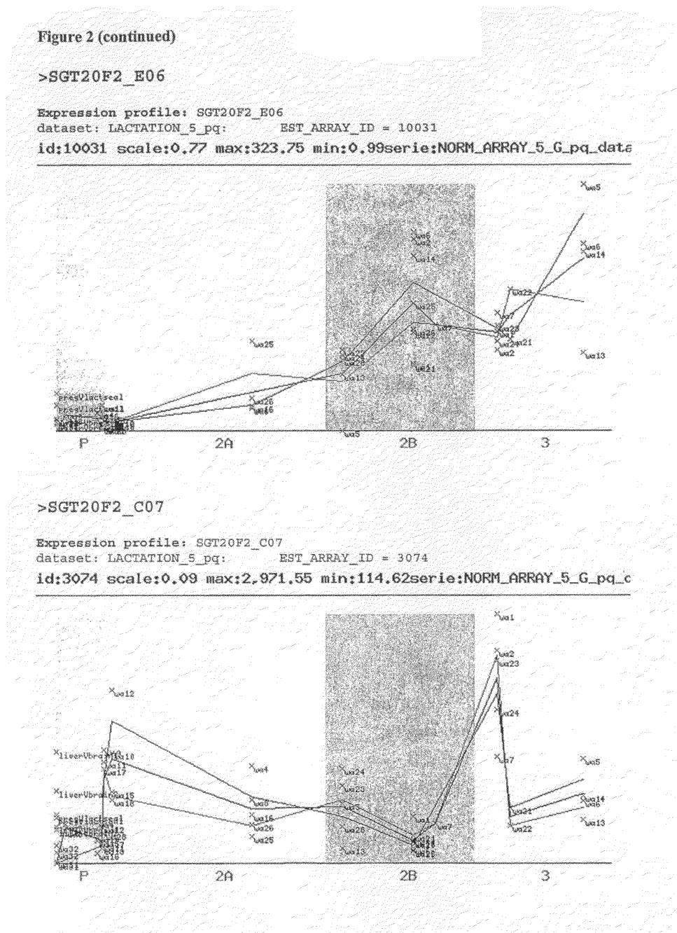

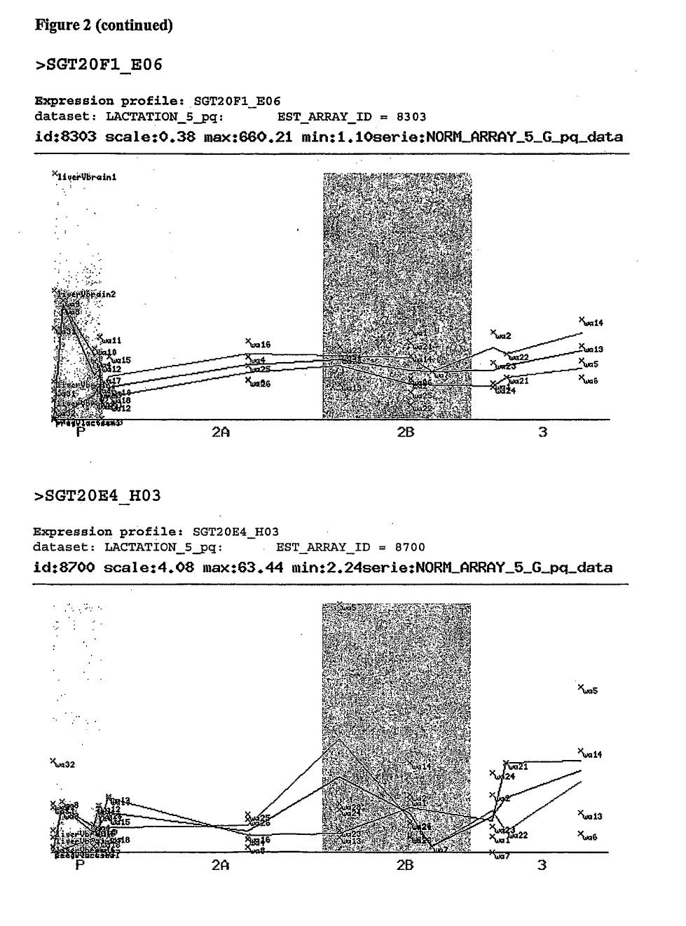

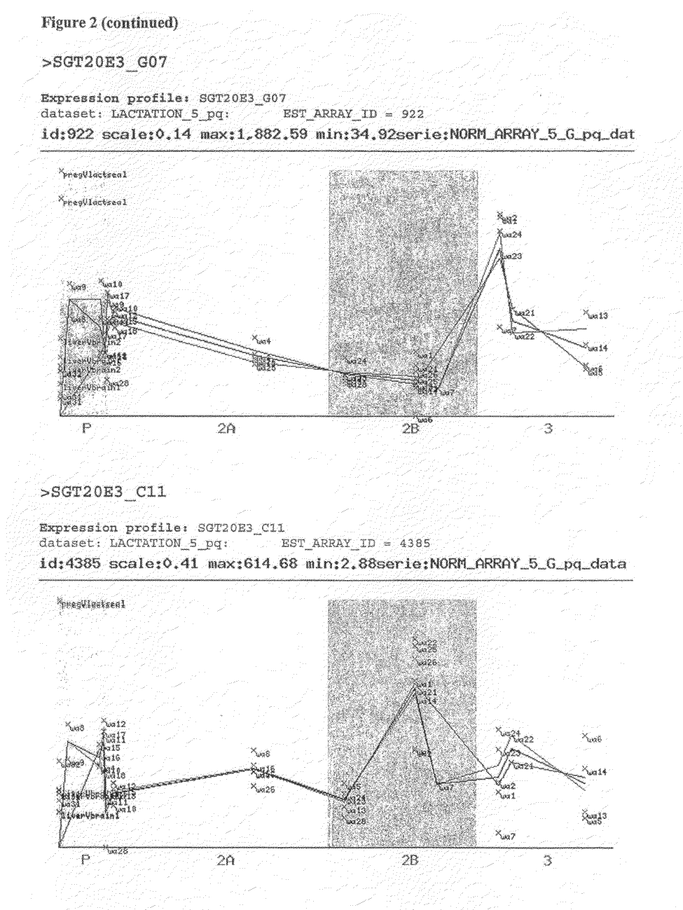

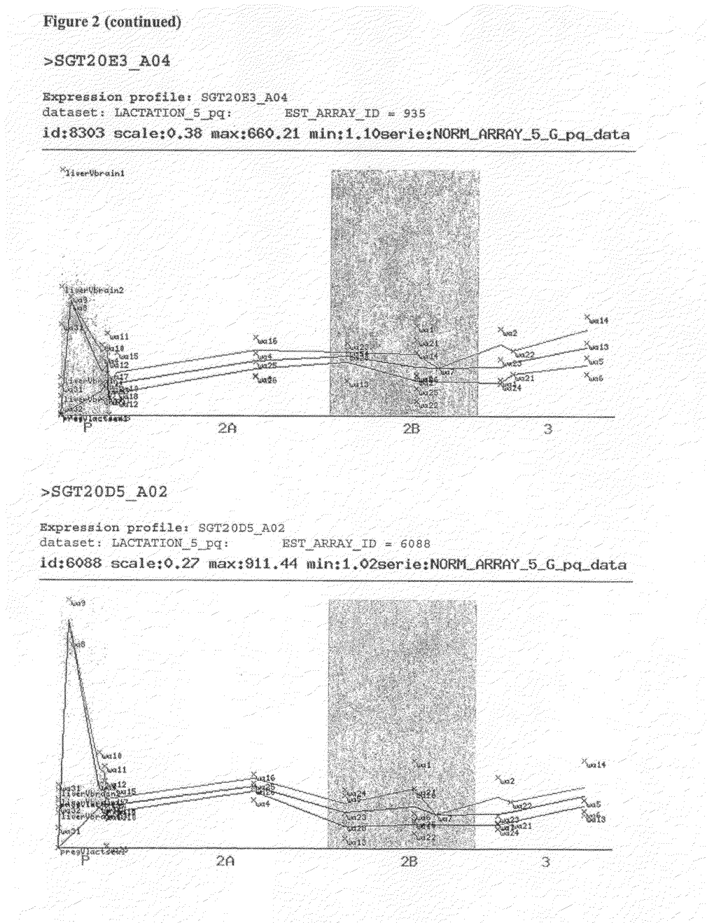

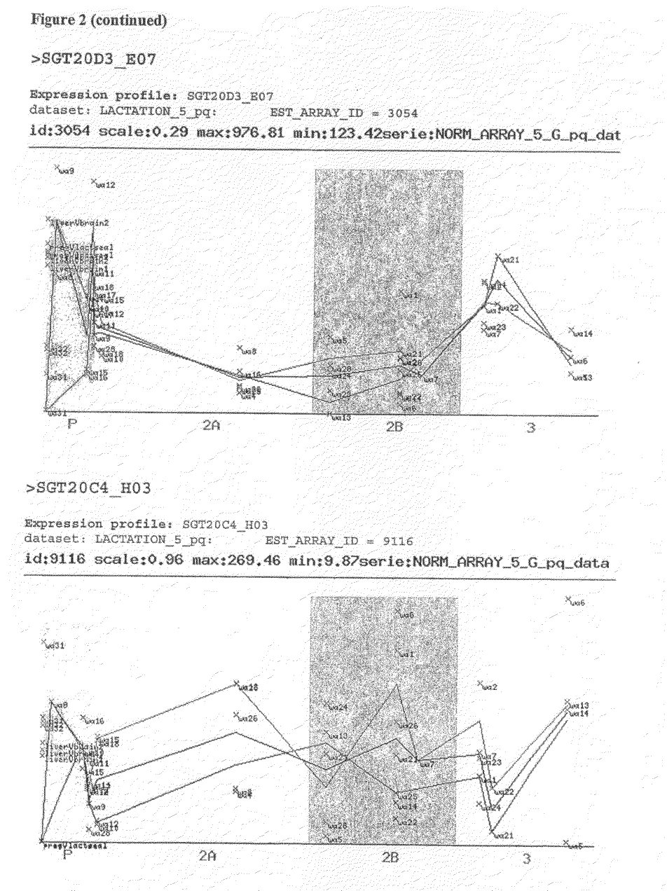

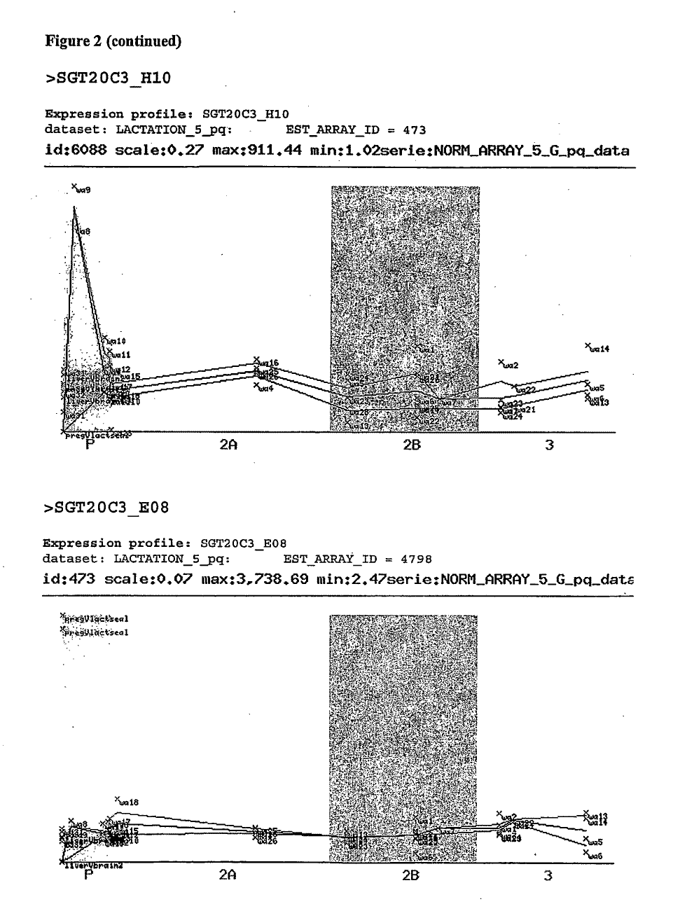

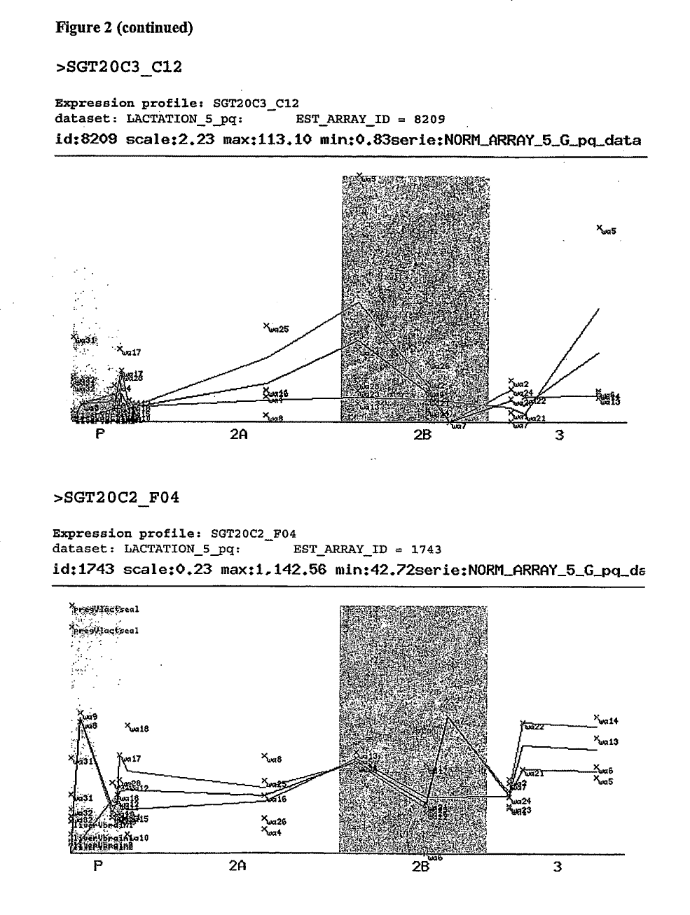

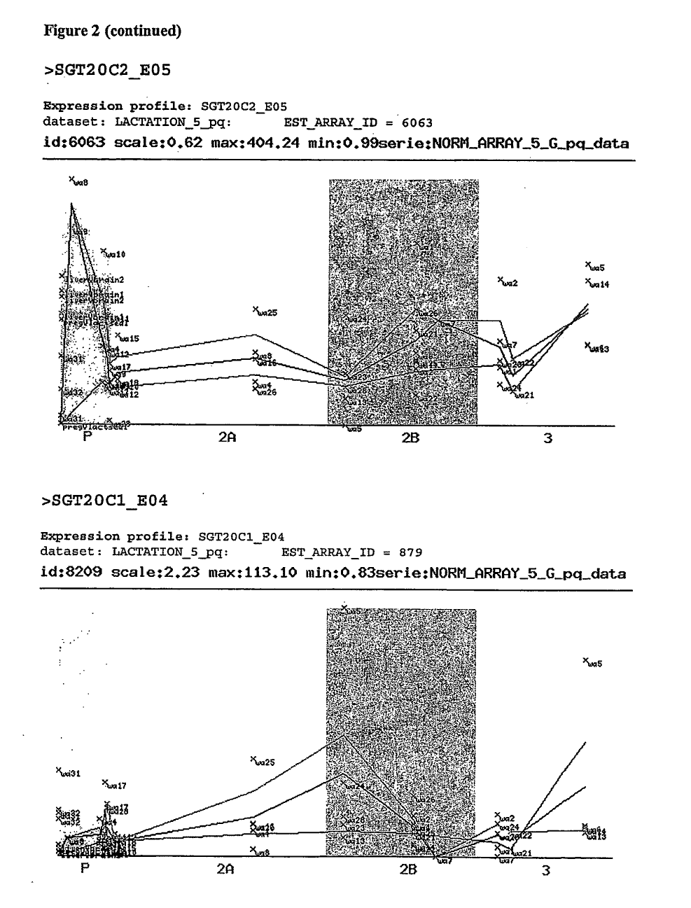

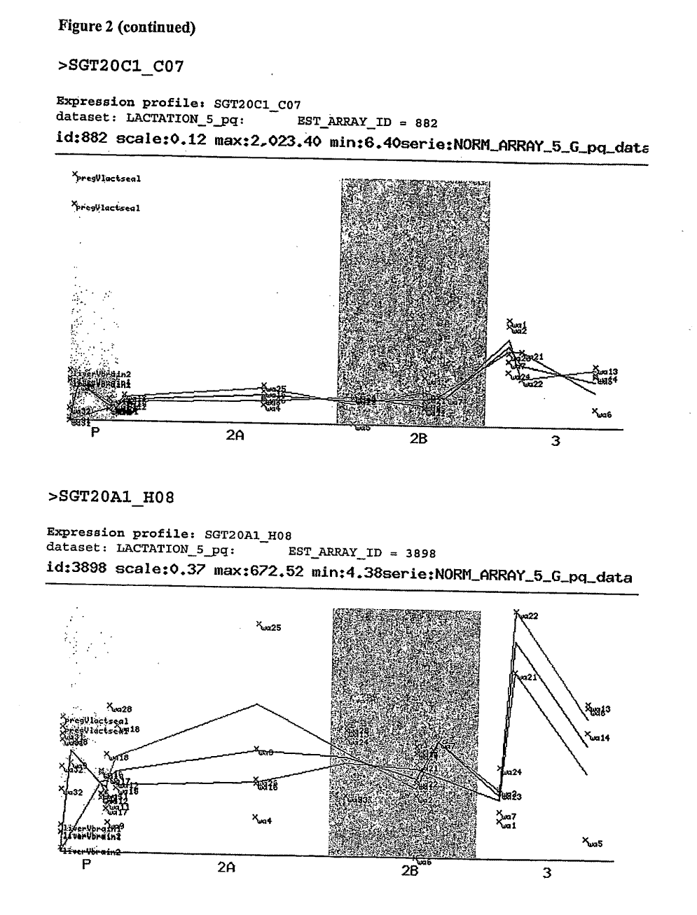

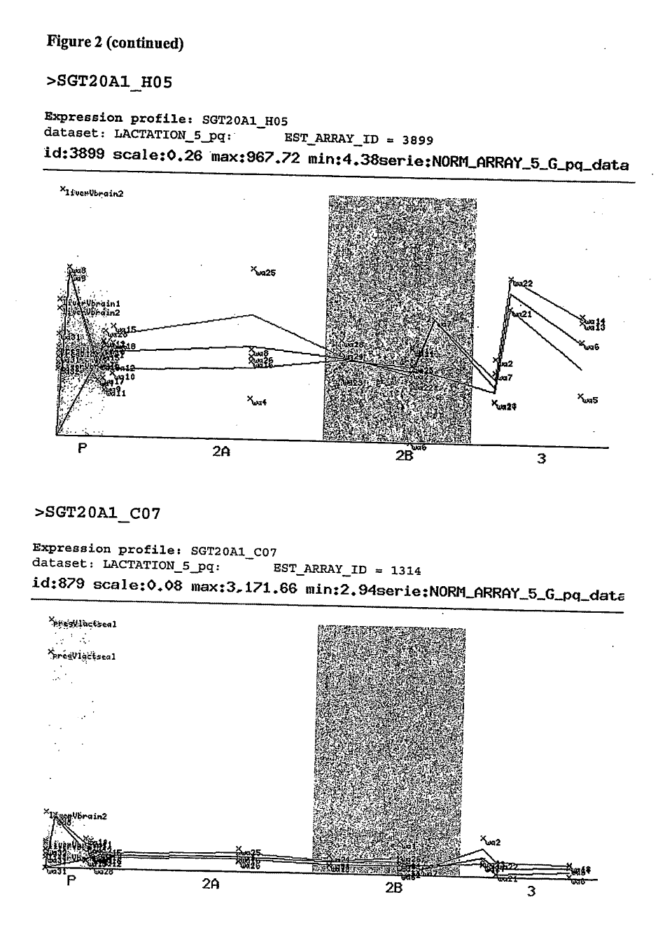

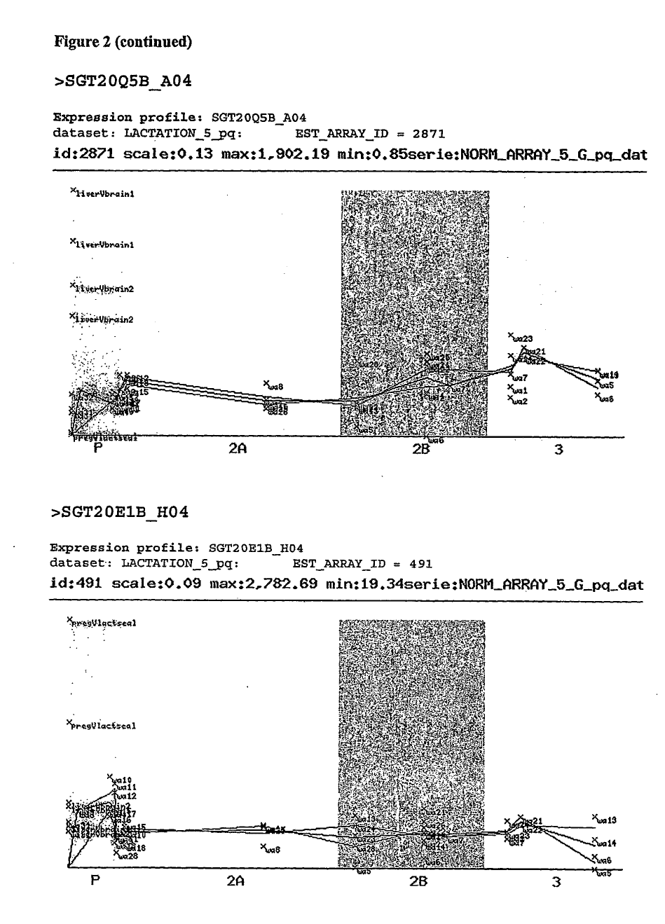

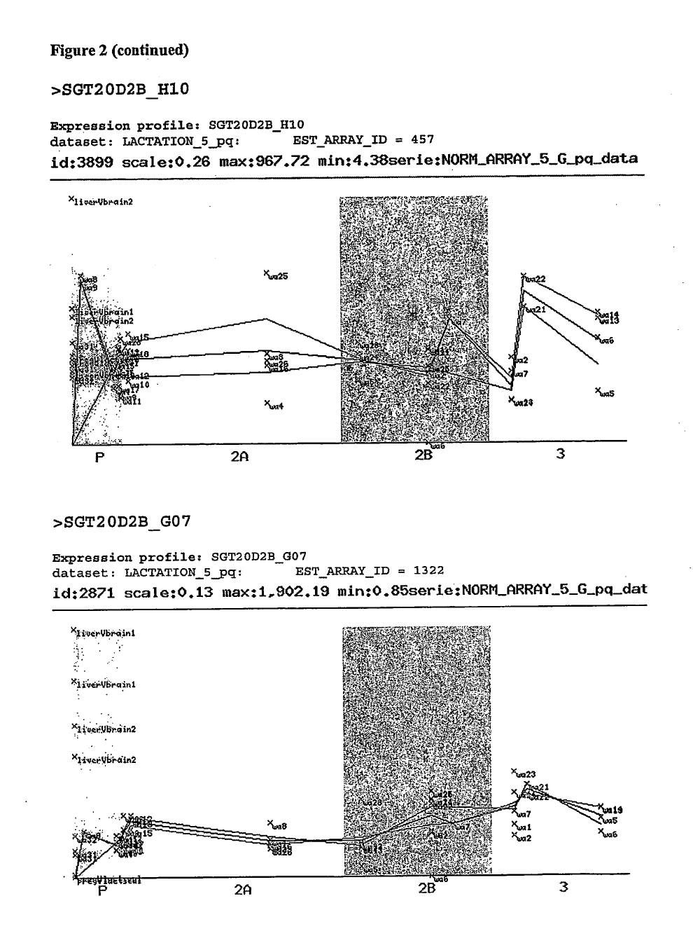

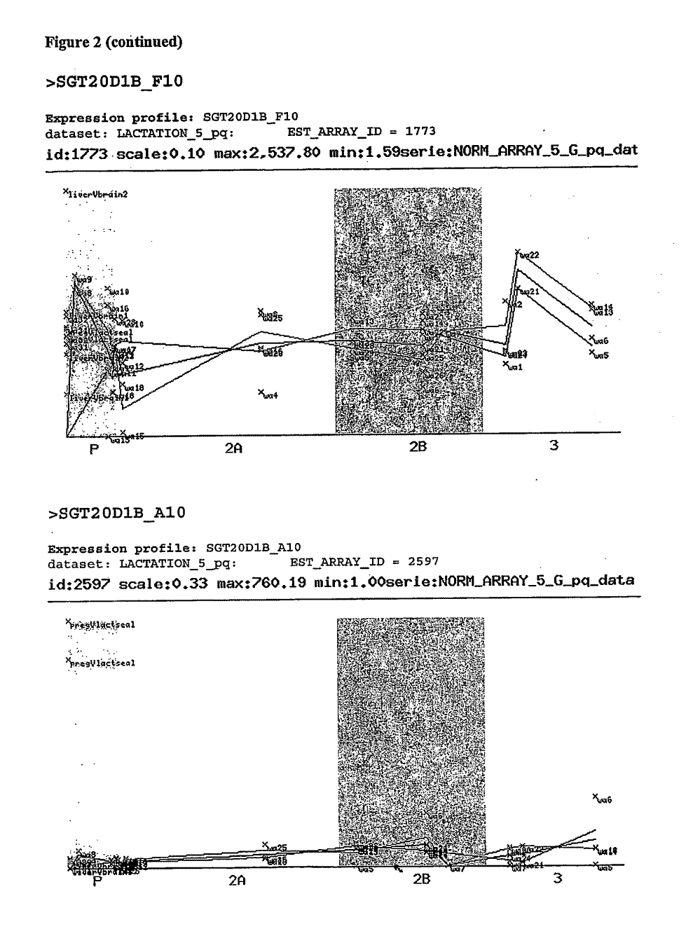

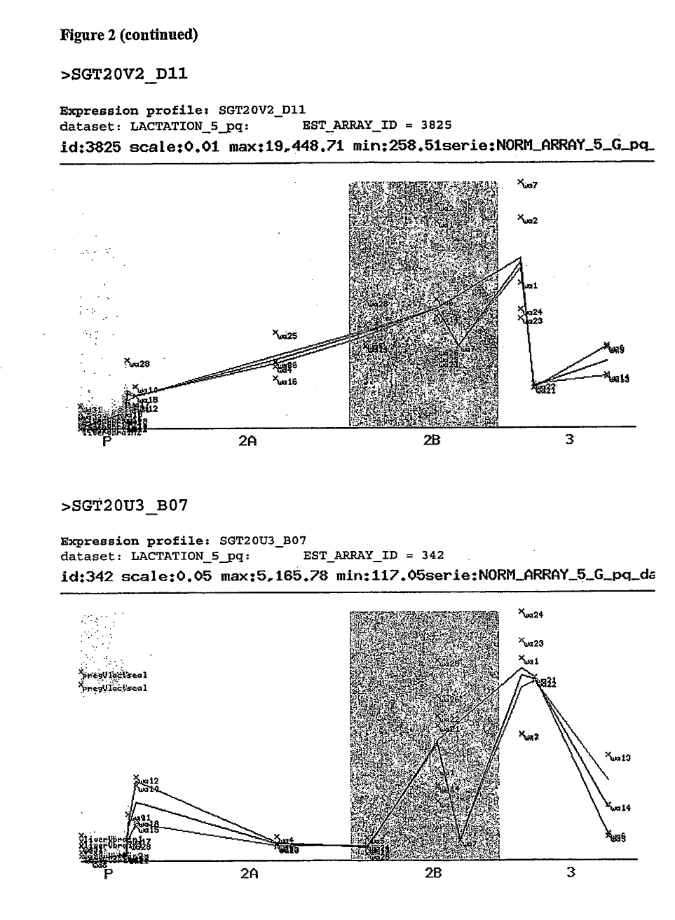

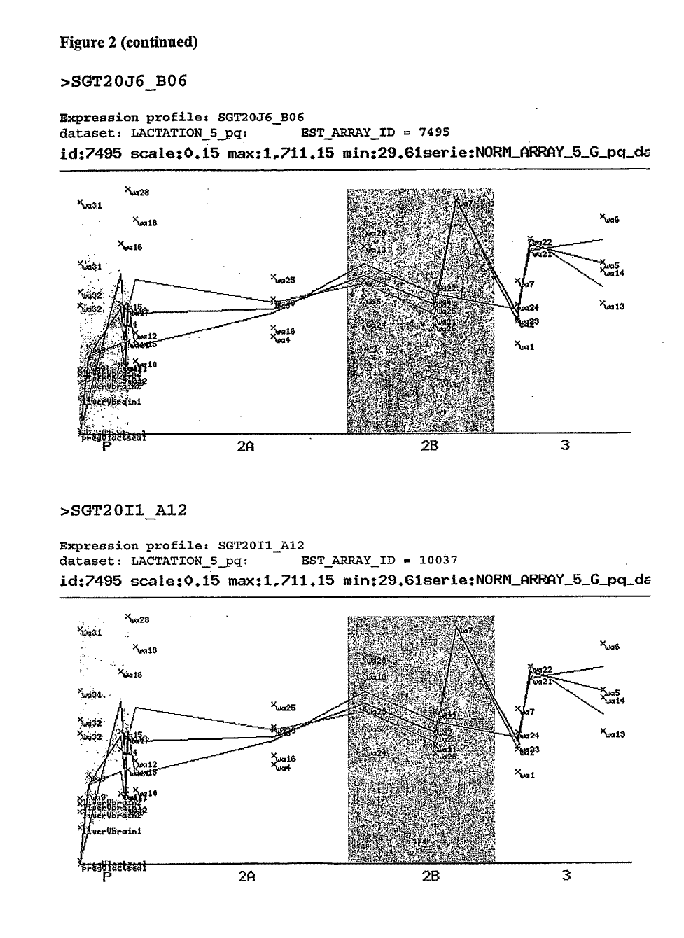

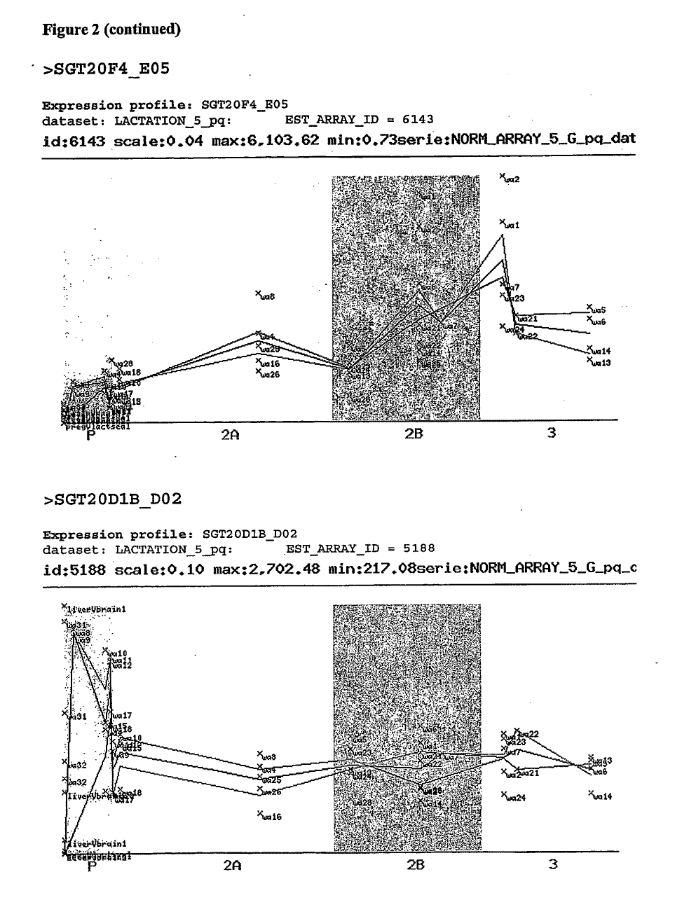

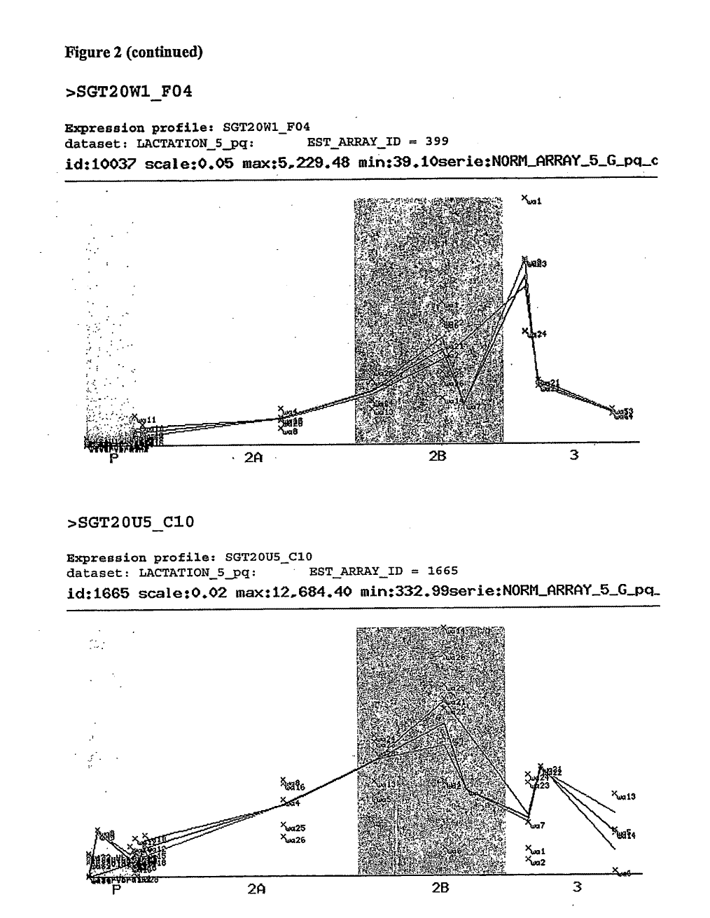

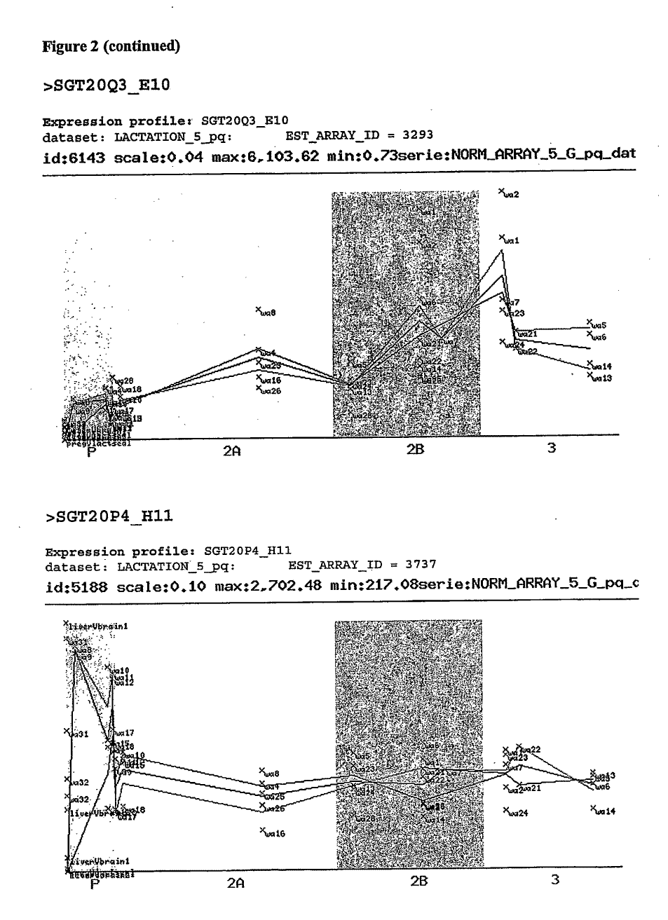

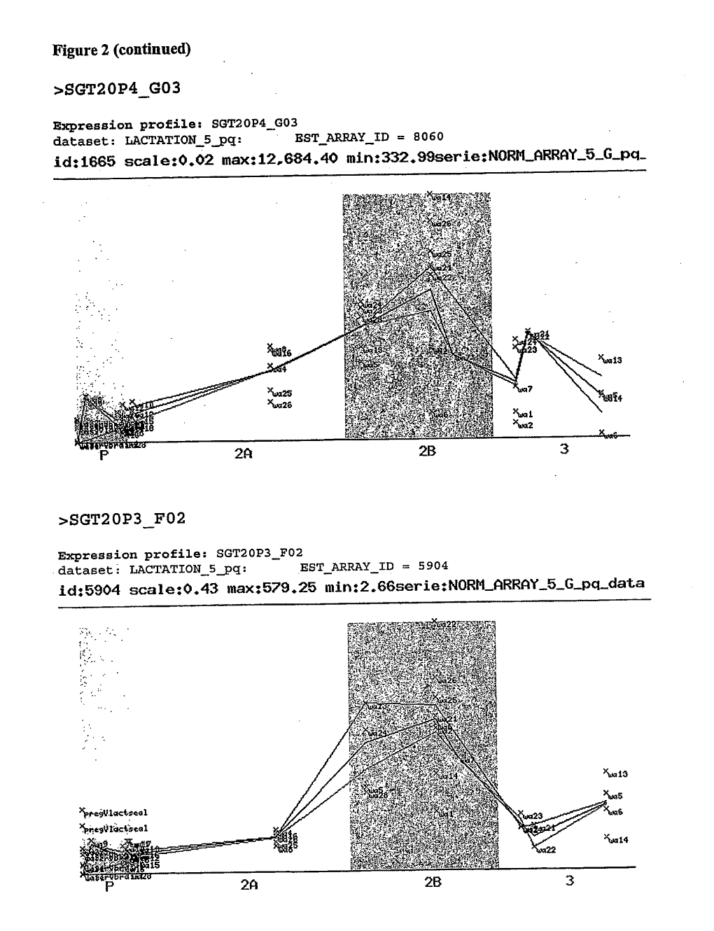

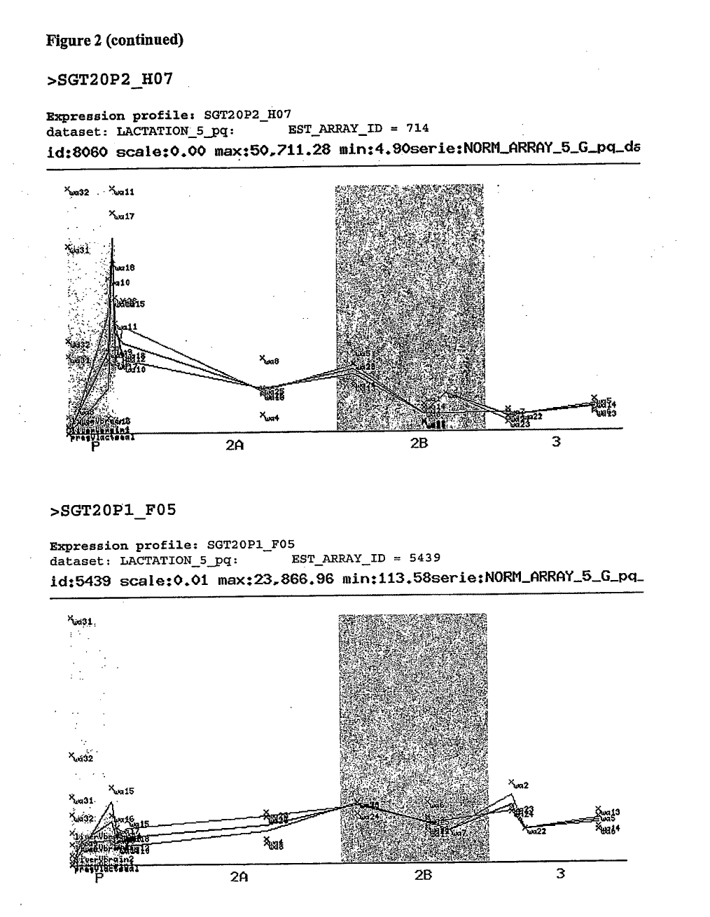

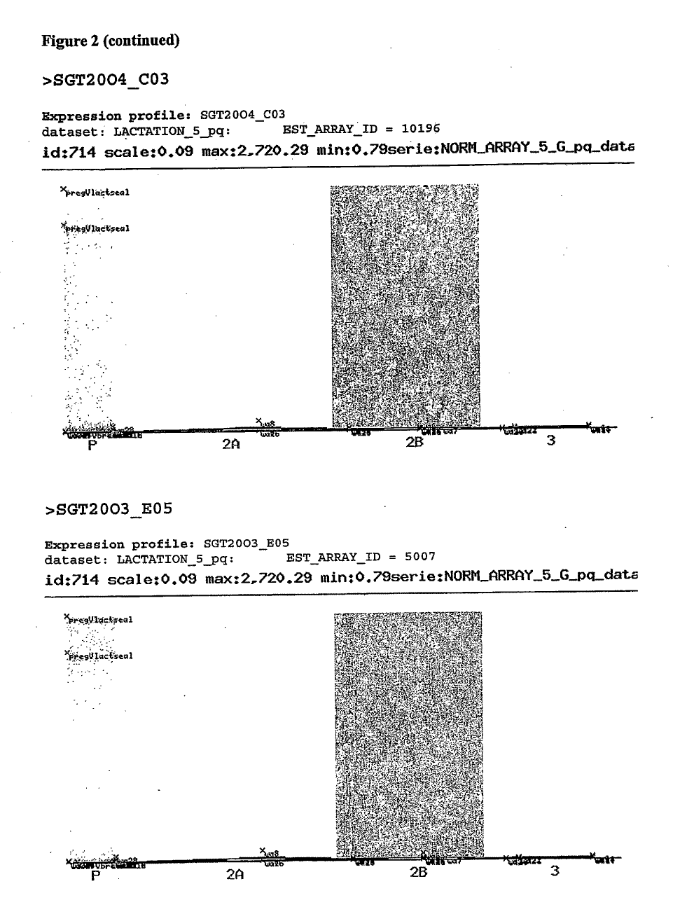

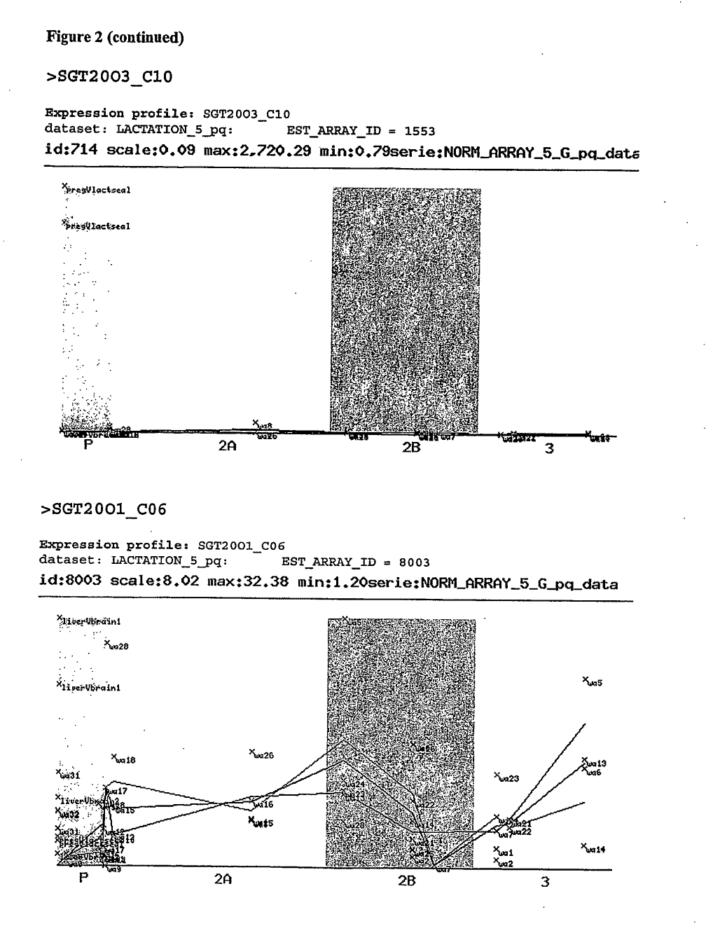

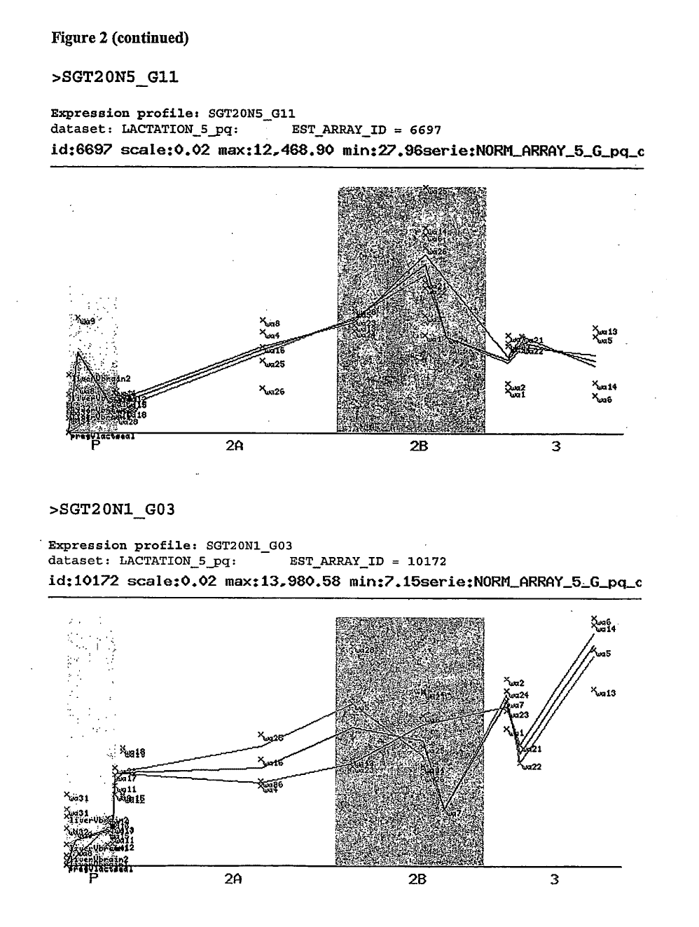

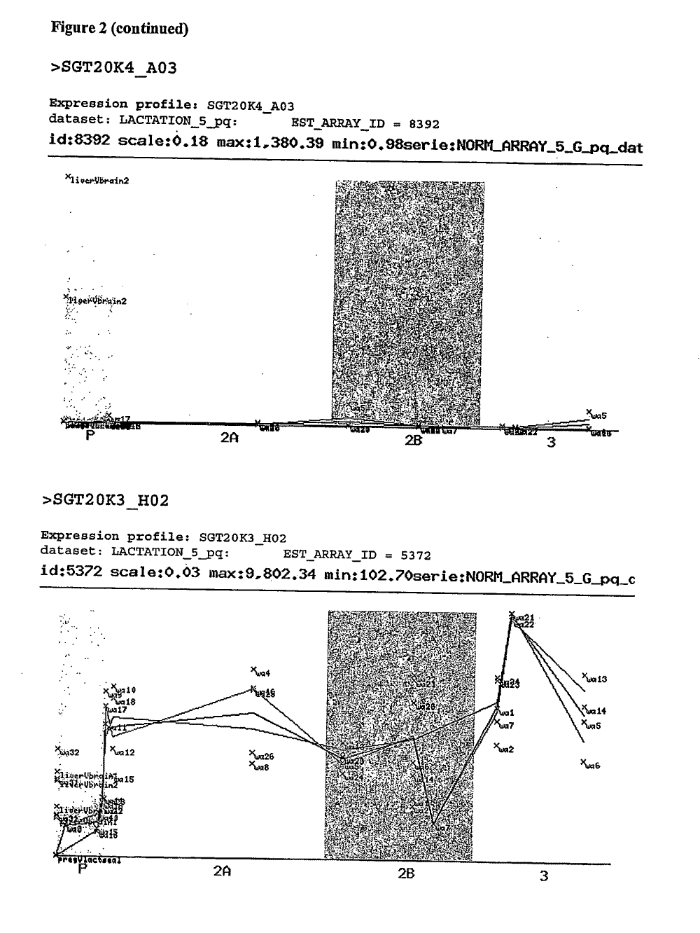

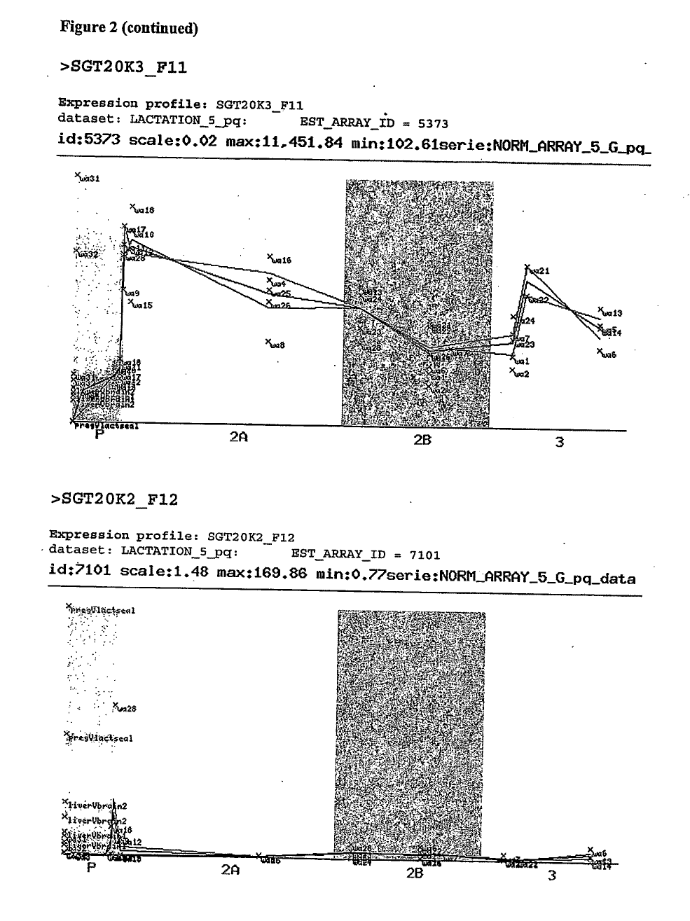

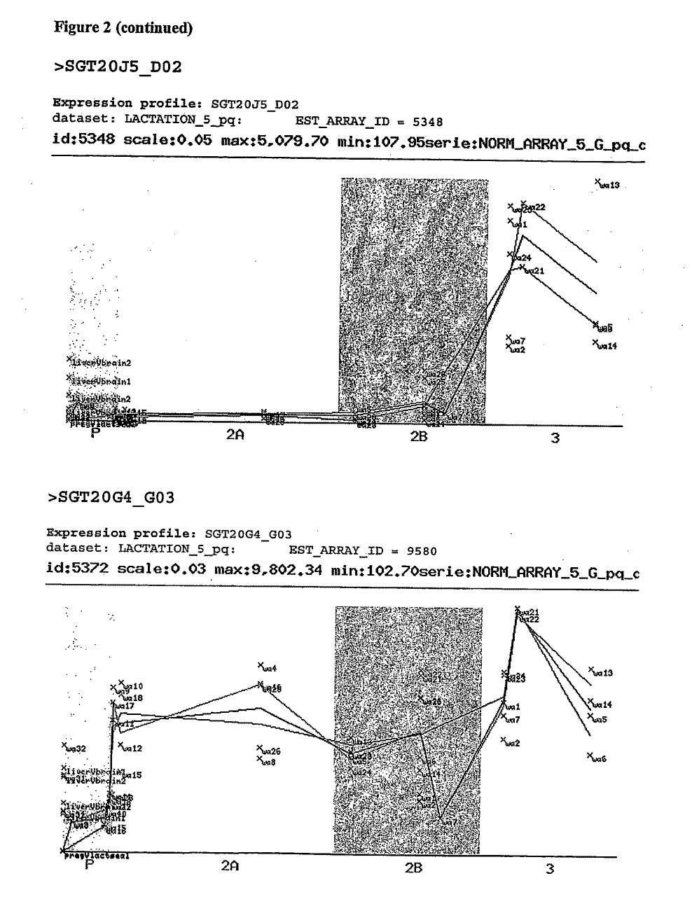

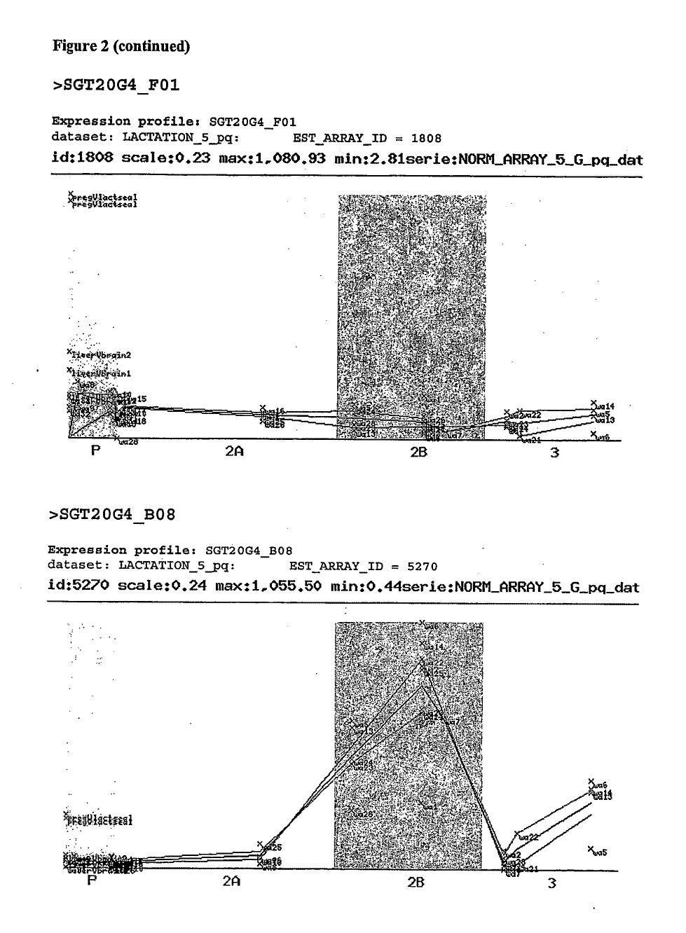

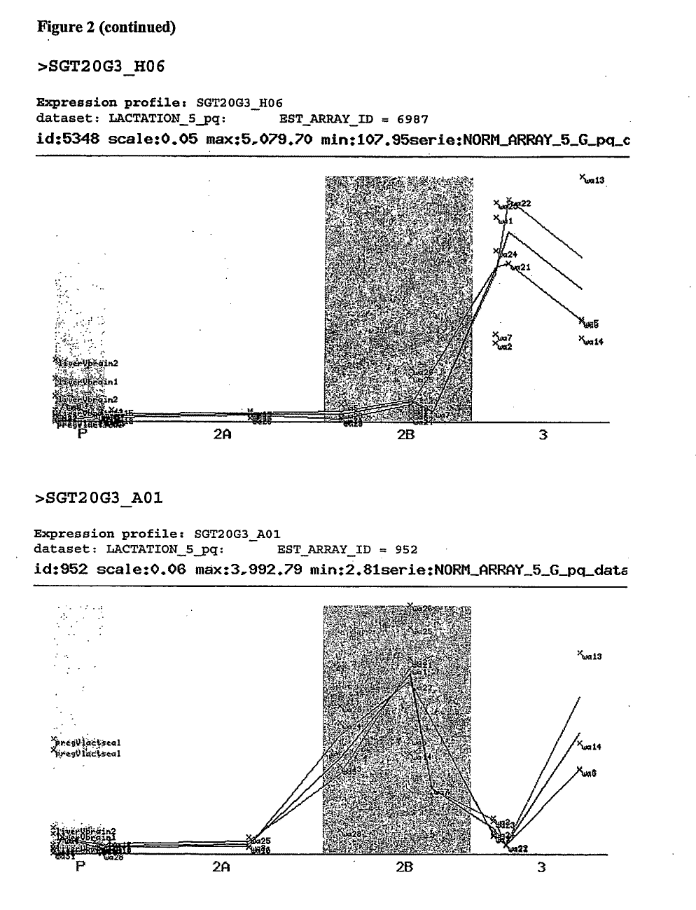

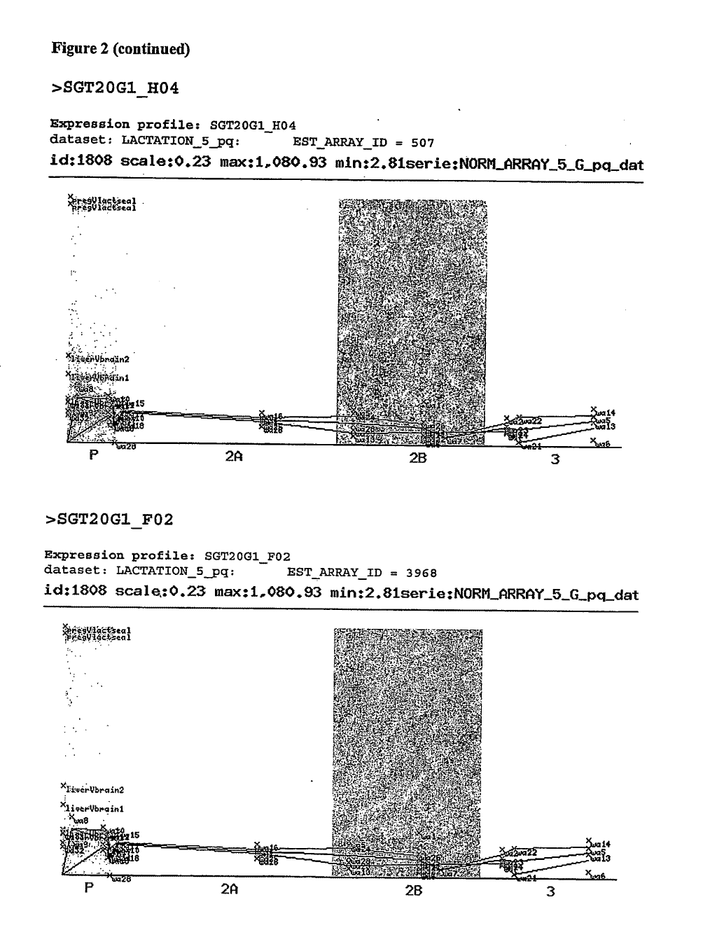

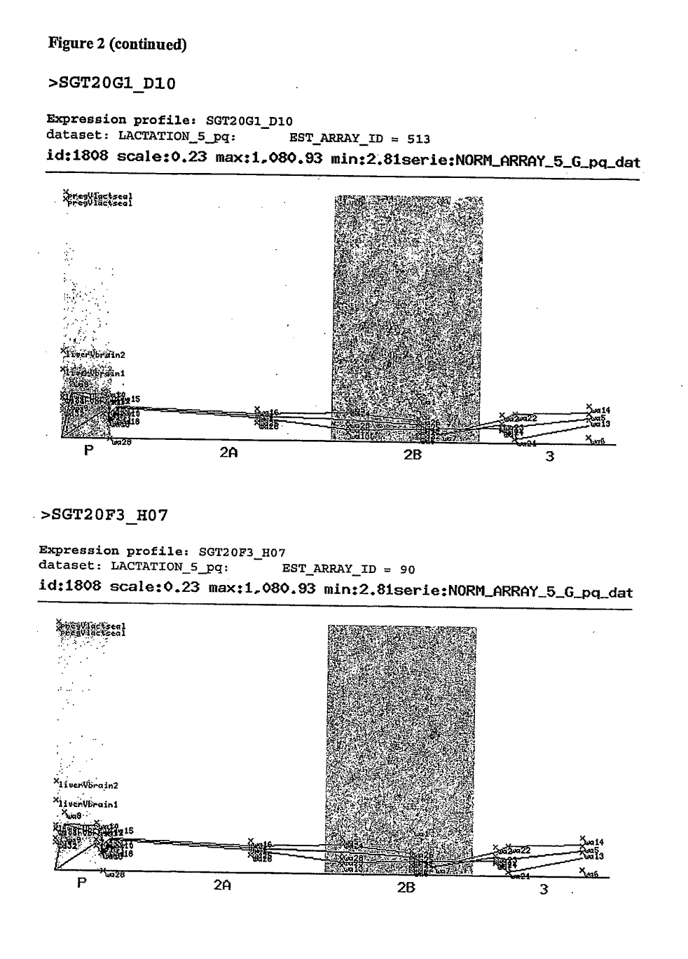

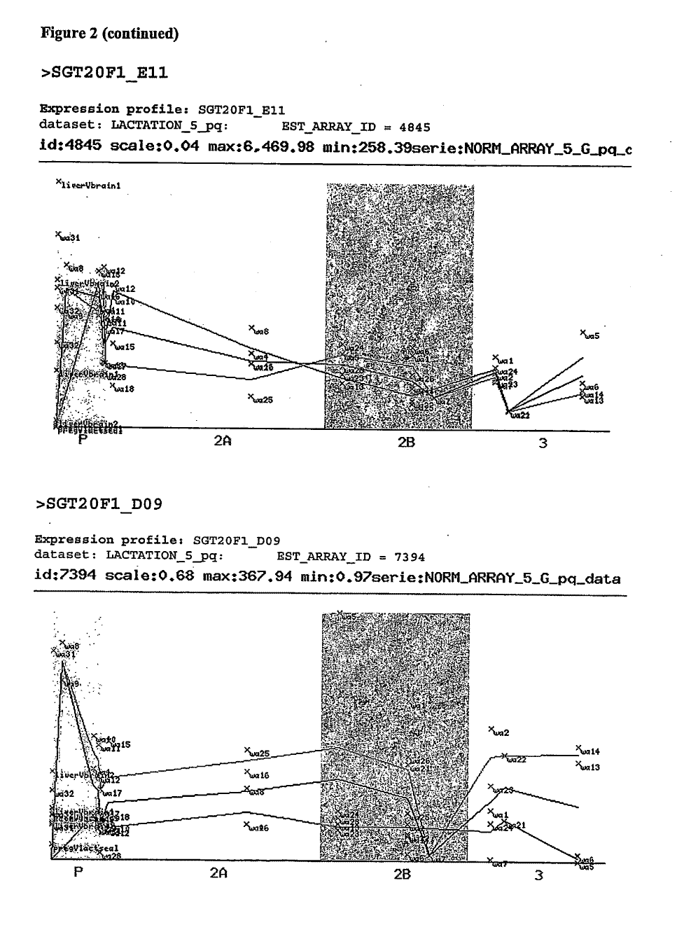

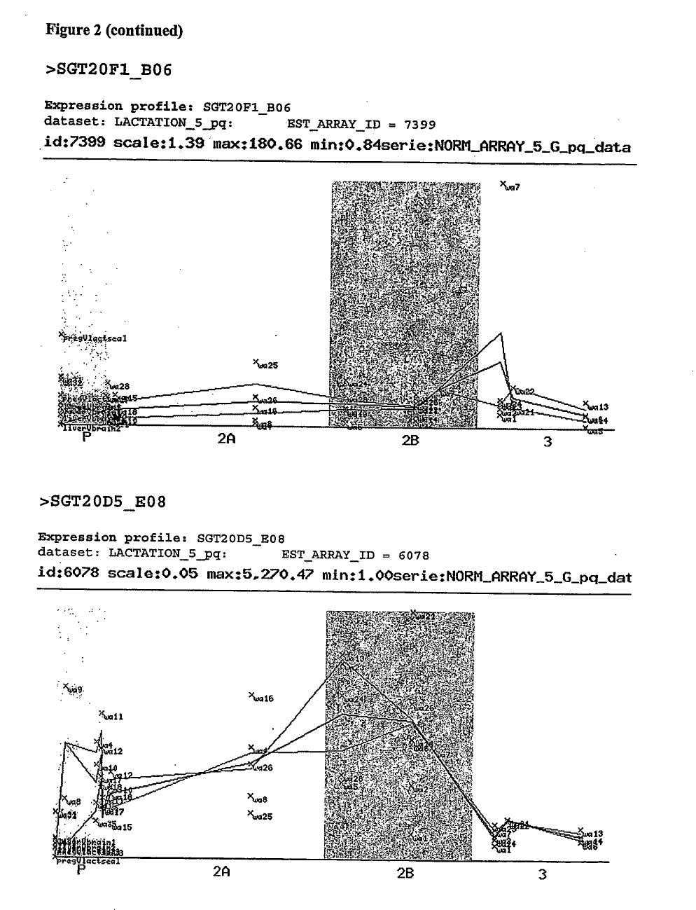

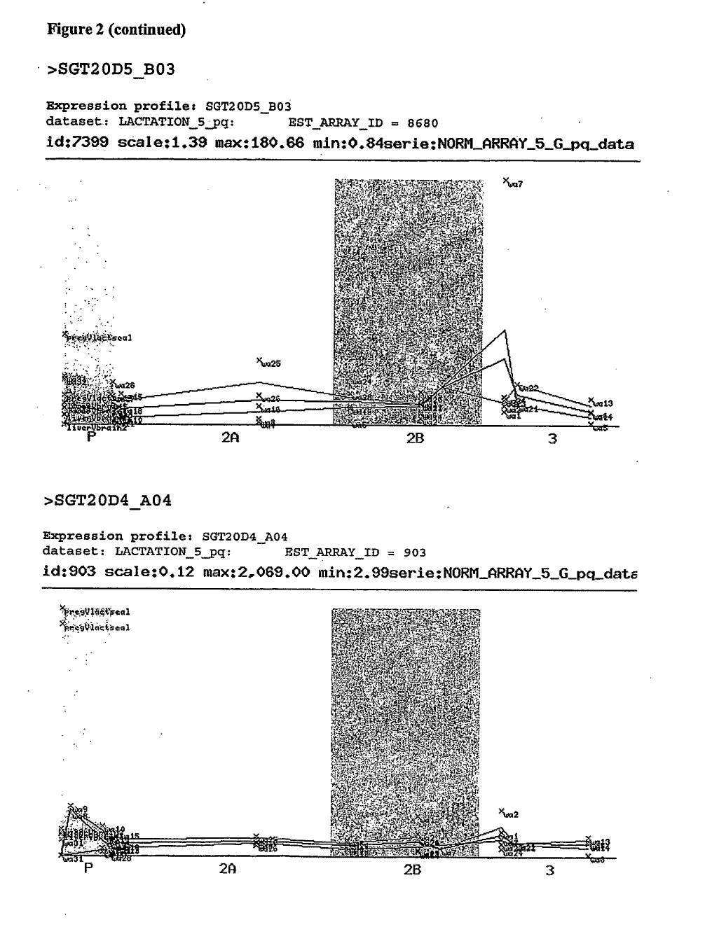

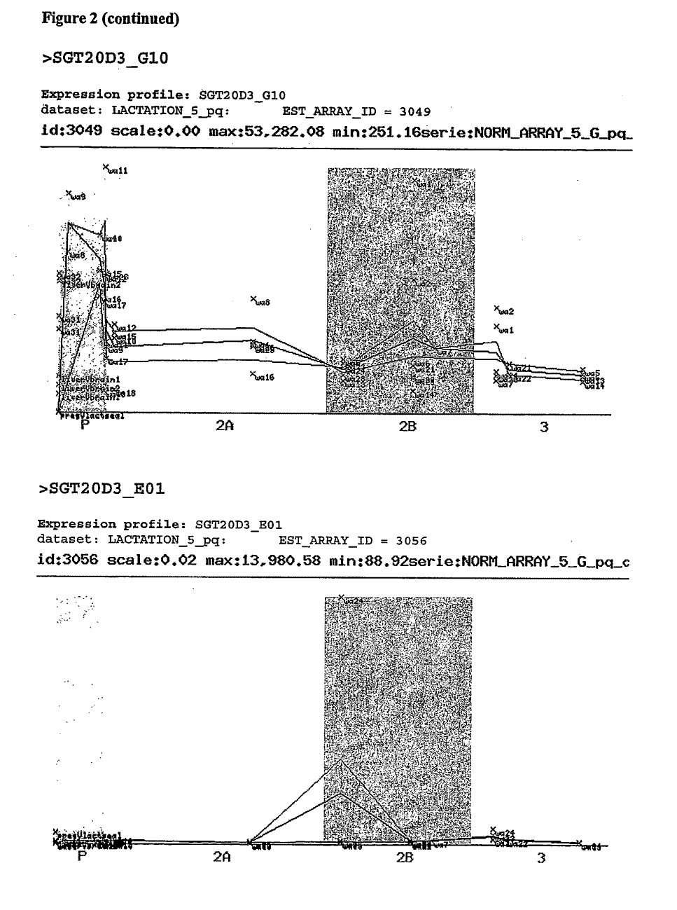

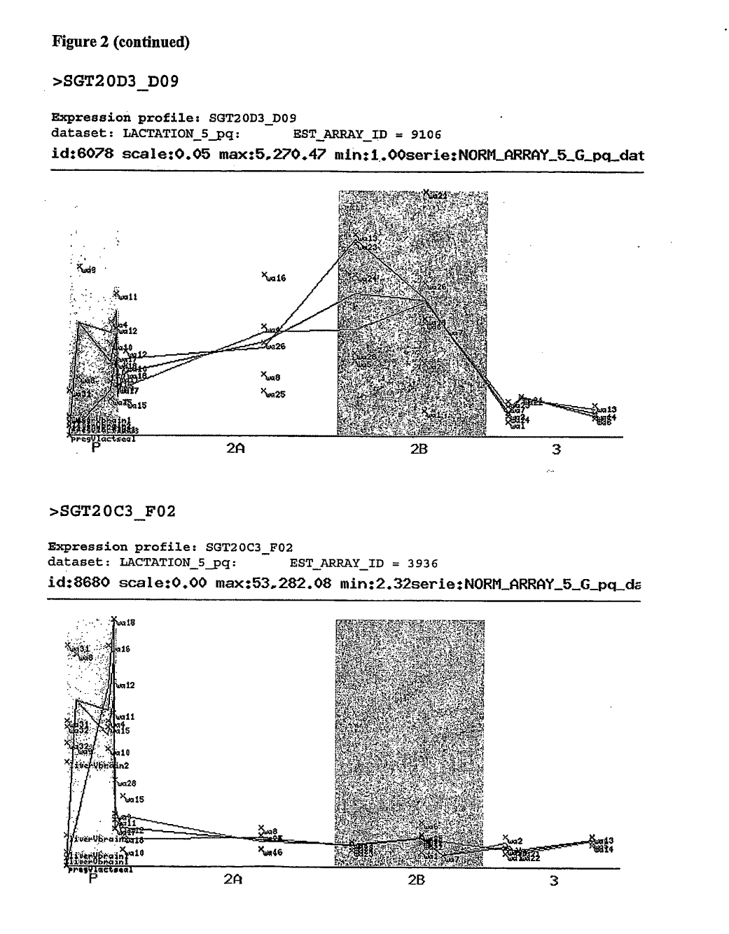

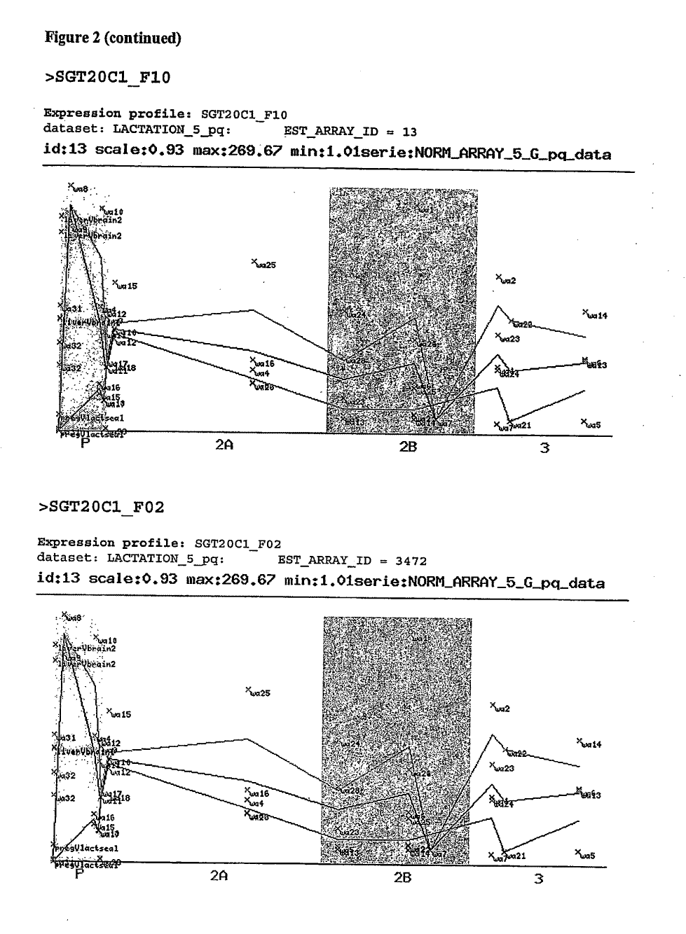

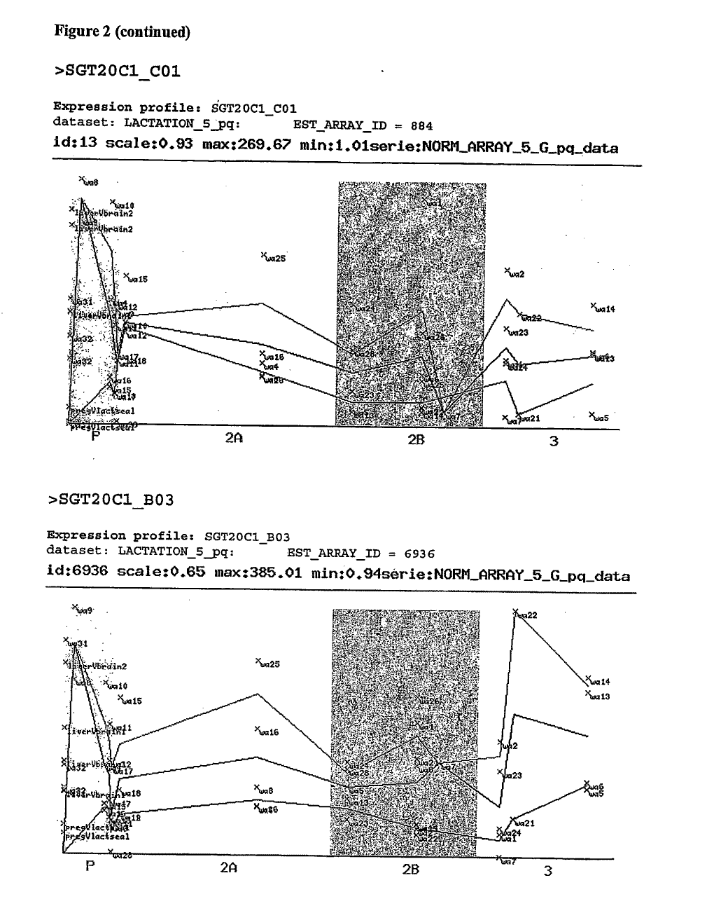

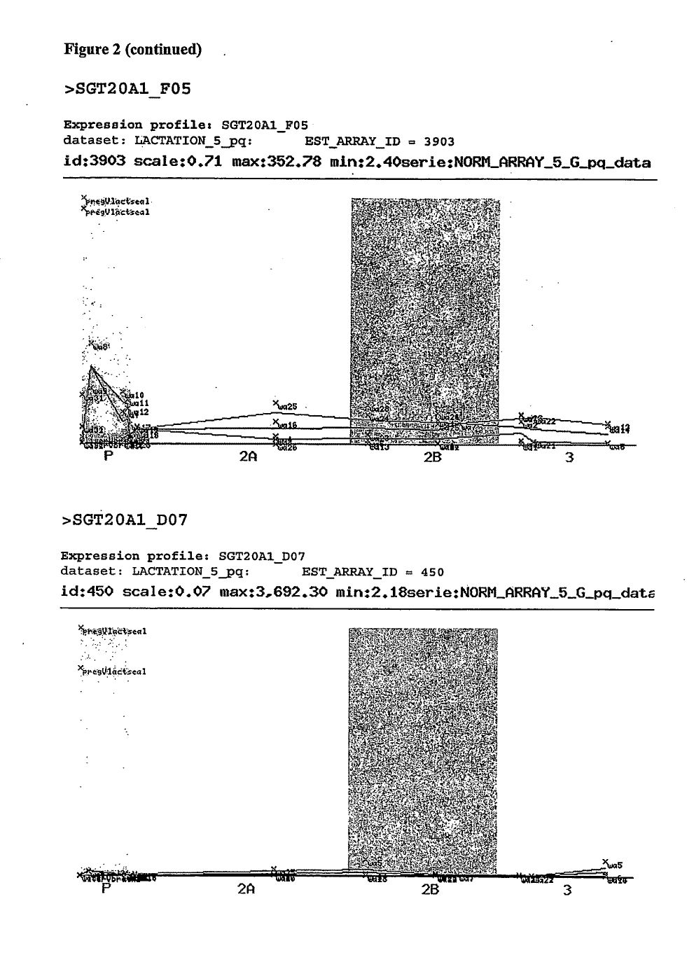

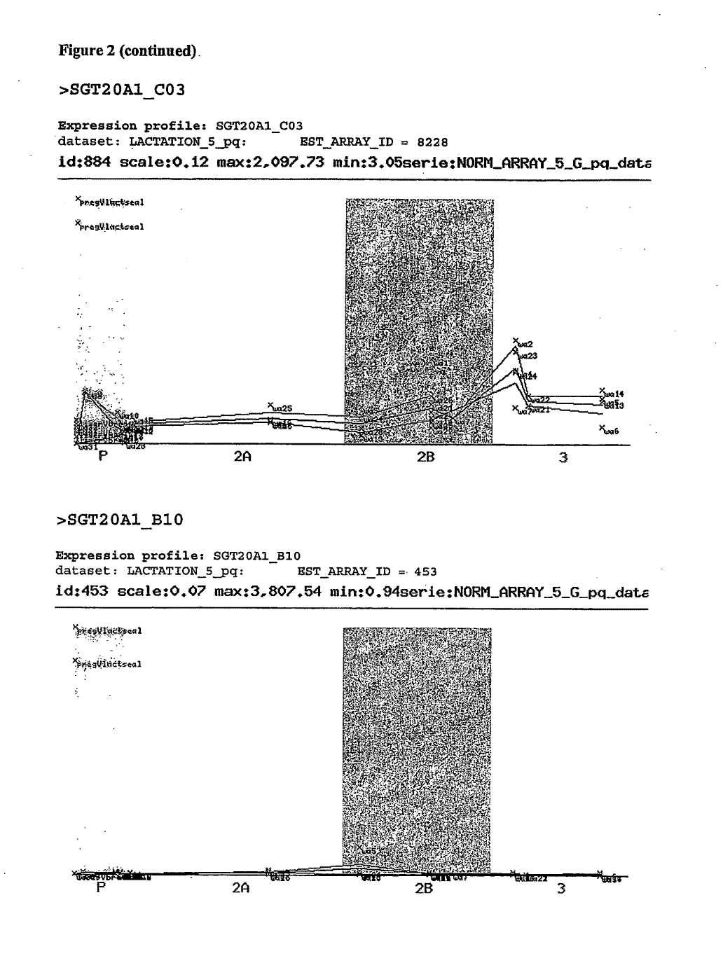

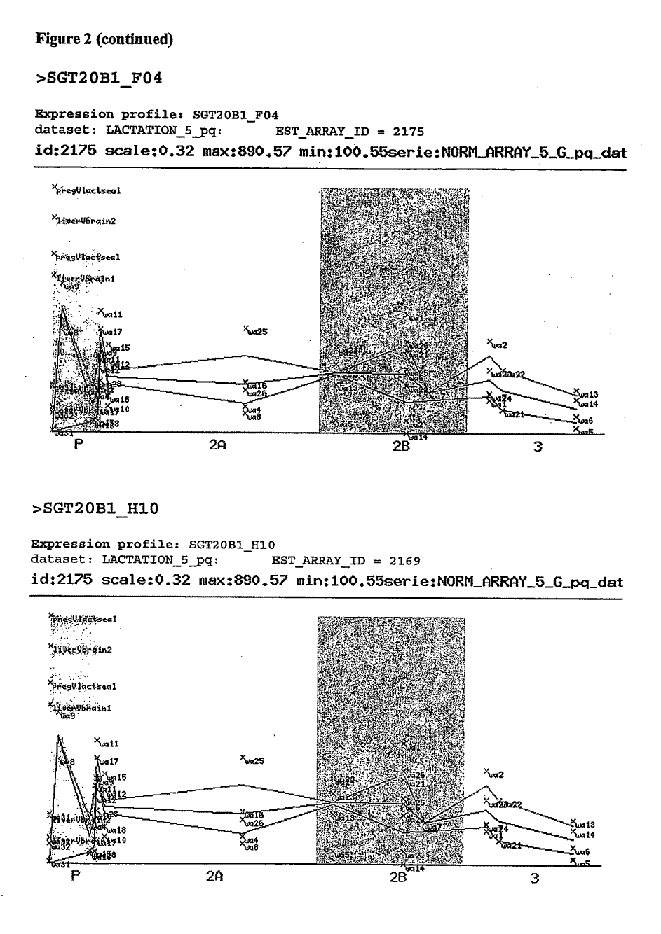

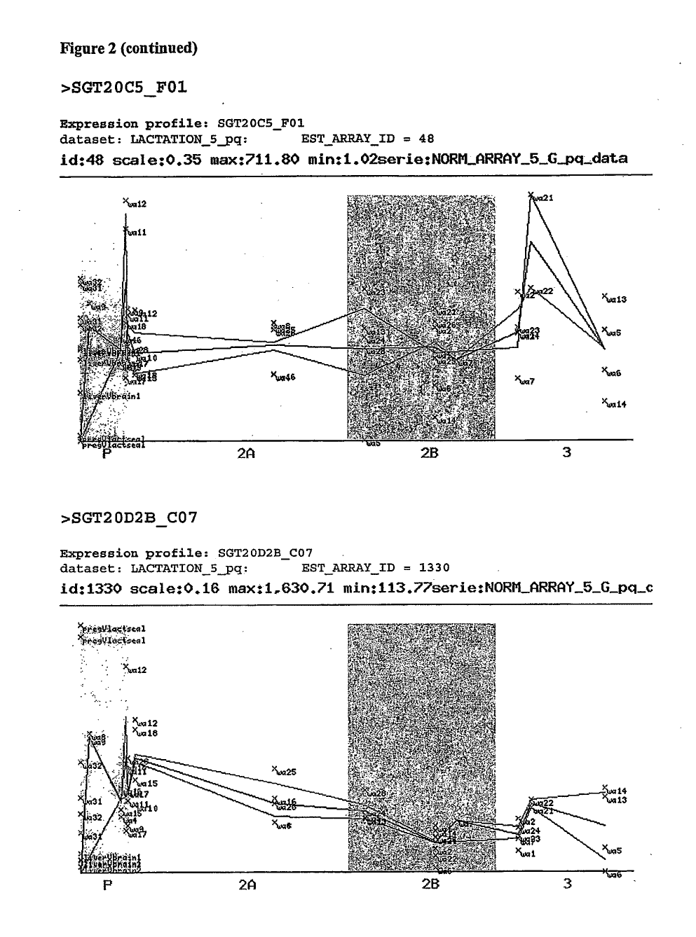

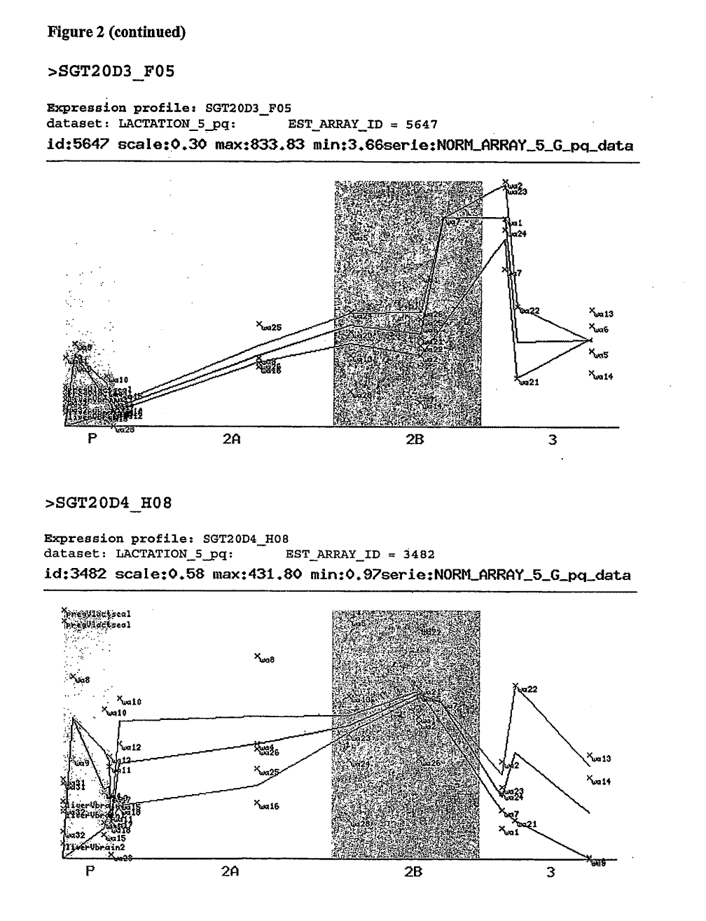

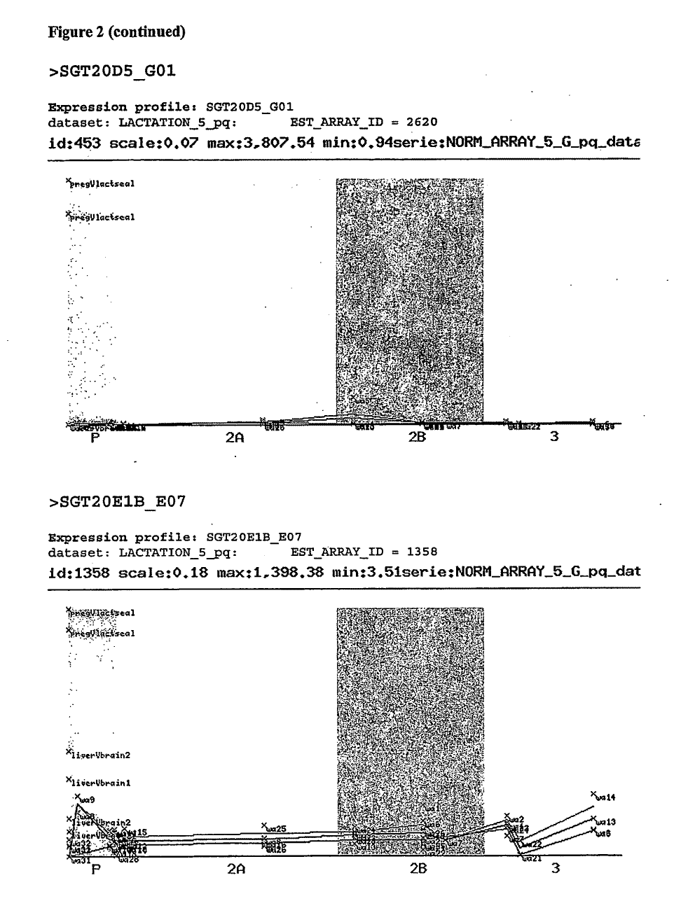

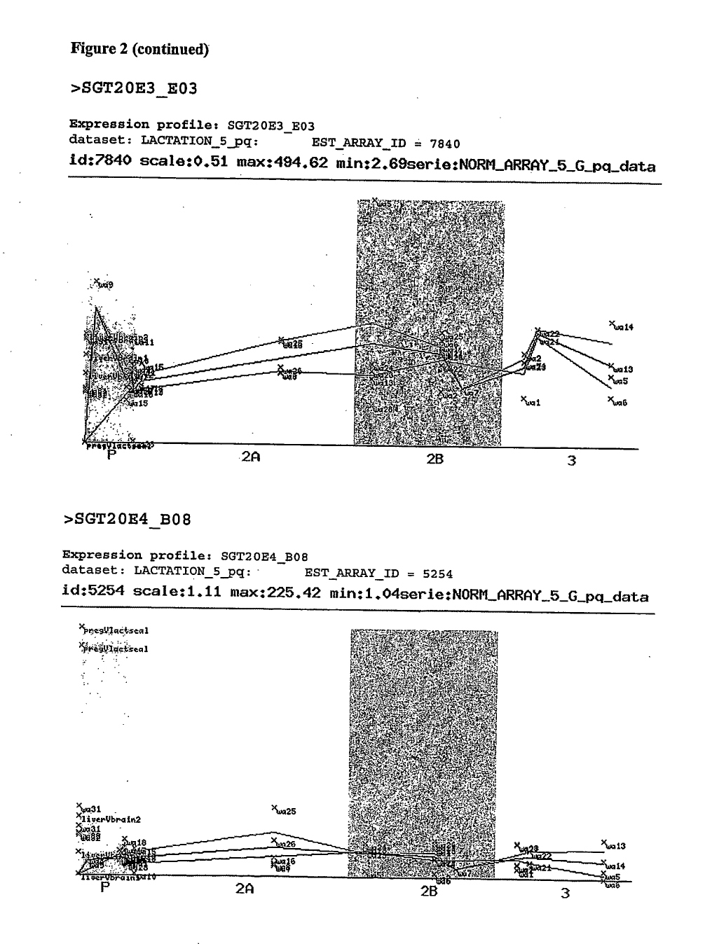

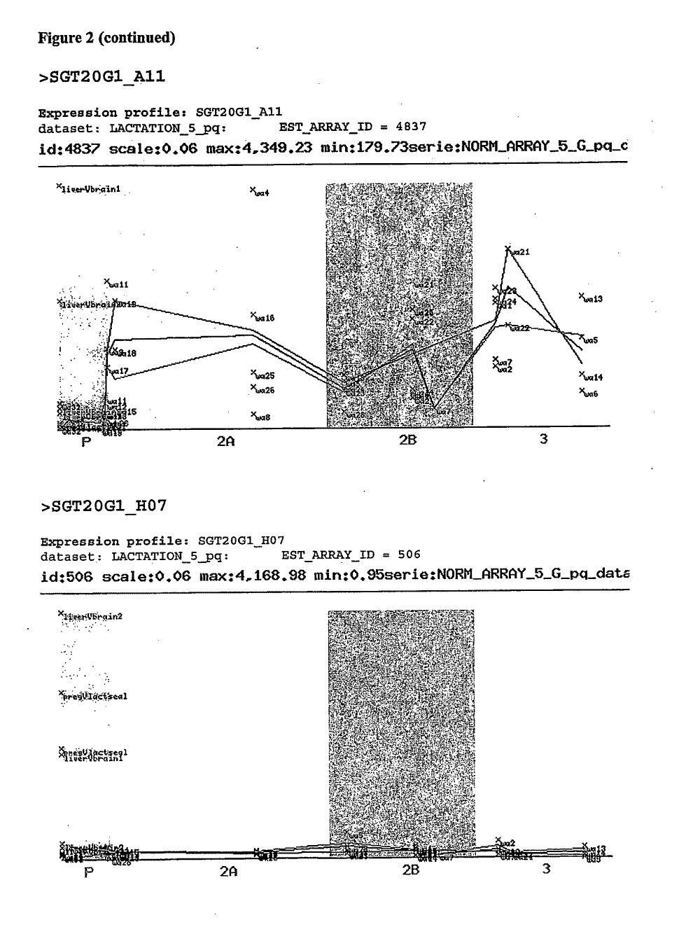

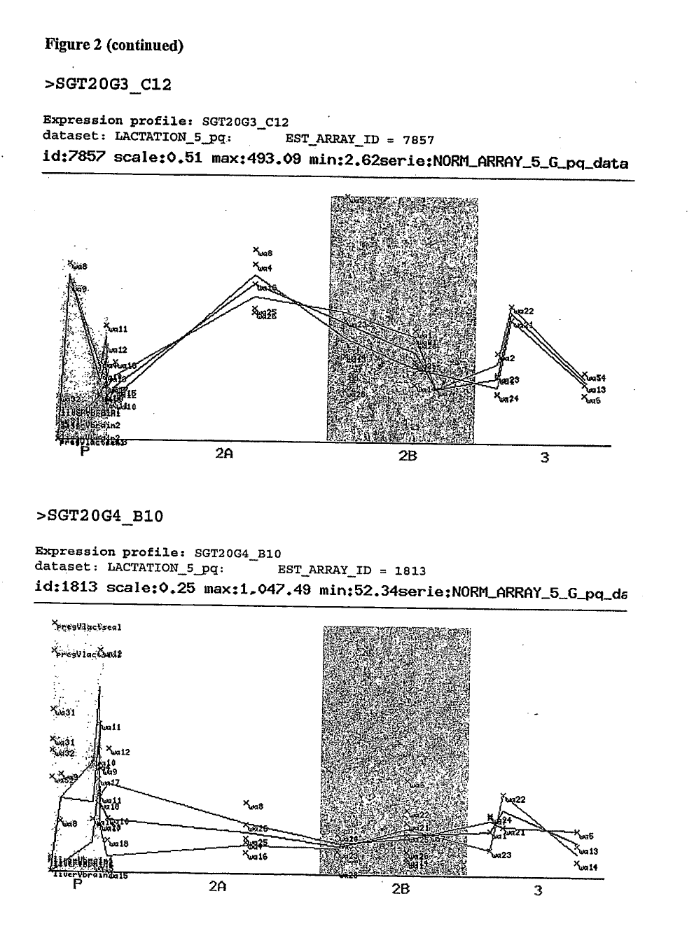

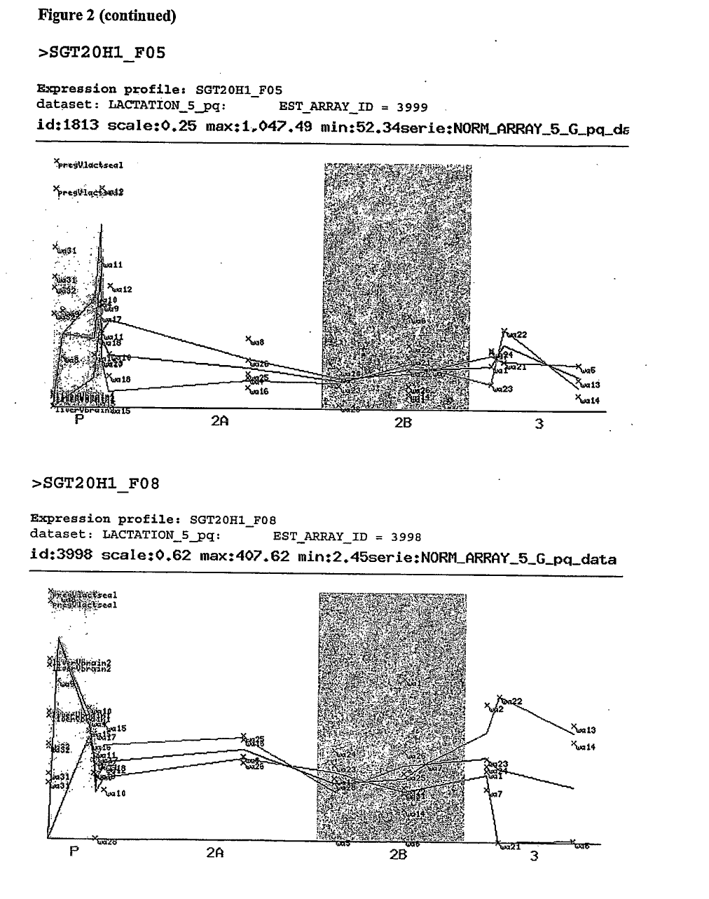

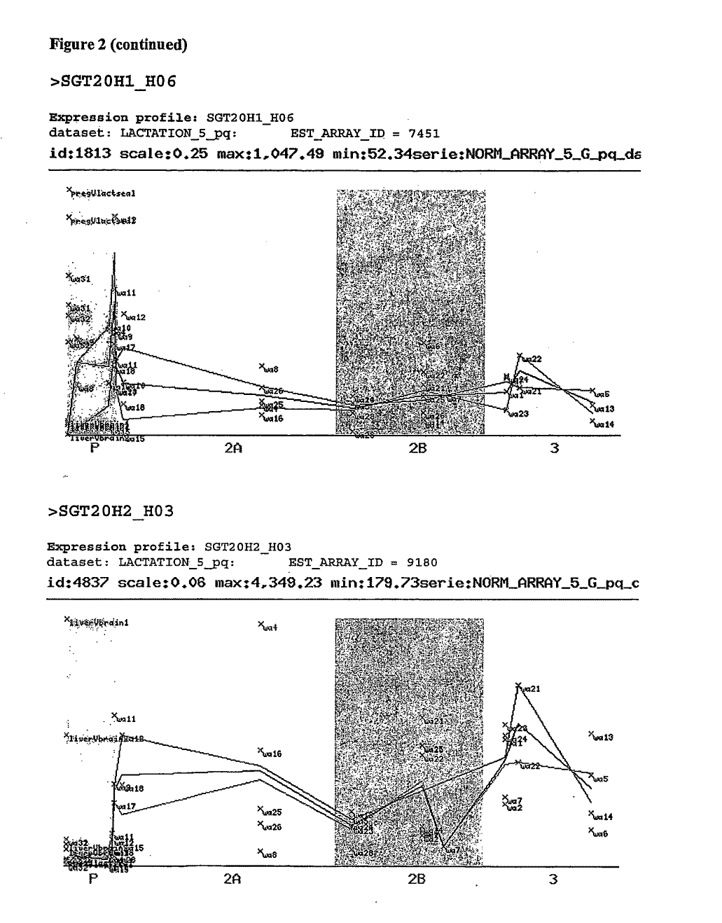

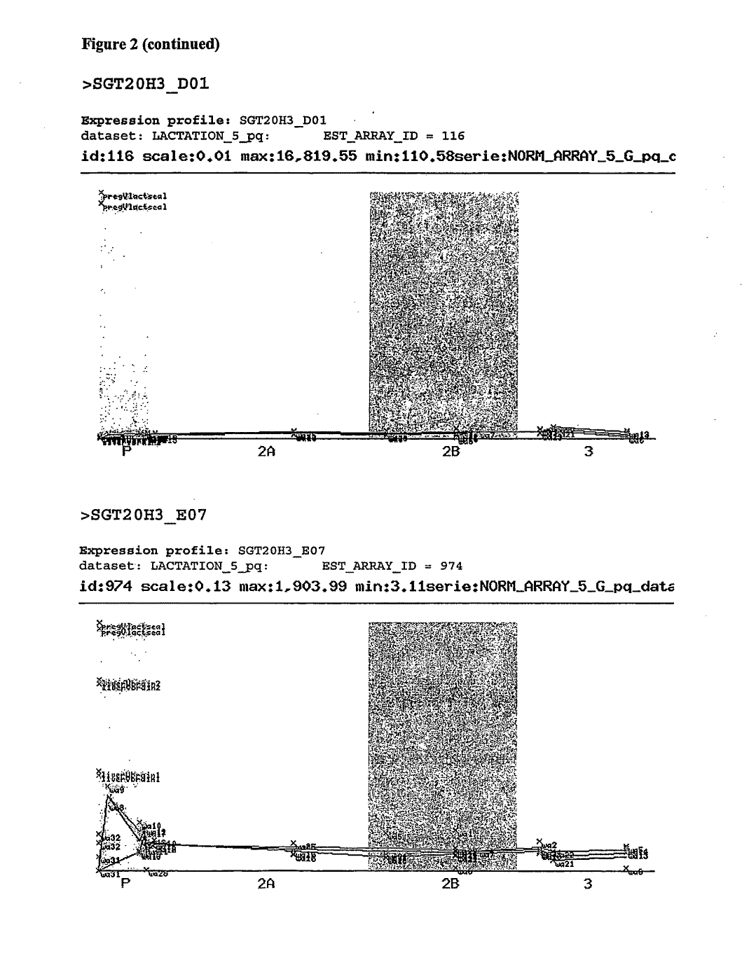

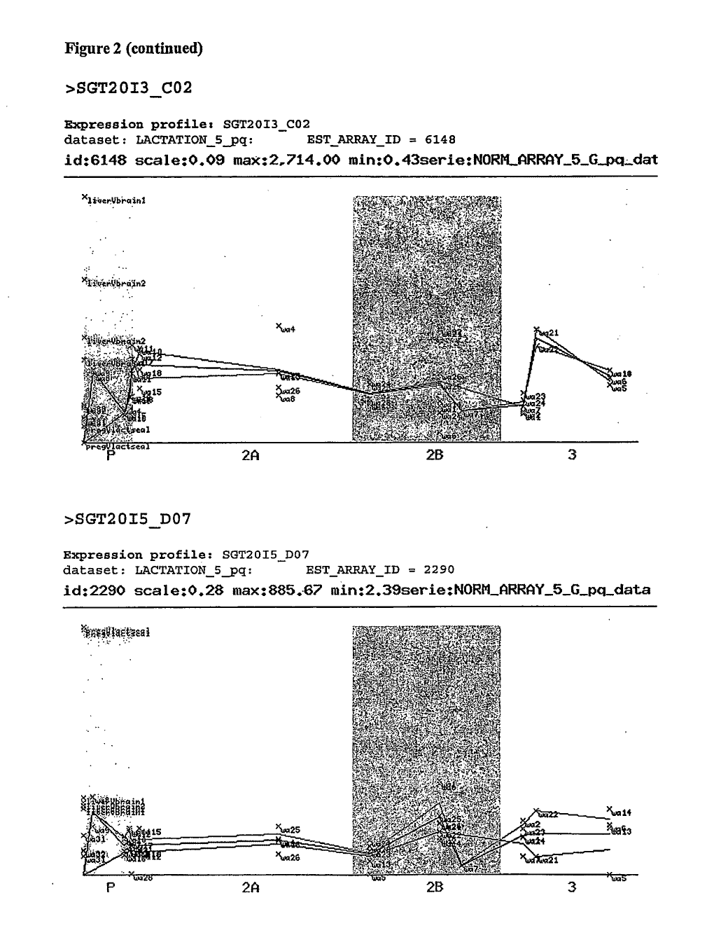

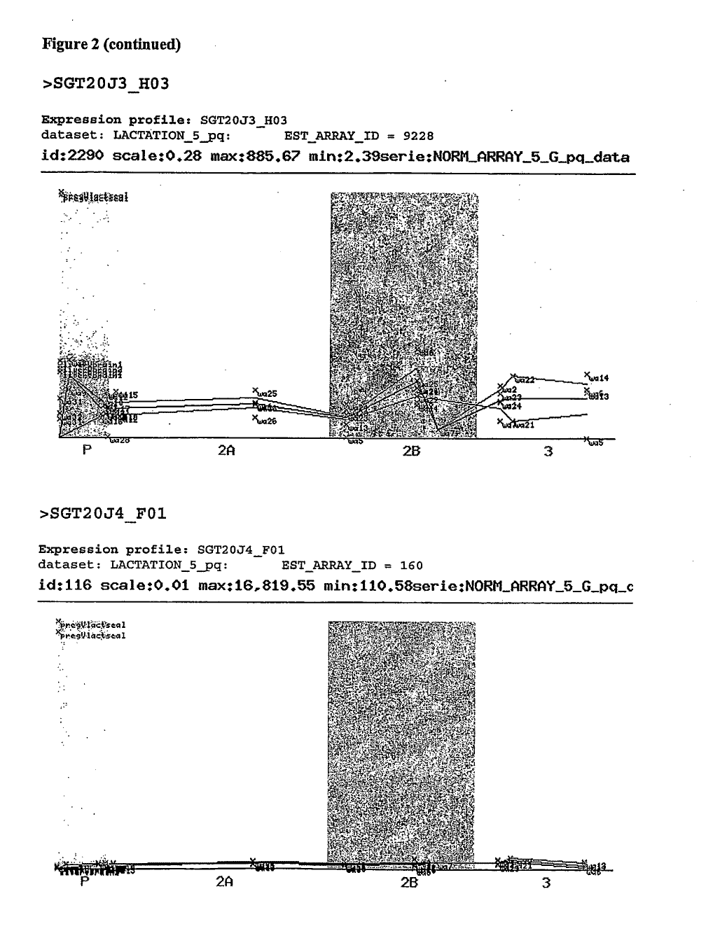

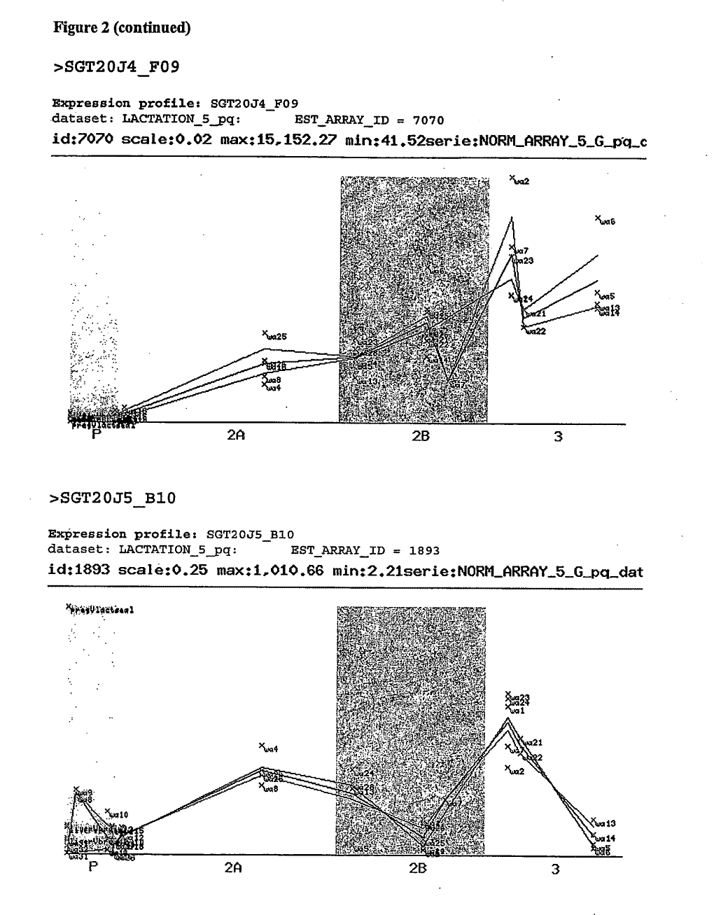

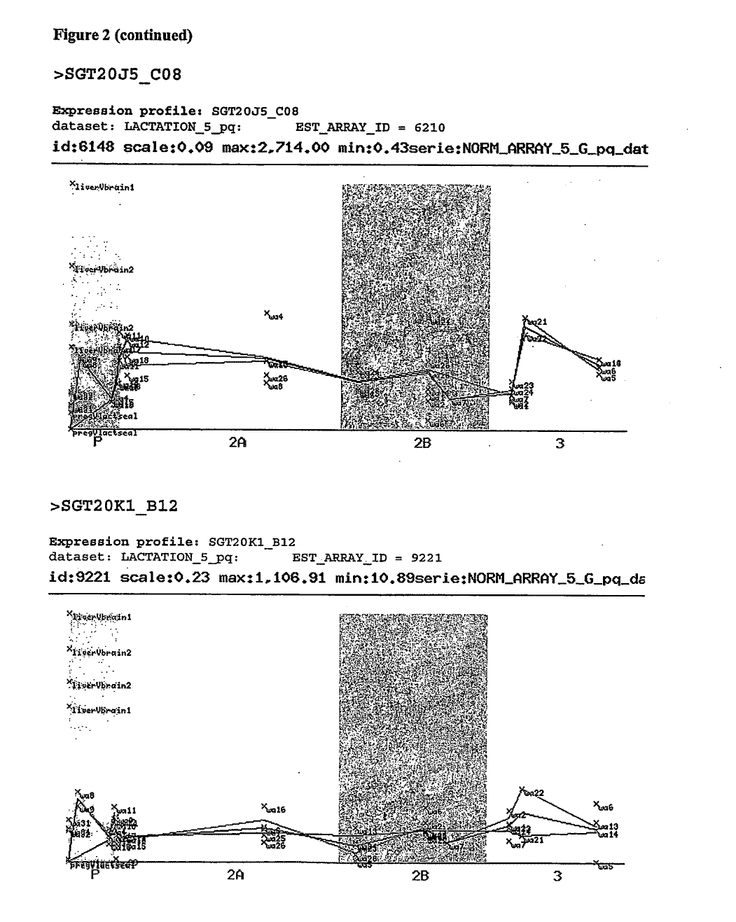

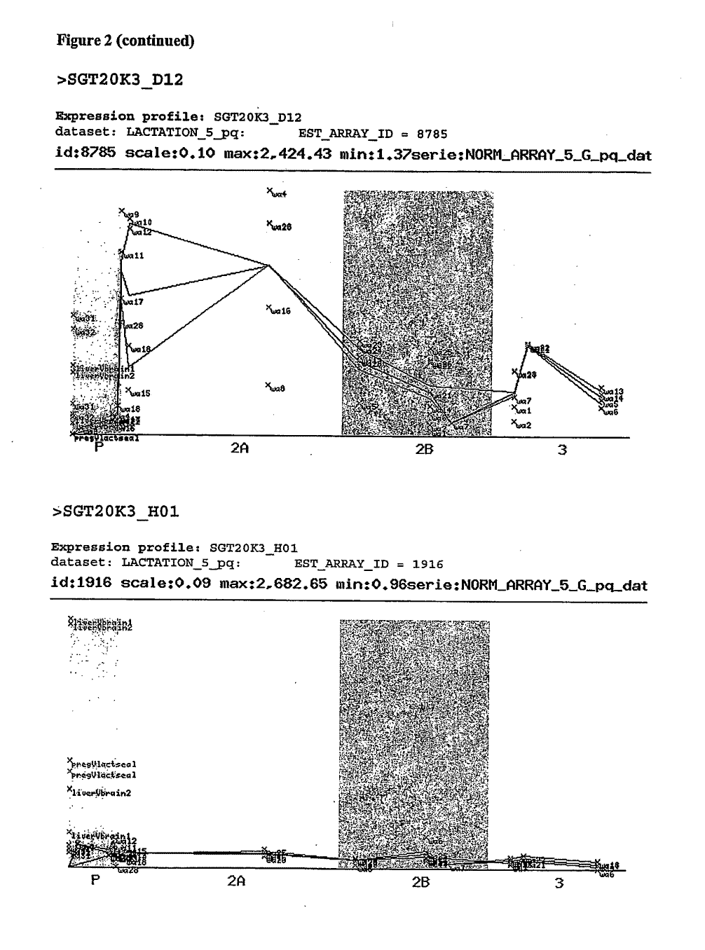

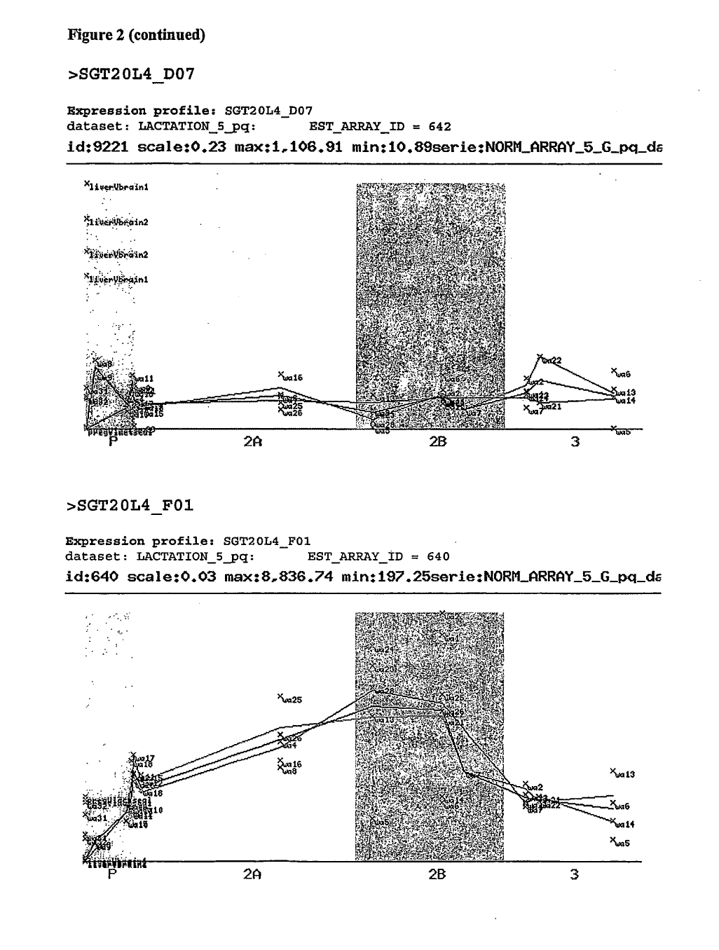

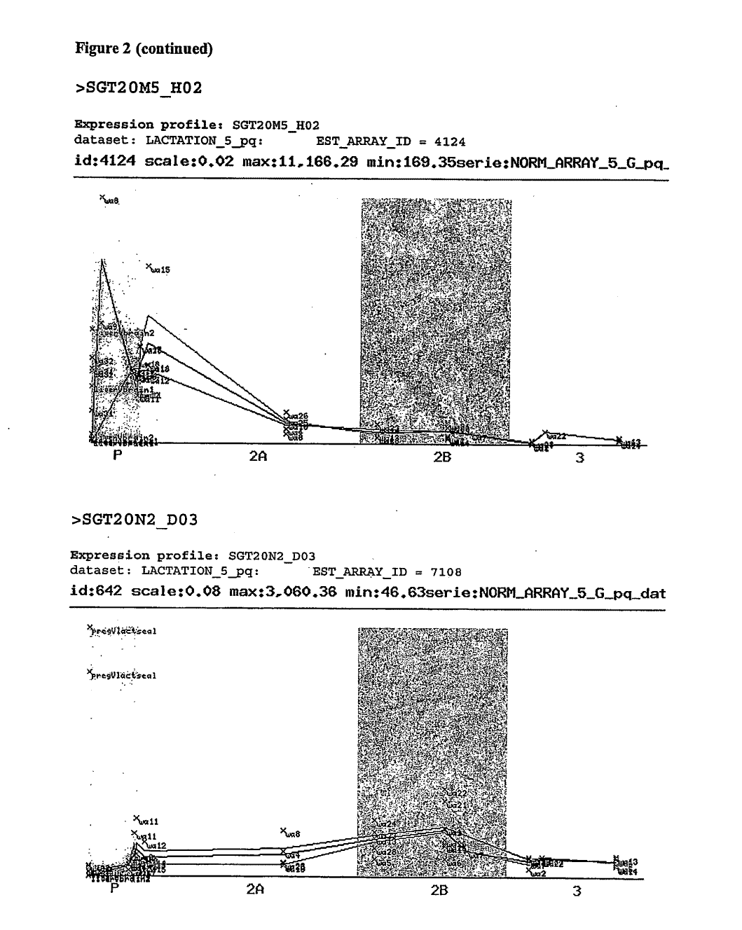

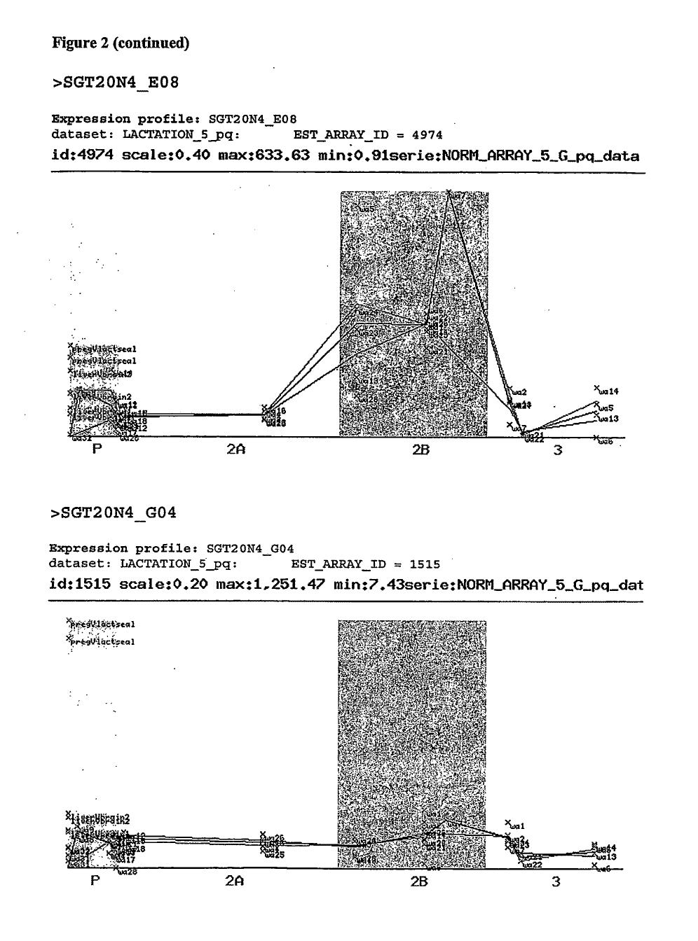

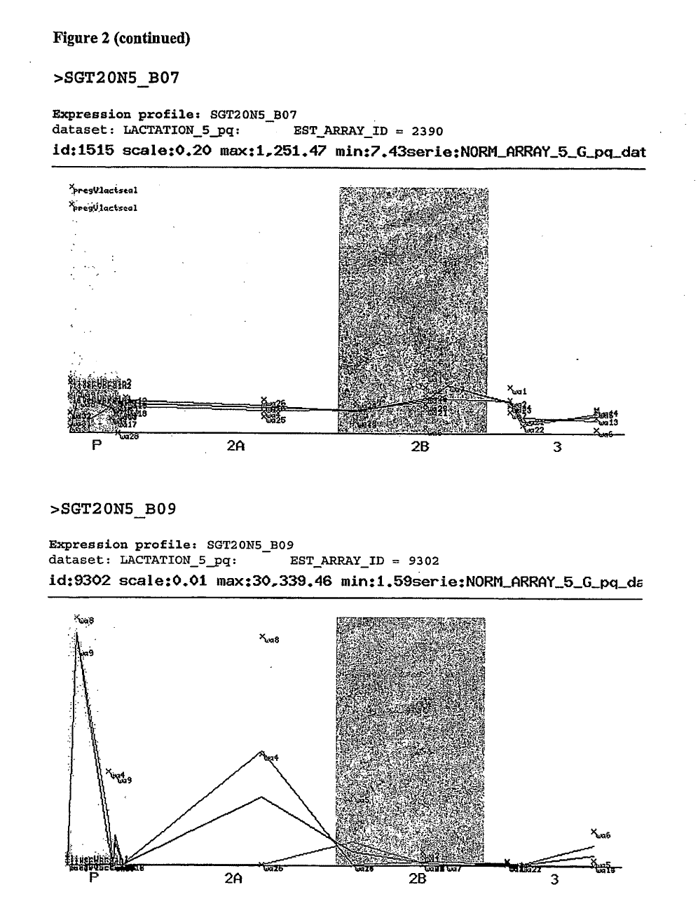

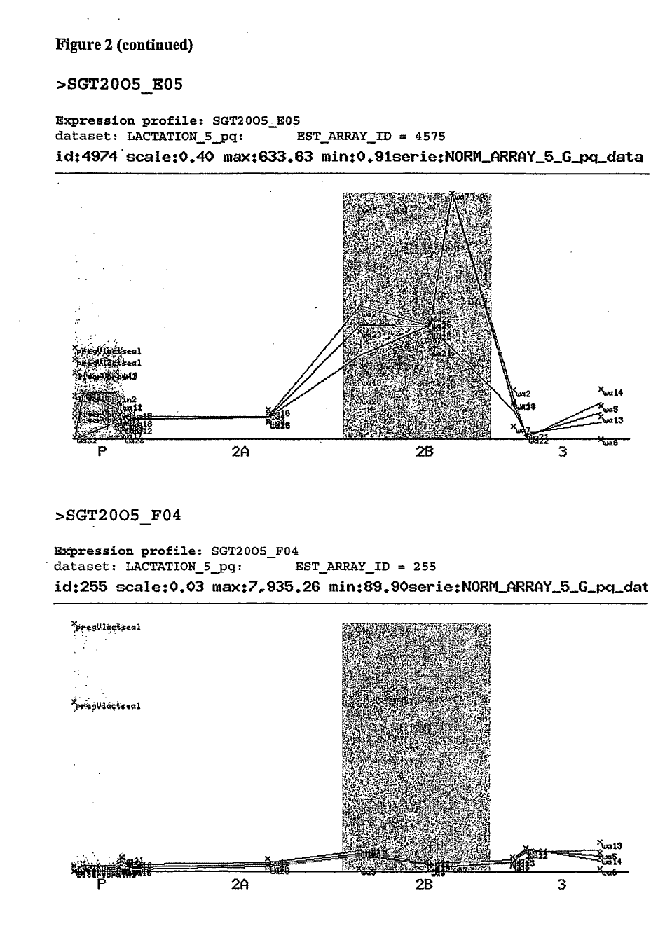

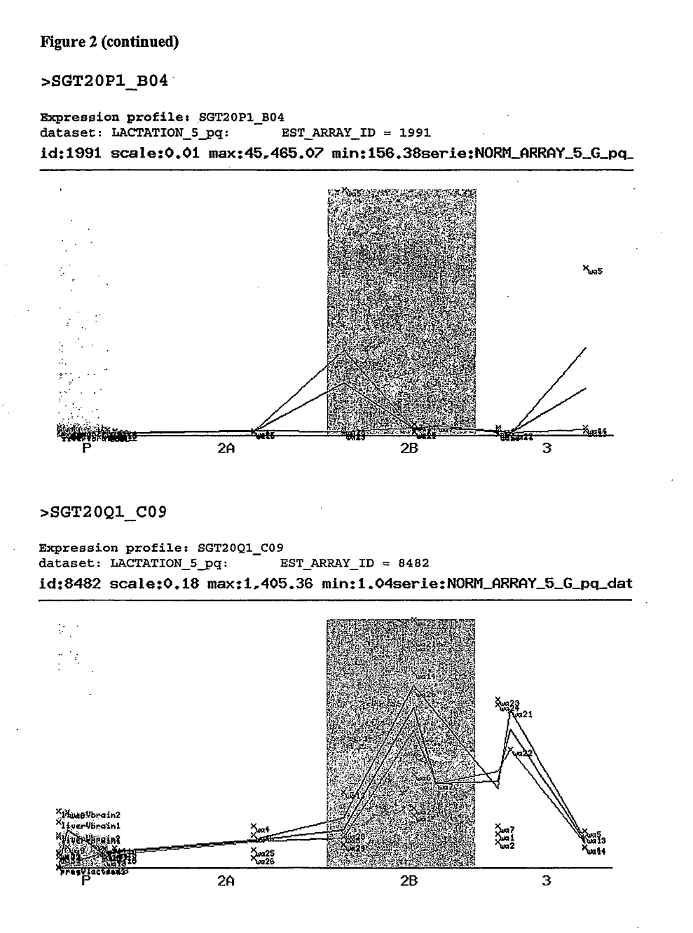

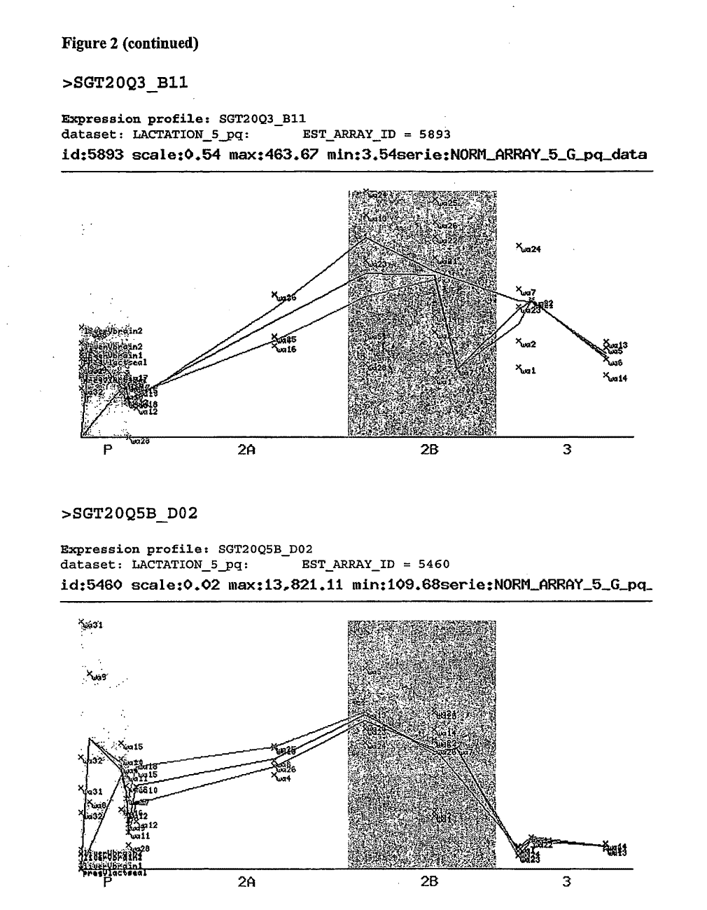

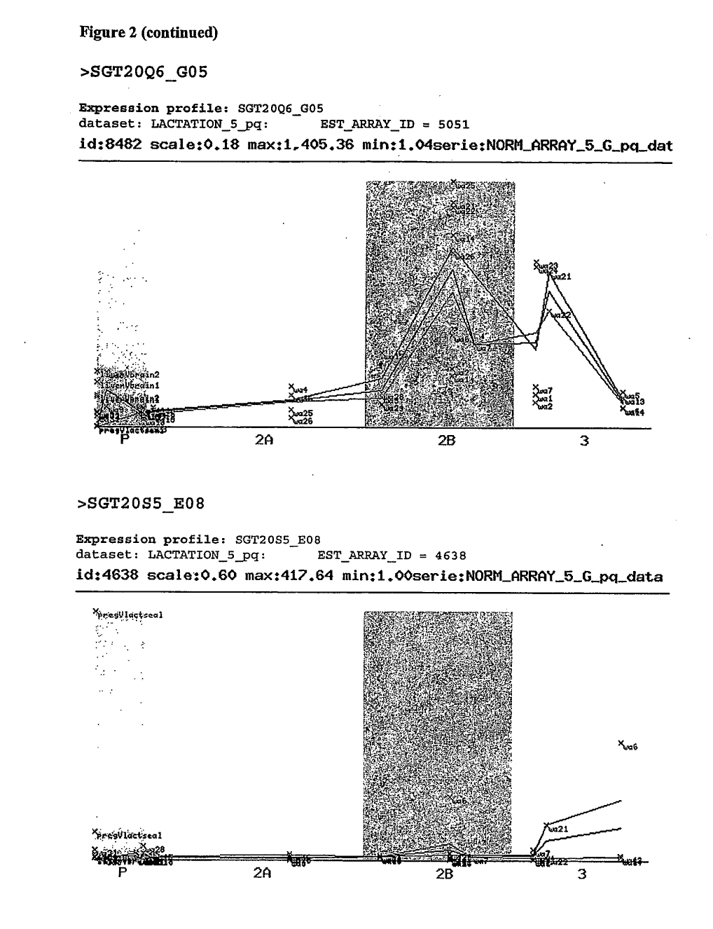

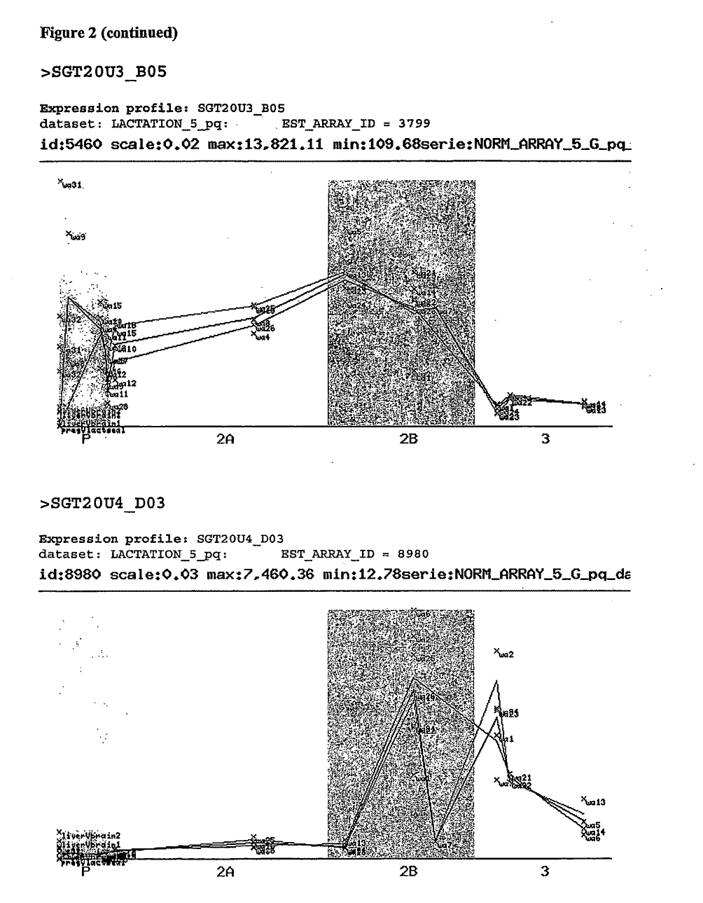

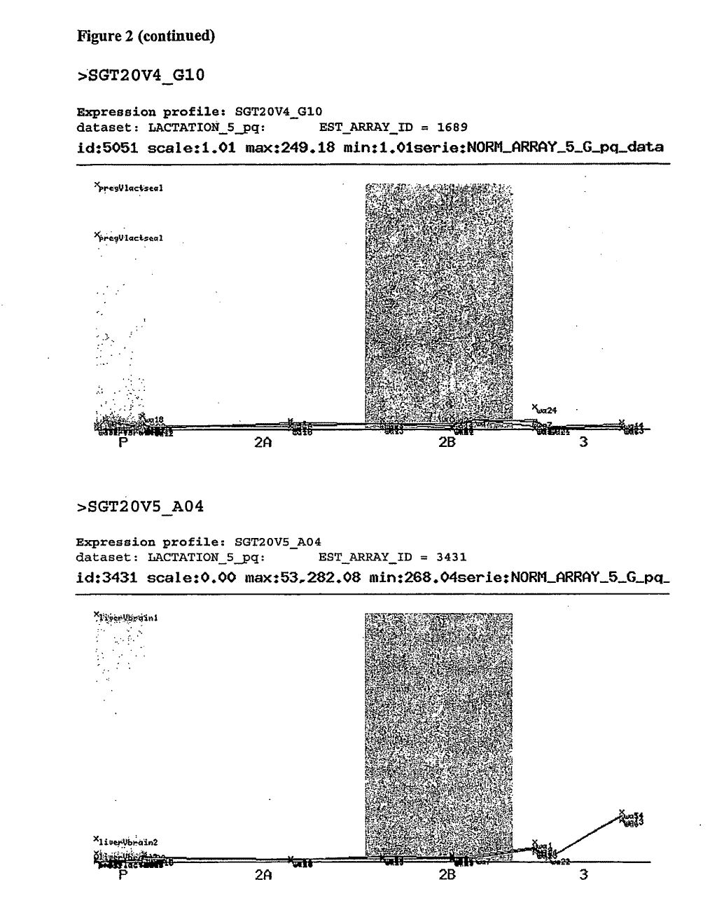

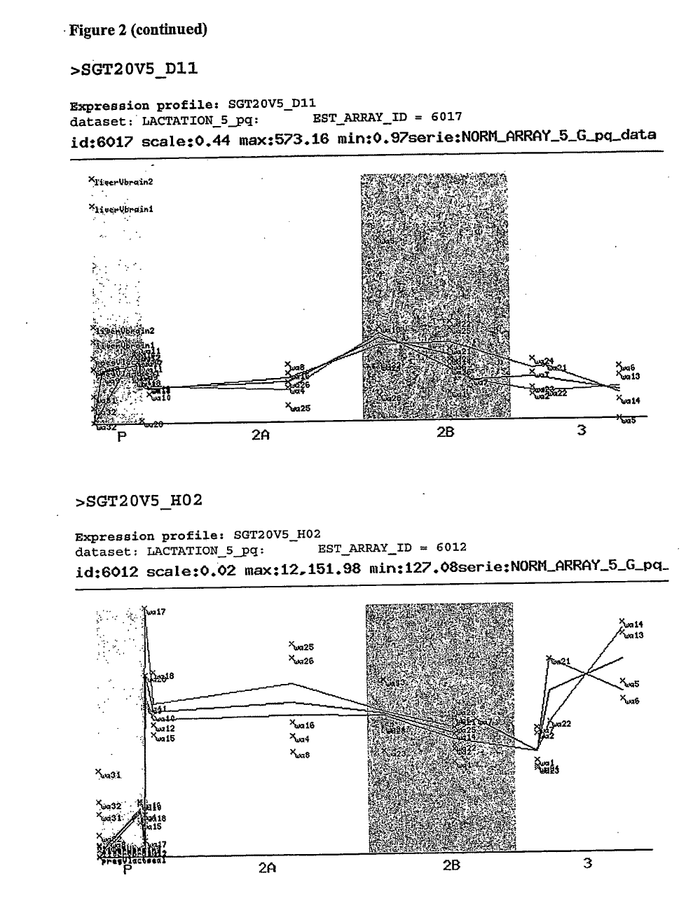

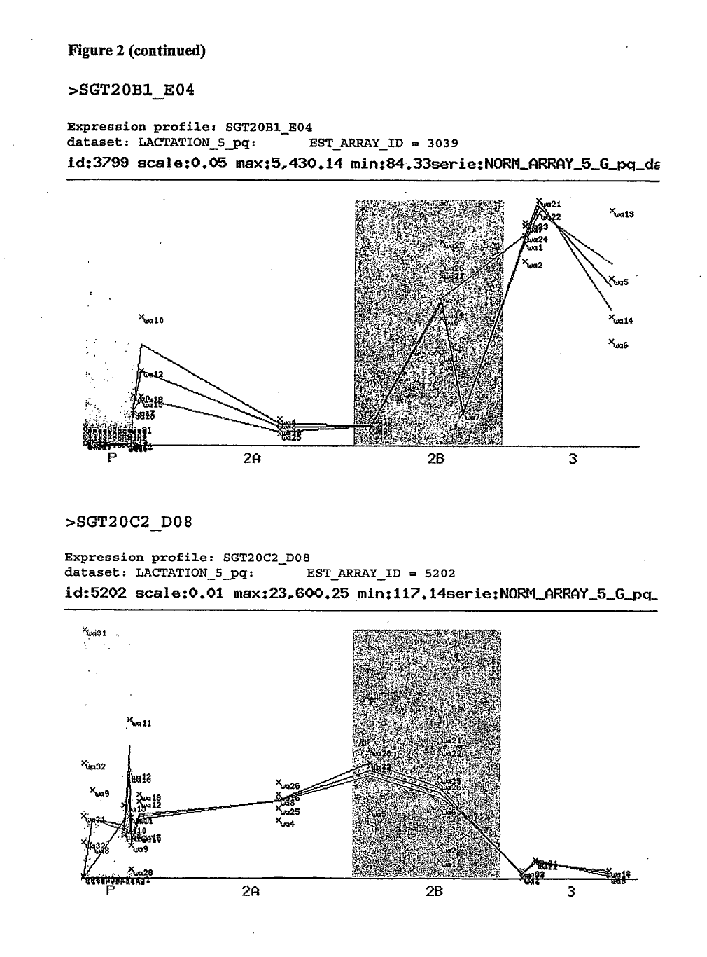

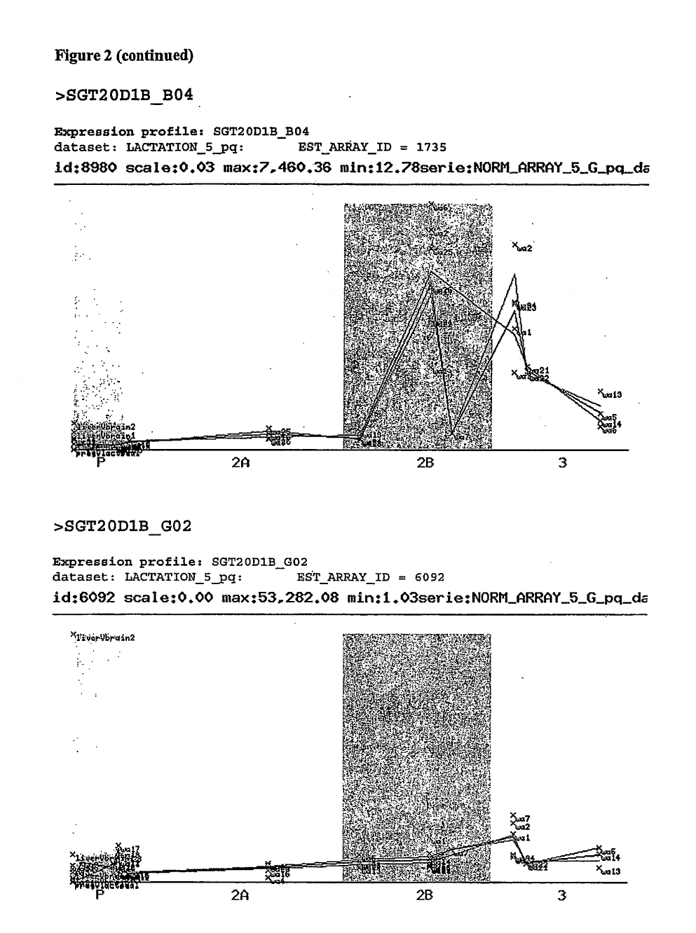

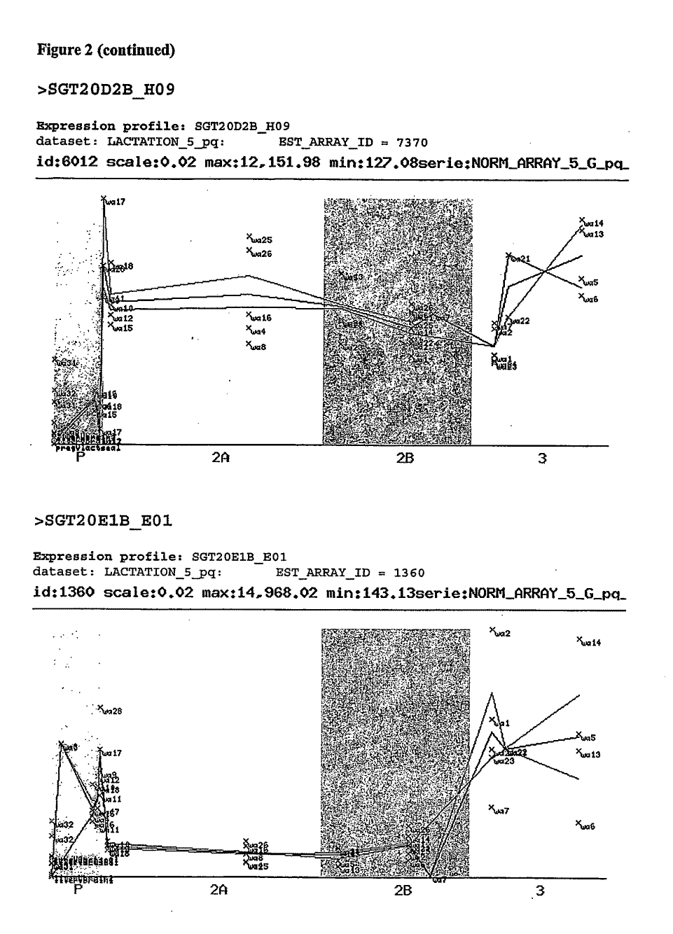

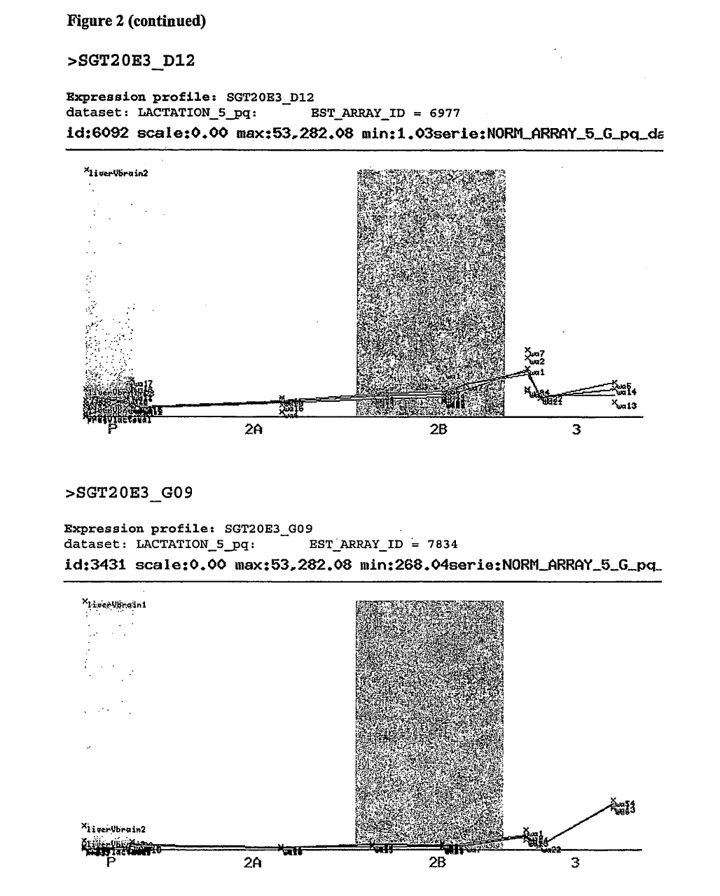

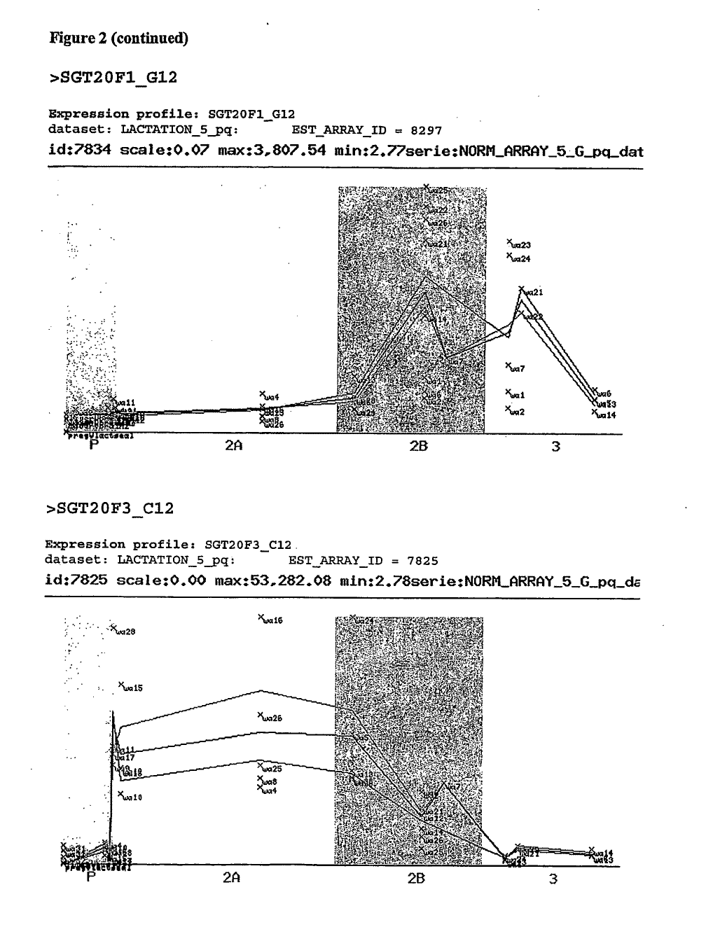

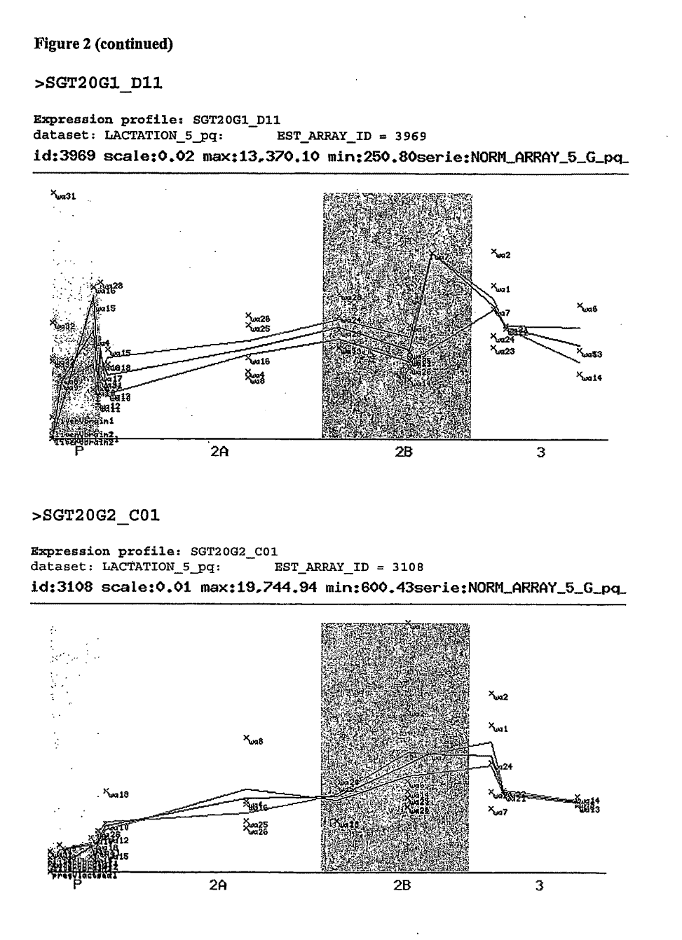

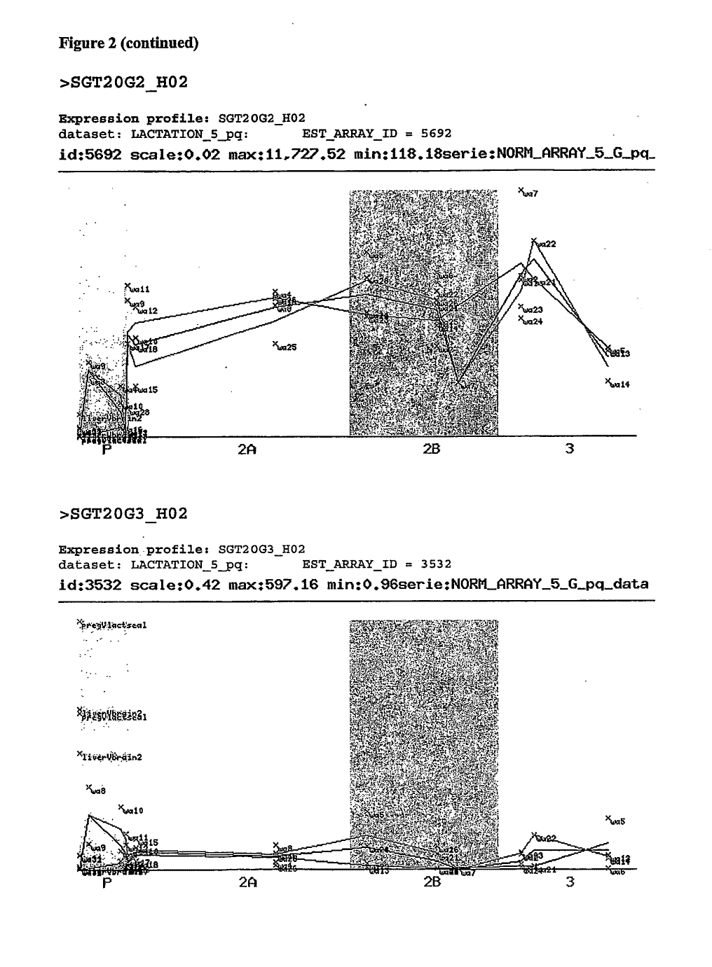

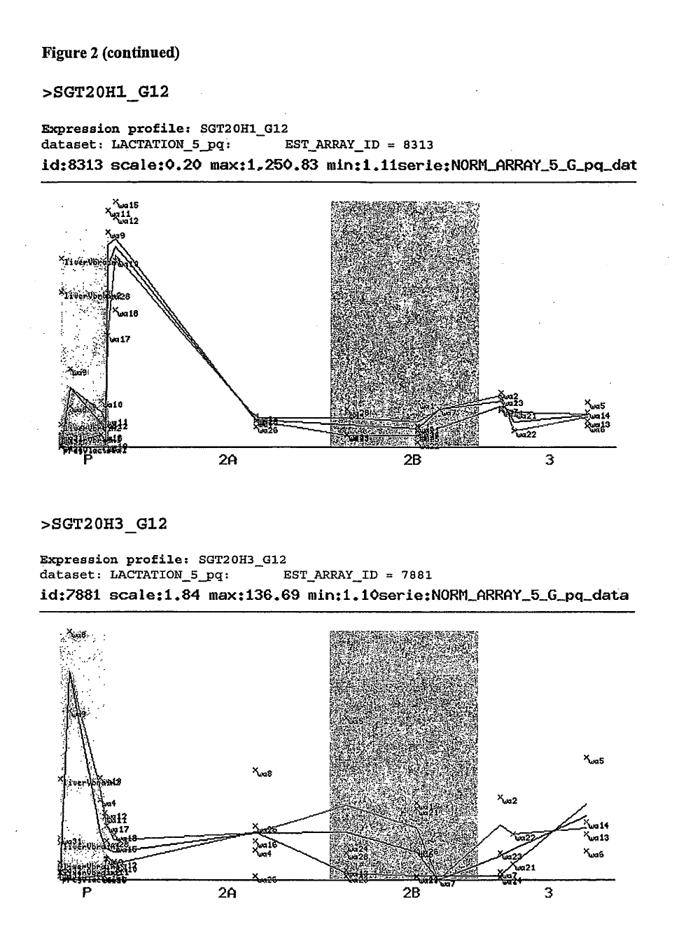

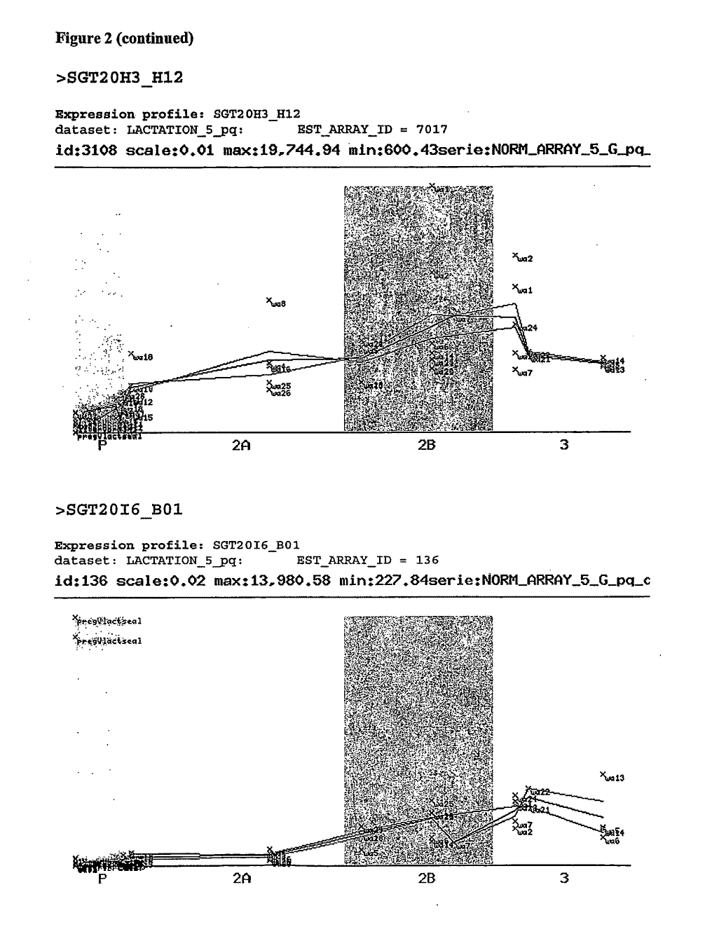

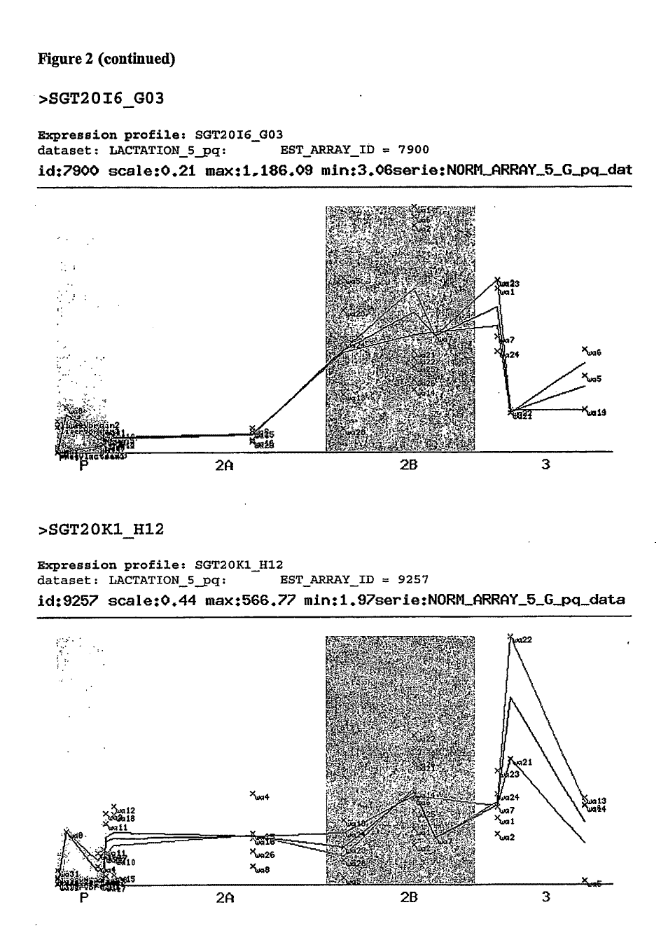

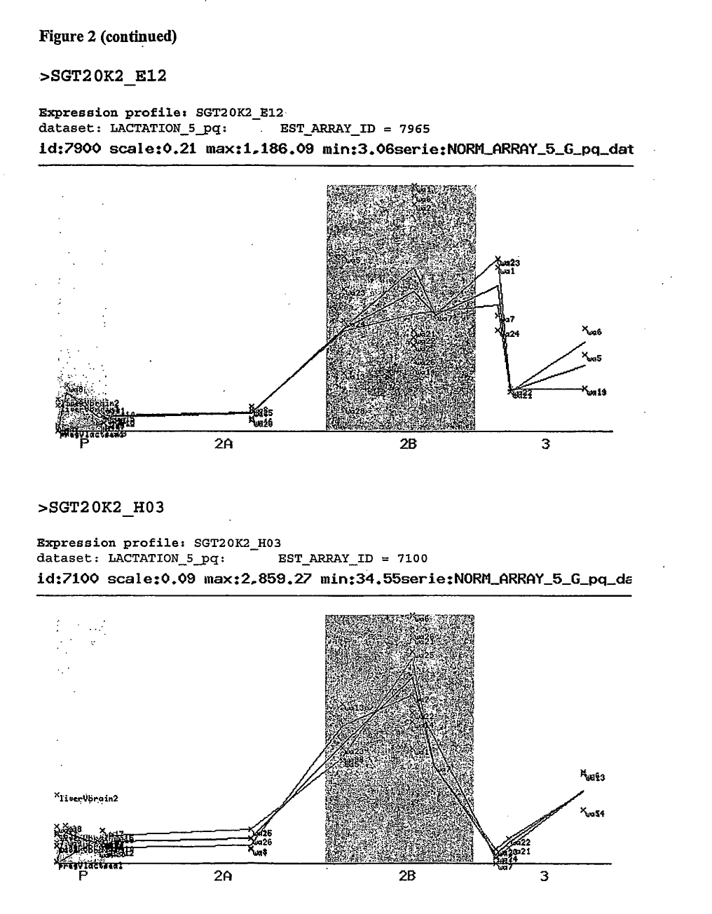

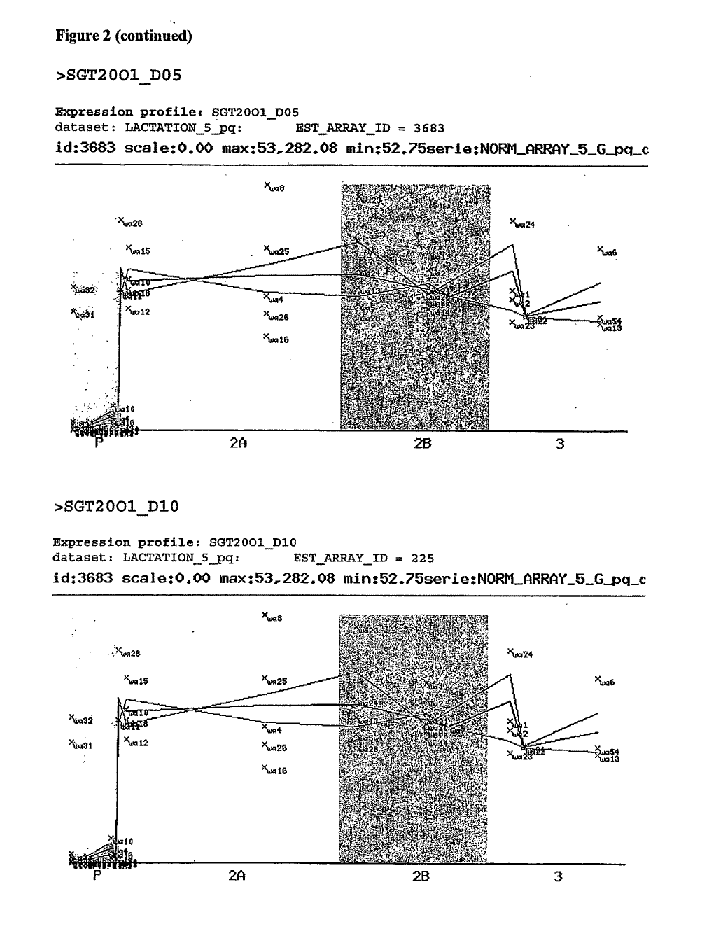

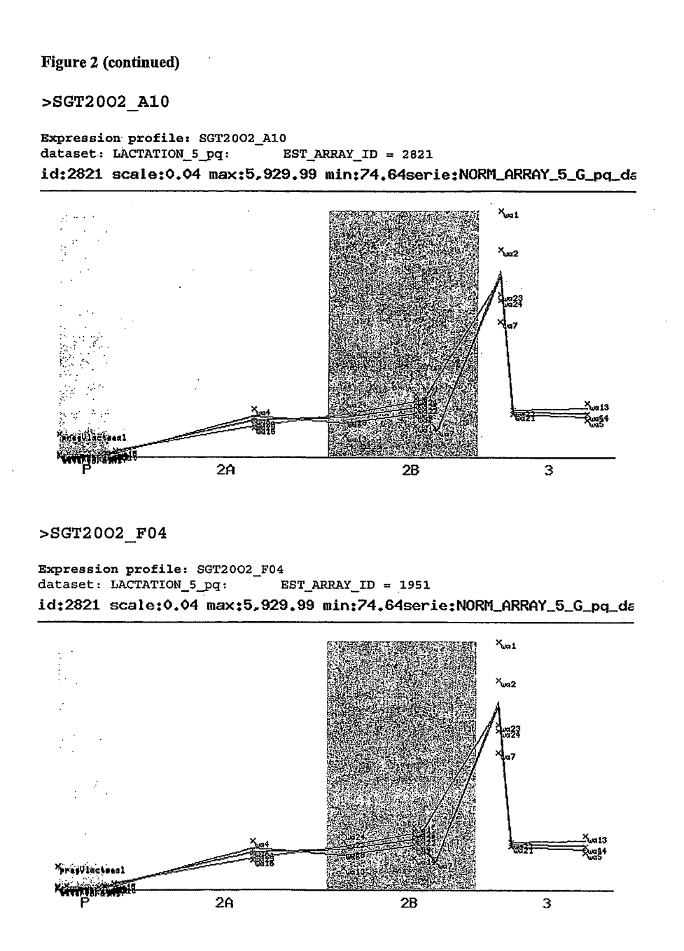

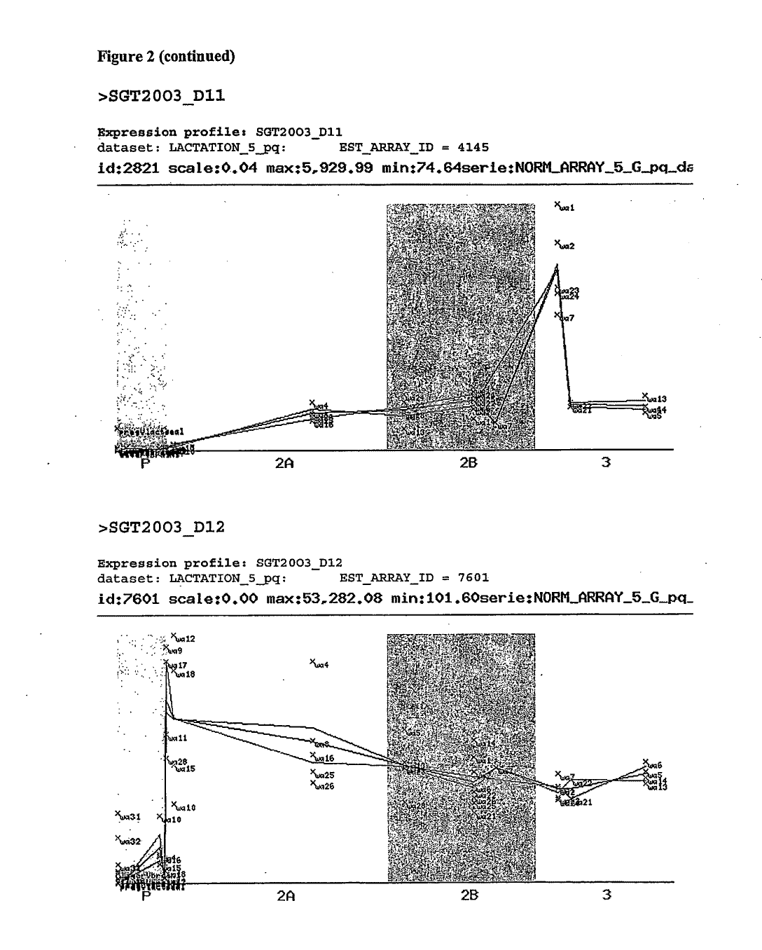

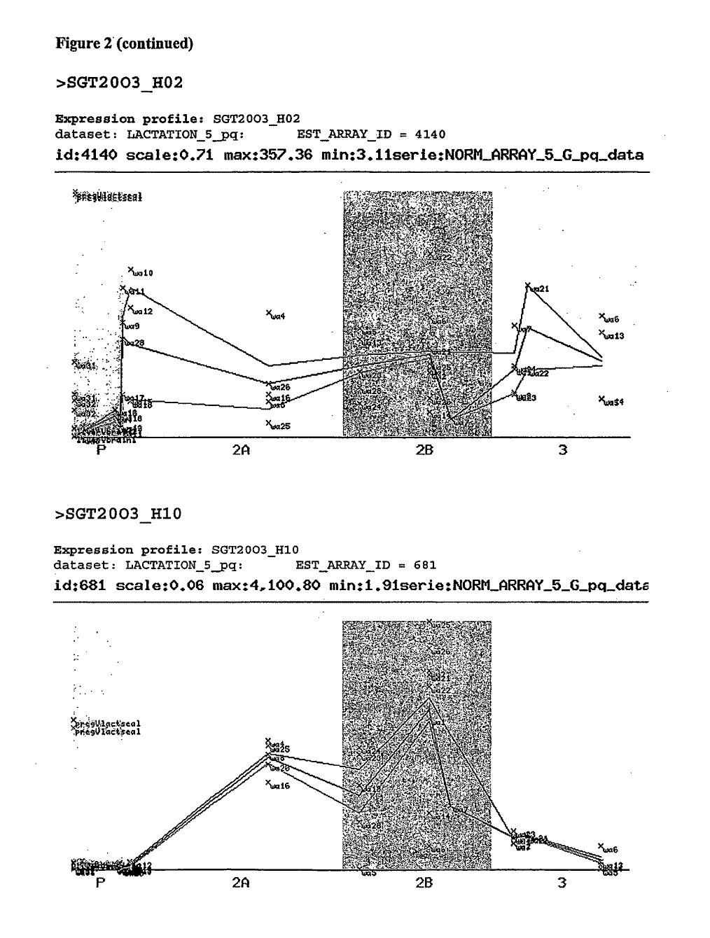

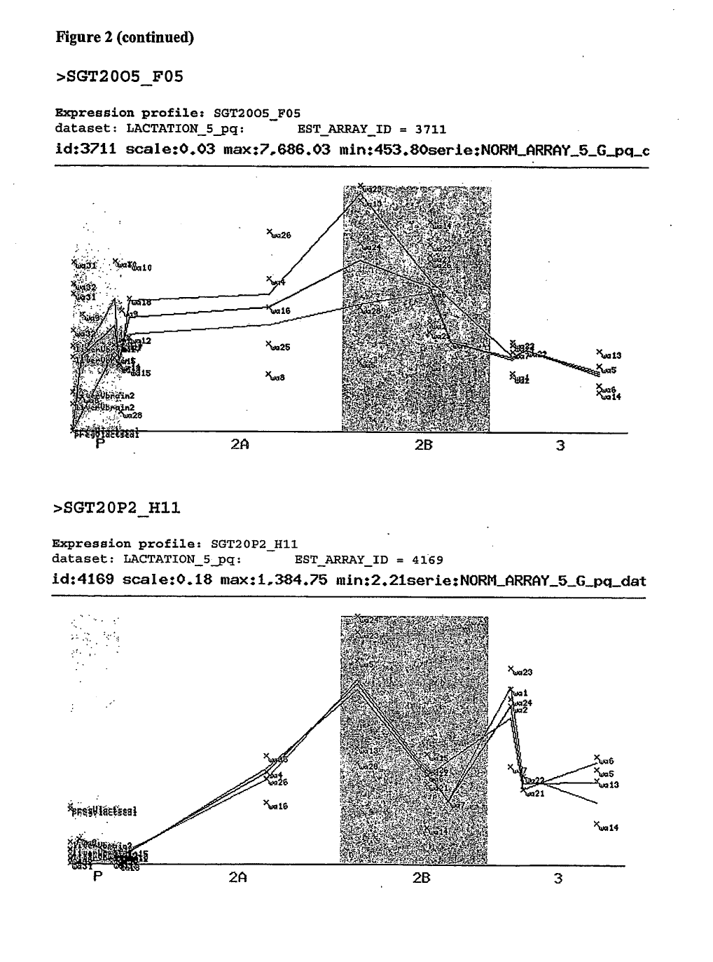

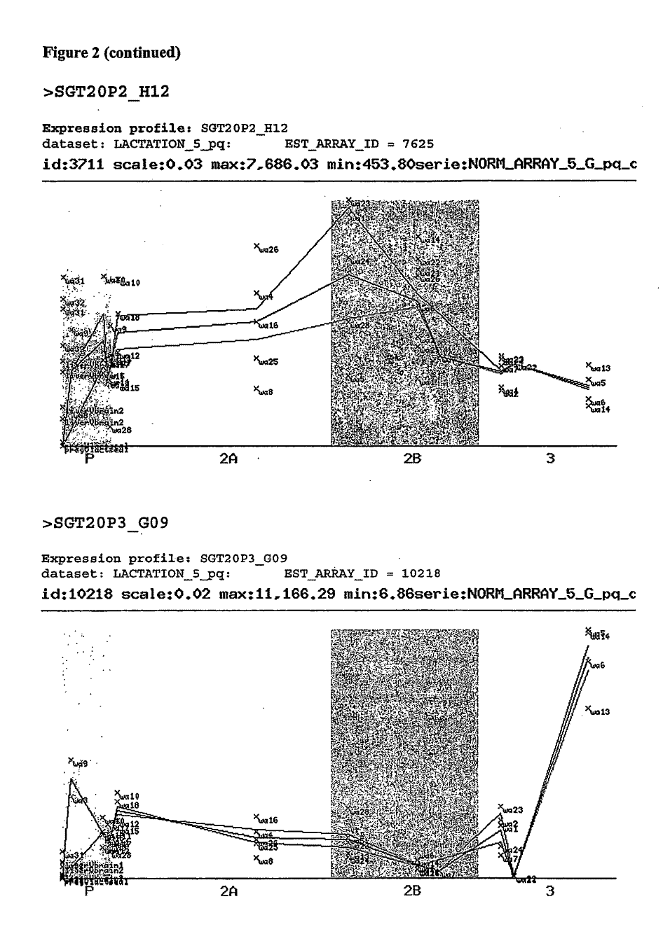

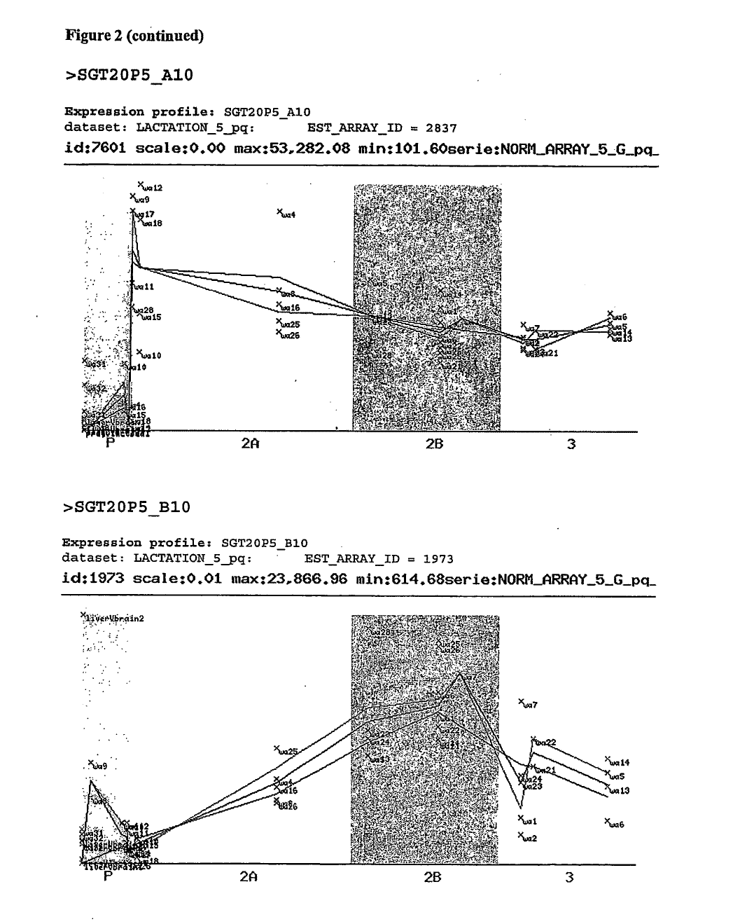

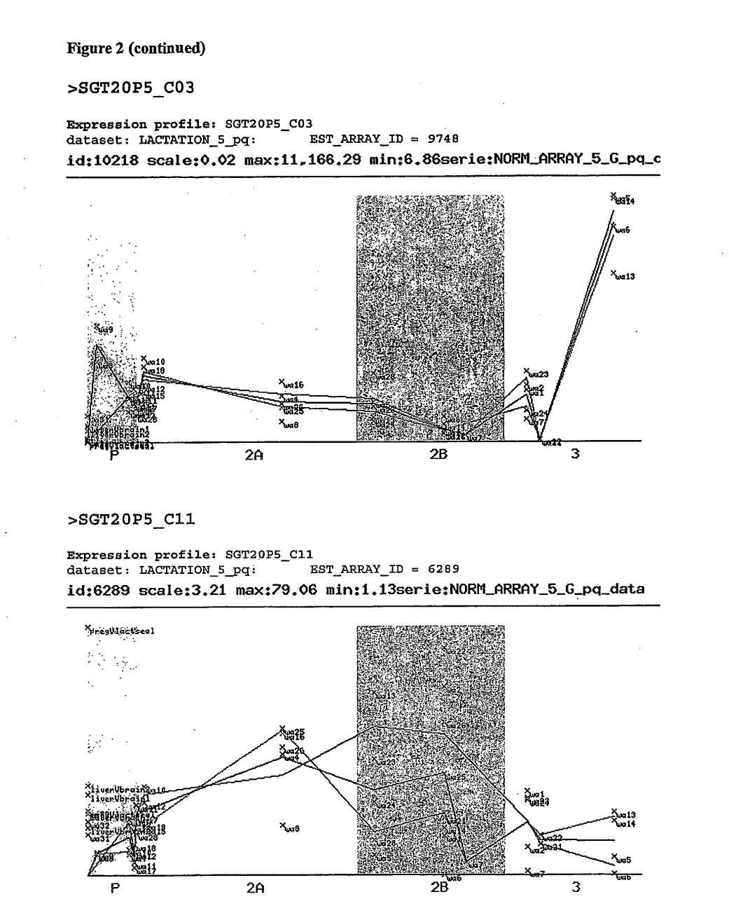

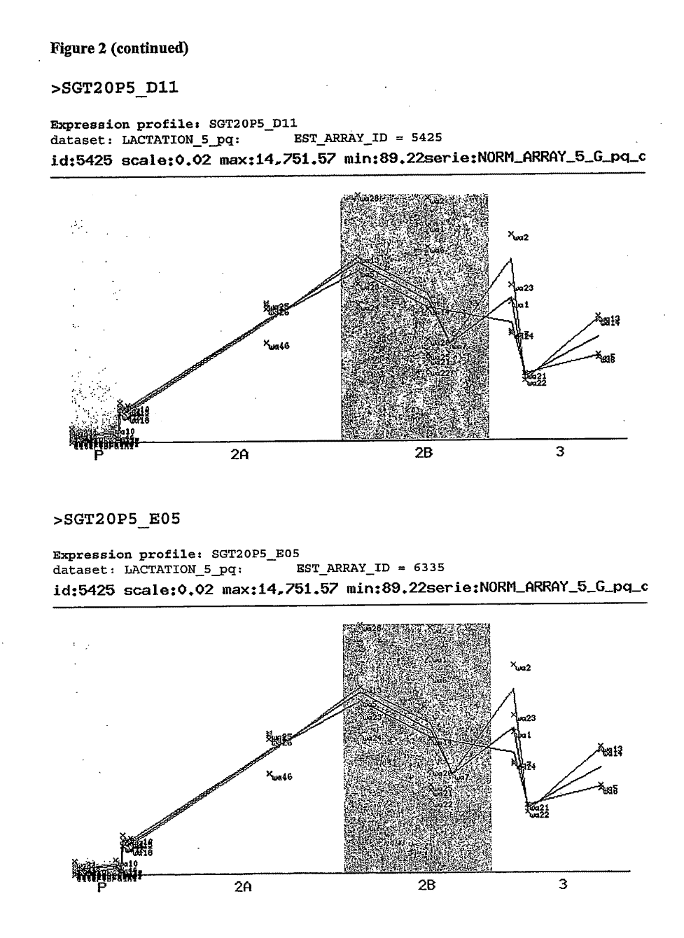

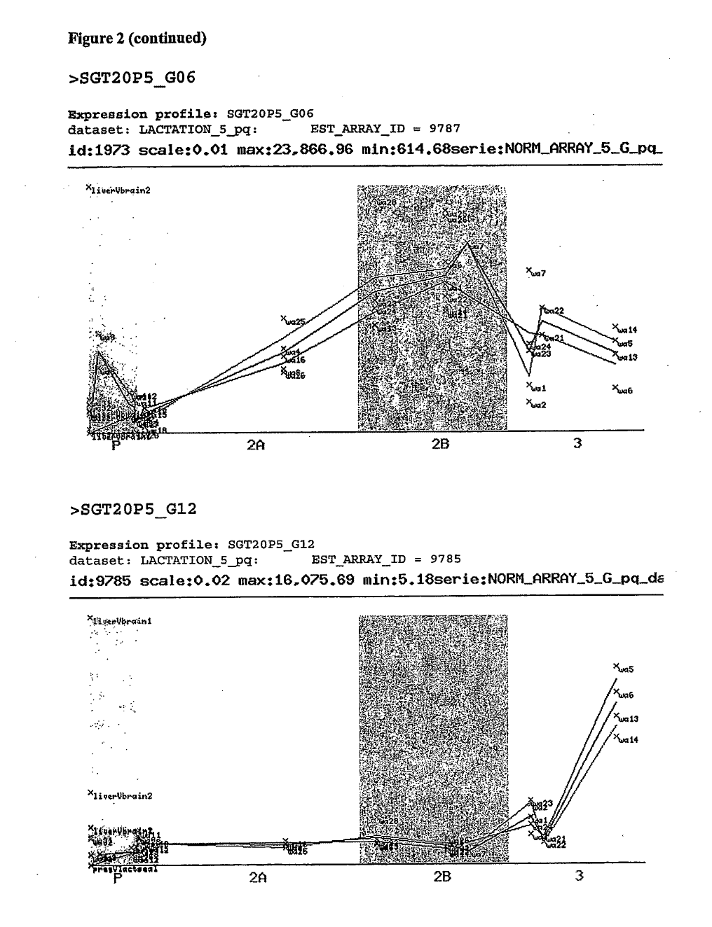

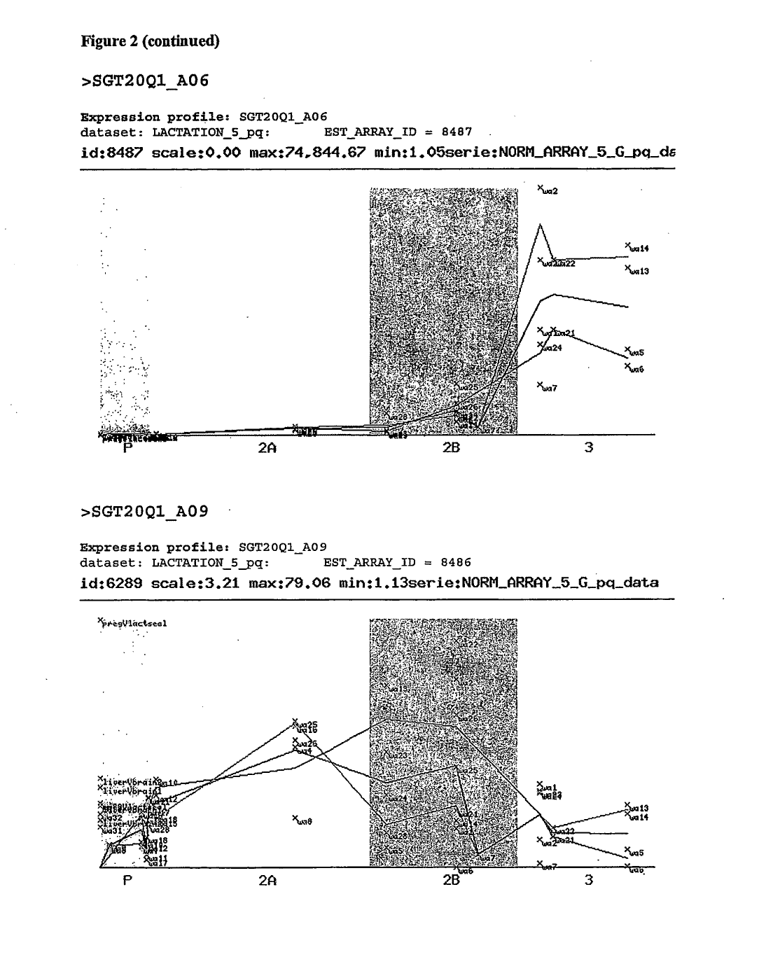

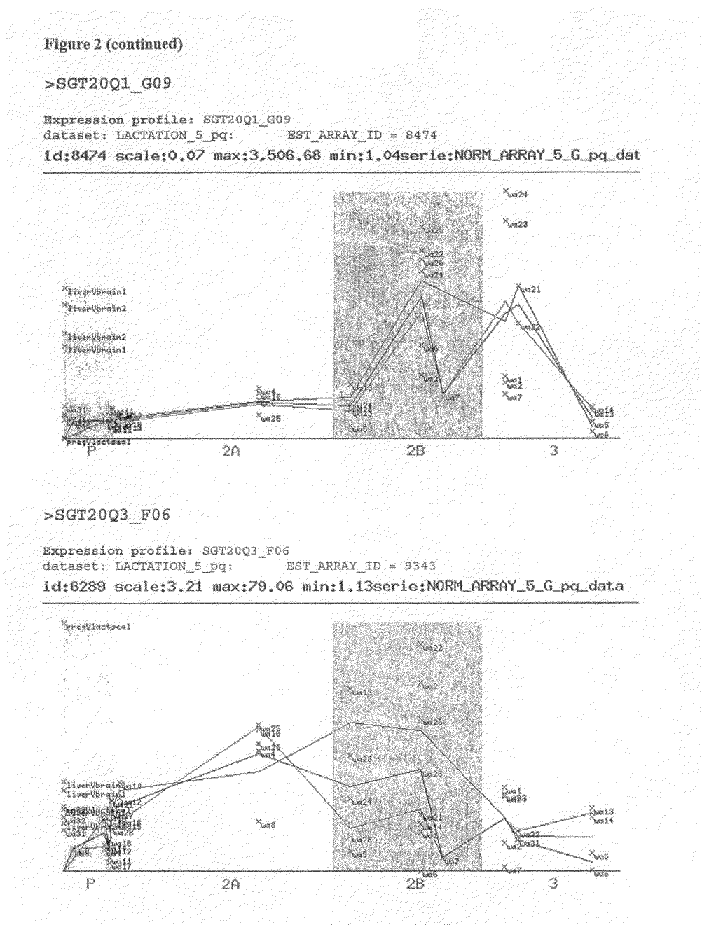

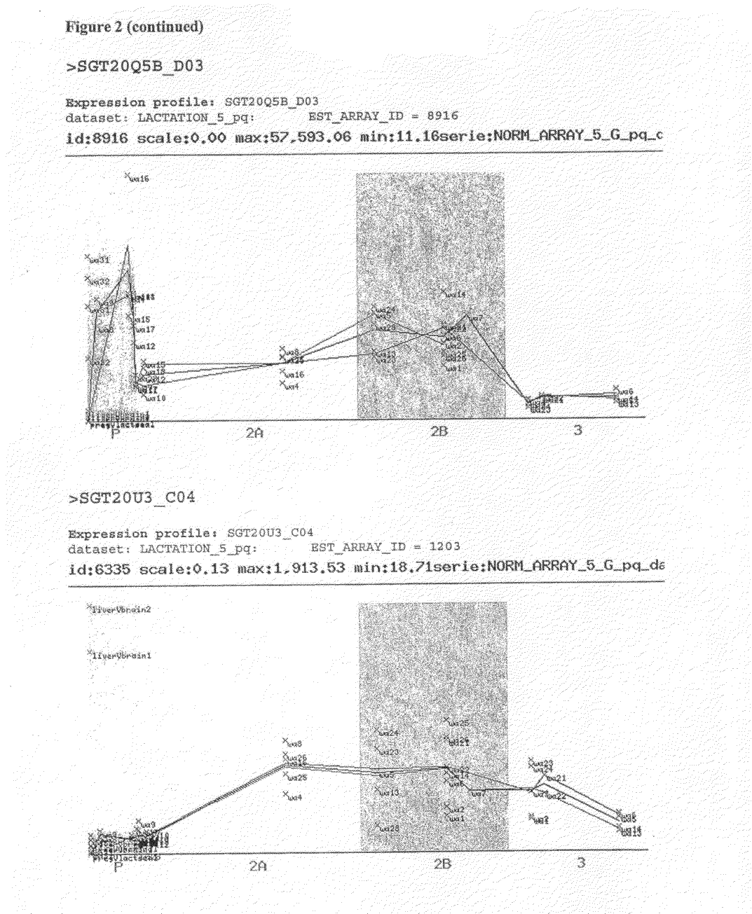

The results of the lactation-associated microarray expression profiling are provided in FIG. 2.

### Example 3

### Leader Sequence Predictions

Expressed sequence tags (ESTs) potentially encoding secreted peptides were identified using a leader sequence prediction algorithm (Bannal et al., 2002, Extensive feature detection of N-terminal protein sorting signals, Bioinformatics, 18:298-305) on peptides deduced from translating sequences from Example 1 in three frames.

EST sequences were annotated by comparisons with databases of all non-redundant GenBank coding sequence translations (+PDB+SwissProt+PIR+PRF), human Unigene and GenBank.

### Example 4

### ESTs

Combining the microarray expression profiling data (Example 2) with the leader sequence predictions (Example 3), 5 groups of lactation-associated sequences have been identified. The representatives of each group including their matches to database sequences are provided in Tables 2 to 6.

Group 1

Comprised of 103 ESTs (Table 2) showing a 10-fold increase in expression across any phase change in any microarray comparison during lactation. The most 5′ element of a contig was selected. Known milk protein genes and genes obviously encoding intracellular proteins were excluded.

Group 2

Comprised of 152 ESTs (Table 3) showing a 5-fold increase in expression across any phase change in any microarray comparison during lactation. The spot intensity for the later lactation sample must be higher than the median spot intensity for that array. The EST sequence must predict a minimum open reading frame of 30 amino acids in the forward direction and contain a putative leader sequence. The most 5′ element of a contig was selected. Known milk protein genes and genes obviously encoding intracellular proteins were excluded.

Group 3

Comprised of 12 ESTs (Table 4) showing a 5-fold increase in expression across two or more phase changes during lactation. Single channel normalized spot intensities were averaged across all samples within a phase. Spot intensities increasing 5-fold from phase 1-2b, 1-3 or 2a-3, representing ESTs with a minimum open reading frame of 30 amino acids in the forward direction and contain a predicted leader sequence were included. The most 5′ element of a contig was selected. Known milk protein genes and genes obviously encoding intracellular proteins were excluded.

Group 4

Comprised of 32 ESTs (Table 5) showing a 10-fold decrease in expression across any phase change in any microarray comparison during lactation. The spot intensity for the former lactation sample must be higher than the median spot intensity for that array. The EST sequence must predict a minimum open reading frame of 30 amino acids in the forward direction and contain a putative leader sequence. The most 5′ element of a contig was selected. Only ESTs with homology with unknown or hypothetical proteins were included.

Group 5

Comprised of 29 ESTs (Table 6). The EST sequence must predict a minimum open reading frame of 100 amino acids in the forward direction and contain a putative leader sequence predicted by both the algorithm in Example 3 and by Nielsen, H. et al. Protein Engineering 10; 1-6 (1997). The most 5′ element of a contig was selected. Only ESTs with homology with unknown or hypothetical proteins were included.

TABLE 2
Group 1 ESTs

| | | Non-redundant protein sequence | |
|---|---|---|---|
| EST clone ID | Unigene match | database match | GenBank match |
| SGT20A1_B10 | unnamed protein product [Homo sapiens], mRNA sequence | unnamed protein product [Homo sapiens] | Homo sapiens cDNA FLJ90460 fis, clone |
| | /cds = (12, 1880)/gb = AK075541 /gi = 22761753 /ug = Hs.367653 | | NT2RP3001858 |
| | /len = 3593 | | |
| SGT20A1_C03 | KIAA0252 protein [Homo sapiens], mRNA sequence | | Macaca fascicularis brain cDNA clone: QtrA-10429, |
| | /cds = (349, 2106)/gb = NM_015138 /gi = 24308004 | | full insert sequence |
| | /ug = Hs.83419 /len = 4412 | | |
| SGT20A1_D07 | hypothetical protein FLJ22875 [Homo sapiens], mRNA | hypothetical protein FLJ22875 [Homo sapiens] | Homo sapiens hypothetical protein FLJ22875 |
| | sequence/cds = (151, 633) /gb = NM_032231 /gi = 15638951 | | (FLJ22875), mRNA |
| | /ug = Hs.406548/len = 1019 | | |
| SGT20A1_F05 | | | Homo sapiens chromosome 8, clone RP11-699F21, |
| | | | complete sequence |
| SGT20B1_E04 | | | |
| SGT20C1_B03 | vasoactive intestinal peptide receptor 1; pituitary adenylate | Vasoactive intestinal polypeptide receptor precursor (VIP-R) | Meleagris gallopavo putative vasoactive intestinal |
| | cyclase activating polypeptide receptor, type II; VIP | (VIPreceptor) | peptide receptor mRNA, complete cds |
| | receptor, type I; vasoactive intestinal peptide receptor; | | |
| | PACAPtype II receptor [Homo sapiens], mRNA | | |
| | sequence/cds = (110, 1483) /gb = NM_004624 /gi = 15619005 | | |
| | /ug = Hs.348500/len = 2771 | | |
| SGT20C1_C01 | KIAA0870 protein [Homo sapiens], mRNA sequence | KIAA0870 protein [Homo sapiens] | Homo sapiens KIAA0870 protein (KIAA0870), mRNA |
| | /cds = (0, 3061)/gb = AB020677 /gi = 6635136 /ug = Hs.18166 | | |
| | /len = 4628 | | |
| SGT20C1_F02 | hypothetical protein BC012331 [Homo sapiens], mRNA | hypothetical protein BC012331 [Homo | Homo sapiens hypothetical protein BC012331 |
| | sequence/cds = (32, 736) /gb = NM_138446 /gi = 19923976 | sapiens] | (LOC115416), mRNA |
| | /ug = Hs.87385/len = 774 | | |
| SGT20C1_F10 | | | Human DNA sequence from clone RP3-380B8 on |
| | | | chromosome 6p24.1-25.3 Contains a gene encoding |
| | | | the protein Neuritin, which is involved in promotion of |
| | | | neurite outgrowth, a Pyruvatekinase (PKM2) |
| | | | pseudogene, a novel mRNA, 4 CpG islands, ESTs, |
| | | | STSs and GSSs, complete sequence |
| SGT20C2_D08 | | | |
| SGT20C3_F02 | unr-interacting protein [Homo sapiens], mRNA sequence | unnamed protein product [Mus musculus] | Homo sapiens unr-interacting protein (UNRIP) |
| | /cds = (296, 1348)/gb = NM_007178 /gi_20149591 /ug_Hs.3727 | | mRNA, complete cds |
| | /len = 1867 | | |
| SGT20D1B_B04 | | | |
| SGT20D1B_D02 | cadherin 1, type 1 preproprotein; calcium-dependent | Epithelial-cadherin precursor (E-cadherin) | Homo sapiens cadherin 1, type 1, E-cadherin |
| | adhesion protein epithelial; cadherin 1, E-cadherin | (Uvomorulin) (Cadherin-1)(ARC-1) | (epithelial) (CDH1), mRNA |
| | (epithelial); uvomorulin; cell-CAM 120/80; Arc-1 [Homo | | |
| | sapiens], mRNA sequence /cds = (124, 2772) /gb = NM_004360 | | |
| | /gi = 14589887/ug = Hs.194657 /len = 4828 | | |
| SGT20D1B_G02 | | | |
| SGT20D2B_H09 | | | |
| SGT20D3_D09 | | | |
| SGT20D3_E01 | hypothetical protein MGC14832 [Homo sapiens], mRNA | hypothetical protein MGC14832 [Homo | Homo sapiens hypothetical protein MGC14832 |
| | sequence/cds = (7, 354) /gb = NM_032339 /gi = 14150125 | sapiens] | (MGC14832), mRNA |
| | /ug = Hs.333526/len = 748 | | |
| SGT20D3_G10 | | | |
| SGT20D4_A04 | hypothetical protein FLJ23293 similar to ARL-6 interacting | 5730596K20Rik protein [Mus musculus] | Homo sapiens, hypothetical protein FLJ23293 similar |
| | protein-2[Homo sapiens], mRNA sequence /cds = (70, 1695) | | to ARL-6interacting protein-2, clone MGC: 13112 |
| | /gb = BC005096/gi = 13477254 /ug = Hs.381206 /len = 2510 | | IMAGE: 4053143, mRNA, complete cds |
| SGT20D5_B03 | tumor protein, translationally-controlled 1; fortilin | tumor protein, translationally-controlled 1; | Homo sapiens tumor protein, translationally-controlled |
| | [Homo sapiens], mRNA sequence /cds = (94, 612) | fortilin; histamine-releasing factor [Homo | 1 (TPT1), mRNA |
| | /gb = NM_003295/gi = 4507668 /ug = Hs.401448 /len = 830 | sapiens] | |
| SGT20D5_E08 | scotin [Homo sapiens], mRNA sequence /cds = (134, 856) | scotin [Homo sapiens] | Homo sapiens chromosome 3 clone RP13-794C1, |
| | /gb = NM_016479/gi = 21703709 /ug = Hs.24220 /len = 2166 | | complete sequence |
| SGT20D5_G01 | | | Human DNA sequence from clone RP11-554F11 on |
| | | | chromosome 10, complete sequence |
| SGT20E1B_E01 | | | |
| SGT20E1B_E07 | amiloride-sensitive cation channel 2, neuronal isoform a; | | Homo sapiens 12 BAC RP11-469H8 (Roswell Park |
| | hBNaC2; Cation channel, amiloride-sensitive, neuronal, 2 | | Cancer Institute Human BAC Library) complete |
| | [Homo sapiens], mRNA sequence /cds = (229, 1953) | | sequence |
| | /gb = NM_020039/gi = 21536350 /ug = Hs.274361 /len = 3923 | | |
| SGT20E3_D12 | | | |
| SGT20E3_G09 | | | |
| SGT20F1_B06 | | | |
| SGT20F1_D09 | | | |
| SGT20F1_E11 | nuclease sensitive element binding protein 1; | nuclease sensitive element binding protein | Bovine transcription factor EF1(A) mRNA, complete |
| | Major histocompatibility complex, class II, Y box- | 1 [Bos taurus] | cds |
| | binding protein I; DNA-binding protein B [Homo sapiens], | | |
| | mRNA sequence /cds = (234, 1202) /gb = NM_004559 | | |
| | /gi = 4758829/ug = Hs.74497 /len = 1474 | | |
| SGT20F3_C12 | | | |
| SGT20F3_H07 | spermidine synthase; Spermidine synthase-1 [Homo | spermidine synthase [Rattus norvegicus] | Homo sapiens, spermidine synthase, clone |
| | sapiens], mRNA sequence /cds = (82, 990) /gb = NM_003132 | | MGC: 45687 IMAGE: 5420683, mRNA, complete cds |
| | /gi = 4507208/ug = Hs.76244 /len = 1238 | | |
| SGT20G1_D10 | | hypothetical protein [Pseudomonas | |
| | | syringae pv. tomato str. DC3000] | |
| SGT20G1_D11 | | | |
| SGT20G1_F02 | transmembrane 4 superfamily member 6; tetraspan TM4SF; | Homo sapiens transmembrane 4 | Homo sapiens transmembrane 4 superfamily member |
| | A15 homolog; tetraspanin TM4-D; tetraspanin 6 [Homo | superfamily member 6 [synthetic construct] | 6 (TM4SF6), mRNA |
| | sapiens], mRNA sequence /cds = (103, 840) /gb = NM_003270 | | |
| | /gi = 21265115/ug = Hs.121068 /len = 2069 | | |
| SGT20G1_H04 | ATP-binding cassette, sub-family G, member 2; breast | unnamed protein product [Homo sapiens] | Sus scrofa mRNA for brain multidrug resistance |
| | cancer resistance protein; mitoxantrone resistance | | protein (BMDP gene) |
| | protein; placenta specific MDR protein [Homo sapiens], | | |
| | mRNA sequence /cds = (204, 2171) /gb = NM_004827 | | |
| | /gi = 4757849/ug = Hs.194720 /len = 2719 | | |
| SGT20G1_H07 | | | |
| SGT20G2_C01 | | | |
| SGT20G2_H02 | | | |
| SGT20G3_A01 | KIAA0985 protein [Homo sapiens], mRNA sequence | Transcobalamin I precursor (TCI) (TC I) | Mus musculus chromosome 5 clone rp23-403I21 |
| | /cds = (329, 2413)/gb = NM_014954 /gi = 7662431 /ug = Hs.21239 | | strain C57BL/6J, complete sequence |
| | /len = 4511 | | |
| SGT20G3_H02 | | | |
| SGT20G3_H06 | ATPase, Ca++ transporting, fast twitch 1 [Homo sapiens], | hypothetical protein [Homo sapiens] | Mus musculus, clone MGC: 28518 IMAGE: 4191741, |
| | mRNA sequence /cds = (0, 2984) /gb = NM_004320 | | mRNA, complete cds |
| | /gi = 10835219/ug = Hs.183075 /len = 3082 | | |
| SGT20G4_B08 | | angiopoietin-like 5; fibrinogen-like [Homo | |
| | | sapiens] | |
| SGT20G4_F01 | hypothetical protein MGC10731 [Homo sapiens], mRNA | hypothetical protein MGC10731 [Homo | Homo sapiens hypothetical protein MGC10731 |
| | sequence/cds = (218, 994) /gb = NM_030907 /gi = 13569861 | sapiens] | (MGC10731), mRNA |
| | /ug = Hs.322487/len = 1361 | | |
| SGT20G4_G03 | calcium binding protein Cab45 precursor [Homo sapiens], | stromal cell derived factor 4 [Mus musculus] | Mus musculus stromal cell derived factor 4 (Sdf4), |
| | mRNA sequence /cds = (293, 1339) /gb = NM_016547 | | mRNA |
| | /gi = 7706572/ug = Hs.42806 /len = 2092 | | |
| SGT20H1_F08 | | | |
| SGT20H1_G12 | | | |
| SGT20H2_H03 | | | |
| SGT20H3_G12 | | | |
| SGT20H3_H12 | | | |
| SGT20I6_G03 | | | |
| SGT20J4_F01 | | | |
| SGT20J5_D02 | GL004 protein [Homo sapiens], mRNA sequence | GL004 protein [Homo sapiens] | Homo sapiens GL004 protein (GL004), mRNA |
| | /cds = (929, 1804)/gb = NM_020194 /gi = 20070305 /ug = Hs.7045 | | |
| | /len = 1886 | | |
| SGT20K1_H12 | | | |
| SGT20K2_E12 | | | |
| SGT20K2_F12 | | leucine-rich repeat extensin family | |
| | | [Arabidopsis thaliana] | |
| SGT20K2_H03 | | | |
| SGT20K3_F11 | KIAA0678 protein [Homo sapiens], mRNA sequence | | Homo sapiens KIAA0678 protein (KIAA0678), mRNA |
| | /cds = (0, 3066)/gb = AB014578 /gi = 3327169 /ug = Hs.12707 | | |
| | /len = 3811 | | |
| SGT20K3_H02 | WW domain-containing binding protein 4; formin binding | WW domain-containing binding protein 4; | Homo sapiens WW domain binding protein 4 (formin |
| | protein 21[Homo sapiens], mRNA sequence | formin binding protein 21 [Homo sapiens] | binding protein 21) (WBP4), mRNA |
| | /cds = (113, 1243)/gb = NM_007187 /gi = 21536424 | | |
| | /ug = Hs.28307 /len = 2354 | | |
| SGT20K4_A03 | emopamil-binding protein (sterol isomerase); 3-beta- | emopamil binding protein (sterol | Homo sapiens emopamil binding protein (sterol |
| | hydroxysteroid-delta-8,delta-7-isomerase; Chondrodypslasia | isomerase); Chondrodysplasiapunctata-2, | isomerase) (EBP), mRNA |
| | punctata-2, X-linked dominant (Happlesyndrome) [Homo | X-linked dominant (Happle | |
| | sapiens], mRNA sequence /cds = (111, 803)/gb = NM_006579 | syndrome); emopamil-binding protein (sterol | |
| | /gi = 5729809 /ug = Hs.75105 /len = 1073 | isomerase); 3-beta-hydroxysteroid-delta- | |
| | | 8,delta-7-isomerase; sterol8-isomerase | |
| | | [Homo sapiens] | |
| SGT20L4_D07 | | | |
| SGT20L4_F01 | | | |
| SGT20M5_H02 | | | |
| SGT20N1_G03 | fatty acid binding protein 3; Fatty acid-binding protein 3, | fatty acid binding protein (heart) like [Bos | Sus scrofa partial mRNA for heart fatty acid-binding |
| | muscle; H-FABP; mammary-derived growth inhibitor [Homo | taurus] | protein (FABP3gene) |
| | sapiens], mRNA sequence /cds = (45, 446) /gb = NM_004102 | | |
| | /gi = 10938020/ug = Hs.49881 /len = 679 | | |
| SGT20N5_B07 | | | |
| SGT20N5_B09 | | | |
| SGT20N5_G11 | hypothetical protein FLJ10597 [Homo sapiens], mRNA | hypothetical protein [Macaca fascicularis] | Homo sapiens, clone IMAGE: 4814781, mRNA |
| | sequence/cds = (62, 799) /gb = NM_018150 /gi = 8922541 | | |
| | /ug = Hs.90375/len = 2494 | | |
| SGT20O1_C06 | ribonuclease/angiogenin inhibitor, Placental ribonuclease | ribonuclease/angiogenin inhibitor 1 [Mus | Homo sapiens ribonuclease/angiogenin inhibitor |
| | inhibitor[Homo sapiens], mRNA sequence | musculus] | (RNH), mRNA |
| | /cds = (1408, 2793)/gb = NM_002939 /gi = 21361546 | | |
| | /ug = Hs.75108 /len = 2982 | | |
| SGT20O1_D05 | | | |
| SGT20O1_D10 | | | |
| SGT20O2_F04 | | | |
| SGT20O3_C10 | | | |
| SGT20O3_D11 | | | |
| SGT20O3_D12 | | | |
| SGT20O3_E05 | peroxiredoxin 1; Proliferation-associated gene | peroxiredoxin 1; natural killer-enhancing | Homo sapiens, peroxiredoxin 1, clone MGC: 24196 |
| | A; proliferation-associated gene A (naturalkiller-enhancing | factor A; proliferation-associated gene A | IMAGE: 3681912, mRNA, complete cds |
| | factor A) [Homo sapiens], mRNA sequence/cds = (60, 659) | [Homo sapiens] | |
| | /gb = NM_002574 /gi = 4505590 /ug = Hs.180909/len = 937 | | |
| SGT20O3_H02 | | | |
| SGT20O3_H10 | | | |
| SGT20O4_C03 | PRO1851 [Homo sapiens], mRNA sequence | Inter-alpha-trypsin inhibitor heavy chain H4 | Homo sapiens PRO1851 mRNA, complete cds |
| | /cds = (304, 2238) /gb = AF119856/gi = 7770148 /ug = Hs.406267 | precursor (ITI heavychain H4) (Inter-alpha- | |
| | /len = 2446 | inhibitor heavy chain 4)(Inter-alpha-trypsin | |
| | | inhibitor family heavy chain-relatedprotein) | |
| | | (IHRP) (Major acute phase protein) (MAP) | |
| SGT20O5_F04 | | | |
| SGT20O5_F05 | | | |
| SGT20P1_F05 | Similar to major histocompatibility complex, class I, F | class I histocompatibility antigen Maru-UB- | Macropus rufogriseus MHC class I protein (Maru- |
| | [Homo sapiens], mRNA sequence /cds = (29, 1069) | 01 alpha chain precursor-red-necked | UB*01) mRNA, complete cds |
| | /gb = BC018925/gi = 17511934 /ug = Hs.283611 /len = 4146 | wallaby | |
| SGT20P2_H07 | hypothetical protein BC012008 [Homo sapiens], mRNA | | Homo sapiens hypothetical protein BC012008 |
| | sequence/cds = (394, 492) /gb = NM_138473 /gi = 19924004 | | (LOC144467), mRNA |
| | /ug = Hs.348374/len = 1510 | | |
| SGT20P2_H11 | | | |
| SGT20P2_H12 | | | |
| SGT20P3_F02 | osteomodulin [Homo sapiens], mRNA sequence | osteomodulin [Homo sapiens] | Homo sapiens osteomodulin (OMD), mRNA |
| | /cds = (100, 1365)/gb = NM_005014 /gi = 4826875 /ug = Hs.94070 | | |
| | /len = 2263 | | |
| SGT20P3_G09 | | | |
| SGT20P4_G03 | | 18K lipopolysaccharide-binding protein | |
| | | precursor - rabbit | |
| SGT20P4_H11 | | hypothetical protein (L1H 3′ region) - human | Homo sapiens chromosome 8, clone RP11-48J8, |
| | | | complete sequence |
| SGT20P5_A10 | | | |
| SGT20P5_B10 | | | |
| SGT20P5_C03 | | | |
| SGT20P5_D11 | | | |
| SGT20P5_E05 | | | |
| SGT20P5_G06 | | | |
| SGT20P5_G12 | | | |
| SGT20Q1_A06 | | | |
| SGT20Q1_A09 | | | |
| SGT20Q1_C09 | 601657005R1 NIH_MGC_67 Homo sapiens cDNA clone | | Homo sapiens hypothetical protein DKFZp547B0714 |
| | IMAGE: 3866184 3′, mRNA sequence | | (DKFZp547B0714), mRNA |
| | /clone = IMAGE: 3866184 /clone_end = 3′/gb = BE963678 | | |
| | /gi = 11767097 /ug = Hs.393377 /len = 670 | | |
| SGT20Q1_G09 | | | |
| SGT20Q3_E10 | | | Wallabia bicolor isolate W15 retroposon CORE-SINE |
| | | | Mar-1 sequence |
| SGT20Q5B_D02 | | | Wallabia bicolor isolate W15 retroposon CORE-SINE |
| | | | Mar-1 sequence |
| SGT20U4_D03 | | freeze tolerance-associated protein FR47 | |
| | | [Rana sylvatica] | |
| SGT20U5_C10 | | | |
| SGT20W1_F04 | | | |

TABLE 3
Group 2 ESTs

| | | Non-redundant protein sequence database | |
|---|---|---|---|
| EST clone ID | Unigene match | match | GenBank match |
| SGT20A1_C07 | acetyl-CoA synthetase isoform a; cytoplasmic acetyl-coenzyme | unnamed protein product [Mus musculus] | Homo sapiens acetyl-Coenzyme A synthetase |
| | Asynthetase; acetate-CoA ligase; acyl-activating enzyme; acetate | | 2 (ADP forming) (ACAS2), transcript variant 2, |
| | thiokinase; acetyl-CoA synthetase [Homo sapiens], mRNA | | mRNA |
| | sequence /cds = (74, 2179) /gb = NM_018677 | | |
| | /gi = 21269869/ug = Hs.14779 /len = 2925 | | |
| SGT20A1_H05 | | | |
| SGT20A1_H08 | | | |
| SGT20B1_H10 | to 78f09.x1 NCI_CGAP_Gas4 Homo sapiens cDNA clone | RIKEN cDNA 1110064A23 [Mus musculus] | Homo sapiens cDNA: FLJ21926 fis, clone |
| | IMAGE: 2184425 3′, mRNA sequence /clone = IMAGE: 2184425 | | HEP04142, highly similar to AB016092 Homo |
| | /clone_end = 3′/gb = AI570375 /gi = 4533749 /ug = Hs.228943 | | sapiens mRNA for RNA binding protein |
| | /len = 390 | | |
| SGT20C1_C07 | | | |
| SGT20C1_E04 | UI-CF-EC1-aca-c-21-0-UI.s1 UI-CF-EC1 Homo sapiens cDNA | RIKEN cDNA 2010208K18 [Mus musculus] | Homo sapiens cDNA FLJ13019 fis, clone |
| | cloneUI-CF-EC1-aca-c-21-0-UI 3′, mRNA sequence/clone = UI-CF- | | NT2RP3000736, highly similar to Human |
| | EC1-aca-c-21-0-UI /clone_end = 3′/gb = BM974250 /gi = 19591841 | | mRNA for KIAA0140 gene |
| | /ug = Hs.421587 /len = 754 | | |
| SGT20C2_E05 | hypothetical protein FLJ25124 [Homo sapiens], mRNA | unnamed protein product [Homo sapiens] | Homo sapiens cDNA FLJ25124 fis, clone |
| | sequence/cds = (73, 3078) /gb = NM_144698 /gi = 24432064 | | CBR06414 |
| | /ug = Hs.133081/len = 3323 | | |
| SGT20C2_F04 | Similar to small inducible cytokine A4 [Homo sapiens], | LAG-1 [Homo sapiens] | Mus musculus chemokine (C-C motif) ligand 4 |
| | mRNA sequence /cds = (65, 250) /gb = BC027961 | | (Ccl4), mRNA |
| | /gi = 20379894/ug = Hs.75703 /len = 1798 | | |
| SGT20C3_C12 | chromosome 14 open reading frame 1 [Homo sapiens], mRNA | HSPC288 [Homo sapiens] | Homo sapiens chromosome 14 open reading |
| | sequence/cds = (72, 494) /gb = NM_007176 /gi = 6005718 | | frame 1 (C14orf1), mRNA |
| | /ug = Hs.15106/len = 2274 | | |
| SGT20C3_E08 | JM1 protein [Homo sapiens], mRNA sequence | DXImx40e protein [Mus musculus] | Homo sapiens, Similar to JM1 protein, clone |
| | /cds = (86, 1969)/gb = NM_014008 /gi = 7661843 /ug = Hs.26333 | | MGC: 15381 IMAGE: 4299954, mRNA, |
| | /len = 2228 | | complete cds |
| SGT20C3_H10 | | | |
| SGT20C4_H03 | MCM3 minichromosome maintenance deficient 3 (S. cerevisiae) | Unknown (protein for IMAGE: 3831362) [Homo | Homo sapiens cDNA FLJ37862 fis, clone |
| | associated protein; minichromosome maintenance 3- | sapiens] | BRSSN2015707, highly similar to 80 KDA |
| | associated protein, 80-kD; minichromosome maintenance deficient | | MCM3-ASSOCIATED PROTEIN |
| | (S. cerevisiae) 3-associated protein; human mRNA for MCM3 | | |
| | import factor, MCM3 im> /cds = (37, 5979)/gb = NM_003906 | | |
| | /gi = 19923190 /ug = Hs.168481 /len = 6114 | | |
| SGT20C5_F01 | | VMP4 protein [Volvox carteri f. nagariensis] | |
| SGT20D1B_A10 | melanoma-associated antigen p97, isoform 1, | melanoma-associated antigen p97 isoform 1, | Homo sapiens antigen p97 (melanoma |
| | precursor; melanotransferrin; melanoma-associated antigen p97 | precursor; melanoma-associated antigen p97; | associated) identified bymonoclonal antibodies |
| | [Homo sapiens], mRNA sequence /cds = (69, 2285) | melanotransferrin [Homo sapiens] | 133.2 and 96.5 (MFI2), transcriptvariant 1, |
| | /gb = NM_005929/gi = 16933549 /ug = Hs.271966 /len = 2377 | | mRNA |
| SGT20D1B_F10 | | | |
| SGT20D2B_C07 | UI-H-ED0-axb-n-02-0-UI.s1 NCI_CGAP_ED0 Homo sapiens | RIKEN cDNA 1110064A23 [Mus musculus] | H. sapiens mRNA for fibrillin |
| | cDNA clone IMAGE: 5826625 3′, mRNA sequence | | |
| | /clone = IMAGE: 5826625/clone_end = 3′ /gb = BM995286 | | |
| | /gi = 19720187 /ug = Hs.433864/len = 1281 | | |
| SGT20D2B_G07 | choline phosphotransferase 1; cholinephosphotransferase | unnamed protein product [Mus musculus] | Homo sapiens choline phosphotransferase 1 |
| | 1; cholinephosphotransferase 1 alpha [Homo sapiens], | | (CHPT1), mRNA |
| | mRNAsequence /cds = (170, 1390) /gb = NM_020244 | | |
| | /gi = 9910383/ug = Hs.171889 /len = 1536 | | |
| SGT20D2B_H10 | | BETA-LACTOGLOBULIN PRECURSOR | M. eugenil mRNA for beta-lactoglobulin |
| SGT20D3_E07 | HSPC043 protein [Homo sapiens], mRNA sequence | HSPC291 [Homo sapiens] | Homo sapiens HSPC043 protein (HSPC043), |
| | /cds = (177, 491)/gb = NM_021218 /gi = 24308268 /ug = Hs.46624 | | mRNA |
| | /len = 1532 | | |
| SGT20D3_F05 | | | Macropus giganteus microsatellite G12-6 |
| | | | sequence |
| SGT20D4_H08 | | | |
| SGT20D5_A02 | | | |
| SGT20E1B_H04 | KIAA1299 protein [Homo sapiens], mRNA sequence | unnamed protein product [Homo sapiens] | Homo sapiens SH2-B homolog (SH2B), |
| | /cds = (3114, 5306)/gb = AB037720 /gi = 7242952 /ug = Hs.15744 | | mRNA |
| | /len = 6043 | | |
| SGT20E3_A04 | seipin [Homo sapiens], mRNA sequence /cds = (506, 1900) | seipin [Homo sapiens] | Homo sapiens Bemardinelli-Selp congenital |
| | /gb = NM_032667/gi = 21362089 /ug = Hs.293981 /len = 2012 | | lipodystrophy 2 (seipin)(BSCL2), mRNA |
| SGT20E3_C11 | | AA589509 protein [Mus musculus] | Rattus norvegicus Mk1 protein (Mk1), mRNA |
| SGT20E3_E03 | | hypothetical protein [Pseudomonas syringae | |
| | | pv. syringae B728a] | |
| SGT20E3_G07 | Homo sapiens cDNA FLJ33231 fis, clone ASTRO2001806, | | Homo sapiens chromosome 11, clone RP11- |
| | mRNA sequence/gb = AK090550 /gi = 21748732 /ug = Hs.198793 | | 265D17, complete sequence |
| | /len = 3750 | | |
| SGT20E4_B08 | | | |
| SGT20E4_H03 | | carbonic anhydrase 15 [Mus musculus] | Mus musculus carbonic anhydrase 15 (Car15), |
| | | | mRNA |
| SGT20F1_E06 | | | |
| SGT20F2_C07 | UDP-N-acteylglucosamine pyrophosphorylase 1; AgX; sperm | Chain A, Crystal Structure Of Human Agx2 | Homo sapiens UDP-N-acteylglucosamine |
| | associatedantigen 2; UDP-N-acteylglucosamine | Complexed With Udpglcnac | pyrophosphorylase 1 (UAP1), mRNA |
| | pyrophosphorylase 1; Sperm associated antigen 2 [Homo | | |
| | sapiens], mRNA sequence/cds = (311, 1828) /gb = NM_003115 | | |
| | /gi = 19923738 /ug = Hs.21293/len = 2332 | | |
| SGT20F2_E06 | Homo sapiens mRNA; cDNA DKFZp686I2113 (from clone | gamma-glutamyltransferase 1 [Homo sapiens] | Homo sapiens gamma-glutamyltransferase 1 |
| | DKFZp686I2113), mRNA sequence /gb = AL832738 /gi = 21733319 | | (GGT1), transcript variant 3, mRNA |
| | /ug = Hs.401847/len = 5325 | | |
| SGT20F2_H03 | oxysterol-binding protein-like protein 5 isoform a; oxysterol- | oxysterol-binding protein-like protein 5 isoform | Homo sapiens, similar to oxysterol binding |
| | binding protein-related protein 5; OSBP-related protein | a; oxysterol-binding protein-related protein 5; | protein-like 5, clone MGC: 48715 |
| | 5; oxysterol-binding protein homologue 1 [Homo sapiens], mRNA | OSBP-related protein 5; oxysterol-binding | IMAGE: 5769002, mRNA, complete cds |
| | sequence /cds = (116, 2755) /gb = NM_020896 | protein homologue 1 [Homo sapiens] | |
| | /gi = 22035607/ug = Hs.112034 /len = 3873 | | |
| SGT20F3_E11 | DKFZP564O243 protein [Homo sapiens], mRNA sequence | DKFZP564O243 protein [Homo sapiens] | Homo sapiens DKFZP564O243 protein |
| | /cds = (77, 892)/gb = NM_015407 /gi = 24475632 /ug = Hs.92700 | | (DKFZP564O243), mRNA |
| | /len = 1102 | | |
| SGT20F4_B09 | | | Wallabia bicolor isolate W51 retroposon |
| | | | CORE-SINE Mar-1 sequence |
| SGT20G1_A05 | coronin, actin binding protein, 1B [Homo sapiens], mRNA | coronin, actin binding protein 1B; coronin 1b; | Oryctolagus cuniculus coronin-like protein |
| | sequence/cds = (61, 1530) /gb = NM_020441 /gi = 14149733 | coronin 2 [Mus musculus] | pp66 mRNA, complete cds |
| | /ug = Hs.6191/len = 1877 | | |
| SGT20G1_A11 | | | |
| SGT20G1_E11 | carbonyl reductase; kidney dicarbonyl reductase [Homo | diacetyl/L-xylulose reductase [Rattus | Homo sapiens dicarbonyl/L-xylulose reductase |
| | sapiens], mRNA sequence /cds = (3, 737) /gb = NM_016286 | norvegicus] | (DCXR), mRNA |
| | /gi = 7705924/ug = Hs.9857 /len = 848 | | |
| SGT20G2_E04 | angiopoietin-like 4 protein; hepatic angiopoietin-related | fasting-induced adipose factor [Mus musculus] | Mus musculus fasting-induced adipose factor |
| | protein; PPARG angiopoietin related protein; fasting- | | mRNA, complete cds |
| | induced adipose factor; hepatic fibrinogen/angiopoietin- | | |
| | related protein [Homo sapiens], mRNA sequence | | |
| | /cds = (195, 1415)/gb = NM_139314 /gi = 21536397 /ug = Hs.9613 | | |
| | /len = 1967 | | |
| SGT20G3_C08 | xanthene dehydrogenase; xanthine oxidase; xanthine | xanthine dehydrogenase [Fells catus] | Fells catus xanthine dehydrogenase (XDH) |
| | dehydrogenase[Homo sapiens], mRNA sequence | | mRNA, complete cds |
| | /cds = (81, 4082)/gb = NM_000379 /gi = 9257259 /ug = Hs.250 | | |
| | /len = 4428 | | |
| SGT20G3_C12 | | pherophorin-dz1 protein [Volvox carteri f. nagariensis] | |
| SGT20H1_D04 | guanine nucleotide-binding protein, beta-2 subunit; G protein, | guanine nuclotide-binding protein, beta-2 | Mus musculus, guanine nucleotide binding |
| | beta-2 subunit; guanine nucleotide-binding protein G(I)/G(S)/G(T) | subunit [Mus musculus] | protein, beta 2, clone MGC: 25597 |
| | beta subunit 2; signal-transducing guanine nucleotide-binding | | IMAGE: 4019292, mRNA, complete cds |
| | regulatory protein beta subunit; transducin beta chain 2 [Homo> | | |
| | /cds = (258, 1280)/gb = NM_005273 /gi = 20357528 /ug = Hs.91299 | | |
| | /len = 1666 | | |
| SGT20H1_D09 | | | |
| SGT20H1_F05 | | OJ1117_G01.23 [Oryza sativa (japonica | |
| | | cultivar-group)] | |
| SGT20H1_H06 | | | Homo sapiens BAC clone CTD-3045A19 from |
| | | | 7, complete sequence |
| SGT20H3_D01 | SMC1 (structural maintenance of chromosomes 1, yeast)-like | | Wallabia bicolor isolate W42 retroposon |
| | 1; Segregation of mitotic chromosomes 1 (SMC1, yeast | | CORE-SINE Mar-1 sequence |
| | human homolog of [Homo sapiens], mRNA sequence | | |
| | /cds = (33, 3734)/gb = NM_006306 /gi = 5453641 /ug = Hs.211602 | | |
| | /len = 5190 | | |
| SGT20H3_E07 | | | |
| SGT20H4_F07 | nuclear receptor subfamily 1, group H, member 2; ubiquitously- | orphan receptor | Mus musculus nuclear receptor subfamily 1, |
| | expressed nuclear receptor [Homo sapiens], mRNA sequence | | group H, member 2(Nr1h2), mRNA |
| | /cds = (244, 1629) /gb = NM_007121 /gi = 11321629/ug = Hs.100221 | | |
| | /len = 2010 | | |
| SGT20H4_G04 | osteoprotegerin precursor; tumor necrosis factor | osteoprotegerin [Homo sapiens] | Homo sapiens tumor necrosis factor receptor |
| | receptor superfamily, member 11b; | | superfamily, member 11b(osteoprotegerin) |
| | osteoprotegerin; osteoclastogenesis inhibitory factor [Homo | | (TNFRSF11B), mRNA |
| | sapiens], mRNA sequence /cds = (251, 1456) /gb = NM_002546 | | |
| | /gi = 22547122/ug = Hs.81791 /len = 2291 | | |
| SGT20H5_D04 | URB [Homo sapiens], mRNA sequence /cds = (145, 2997) | similar to URB [Homo sapiens] | Homo sapiens likely ortholog of mouse Urb |
| | /gb = AF506819/gi = 21039408/ug = Hs.356289 /len = 3320 | | (URB), mRNA |
| SGT20I1_D07 | EBNA-2 co-activator (100 kD) [Homo sapiens], mRNA | Unknown (protein for MGC: 790) [Homo | Homo sapiens EBNA-2 co-activator (100 kD) |
| | sequence/cds = (267, 2924) /gb = NM_014390 /gi = 7657430 | sapiens] | (p100), mRNA |
| | /ug = Hs.79093/len = 3480 | | |
| SGT20I3_C02 | | | |
| SGT20I3_E02 | KIAA1723 protein [Homo sapiens], mRNA sequence | KIAA1723 protein [Homo sapiens] | Homo sapiens deleted in liver cancer 1 |
| | /cds = (252, 4916)/gb = AB051510 /gi = 12697990 /ug = Hs.8700 | | (DLC1), mRNA |
| | /len = 7365 | | |
| SGT20I4_B04 | transcription factor binding to IGHM enhancer 3; Transcription | TFE3 transcription factor [Homo sapiens] | Homo sapiens transcription factor binding to |
| | factor for IgH enhancer [Homo sapiens], mRNA | | IGHM enhancer 3 (TFE3), mRNA |
| | sequence/cds = (238, 1965) /gb = NM_006521 /gi = 21359903 | | |
| | /ug = Hs.274184/len = 3431 | | |
| SGT20I5_D07 | | | |
| SGT20J1_G03 | | | Didelphis virginiana isolate O40 retroposon |
| | | | CORE-SINE Mar-1sequence |
| SGT20J1_G07 | | | |
| SGT20J3_F01 | | | |
| SGT20J3_F04 | tz76e06.x1 NCI_CGAP_Pan1 Homo sapiens cDNA clone | A ‘c’ was inserted after nt 369 (=nt 10459 in | Mus musculus G protein-coupled receptor 84 |
| | IMAGE: 22945303′, mRNA sequence /clone = IMAGE: 2294530 | genomic sequence(M10126)) to correct-1 | (Gpr84), mRNA |
| | /clone_end = 3′/gb = AI913173 /gi = 5633116 /ug = Hs.413861 | frameshift probably due to gelcompression | |
| | /len = 441 | | |
| SGT20J3_G01 | | | |
| SGT20J3_H03 | | | |
| SGT20J4_F09 | | | |
| SGT20J5_B10 | AL515111 LTI_NFL006_PL2 Homo sapiens cDNA clone | hydroxyproline-rich glycoprotein DZ-HRGP | BAC sequence from the SPG4 candidate |
| | CL0BB022ZB11 3prime, mRNA sequence | [Volvox carteri f. nagariensis] | region at 2p21-2p22 BAC 367K01 of library |
| | /clone = CL0BB022ZB11 /clone_end = 3′/gb = AL515111 | | CITB_978_SKB from chromosome 2 of Homo |
| | /gi = 12778604 /ug = Hs.331862 /len = 460 | | sapiens (Human) |
| SGT20J5_C08 | | | Homo sapiens Xp BAC RP11-459A10 |
| | | | (Roswell Park Cancer Institute Human |
| | | | BAC Library) complete sequence |
| SGT20J6_B08 | Homo sapiens, Similar to hypothetical protein FLJ14642, | hypothetical protein FLJ14642 [Homo | Homo sapiens, Similar to hypothetical protein |
| | clone IMAGE: 5266209, mRNA, mRNA sequence | sapiens] | FLJ14642, clone IMAGE: 5266209, mRNA |
| | /gb = BC038673/gi = 24270879 /ug = Hs.245342 /len = 4512 | | |
| SGT20J6_F03 | Homo sapiens, Similar to myeloid/lymphoid or mixed-lineage | nucleolar and coiled-body phosphoprotein 1 | Cepaea nemoralis microsatellite Cne1 |
| | leukemia (trithorax (Drosophila) homolog); translocated to, 3, clone | [Mus musculus] | sequence |
| | IMAGE: 5212069, mRNA, mRNA sequence | | |
| | /gb = BC030550/gi = 22539718 /ug = Hs.382134 /len = 2059 | | |
| SGT20J6_H10 | signal peptidase complex (18 kD) [Homo sapiens], mRNA | signal peptidase complex; sid2895p; signal | Homo sapiens signal peptidase complex |
| | sequence/cds = (77, 616) /gb = NM_014300 /gi = 7657608 | peptidase complex (18 kD) [Mus musculus] | (18 kD) (SPC18), mRNA |
| | /ug = Hs.9534/len = 1105 | | |
| SGT20K1_B08 | hypothetical protein MGC4618 [Homo sapiens], mRNA | unnamed protein product [Mus musculus] | Mus musculus, RIKEN cDNA 3010001K23 |
| | sequence/cds = (107, 1621) /gb = NM_032326 /gi = 14150103 | | gene, clone MGC: 8187 IMAGE: 3590497, |
| | /ug = Hs.89072/len = 1818 | | mRNA, complete cds |
| SGT20K1_B12 | | | |
| SGT20K1_H09 | hypothetical protein MGC11275; likely ortholog of mouse | similar to RIKEN cDNA 2610042J20; | Homo sapiens chromosome 16 clone RP11- |
| | syndesmos[Homo sapiens], mRNA sequence | expressed sequence N28182 [Mus musculus] | 709D24, complete sequence |
| | /cds = (21, 656)/gb = NM_032349 /gi = 14150146 /ug = Hs.6949 | [Rattus norvegicus] | |
| | /len = 1350 | | |
| SGT20K2_H10 | | | |
| SGT20K3_D12 | | | Homo sapiens chromosome 7 clone RP11- |
| | | | 707A19, complete sequence |
| SGT20K3_E10 | solute carrier family 25 (mitochondrial carrier; citrate transporter), | citrate transporter protein - human | Rattus norvegicus solute carrier family 25, |
| | member 1; solute carrier family 20 (mitochondrial citrate | | member 1 (Slc25a1), nuclear gene encoding |
| | transporter), member 3 [Homo sapiens], mRNA sequence | | mitochondrial protein, mRNA |
| | /cds = (99, 1034) /gb = NM_005984/gi = 21389314 /ug = Hs.111024 | | |
| | /len = 1619 | | |
| SGT20K3_G09 | hypothetical protein FLJ25333 [Homo sapiens], mRNA | unnamed protein product [Homo sapiens] | Homo sapiens hypothetical protein FLJ25333 |
| | sequence/cds = (160, 1404) /gb = NM_152548 /gi = 22749142 | | (FLJ25333), mRNA |
| | /ug = Hs.127206/len = 1645 | | |
| SGT20K3_H01 | | | Homo sapiens chromosome 4 clone CTD- |
| | | | 2314I6, complete sequence |
| SGT20K4_C10 | KIAA0409 [Homo sapiens], mRNA sequence /cds = (0, 1394) | RIKEN cDNA 1500003O22 [Mus musculus] | Homo sapiens KIAA0409 protein (KIAA0409), |
| | /gb = AB007869/gi = 2662098 /ug = Hs.5158 /len = 6469 | | mRNA |
| SGT20K4_H08 | solute carrier family 9, member 7; nonselective | solute carrier family 9, member 7; | Homo sapiens solute carrier family 9 |
| | sodiumpotassium/proton exchanger; sodium/hydrogen exchanger | nonselective sodiumpotassium/proton | (sodium/hydrogen exchanger), isoform 7 |
| | 7 [Homo sapiens], mRNA sequence /cds = (8, 2185) | exchanger; sodium/hydrogen exchanger | (SLC9A7), mRNA |
| | /gb = NM_032591/gi = 14211918 /ug = Hs.154353 /len = 2200 | 7 [Homo sapiens] | |
| SGT20L1_A11 | zizimin1 [Homo sapiens], mRNA sequence /cds = (55, 6264) | Unknown (protein for IMAGE: 6156949) [Homo | Mus musculus, Similar to hypothetical protein |
| | /gb = NM_015296/gi = 24308028 /ug = Hs.8021 /len = 7522 | sapiens] | FLJ20220, clone MGC: 11827 IMAGE: 3596515, |
| | | | mRNA, complete cds |
| SGT20L1_C05 | small inducible cytokine A28 precursor; CC chemokine | chemokine CCL28/MEC [Macaca mulatta] | Homo sapiens chemokine (C-C motif) ligand |
| | CCL28; mucosae-associated epithelial chemokine; small | | 28 (CCL28), transcript variant 2, mRNA |
| | inducible cytokine subfamily A (Cys-Cys), member 28 | | |
| | [Homo sapiens], mRNA sequence /cds = (54, 437) | | |
| | /gb = NM_019846/gi = 22538809 /ug = Hs.283090 /len = 1349 | | |
| SGT20L4_E06 | kinesin-related protein [Homo sapiens], mRNA | | Human DNA sequence from clone RP4- |
| | sequence/cds = (1389, 5555) /gb = AB017133 /gi = 15822815 | | 736L20 on chromosome 1p36.12-36.23, |
| | /ug = Hs.375193/len = 8776 | | complete sequence |
| SGT20M3_C02 | | | |
| SGT20M3_E09 | RAB11B, member RAS oncogene family; RAB11B, member of | Similar to RAB11B, member RAS oncogene | Rattus norvegicus RAB11B, member RAS |
| | RAS oncogenefamily [Homo sapiens], mRNA sequence | family [Xenopus laevis] | oncogene family (Rab11b), mRNA |
| | /cds = (6, 662)/gb = NM_004218 /gi = 4758985 /ug = Hs.239018 | | |
| | /len = 701 | | |
| SGT20M4_G11 | | similar to hypothetical protein FLJ10143 [Mus | |
| | | musculus] | |
| SGT20M5_D02 | hypothetical protein 24432 [Homo sapiens], mRNA | Similar to hypothetical protein 24432 [Homo | Homo sapiens hypothetical protein 24432 |
| | sequence/cds = (332, 1957) /gb = NM_022914 /gi = 12597658 | sapiens] | (24432), mRNA |
| | /ug = Hs.78019/len = 2034 | | |
| SGT20M5_G11 | 602345225F1 NIH_MGC_89 Homo sapiens cDNA clone | RIKEN cDNA 1110064A23 [Mus musculus] | Hepatitis C virus gene for polyprotein, |
| | IMAGE: 4455079 5′, mRNA sequence /clone = IMAGE: 4455079 | | complete cds, isolate: HCVT142 |
| | /clone_end = 5′/gb = BG168549 /gi = 12675252 /ug = Hs.421771 | | |
| | /len = 211 | | |
| SGT20M5_H01 | diacylglycerol O-acyltransferase homolog 2; GS1999full | hypothetical protein [Homo sapiens] | Homo sapiens diacylglycerol O- |
| | [Homo sapiens], mRNA sequence /cds = (777, 1670) | | acyltransferase homolog 2 (mouse)(DGAT2), |
| | /gb = NM_032564/gi = 14211870 /ug = Hs.334305 /len = 2713 | | mRNA |
| SGT20N2_D03 | | | Mus musculus chromosome 7 clone RP24- |
| | | | 63N24, complete sequence |
| SGT20N2_H05 | TPA regulated locus; uncharacterized hypothalamus protein | TPARDL [Mus musculus] | Homo sapiens transmembrane protein mRNA, |
| | HTMP[Homo sapiens], mRNA sequence | | complete cds |
| | /cds = (194, 1168)/gb = NM_018475 /gi = 8923860 /ug = Hs.236510 | | |
| | /len = 1913 | | |
| SGT20N3_A01 | | | Homo sapiens TRAM-like protein (KIAA0057), |
| | | | mRNA |
| SGT20N3_A02 | | envelope protein [Caprine nasal tumour virus] | |
| SGT20N3_H03 | | lipopolysaccharide receptor; CD14 [Equus | |
| | | caballus] | |
| SGT20N4_A10 | hypothetical protein FLJ13840 [Homo sapiens], mRNA | hypothetical protein FLJ13840 [Homo | Homo sapiens hypothetical protein FLJ13840 |
| | sequence/cds = (643, 2232) /gb = NM_024746 /gi = 21362001 | sapiens] | (FLJ13840), mRNA |
| | /ug = Hs.123515/len = 2514 | | |
| SGT20N4_E08 | | | |
| SGT20N4_G04 | | | |
| SGT20O1_E03 | ubiquitin specific protease 8 [Homo sapiens], mRNA | hypothetical protein [Homo sapiens] | Homo sapiens ubiquitin specific protease 8 |
| | sequence/cds = (317, 3673) /gb = NM_005154 /gi = 4827053 | | (USP8), mRNA |
| | /ug = Hs.152818/len = 4359 | | |
| SGT20O3_F12 | sirtuin 2, isoform 1; SIR2 (silent mating type information | SIR2L2 [Mus musculus] | Mus musculus sirtuin 2 (silent mating type |
| | regulation2, S. cerevisiae, homolog)-like; sirtuin (silent mating type | | information regulation 2, homolog) 2 (S. cerevisiae) |
| | information regulation 2, S. cerevisiae, homolog) 2; silencing | | (Sirt2), mRNA |
| | information regulator 2-like; SIR2 (silent mating type inform> | | |
| | /cds = (200, 1369) /gb = NM_012237/gi = 13775599 /ug = Hs.375214 | | |
| | /len = 1963 | | |
| SGT20O4_A02 | suppressor of Ty 6 homolog (S. cerevisiae); suppressor of | similar to suppressor of Ty 6 homolog (S. cerevisiae) | Homo sapiens suppressor of Ty 6 homolog (S. cerevisiae) |
| | Ty (S. cerevisiae) 6 homolog [Homo sapiens], mRNA | [Mus musculus] | (SUPT6H), mRNA |
| | sequence/cds = (1164, 5975) /gb = NM_003170 /gi = 11321572 | | |
| | /ug = Hs.12303/len = 6603 | | |
| SGT20O4_G04 | S-adenosylhomocysteine hydrolase; adenosylhomocysteinase | adenosylhomocysteinase [Streptomyces | Mus musculus S-adenosylhomocysteine |
| | [Homo sapiens], mRNA sequence /cds = (47, 1345) | coelicolor A3(2)] | hydrolase (Ahcy), mRNA |
| | /gb = NM_000687/gi = 9951914 /ug = Hs.172673 /len = 2110 | | |
| SGT20O5_D01 | solute carrier family 3 (activators of dibasic and neutral amino | blood-brain barrier large neutral amino acid | Homo sapiens solute carrier family 3 |
| | acid transport), member 2; 4F2; 4T2HC; Antigen identified | transporter heavychain 4F2 [Oryctolagus | (activators of dibasic and neutral amino acid |
| | bymonoclonal antibodies 4F2, TRA1.10, TROP4, and; | cuniculus] | transport), member 2 (SLC3A2), mRNA |
| | antigenidentified by monoclonal antibodies 4F2, TRA1.10, | | |
| | TROP4, and T43 [Homo> /cds = (480, 2069) | | |
| | /gb = NM_002394/gi = 21361343 /ug = Hs.79748 /len = 2188 | | |
| SGT20P1_B06 | | sv8-MUC4 apomucin [Homo sapiens] | |
| SGT20P3_C08 | AGENCOURT_8745191 Lupski_sciatic_nerve Homo sapiens | Early lactation protein | Macropus eugenii mRNA for early lactation |
| | cDNA cloneIMAGE: 6205346 5′, mRNA sequence | | protein (ELP) |
| | /clone = IMAGE: 6205346/clone_end = 5′ /gb = BQ942584 | | |
| | /gi = 22358062 /ug = Hs.401236/len = 895 | | |
| SGT20P3_C09 | | | |
| SGT20P4_E05 | | | |
| SGT20P5_C11 | | | |
| SGT20Q3_B11 | | | |
| SGT20Q3_F06 | | | |
| SGT20Q3_H03 | Homo sapiens solute carrier family 7, (cationic amino | solute carrier family 7, (cationic amino acid | Rattus norvegicus solute carrier family 7, |
| | acid transporter, y+ system) member 10 (SLC7A10), | transporter, y+system) member 10 [Rattus | (cationic amino acid transporter, y+ system) |
| | mRNA/cds = (99, 1670) /gb = NM_019849 /gi = 9790234 | norvegicus] | member 10 (Slc7a10), mRNA |
| | /ug = Hs.58679/len = 1918 | | |
| SGT20Q4_A02 | KIAA1541 protein [Homo sapiens], mRNA sequence | Similar to DNA segment, Chr 7, ERATO Doi | Homo sapiens mRNA for KIAA1541 protein, |
| | /cds = (908, 2341)/gb = AB040974 /gi = 7959348 /ug = Hs.380372 | 753, expressed [Xenopus laevis] | partial cds |
| | /len = 6206 | | |
| SGT20Q4_F08 | hypothetical protein MGC31963 [Homo sapiens], mRNA | kidney predominant protein NCU-G1 [Mus | Mus musculus, RIKEN cDNA 0610031J06 |
| | sequence/cds = (13, 1233) /gb = NM_144580 /gi = 24307870 | musculus] | gene, clone MGC: 27637IMAGE: 4507218, |
| | /ug = Hs.293984/len = 1603 | | mRNA, complete cds |
| SGT20Q4_G04 | | | |
| SGT20Q4_G09 | | | |
| SGT20Q4_H09 | KIAA1668 protein [Homo sapiens], mRNA sequence | hypothetical protein [Homo sapiens] | Mus musculus similar to hypothetical protein |
| | /cds = (0, 2376)/gb = AB051455 /gi = 13359208 /ug = Hs.8535 | | [Homo sapiens](LOC278699), mRNA |
| | /len = 5779 | | |
| SGT20Q5B_A04 | splicing factor 1; zinc finger protein 162 [Homo sapiens], | zinc finger protein 162 [Mus musculus] | Homo sapiens clone B4 transcription factor |
| | mRNA sequence /cds = (382, 2253) /gb = NM_004630 | | ZFM1 mRNA, complete cds |
| | /gi = 4759339/ug = Hs.180677 /len = 3131 | | |
| SGT20Q5B_D03 | | | |
| SGT20R1_A02 | | GM2 activator protein | Mus musculus GM2 ganglioside activator |
| | | | protein (Gm2a), mRNA |
| SGT20R1_B04 | hypothetical protein FLJ23024 [Homo sapiens], mRNA | unnamed protein product [Mus musculus] | Homo sapiens hypothetical protein FLJ23024 |
| | sequence/cds = (7, 846) /gb = NM_024936 /gi = 13376409 | | (FLJ23024), mRNA |
| | /ug = Hs.278945/len = 2083 | | |
| SGT20R2_E12 | | Chain B, Human Zinc-Alpha-2-Glycoprotein | |
| SGT20R2_G07 | Homo sapiens TGFB-induced factor (TALE family homeobox) | TG-interacting factor isoform b; homeobox | Homo sapiens TGFB-induced factor (TALE |
| | (TGIF), mRNA/cds = (303, 1508) /gb = NM_170695 /gi = 24850134 | protein TGIF; 5′-TG-3′interacting factor; TALE | family homeobox) (TGIF), mRNA |
| | /ug = Hs.90077/len = 1992 | homeobox TG-interacting factor; transforming | |
| | | growth factor-beta-induced factor | |
| | | [Homo sapiens] | |
| SGT20R3_B03 | | | Homo sapiens 12q BAC RP11-489P6 |
| | | | (Roswell Park Cancer Institute Human BAC |
| | | | Library) complete sequence |
| SGT20R3_C12 | hypothetical protein FLJ20487 [Homo sapiens], mRNA | hypothetical protein FLJ20487 [Homo | Homo sapiens hypothetical protein FLJ20487 |
| | sequence/cds = (22, 522) /gb = NM_017841 /gi = 8923449 | sapiens] | (FLJ20487), mRNA |
| | /ug = Hs.313247/len = 1250 | | |
| SGT20R3_D04 | | | |
| SGT20R3_H03 | hypothetical protein FLJ23342 [Homo sapiens], mRNA | hypothetical protein [Homo sapiens] | Homo sapiens mRNA; cDNA DKFZp667A213 |
| | sequence/cds = (23, 1546) /gb = NM_024631 /gi = 13375859 | | (from clone DKFZp667A213) |
| | /ug = Hs.38592/len = 2253 | | |
| SGT20R3_H09 | | | |
| SGT20S5_E08 | | | |
| SGT20T3_G12 | alkaline phosphatase precursor (AA −17 to 507) [Homo sapiens], | tissue non-specific alkaline phosphatase | Felis catus alkaline phosphatase (alpl) mRNA, |
| | mRNA sequence /cds = (400, 1974) /gb = X14174 | [Canis familiaris] | complete cds |
| | /gi = 28737/ug = Hs.381706 /len = 2339 | | |
| SGT20U1_A04 | transgelin; smooth muscle protein 22-alpha; 22 kDa actin- | Transgelin (Smooth muscle protein 22-alpha) | Homo sapiens transgelin (TAGLN), mRNA |
| | binding protein; SM22-alpha [Homo sapiens], mRNA | (SM22-alpha) (WS3-10) (22 kDa actin-binding | |
| | sequence/cds = (75, 680) /gb = NM_003186 /gi = 12621918 | protein) | |
| | /ug = Hs.433399/len = 1085 | | |
| SGT20U1_C08 | FLJ00071 protein [Homo sapiens], mRNA sequence | unnamed protein product [Homo sapiens] | Homo sapiens, clone MGC: 8832 |
| | /cds = (3020, 3772)/gb = AK024478 /gi = 10440469 /ug = Hs.7049 | | IMAGE: 3869275, mRNA, complete cds |
| | /len = 4194 | | |
| SGT20U2_F07 | | | |
| SGT20U3_A09 | homeo box D9; homeobox protein Hox-D9; Hox-4.3, mouse, | Similar to homeo box D9 [Homo sapiens] | Mus musculus homeo box D9 (Hoxd9), mRNA |
| | homolog of [Homo sapiens], mRNA sequence | | |
| | /cds = (439, 1467)/gb = NM_014213 /gi = 23397673 /ug = Hs.236646 | | |
| | /len = 2089 | | |
| SGT20U3_A10 | ribophorin I [Homo sapiens], mRNA sequence | ribophorin I [Sus scrofa] | Sus scrofa mRNA for ribophorin I |
| | /cds = (137, 1960)/gb = NM_002950 /gi = 4506674 /ug = Hs.2280 | | |
| | /len = 2397 | | |
| SGT20U3_B05 | | | |
| SGT20U3_C05 | translocase of inner mitochondrial membrane 8 homolog | translocase of inner mitochondrial membrane | Mus musculus translocase of inner |
| | A; deafness/dystonia peptide; translocase of innermitochondrial | 8 homolog A; deafness/dystonia peptide; | mitochondrial membrane 8 homologa (yeast) |
| | membrane 8 (yeast) homolog A [Homo sapiens], mRNA sequence | translocase of innermitochondrial membrane 8 | (Timm8a), mRNA |
| | /cds = (35, 328) /gb = NM_004085/gi = 6138974 /ug = Hs.125565 | (yeast) homolog A [Homo sapiens] | |
| | /len = 1168 | | |
| SGT20U3_D09 | hypothetical protein LOC51234 [Homo sapiens], mRNA | RIKEN cDNA 2610318K02 [Mus musculus] | Mus musculus RIKEN cDNA 2610318K02 |
| | sequence/cds = (71, 622) /gb = NM_016454 /gi = 24475963 | | gene (2610318K02Rik), mRNA |
| | /ug = Hs.250905/len = 1013 | | |
| SGT20U3_F03 | | | |
| SGT20U4_B08 | | similar to capicua protein; capicua [Mus | Homo sapiens chromosome 19 clone CTC- |
| | | musculus] [Rattus norvegicus] | 565M22, complete sequence |
| SGT20U4_H06 | | | |
| SGT20U5_D06 | | | |
| SGT20U5_E09 | | | Plasmodium falciparum 3D7 chromosome 12 |
| | | | section 6 of 9 of the complete sequence |
| SGT20V2_D09 | FLJ00006 protein [Homo sapiens], mRNA sequence | RIKEN cDNA 1810012I01 [Mus musculus] | Homo sapiens hypothetical protein |
| | /cds = (146, 1351)/gb = AK000006 /gi = 7209312 /ug = Hs.22129 | | DJ1042K10.2 (DJ1042K10.2), mRNA |
| | /len = 4219 | | |
| SGT20V2_E09 | | | Human chromosome 14 DNA sequence BAC |
| | | | R-431H16 of library RPCI-11 |
| | | | from chromosome 14 of Homo sapiens |
| | | | (Human), complete sequence |
| SGT20V2_H08 | | TRICHOSURIN PRECURSOR | Trichosurus vulpecula lipocalin trichosurin |
| | | | mRNA, complete cds |
| SGT20V4_A09 | hypothetical protein FLJ23342 [Homo sapiens], mRNA | similar to cDNA sequence BC024479 [Mus | Homo sapiens mRNA; cDNA DKFZp667A213 |
| | sequence/cds = (23, 1546) /gb = NM_024631 /gi = 13375859 | musculus] [Rattus norvegicus] | (from clone DKFZp667A213) |
| | /ug = Hs.38592/len = 2253 | | |
| SGT20V4_D01 | succinate dehydrogenase complex, subunit B, iron sulfur (lp); iron- | unnamed protein product [Mus musculus] | Mus musculus, RIKEN cDNA 0710008N11 |
| | sulfur subunit [Homo sapiens], mRNA sequence/cds = (134, 976) | | gene, clone MGC: 19177IMAGE: 4225025, |
| | /gb = NM_003000 /gi = 9257241 /ug = Hs.64/len = 1100 | | mRNA, complete cds |
| SGT20V4_F10 | | | |
| SGT20V4_G10 | | | Homo sapiens chromosome 19 clone CTD- |
| | | | 3131K8, complete sequence |
| SGT20V4_H06 | hypothetical protein MGC13016 [Homo sapiens], mRNA | unnamed protein product [Mus musculus] | Homo sapiens hypothetical protein MGC13016 |
| | sequence/cds = (38, 745) /gb = NM_032343 /gi = 14150133 | | (MGC13016), mRNA |
| | /ug = Hs.84120 /len = 984 | | |
| SGT20V5_A09 | Homo sapiens cDNA FLJ10946 fis, clone PLACE1000005, mRNA | unnamed protein product [Mus musculus] | Ictalurid herpes virus 1 (channel catfish virus |
| | sequence/gb = AK001808 /gi = 7023310 /ug = Hs.296544 /len = 1753 | | (CCV)), strain aubum 1, complete genome |
| SGT20V5_D11 | | | Rattus norvegicus Flap structure-specific |
| | | | endonuclease 1 (Fen1), mRNA |
| SGT20V5_H02 | | | |
| SGT20W5_A12 | selenoprotein SelM [Homo sapiens], mRNA sequence | Selenoprotein M precursor (SelM protein) | Homo sapiens, clone IMAGE: 3890282, mRNA |
| | /cds = (89, 526)/gb = NM_080430 /gi = 17975596 /ug = Hs.55940 | | |
| | /len = 718 | | |
| SGT20x1_E03 | chaperonin containing TCP1, subunit 3 (gamma); TCP1 (t- | similar to chaperonin subunit 3 (gamma) [Mus | Homo sapiens chaperonin containing TCP1, |
| | complex-1) ring complex, polypeptide 5 [Homo sapiens], mRNA | musculus] [Rattus norvegicus] | subunit 3 (gamma) (CCT3), mRNA |
| | sequence/cds = (0, 1634) /gb = NM_005998 /gi = 5174726 | | |
| | /ug = Hs.1708/len = 1901 | | |
| SGT20x1_C10 | hypothetical protein [Homo sapiens], mRNA sequence | | Human DNA sequence from clone RP5- |
| | /cds = (412, 1617)/gb = AL833978 /gi = 21739573 /ug = Hs.142442 | | 1102M4 on chromosome 1, |
| | /len = 3749 | | complete sequence |

TABLE 4
Group 3 ESTs

| | | Non-redundant protein sequence | |
|---|---|---|---|
| EST clone ID | Unigene match | database match | GenBank match |
| SGT20V5_A04 | | | Homo sapiens chromosome 17, clone RP11- |
| | | | 283C24, complete sequence |
| SGT20V2_D11 | | | |
| SGT20U3_C04 | | | |
| SGT20U3_B07 | CTL2 gene [Homo sapiens], mRNA sequence | unnamed protein product [Mus | Homo sapiens, clone IMAGE: 3848854, |
| | /cds = (0, 2120) /gb = NM_020428/gi = 9966908 | musculus] | mRNA |
| | /ug = Hs.105509 /len = 2121 | | |
| SGT20P1_B04 | | | Homo sapiens 3 BAC RP11-59J16 (Roswell |
| | | | Park Cancer Institute Human BAC Library) |
| | | | complete sequence |
| SGT20O5_E05 | | | |
| SGT20O2_A10 | | | |
| SGT20J6_B06 | | | Mus musculus Strain C57BL6/J Chromosome |
| | | | 11 BAC, RP23-193K14, complete sequence |
| SGT20I6_B01 | | | |
| SGT20I1_A12 | | | Homo sapiens chromosome 16 clone RP11- |
| | | | 107C10, complete sequence |
| SGT20F4_E05 | | | |
| SGT20F1_G12 | | | |

TABLE 5
Group 4 ESTs

| EST clone ID | Unigene match | Non-redundant protein sequence database match | GenBank match |
|---|---|---|---|
| SGT20A1_G07 | Unknown (protein for IMAGE: 4544931) [Homo sapiens] | Homo sapiens cDNA: FLJ22947 fis, clone KAT09234, mRNA | Homo sapiens cDNA: FLJ22947 fis, clone KAT09234 |
| | | sequence/gb = AK026600 /gi = 10439488 /ug = Hs.389624 | |
| | | /len = 861 | |
| SGT20B1_F04 | hypothetical protein XP_238162 [Rattus | protein tyrosine phosphatase, receptor type, f polypeptide | Homo sapiens protein tyrosine phosphatase, receptor |
| | norvegicus] | (PTPRF), interacting protein (liprin), alpha 1 [Homo | type, fpolypeptide (PTPRF), interacting protein (liprin), |
| | | sapiens], mRNA sequence /cds = (229, 3837) /gb = NM_003626 | alpha1 (PPFIA1), mRNA |
| | | /gi = 4505982/ug = Hs.183648 /len = 4313 | |
| SGT20C1_H12 | hypothetical protein MGC30714 [Mus | Homo sapiens cDNA FLJ20201 fis, clone COLF1210, mRNA | Mus musculus, Similar to transmembrane 4 |
| | musculus] | sequence/gb = AK000208 /gi = 7020141 /ug = Hs.27267 | superfamily member (tetraspan NET-7), clone |
| | | /len = 1720 | MGC: 30714 IMAGE: 3981492, mRNA, complete cds |
| SGT20C5_D01 | similar to hypothetical protein [Homo sapiens] | | |
| SGT20D2B_B02 | alpha 2 actin; alpha-cardiac actin [Homo | alpha 2 actin; alpha-cardiac actin [Homo sapiens], mRNA | Homo sapiens actin, alpha 2, smooth muscle, aorta |
| | sapiens] | sequence/cds = (47, 1180)/gb = NM_001613 /gi = 4501882 | (ACTA2), mRNA |
| | | /ug = Hs.195851/len = 1330 | |
| SGT20D3_B06 | ribosomal protein S6 [Mus musculus] | ribosomal protein S6; 40S ribosomal protein S6; | Rattus norvegicus ribosomal protein S6 (Rps6), |
| | | phosphoprotein NP33[Homo sapiens], mRNA sequence | mRNA |
| | | /cds = (42, 791)/gb = NM_001010 /gi = 17158043 /ug = Hs.380843 | |
| | | /len = 829 | |
| SGT20D3_G06 | hypothetical protein [Homo sapiens] | neuronal amiloride-sensitive cation channel 1; degenerin | Homo sapiens amiloride-sensitive cation channel 1, |
| | | [Homo sapiens], mRNA sequence /cds = (274, 1812) | neuronal(degenerin) (ACCN1), mRNA |
| | | /gb = NM_001094/gi = 21536347 /ug = Hs.6517 /len = 2747 | |
| SGT20D4_C07 | hypothetical protein MGC11770 [Mus | hypothetical protein MGC2744 [Homo sapiens], mRNA | Homo sapiens hypothetical protein MGC2744 |
| | musculus] | sequence/cds = (154, 1731) /gb = NM_025267 /gi = 13376885 | (MGC2744), mRNA |
| | | /ug = Hs.317403/len = 1844 | |
| SGT20D5_C07 | My004 protein [Homo sapiens] | HSPC042 protein [Homo sapiens], mRNA sequence | Homo sapiens HSPC042 protein (LOC51122), mRNA |
| | | /cds = (41, 388)/gb = NM_016094 /gi = 7705814 /ug = Hs.265540 | |
| | | /len = 949 | |
| SGT20E1B_C05 | hypothetical protein DKFZp434K1772.1 - | hyothetical protein [Homo sapiens], mRNA sequence | Mus musculus, Similar to hypothetical protein |
| | human (fragment) | /cds = (678, 1952)/gb = NM_019032 /gi = 24308134 | FLJ13710, clone MGC: 28749 IMAGE: 4482484, |
| | | /ug = Hs.96657 /len = 2704 | mRNA, complete cds |
| SGT20E2_E03 | similar to KIAA0560 protein [Homo sapiens] | KIAA0560 protein [Homo sapiens], mRNA sequence | Homo sapiens, clone IMAGE: 5109629, mRNA |
| | | /cds = (42, 4712)/gb = AB011132 /gi = 6635202 /ug = Hs.129952 | |
| | | /len = 5956 | |
| SGT20E2_G07 | hypothetical protein FLJ23751 [Homo sapiens] | hypothetical protein FLJ23751 [Homo sapiens], mRNA | Homo sapiens hypothetical protein FLJ23751 |
| | | sequence/cds = (120, 1562) /gb = NM_152282 /gi = 22748648 | (FLJ23751), mRNA |
| | | /ug = Hs.37443/len = 2994 | |
| SGT20G3_H05 | unnamed protein product [Mus musculus] | Sec23 (S. cerevisiae) homolog B; SEC23-like protein B; | Homo sapiens, clone IMAGE: 3456202, mRNA |
| | | protein transport protein SEC23B; SEC23-related protein | |
| | | B; transport protein Sec23 isoform B [Homo sapiens], | |
| | | mRNA sequence /cds = (112, 2415) /gb = NM_032986 | |
| | | /gi = 16905503/ug = Hs.173497 /len = 2814 | |
| SGT20G4_B10 | hypothetical protein XP_284029 [Mus | Homo sapiens cDNA FLJ38845 fis, clone MESAN2003709, | Homo sapiens chromosome 8, clone CTA-204B4, |
| | musculus] | mRNA sequence/gb = AK096164 /gi = 21755585 | complete sequence |
| | | /ug = Hs.356093 /len = 2289 | |
| SGT20H2_E10 | hypothetical protein FLJ14466 [Homo sapiens] | hypothetical protein FLJ14466 [Homo sapiens], mRNA | Homo sapiens hypothetical protein FLJ14466 |
| | | sequence/cds = (126, 842) /gb = NM_032790 /gi = 14249459 | (FLJ14466), mRNA |
| | | /ug = Hs.55148/len = 1877 | |
| SGT20I6_B05 | hypothetical protein DKFZp434D0127 [Homo | hypothetical protein DKFZp434D0127 [Homo sapiens], | Homo sapiens, hypothetical protein |
| | sapiens] | mRNA sequence/cds = (250, 2388) /gb = NM_032147 | DKFZp434D0127, clone |
| | | /gi = 14149816 /ug = Hs.154848/len = 2871 | MGC: 26981 IMAGE: 4825887, mRNA, complete cds |
| SGT20I6_H05 | unnamed protein product [Mus musculus] | hypothetical protein FLJ12572 [Homo sapiens], mRNA | Homo sapiens cDNA FLJ12572 fis, clone |
| | | sequence/cds = (439, 1620) /gb = NM_022905 /gi = 21362085 | NT2RM4000971 |
| | | /ug = Hs.139709/len = 3599 | |
| SGT20J1_C07 | hypothetical protein DKFZp586D0920.1 - | E1B-55 kDa-associated protein 5 isoform a [Homo sapiens], | Homo sapiens E1B-55 kDa-associated protein 5 (E1B- |
| | human (fragment) | mRNA sequence /cds = (173, 2743) /gb = NM_007040 | AP5), transcript variant 3, mRNA |
| | | /gi = 21536325/ug = Hs.155218 /len = 3872 | |
| SGT20K2_C10 | hypothetical protein XP_164784 [Mus | | |
| | musculus] | | |
| SGT20K3_B07 | hypothetical protein DKFZp564D0478 [Homo | hypothetical protein DKFZp564D0478 [Homo sapiens], | Homo sapiens hypothetical protein SB71 mRNA, |
| | sapiens] | mRNA sequence/cds = (27, 593) /gb = NM_032125 | complete cds |
| | | /gi = 14149778 /ug = Hs.321214/len = 1547 | |
| SGT20K4_C03 | similar to hypothetical protein MGC14327 | hypothetical protein MGC14327 [Homo sapiens], mRNA | Homo sapiens hypothetical protein MGC14327 |
| | [Homo sapiens] [Rattus norvegicus] | sequence/cds = (224, 634) /gb = NM_053045 /gi = 16596685 | (MGC14327), mRNA |
| | | /ug = Hs.231029/len = 1576 | |
| SGT20K4_G06 | unnamed protein product [Mus musculus] | NPD002 protein [Homo sapiens], mRNA sequence | Mus musculus similar to NPD002 protein [Homo |
| | | /cds = (88, 1953)/gb = NM_014049 /gi = 21361496 /ug = Hs.7010 | sapiens] (LOC229211), mRNA |
| | | /len = 2494 | |
| SGT20M5_C08 | hypothetical protein LOC92922 [Homo sapiens] | hypothetical protein MGC13119 [Homo sapiens], mRNA | Homo sapiens hypothetical gene supported by |
| | | sequence/cds = (222, 1874) /gb = NM_033212 /gi = 15082249 | BC004307; BC008285(MGC10992), mRNA |
| | | /ug = Hs.129126/len = 2470 | |
| SGT20N1_G01 | unnamed protein product [Mus musculus] | ribosomal protein S24 isoform a; 40S ribosomal protein S24 | Homo sapiens ribosomal protein S24 (RPS24), |
| | | [Homo sapiens], mRNA sequence /cds = (37, 429) | transcript variant 1, mRNA |
| | | /gb = NM_033022/gi = 14916500 /ug = Hs.180450 /len = 537 | |
| SGT20N4_G07 | ribosomal protein S3 [Mus musculus] | myo-inositol 1-phosphate synthase A1 [Homo sapiens], | Homo sapiens, ribosomal protein S3, clone |
| | | mRNA sequence/cds = (48, 1724) /gb = BC017189 | MGC: 32779 IMAGE: 4665438, mRNA, complete cds |
| | | /gi = 16877928 /ug = Hs.381118/len = 2760 | |
| SGT20Q5B_G02 | Similar to hypothetical protein dJ37E16.5 | hypothetical protein dJ37E16.5 [Homo sapiens], mRNA | Homo sapiens hypothetical protein dJ37E16.5 |
| | [Homo sapiens] | sequence/cds = (61, 951) /gb = NM_020315 /gi = 19923561 | (DJ37E16.5), mRNA |
| | | /ug = Hs.5790/len = 2053 | |
| SGT20R2_B09 | similar to hypothetical protein RP1-317E23 | hypothetical protein RP1-317E23 [Homo sapiens], mRNA | Homo sapiens hypothetical protein RP1-317E23 |
| | [Homo sapiens] | sequence/cds = (310, 1188) /gb = NM_019557 /gi = 24475811 | (LOC56181), mRNA |
| | | /ug = Hs.323396/len = 2119 | |
| SGT20T3_G11 | Unknown (protein for MGC: 32686) [Homo | Unknown (protein for MGC: 32686) [Homo sapiens], mRNA | Homo sapiens, clone MGC: 32686 IMAGE: 4051739, |
| | sapiens] | sequence/cds = (75, 491) /gb = BC029430 /gi = 20810228 | mRNA, complete cds |
| | | /ug = Hs.44205/len = 824 | |
| SGT20T4_D12 | similar to hypothetical protein MGC4266 [Homo | | Homo sapiens cDNA FLJ90699 fis, clone |
| | sapiens] [Rattus norvegicus] | | PLACE1007040 |
| SGT20T5_F01 | unnamed protein product [Mus musculus] | osteoblast specific factor 2 (fasciclin I-like) [Homo sapiens], | Homo sapiens osteoblast specific factor 2 (fasciclin I- |
| | | mRNA sequence /cds = (11, 2521) /gb = NM_006475 | like) (OSF-2), mRNA |
| | | /gi = 5453833 /ug = Hs.136348 /len = 3213 | |
| SGT20U1_G06 | N-myc downstream-regulated gene 2 [Rattus | Homo sapiens, clone IMAGE: 4156252, mRNA, mRNA | Homo sapiens NDRG family member 2 (NDRG2), |
| | norvegicus] | sequence /gb = BC013209/gi = 15301454 /ug = Hs.400790 | mRNA |
| | | /len = 2731 | |
| SGT20U5_E01 | unnamed protein product [Mus musculus] | Similar to hypothetical protein FLJ22405 [Homo sapiens], | Homo sapiens clone pp8153 unknown mRNA |
| | | mRNA sequence /cds = (63, 2015) /gb = BC035690 | |
| | | /gi = 23274205/ug = Hs.406601 /len = 2500 | |

TABLE 6
Group 5 ESTs

| | | Non-redundant protein sequence database | |
|---|---|---|---|
| EST clone ID | Unigene match | match | GenBank match |
| SGT20B1_C12 | Homo sapiens mRNA; cDNA DKFZp666J217 (from | hypothetical protein DKFZp566N034 [Homo | Homo sapiens hypothetical protein |
| | clone DKFZp666J217), mRNA sequence /gb = AL833765 | sapiens] | DKFZp566N034 (DKFZP566N034), mRNA |
| | /gi = 21734415 /ug = Hs.331633/len = 5097 | | |
| SGT20C3_G04 | hypothetical protein IMAGE3455200 [Homo sapiens], | similar to hypothetical protein IMAGE3455200 | Homo sapiens, clone IMAGE: 3455200, mRNA |
| | mRNA sequence/cds = (47, 538) /gb = NM_024006 | [Homo sapiens] [Rattus norvegicus] | |
| | /gi = 13124769 /ug = Hs.324844/len = 871 | | |
| SGT20D3_H05 | | hypothetical protein FLJ12089 [Homo sapiens] | |
| SGT20E2_D10 | unknown [Homo sapiens], mRNA sequence | unknown [Homo sapiens] | Mus musculus prion protein interacting protein 1 |
| | /cds = (0, 1195) /gb = AF007157/gi = 2852639 | | (Pmpip1), mRNA |
| | /ug = Hs.151032 /len = 1710 | | |
| SGT20H3_B08 | accessory protein BAP31 [Homo sapiens], mRNA | similar to B-cell receptor-associated protein 31 | Homo sapiens accessory protein BAP31 |
| | sequence/cds = (136, 876) /gb = NM_005745 | [Mus musculus] [Rattus norvegicus] | (DXS1357E), mRNA |
| | /gi = 10047078 /ug = Hs.291904/len = 1314 | | |
| SGT20I6_D09 | KIAA0710 gene product [Homo sapiens], mRNA | 1200014O24Rik protein [Mus musculus] | Homo sapiens, KIAA0710 gene product, clone |
| | sequence /cds = (203, 3550)/gb = NM_014871 | | MGC: 1971 IMAGE: 3357890, mRNA, complete |
| | /gi = 7662257 /ug = Hs.273397 /len = 4607 | | cds |
| SGT20J6_C08 | apoptosis related protein APR-3; p18 protein [Homo | Unknown (protein for MGC: 13322) [Homo | Homo sapiens HSPC013 mRNA, complete cds |
| | sapiens], mRNA sequence /cds = (335, 850) | sapiens] | |
| | /gb = NM_016085 /gi = 18105011/ug = Hs.9527 /len = 1086 | | |
| SGT20J6_F11 | hypothetical protein CAB56184 [Homo sapiens], mRNA | hypothetical protein CAB56184 [Homo sapiens] | Mus musculus similar to hypothetical protein |
| | sequence/cds = (0, 917) /gb = NM_032520 /gi = 14249737 | | CAB56184 [Homo sapiens] (LOC214505), mRNA |
| | /ug = Hs.241575/len = 918 | | |
| SGT20K3_B06 | FLJ00196 protein [Homo sapiens], mRNA sequence | Lcn7 protein [Mus musculus] | Mus musculus, clone MGC: 11828 |
| | /cds = (1839, 2693)/gb = AK074124 /gi = 18676595 | | IMAGE: 3596560, mRNA, complete cds |
| | /ug = Hs.173508 /len = 4761 | | |
| SGT20L4_A12 | sterol carrier protein 2 [Homo sapiens], mRNA | Nonspecific lipid-transfer protein, mitochondrial precursor (NSL-TP) | Oryctolagus cuniculus sterol carrier protein X |
| | sequence /cds = (21, 1664)/gb = NM_002979 | (Sterol carrier protein 2) | (SCP2) mRNA, complete cds |
| | /gi = 19923232 /ug = Hs.75760 /len = 2572 | (SCP-2) (Sterol carrier protein X) (SCP-X) | |
| | | (SCPX) | |
| SGT20L4_H04 | 602268464F1 NIH_MGC_81 Homo sapiens cDNA clone | Unknown (protein for MGC: 64538) [Xenopus | Homo sapiens interferon induced |
| | IMAGE: 4356734 5′, mRNA sequence | laevis] | transmembrane protein 3 (1-8U) (IFITM3), mRNA |
| | /clone = IMAGE: 4356734 /clone_end = 5′/gb = BF965170 | | |
| | /gi = 12332385 /ug = Hs.433414 /len = 1549 | | |
| SGT20N3_F12 | presenilins associated rhomboid-like protein; | presenilins associated rhomboid-like protein | Homo sapiens PRO2207 mRNA, complete cds |
| | hypotheilcal protein PRO2207 [Homo sapiens], mRNA | [Homo sapiens] | |
| | sequence /cds = (29, 1168)/gb = NM_018622 | | |
| | /gi = 20127651 /ug = Hs.13094 /len = 1393 | | |
| SGT20N4_E01 | stromal cell-derived factor 2 precursor [Homo sapiens], | similar to stromal cell-derived factor 2 precursor | Homo sapiens, Similar to stromal cell-derived |
| | mRNA sequence /cds = (39, 674) /gb = NM_006923 | [Homo sapiens] [Rattus norvegicus] | factor 2, clone MGC: 2977 IMAGE: 3140716, |
| | /gi = 14141194/ug = Hs.118684 /len = 1075 | | mRNA, complete cds |
| SGT20O1_D01 | nucleotide binding protein 2 (MinD homolog, E. coli); | nucleotide binding protein 2 [Mus musculus] | Mus musculus, Similar to nucleotide binding |
| | nucleotide binding protein 2 (E. coli MinD like) [Homo | | protein 2, clone MGC: 13715 IMAGE: 4038123, |
| | sapiens], mRNA sequence /cds = (63, 878) | | mRNA, complete cds |
| | /gb = NM_012225 /gi = 6912539/ug = Hs.256549 /len = 1351 | | |
| SGT20O5_G11 | Homo sapiens cDNA FLJ32555 fis, clone | Unknown (protein for IMAGE: 6879877) | Mus musculus, signal sequence receptor, delta, |
| | SPLEN1000116, moderately similar to TRANSLOCON- | [Xenopus laevis] | clone MGC: 6004 IMAGE: 3481948, mRNA, |
| | ASSOCIATED PROTEIN, DELTA | | complete cds |
| | SUBUNIT PRECURSOR, mRNA sequence | | |
| | /gb = AK057117 /gi = 16552704/ug = Hs.102135 /len = 2481 | | |
| SGT20P2_B04 | | Prostatic spermine-binding protein precursor | |
| | | (SBP) | |
| SGT20Q4_F02 | Homo sapiens cDNA FLJ37835 fis, clone | AES-1 protein-human (fragment) | Homo sapiens amino-terminal enhancer of split |
| | BRSSN2010110, weakly similar toGRG PROTEIN, | | (AES), mRNA |
| | mRNA sequence /gb = AK095154 | | |
| | /gi = 21754354/ug = Hs.375592 /len = 3276 | | |
| SGT20Q6_A08 | | ZW10 interactor (ZW10 interacting protein-1) | |
| | | (Zwint-1) | |
| SGT20Q6_B07 | SON DNA-binding protein isoform E; NRE-binding | unnamed protein product [Mus musculus] | Mus musculus Son cell proliferation protein |
| | protein; chromosome 21 open reading frame 50; SON | | (Son), mRNA |
| | protein; negative regulatory element-binding protein; Bax | | |
| | antagonist selected in Saccharomyces 1 [Homo | | |
| | sapiens], mRNA sequence/cds = (49, 6375) | | |
| | /gb = NM_058183 /gi = 21040317 /ug = Hs.92909/len = 8482 | | |
| SGT20Q6_E03 | hypothetical protein MGC32124 [Homo sapiens], mRNA | hypothetical protein MGC32124 [Homo sapiens] | Homo sapiens hypothetical protein MGC32124 |
| | sequence/cds = (40, 834) /gb = NM_144611 /gi = 21389420 | | (MGC32124), mRNA |
| | /ug = Hs.284163/len = 1370 | | |
| SGT20Q6_G05 | endothelial PAS domain protein 1 [Homo sapiens], | endothelial PAS domain protein 1 [Bos taurus] | Bos taurus mRNA for endothelial PAS domain |
| | mRNA sequence/cds = (149, 2761) /gb = NM_001430 | | protein1/hypoxia-inducible factor-2 alpha, |
| | /gi = 4503576 /ug = Hs.374409/len = 2818 | | complete cds |
| SGT20R3_B11 | nudix (nucleoside diphosphate linked moiety X)-type | nudix (nucleoside diphosphate linked moiety X)- | Homo sapiens nudix (nucleoside diphosphate |
| | mofif 9; ADP-ribose pyrosphosphatase NUDT9 [Homo | type motif 9 [Mus musculus] | linked moiety X)-type motif 9 (NUDT9), mRNA |
| | sapiens], mRNA sequence /cds = (325, 1377) | | |
| | /gb = NM_024047 /gi = 20127621/ug = Hs.301789 | | |
| | /len = 1718 | | |
| SGT20R4_C09 | Homo sapiens mRNA; cDNA DKFZp686P07111 (from | jumonji domain containing 1; zinc finger protein; | Homo sapiens zinc finger protein (TSGA), mRNA |
| | clone DKFZp686P07111), mRNA sequence | testis-specific protein A [Homo sapiens] | |
| | /gb = AL832150 /gi = 21732694 /ug = Hs.321707/len = 6587 | | |
| SGT20S1_B03 | NICE-3 protein [Homo sapiens], mRNA sequence | Similar to DKFZP586G1722 protein [Homo | Homo sapiens, Similar to DKFZP586G1722 |
| | /cds = (210, 869)/gb = NM_015449 /gi = 14149687 | sapiens] | protein, clone MGC: 5332 IMAGE: 2901006, |
| | /ug = Hs.355906 /len = 1636 | | mRNA, complete cds |
| SGT20S5_F10 | | | |
| SGT20T3_B11 | | cysteine-rich protein 2; Cystein-rich intestinal | Rattus norvegicus cysteine rich protein 2 |
| | | protein [Homo sapiens] | (Csrp2), mRNA |
| SGT20T5_E09 | ras homolog gene family, member A; Ras homolog gene | ras homolog gene family, member A; Aplysia | Homo sapiens ras homolog gene family, |
| | family, memberA (oncogene RHO H12); Aplysia ras- | ras-related homolog 12; Rho12; RhoA; Ras | member A (ARHA), mRNA |
| | related homolog 12; Rho12; RhoA [Homo sapiens], | homolog gene family, member A (oncogene RHO | |
| | mRNA sequence /cds = (151, 732)/gb = NM_001664 | H12) [Homo sapiens] | |
| | /gi = 10835048 /ug = Hs.77273 /len = 1777 | | |
| SGT20U3_E10 | CGI-135 protein [Homo sapiens], mRNA sequence | Chain A, Solution Structure Of Rsgi Ruh-001, A | Mus musculus, RIKEN cDNA 2010003O14 |
| | /cds = (81, 539)/gb = NM_016068 /gi = 7705631 | Fis1p-Like And Cgi-135 Homologous Domain | gene, clone MGC: 18717 IMAGE: 4221162, |
| | /ug = Hs.423968 /len = 735 | From A Mouse Cdna | mRNA, complete cds |
| SGT20W5_E11 | RelA-associated inhibitor [Homo sapiens], mRNA | Unknown (protein for IMAGE: 4413052) [Homo | Mus musculus similar to RelA-associated |
| | sequence/cds = (943, 1998) /gb = NM_006663 | sapiens] | inhibitor [Homo sapiens](LOC243869), mRNA |
| | /gi = 5730000 /ug = Hs.324051/len = 2620 | | |

### Example 5

### Three Lactation-Associated Polynucleotide and Polypeptide Sequences

By way of exemplification, the following data for three lactation-associated sequences identified herein is illustrative of the results obtained for lactation-associated sequences in the present study. The three clones are designated SGT20R3_C12, SGT20R1_B04 and SGT20K1_B08 (each belonging to Group 2 as described in Example 4).

RNA, translated peptide sequence and leader sequence prediction of candidate
genes
SGT20R3_C12
CACGCAGCACGCACGCGCGCCCAGAGCCGCCTCTCCCACCTCCCCTCCGAGGCCTCTCGGGCTCGTCGGGGCCTGCGGGA
GGTCCCCGGATGTGGTGAGCAGACGGGCTTCCGGCCGGGCCTGAGCGGAAATGGCGGCGGCGGCGGCGGCGGCTGCAGCT
GCTCCCGCAGTTCGGCTTCTTGCCTTGTCCAGGCACACTCTTGTGTCTCCCTTTGTGGCTAGTTCACTGTTGAGACGATT
CTACCGAGGGGACAGCCCATCAGACTCTCAAAAGGATATGCTTGAAATCCCCTTACCCCCATGGGAAGAGCGAACAGATG
AACCCATTGAAACCAAGAGGGCTCGCCTGCTTTATGAGAGCAGAAAAAGAGGCATGCTGGAGAACTGCATCCTGCTCAGT
CTCTTTGCCAAGGAGAATCTACAGCAAATGACGGAGAGGCAGCTGAACCTCTACGACCGGCTAATCAATGAGCCCAGTAA
TGACTGGGATATCTACTACTGGGCGACAGAAGCAAAGCCAGCCCCCCAAGGTCTTGAAAACGATGTCATGGTGATGCTGA
GAGACTTTGCTAAGAACANAAAGAAAGAGCAGAGGTTGCGGGCCCCAGATCTCGAGTACCTCTTTGAGAAACCAGCCTGA
GCTCCATTCTGGCCTGACCCGCAGGCAGGGCCCTGCANGGACACAGTAGACCCCGGTCACCTGCTGCTTNCCACTACCAT
CCCAGAGCATGGTCTCACTCACGTCATGTCTCAGAAAAGGACTCCTTGTGTCT
peptide prediction
MAAAAAAAAAAPAVRLLALSRHTLVSPFVASSLLRRFYRGDSPSDSQKDMLEIPLPPWEERTDEPIETKRARLLYESRKR
GMLENCILLSLFAKENLQQMIERQLNLYDRLINEPSNDWDIYYWATEAKPAPKVFENDVMVMLRDFAKNXKKEQRLRAPD
LEYLFEKPA
localisation prediction: Signal Peptide
SGT20R1_B04
CAGGGAAAGTTTTCTTTGATAATTTCGTGGAAGATAATGTCTAGGCTCTTTTTTTTTTGATCATGGCTTTCTAGTGACAA
TTTATTGCATTGTAGGCCTCCTTGTCACCAGATTAAAAATTAACTGTTGCTTTTTTCATAGTTATTTAATAAAATGGCTT
TTCTTAATTTGCTTTAATTTATAACTTTTTATTGAAGTTTTTACATTTATTTGTTGATTTTAATAACAATGTATGTTCTT
TTATTTAAATAAATTCTTATGCTTACATTTTCAACTTTCTAGGTAGATTATGATAATCATGCACTTTTTAAATATGGAAA
AACAGGTAAAAAAAAATCTCCTGTGCGTATTTTCACCAATATTCCTCCCAGAAAAATAATTCTTCCAGCAGAAGAAGGAT
ACAGGTTTTGTACTGTGTGTCAGCGTTATGTTTCTTTAGAGAACCAGCACTGTGAGATCTGCAATTCATGTACGTCTAAG
GATGGCAGGAGGTGGAAGCATTGCCTTCTTTGCAAAAAATGTGTCAAGCCCTCTTGGATTCACTGCAGCATTTGCAATTA
CTGTGCCCTTCCAATCATTCATGTGCAGATGCTAAAGATGGTTGCTTTATATGTGGTGAAGTAGATCACANACGTAGTAT
GTGTCCTAATTTCTCTGCATCTAANNAGAGCTACANGGCTGTCAGGAGACAGAAGCCAAAAAAAAAGTAACCAGATTGAA
ATGGAGACCACTAAAGGACCATCTATGAATCATGCAG
peptide prediction
MAGGGSIAFFAKNVSSPLGFTAAFAITVPFQSFSADAKDGCFICGEVDHXVVCVLISLHLXRATXLSGDRSQKKSNQIEM
ETTKGPSMNHAX
localisation prediction: Mitochondrial Transit Peptide
SGT20K1_B08
TCTGGCCTTGCTAAACCTGGCCTGTATGATGATTATTACTTTCTTGCCATACACGTTTTCCTTAATGGCCTCCTTTCCTG
ATGTGCCTTTGGGTATTTTCCTGTTTTGCATTTGTGTCATTGCCATTGGCCTCAGTCAGGCAGCAATTGTGACCTATGGG
TTCCATTACCCATACTTACTGAATCGCCAGATCCGACAGTCAGAGAACAAGGCCTTCTACAAGCACCATATCTTAAATAT
TATACTCAGGGGGCCAGCCCTGTGCTTTTTTGCGGCCATCTTCTCCTTTTTCTTTTTTCCTGTGTCTTACCTCCTTCTTG
GCCTTGTCATCTTCCTCCCCTACATCAATAGATTCATCACGTGGTGCAGAGACAAACTTGTTGGTACCAAATCAGAAGAG
CAACCTCAGAGCTTAGAGTTTTTTACTTTTAATATCCATGAACCCCTAAGTAAGGAGCGAGTAGAAGCCTTCAGTGATGG
TGTGTATGCCATTGTAGCAACCCTCCTCATCCTGGACATTTGTGAGGATAATGTTCCTGATGCCAAAGAAGTTAAAGAAA
AATTTCATGGTGACCTTGTTGAAGCACTGAGAGAATATGGACCAAACTTCCTGCCCTATTTTGCGCTCCTTTGTAACCAT
TGGTCTCCTGTGGCTTGTCCACCACTCCCTCTTTCTTCATGTGAGAAAGACAACCCAGNTCATGGGCCTG
peptide prediction
SGLAKPGLYDDYYFLAITFSLMASFPDVPLGIFLFCICVIAIGLSQAAIVTYGFHYPYLLNRQIRQSENKAFYKHHILNI
ILRGPALCFFAAIFSFFFFPVSYLLLGLVIFLPYINRFITWCRDKLVGTKSEEQPQSLEFFTFNIHEPLSKERVEAFSDG
VYAIVATLLILDICEDNVPDAKEVKEKFHGDLVEALREYGPNFLPYFALLCNHWSPVACPPLPLSSCEKDNPXHGPX
localisation prediction: Other

Blast hits of 3 candidate genes

| EST Clone ID | Unigene | Non Redundant Protein | Genbank |
|---|---|---|---|
| SGT20K1_B08 | hypothetical protein MGC4618 [Homo | unnamed protein product [Mus | Mus musculus, RIKEN |
| | sapiens], mRNA | musculus] | cDNA 3010001K23 gene, |
| | sequence/cds = (107, 1621)/ | | clone |
| | gb = NM_032326/gi = 14150103/ | | MGC:8187IMAGE:3590497, |
| | ug = Hs.89072/len = 1818 | | mRNA, complete cds |
| SGT20R1_B04 | hypothetical protein FLJ23024 [Homo | unnamed protein product [Mus | Homo sapiens hypothetical |
| | sapiens], mRNA sequence/cds = (7, 846)/ | musculus] | protein FLJ23024 |
| | gb = NM_024936/gi = 13376409/ | | (FLJ23024), mRNA |
| | ug = Hs.278945/len = 2083 | | |
| SGT20R3_C12 | hypothetical protein FLJ20487 [Homo | hypothetical protein FLJ20487 | Homo sapiens hypothetical |
| | sapiens], mRNA | [Homo sapiens] | protein FLJ20487 |
| | sequence/cds = (22, 522)/ | | (FLJ20487), mRNA |
| | gb = NM_017841/gi = 8923449/ | | |
| | ug = Hs.313247/len = 1250 | | |

Normalised average intensities of microarray spots of candidate genes

| | day −21 | day −4 | day −1 | day 1 | day 5 | day 80 | day 130 | day 168 | day 213 | day 220 | day 260 |
|---|---|---|---|---|---|---|---|---|---|---|---|
| SGT20r3_C12 | 435 | 10120 | 7329 | 9560 | 9392 | 12296 | 48821 | 64342 | 55262 | 50417 | 75551 |
| SGT20r1_B04 | 175 | 2614 | 3029 | 1932 | 2509 | 4388 | 12595 | 13524 | 9253 | 16839 | 16585 |
| SGT20k1_B08 | 238 | 4112 | 4049 | 3256 | 3745 | 6041 | 19800 | 19738 | 18028 | 26733 | 21082 |

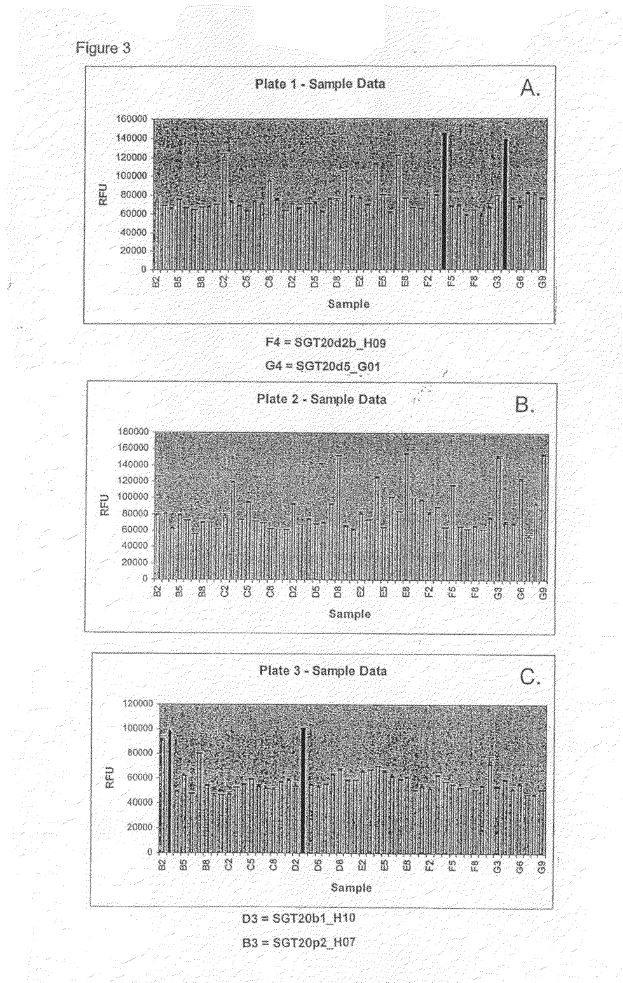

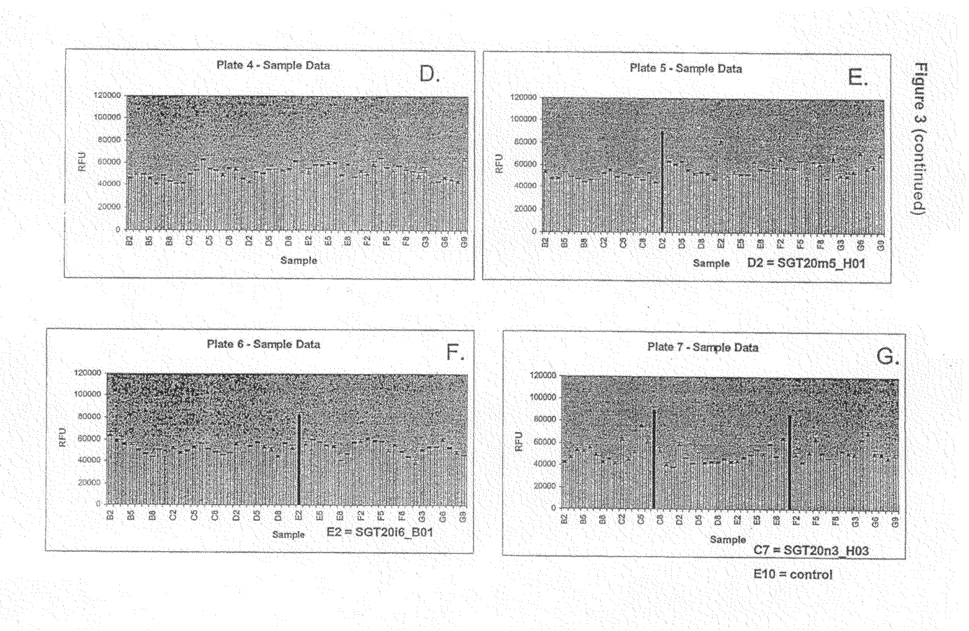

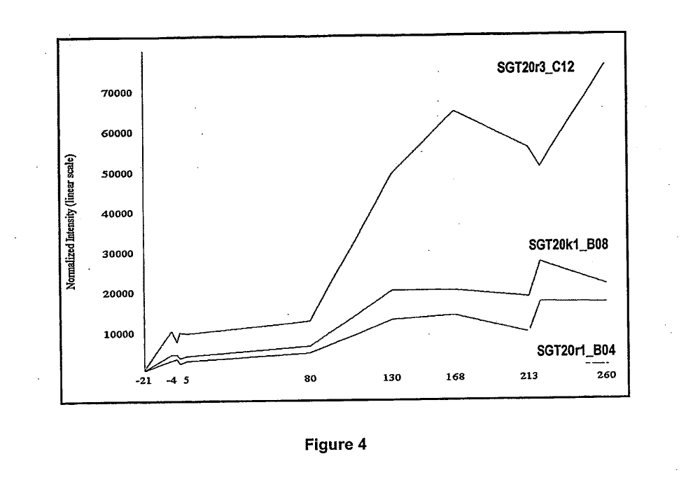

A graph of this data is shown in FIG. 4. This shows the normalized spot intensities for each EST from 21 days before parturition (day five pregnant) to day 260 of lactation. Each of SGT20R3_C12, SGT20R1_B04 and SGT20K1_B08 showed at least a 5-fold increase in expression across at least one phase change in lactation.

### Example 6

### Isolation of Secreted Polypeptides

Plasmids containing ESTs directionally cloned into the expression vector pCMV Sport 6.0 were transfected into the human kidney cell line HK293. A total of 1 ug of EST plasmid DNA and 10 ng of pEGFP-C1 plasmid was introduced into 70% confluent HK293 cells in 2 cm² wells containing 500 ul of opti-MEM-1 media. Transfection success was assessed by observing green fluorescence of cells by fluorescent microscopy. After 48 hours conditioned media containing the secreted peptide was collected and frozen at −20° C. The media containing the secreted polypeptides can then be used directly in a number of bioactivity assays, including those described below.

### Example 7

### Assays for Biological Activity of Secreted Polypeptides

Samples of the secreted polypeptides prepared according to Example 6 can be used in a variety of assays in screening for biological activity. The assays may be high-throughput screening assays.

In accordance with the best mode of performing the invention provided herein, specific examples of biological activity assays are outlined below. The following are to be construed as merely illustrative examples of assays and not as a limitation of the scope of the present invention in any way.

Typically samples of secreted polypeptides will be aliquoted into individual wells of a 96 or 384 well plate and stored prior to assaying either frozen or lyophilized.

### Example 7A

### Assay for Cell Growth-Promoting Activity

Extracellular signal-regulated protein kinase (ERK) is a common and central signal transduction pathway component of tyrosine kinase receptor. Activation of ERK is indicative of an extracellular proliferation signal and provides an index of a growth promoting agent.

Swiss 3T3 fibroblast cells were plated into 384 well plates, grown to confluence and starved overnight with serum-free medium. Cells were then treated for 10 minutes with the secreted polypeptide samples. Cells were then lysed and assayed for activation of ERK. Samples were assessed for changes in the activity of ERK. Activation of ERK by increasing concentrations of betacellulin was used a positive control in each case (data not shown).

The results of ERK activation assays are shown in FIG. 3 as RFU (relative fluorescence units) produced by each sample. A number of clones produced levels of ERK activation significantly above the mean, indicating a growth-promoting activity. Those of most significance are indicated by black bars in FIG. 3, with activation greater than or equal to 3 standard deviations above the mean.

### Example 7B

### Cell Viability Assay to Assess Anti-Apoptotic Effects

Vinblastine is a commonly used cytotoxic agent used in chemotherapy. It induces apoptosis in a wide variety of cell types. Caspase activation and DNA fragmentation are hallmarks of the apoptotic process.

Aliquots of the secreted polypeptide samples in 96 well plates can be pipetted onto HSC-2 oral epithelial cells and cells left for 24 hours. After this time, cells are treated with vinblastine to induce apoptosis. After 48 hours, cells are analyzed for survival using a vital dye. Internal controls for the activation of apoptosis may use 7×96 well plates of cells to assess all samples and controls. Cell survival measurements with this technique reflect the degree of apoptosis. If desired, other more direct assays of apoptosis, such as caspase activation or DNA fragmentation can be undertaken to verify the data obtained.

### Example 7C

### Cell Viability Assay to Assess Pro-Apoptotic Effects

Using the same method of assaying cell viability as indicated in Example 7B, the secreted polypeptide samples can be pipetted onto HSC-2 cells and the degree of cell viability 48 hours later assessed. Internal controls for induction of cell death via apoptosis as well as assay performance are typically also included on each plate.

### Example 7D

### Assay for Pro-Inflammatory Activity

p38 MAP kinase (MAPK) is also known as Mitogen-Activated Protein Kinase 14, MAP Kinase p38, p38 alpha, Stress Activated Protein Kinase 2A (SAPK2A), RK, MX12, CSBP1 and CSBP2. p38 is involved in a signaling system that controls cellular responses to cytokines and stress and p38 MAP Kinase is activated by a range of cellular stimuli including osmotic shock, lipopolysaccharides (LPS), inflammatory cytokines, UV light and growth factors.

RAW macrophage cells can be plated into 384 well plates, grown to confluence, starved for 3 hours with serum-reduced medium, and then treated for 30 minutes with the secreted polypeptide samples. Cells are then lysed and assayed for p38 mitogen-activated protein kinase (MAPK) activation. Internal controls for cell activation of p38 MAPK and assay performance are typically also included in unused wells.

### Example 7E

### Assay for Anti-Inflammatory Activity

RAW macrophage cells can be grown in 384 well plates, as described above, pre-treated with secreted polypeptide samples for 30 minutes. The cells are then treated with LPS (lipopolysaccharide) for 30 minutes to stimulate p38 MAPK. After this time, cells are lysed and assayed for p38 MAPK activation. Internal controls for cell activation of p38 MAPK and assay performance are typically also included in unused wells.

### Example 7F

### Assay for Increased Protein Secretion

### ³⁵S-Methionine Protein Synthesis Assay

Bovine mammary epithelial cells can be plated onto extracellular matrix in 96 well plates. After 5 days in culture, cells are incubated in methionine free medium for 1 h and then labeled with ³⁵S-methionine for a 4 h period. Cells are exposed to the expressed peptides during this time. Cell media is then collected and protein precipitated from the media. Cells are also harvested. Cell extracts and protein precipitated from the media are then counted using liquid scintillation counting. This enables both cellular and secreted protein synthesis to be determined relative to an appropriate control.

### Example 7G

### Antibacterial Assays

Bacteria can be cultured in the presence of the conditioned media, and the effects on growth and viability of the organisms assessed. Target organisms can include human pathogens such as Helicobacter pylori, which is the major cause of gastric ulcers and gastric cancer.

### Example 7H

### Induction of Trefoil Proteins

Trefoil proteins have been demonstrated to significantly accelerate gut repair after infection and injury. The intestinal epithelial cell line AGS can be transfected with a GFP reporter gene under the control of the trefoil gene promoter. Cells will be exposed to secreted proteins and promoter activity determined by GFP fluorescence.

### Example 7I

### Regulation of Cell Fate and Differentiation

A significant requirement for stem cell therapeutics and cloning is to manipulate pluripotency and differentiation in vitro. The OCT4 gene is a characterized marker for pluripotency.

Mouse embryonic stem cells will be cultured in the presence of the secreted peptides and cellular differentiation microscopically. Cell lines with the GFP reporter gene under the control of the OCT4 promoter will be exposed to secreted proteins and promoter activity determined by GFP fluorescence.

### Example 7J

### Regulation of Cell Fate and Differentiation

The morphology of mammary epithelium changes significantly as it moves from a non-milk secreting epithelium to a highly secretory epithelium. Polypeptides able to regulate the function and differentiation of the mammary gland can be screened by culturing primary mammary epithelium in the presence of the secreted polypeptides. Cells will be examined microscopically for gross morphological changes.

Secreted polypeptides with growth promoting activity (example 7A), pro and anti-apoptotic effects (Examples 7C and 7B respectively), able to influence the differentiation of mammary epithelium (present Example), or able to effect the level of protein secretion (Example 7F) may regulate mammary gland physiology and the duration and degree of milk production.

Polypeptides with antibacterial properties (Example 7G), or anti or pro inflammatory properties (Examples 7E or 7D respectively) potentially influence the susceptibility and degree of mastitis.

1.-24. (canceled)

25. A peptide comprising an amino acid sequence represented by SEQ ID NO: 370.

26. A peptide having at least 75% amino acid homology with the peptide according to claim 25.

27. A peptide having at least 85% amino acid homology with the peptide according to claim 25.

28. A peptide having at least 90% amino acid homology with the peptide according to claim 25.

29. A peptide having at least 95% amino acid homology with the peptide according to claim 25.

30. A peptide having at least 99% amino acid homology with the peptide according to claim 25.

31. A peptide comprising an amino acid sequence that only differs from SEQ ID NO: 370 in the conservative substitution of one or more amino acids.

32. A bovine homologue of a peptide comprising an amino acid sequence represented by SEQ ID NO: 370.

33. A host cell that contains the peptide according to claim 26.

34. A composition comprising a peptide according to claim 26 together with one or more pharmaceutically acceptable carriers, diluents or adjuvants.
